(19) United States
(12) Patent Application Publication
(10) Pub. No.: US 2005/0070542 A1
(43) Pub. Date: Mar. 31, 2005

(54) 5-Aryl-pyrazolo[4,3-d]pyrimidines, pyridines, and pyrazines and related compounds

(76) Inventors: Kevin J. Hodgetts, Kilingworth, CT (US); Stanly John, Basking Ridge, NJ (US); Neil Moorcroft, Bloomsbury, NJ (US); Greg Shutske, Pittstown, NJ (US); Bernd Kaiser, Wallingford, CT (US); Yasuchika Yamaguchi, Guilford, CT (US); Ping Ge, Durham, CT (US); Raymond F. Horvath, Guilford, CT (US)

Correspondence Address: EDWARDS & ANGELL, LLP, P.O. BOX 55874, BOSTON, MA, 02205, US

(21) Appl. No.: 10/933,700

(22) Filed: Sep. 3, 2004

Related U.S. Application Data
(60) Provisional application No. 60/500,033, filed on Sep. 3, 2003.

(51) Int. Cl. A61K 31/53; A61K 31/519; C07D 487/02

(52) U.S. Cl. 514/243; 514/261.1; 514/260.1; 514/262.1; 544/184; 544/262

(57) ABSTRACT

5-aryl-Pyrazolo[4,3-d]pyrimidines, 6-aryl-Pyrazolo[3,4-d]pyrimidines and related compounds, as well as other chemically related compounds, that act as selective modulators of CRF 1 receptors are provided. These compounds are useful in the treatment of a number of CNS and periphereal disorders, particularly stress, anxiety, depression, cardiovascular disorders, and eating disorders. Methods of treatment of such disorders and well as packaged pharmaceutical compositions are also provided. Compounds of the invention are also useful as probes for the localization of CRF receptors and as standards in assays for CRF receptor binding. Methods of using the compounds in receptor localization studies are given.

This application claims priority from U.S. Provisional Application Ser. No. 60/500,033 filed on Sep. 3, 2003.

### FIELD OF THE INVENTION

The present invention relates to novel 5-aryl-Pyrazolo[4,3-d]pyrimidines, 6-aryl-Pyrazolo[3,4-d]pyrimidines and related compounds that bind with high selectivity and/or high affinity to CRF receptors (Corticotropin Releasing Factor Receptors). This invention also relates to pharmaceutical compositions comprising such compounds and to the use of such compounds in treatment of psychiatric disorders and neurological diseases, including major depression, anxiety-related disorders, post-traumatic stress disorder, supranuclear palsy and feeding disorders, as well as treatment of immunological, cardiovascular or heart-related diseases and colonic hypersensitivity associated with psychopathological disturbance and stress. Additionally this invention relates to the use such compounds as probes for the localization of CRF receptors in cells and tissues. Preferred CRF receptors are CRF1 receptors.

### BACKGROUND OF THE INVENTION

Corticotropin releasing factor (CRF), a 41 amino acid peptide, is the primary physiological regulator of proopiomelanocortin (POMC) derived peptide secretion from the anterior pituitary gland. In addition to its endocrine role at the pituitary gland, immunohistochemical localization of CRF has demonstrated that the hormone has a broad extrahypothalamic distribution in the central nervous system and produces a wide spectrum of autonomic, electrophysiological and behavioral effects consistent with a neurotransmitter or neuromodulator role in brain. There is also evidence that CRF plays a significant role in integrating the response of the immune system to physiological, psychological, and immunological stressors.

Clinical data provide evidence that CRF has a role in psychiatric disorders and neurological diseases including depression, anxiety-related disorders and feeding disorders. A role for CRF has also been postulated in the etiology and pathophysiology of Alzheimer's disease, Parkinson's disease, Huntington's disease, progressive supranuclear palsy and amyotrophic lateral sclerosis as they relate to the dysfunction of CRF neurons in the central nervous system.

In affective disorder, or major depression, the concentration of CRF is significantly increased in the cerebral spinal fluid (CSF) of drug-free individuals. Furthermore, the density of CRF receptors is significantly decreased in the frontal cortex of suicide victims, consistent with a hypersecretion of CRF. In addition, there is a blunted adrenocorticotropin (ACTH) response to CRF (i.v. administered) observed in depressed patients. Preclinical studies in rats and non-human primates provide additional support for the hypothesis that hypersecretion of CRF may be involved in the symptoms seen in human depression. There is also preliminary evidence that tricyclic antidepressants can alter CRF levels and thus modulate the numbers of CRF receptors in brain.

CRF has also been implicated in the etiology of anxiety-related disorders. CRF produces anxiogenic effects in animals and interactions between benzodiazepine/non-benzodiazepine anxiolytics and CRF have been demonstrated in a variety of behavioral anxiety models. Preliminary studies using the putative CRF receptor antagonist α-helical ovine CRF (9-41) in a variety of behavioral paradigms demonstrate that the antagonist produces “anxiolytic-like” effects that are qualitatively similar to the benzodiazepines. Neurochemical, endocrine and receptor binding studies have all demonstrated interactions between CRF and benzodiazepine anxiolytics providing further evidence for the involvement of CRF in these disorders. Chlordiazepoxide attenuates the “anxiogenic” effects of CRF in both the conflict test and in the acoustic startle test in rats. The benzodiazepine receptor antagonist Ro 15-1788, which was without behavioral activity alone in the operant conflict test, reversed the effects of CRF in a dose-dependent manner, while the benzodiazepine inverse agonist FG 7142 enhanced the actions of CRF.

CRF has also been implicated in the pathogeneisis of certain immunological, cardiovascular or heart-related diseases such as hypertension, tachycardia and congestive heart failure, stroke and osteoporosis, as well as in premature birth, psychosocial dwarfism, stress-induced fever, ulcer, diarrhea, post-operative ileus and colonic hypersensitivity associated with psychopathological disturbance and stress.

The mechanisms and sites of action through which conventional anxiolytics and antidepressants produce their therapeutic effects remain to be fully elucidated. It has been hypothesized however, that they are involved in the suppression of CRF hypersecretion that is observed in these disorders. Of particular interest are that preliminary studies examining the effects of a CRF receptor antagonist peptide (a-helical CRF₉₋₄₁) in a variety of behavioral paradigms have demonstrated that the CRF antagonist produces “anxiolytic-like” effects qualitatively similar to the benzodiazepines.

### DESCRIPTION OF THE RELATED ART

Madronero et al. (An. R. Acad. Farm. 1988, 31, 1309-1314) described the preparation of a series of optionally substituted pyrazolo[3,4-d]pyrimidines of general formula: 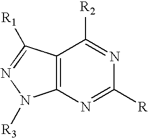
wherein:
R is phenyl or p-ClC₆H₄, R¹ is H, methyl, or phenyl; R₂ is alkyl, aryl, benzyl, alkylthio, or PhS; and R³ is methyl.
Breuer et al. (U.S. Pat. No. 3,732,225) disclosed as hypoglycemic agents pyrazolo[3,4-d]pyrimidines of formula: 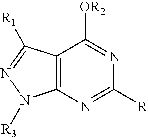
   wherein:
R is (un)substituted phenyl or cycloalkyl; R₁ is hydrogen, lower-alkyl, cycloalkyl, (un)substituted phenyl; R₂is H or lower-alkyl; R₃is lower-alkyl, cycloalkyl, phenyl or substituted phenyl.
Bacon et al. (WO 9628448) disclosed for the treatment of heart failure and/or hypertension 6-arylpyrazolo[3,4-d]pyrimidin-4-ones of formula: 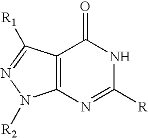
   wherein:
R is (un)substituted phenyl; R₁ is lower alkyl, phenyl-lower alkyl; R₂ is tBu or cyclopentyl.

Bunnage et al. (EP 995751) disclosed as cGMP PDE5 inhibitors for the treatment of sexual dysfunction pyrazolopyrimidinones of formula: 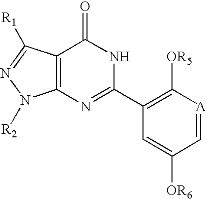
wherein:
A is CH or N; R₁ is defined the same as R₂ and is H, (un)substituted alkyl, or (un)substituted heterocycle; R₅ is H or (un)substituted alkyl; R₆ is SO₂NR¹²R¹³; NR¹²R¹³ is Het; Het is a 4-12 membered heterocyclic group containing at least one N atom and, optionally, one or more heteroatoms selected from N, S, and O.

Jonas et al., WO 0118004 has disclosed pyrazolo[4,3-d]pyrimidines of formula: 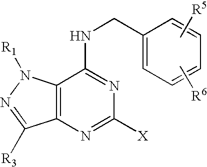
phospodiesterase V inhibitors for the treatment of cardiovascular disease and impotence, wherein
R⁵ and R⁶ may be H, A, OH, OA or halo; R⁵ and R⁶ are alkylene, OCH₂CH₂, CH₂OCH₂, OCH₂O, or OCH₂CH₂O; R¹ and R³ are H or A; X is R¹⁰-substituted R⁷, R⁸, or R⁹; R⁷ is alkylene or alkenylene; R⁸ is cycloalkyl or cycloalkylalkylene; R⁹ is phenyl, phenylCH₂; R¹⁰ is CO₂H, CO₂A, CONH₂, CONHA, CONA₂ or cyano; and A is alkyl.

DeWald et al., J. Med. Chem. 1988, 31(2), 454-461, describe the synthesis of substituted 3-methyl-1H-pyrazolo[4,3-d]pyrimidines of general formula: 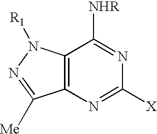
wherein:
R is alkyl or alkoxy; R¹ is alkyl; and X is H, alkyl, phenyl, or benzyl.

Ratajczyk et al., U.S. Pat. No. 3,939,161, disclosed compounds with anti-convulsant, sedative, anti-inflammatory, gastric anti-secretory and central nervous system activities, pyrazolopyrimidinones of general formula: 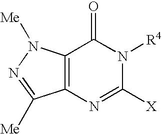
wherein:
R⁴ is H, methyl, phenyl, substituted phenyl; X is H, methyl, ClCH₂, morpholinomethyl, or piperidinomethyl.

### SUMMARY OF THE INVENTION

The invention provides novel compounds of Formula I (shown below), and pharmaceutical compositions comprising compounds of Formula I and at least one pharmaceutically acceptable carrier or excipient. Such compounds bind to cell surface receptors, preferably G-coupled protein receptors, especially CRF receptors (including CRF1 and CRF2 receptors) and most preferably CRF 1 receptors. Preferred compounds of the invention exhibit high affinity for CRF receptors, preferably CRF 1 receptors. Additionally, preferred compounds of the invention also exhibit high specificity for CRF receptors (i.e., they exhibit high selectivity compared to their binding to non-CRF receptors). Preferably they exhibit high specificity for CRF 1 receptors.

Thus, a broad embodiment of the invention is directed to compounds Formula I: 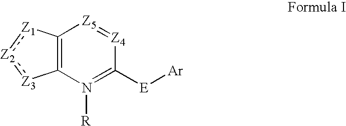
and the pharmaceutically acceptable salt thereof, wherein:
E is a single bond, O, S(O)ₘ, NR₁₀ or CR₁₀R₁₁;
Ar is chosen from:
   phenyl which is mono-, di-, or tri-substituted;
   1-naphthyl and 2-naphthyl, each of which is optionally mono-, di-, or tri-substituted; and
   optionally mono-, di-, or tri-substituted heteroaryl, said heteroaryl having from 1 to 3 rings, 5 to 7 ring members in each ring and, in at least one of said rings, from 1 to about 3 heteroatoms selected from the group consisting of N, O, and S;
   wherein in Ar, at least one of the positions ortho to the point of attachment of Ar shown in Formula I is substituted;
R is independently selected at each occurrence to be absent or oxygen;
the group: 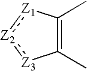
   represents a saturated, unsaturated or aromatic 5-membered ring system containing 2 or 3 nitrogen atoms, wherein:
Z₁ is CR₁, CR₁R₁′, or NR₁″;
Z₂ is nitrogen, or NR₂″,
Z₃ is CR₃, CR₃R₃′ nitrogen, NR₃″, oxygen, sulfur, sulfoxide or sulfone;
R¹ is chosen from halogen, hydroxy, cyano, amino, optionally substituted alkyl, optionally substituted alkenyl, optionally substituted alkynyl, optionally substituted alkoxy, optionally substituted mono or dialkylamino, optionally substituted heterocycloalkyl, optionally substituted (cycloalkyl)alkyl, optionally substituted (heterocycloalkyl)alkyl, optionally substituted alkylthio, optionally substituted alkylsulfinyl, optionally substituted alkylsulfonyl, optionally substituted mono- or dialkylcarboxamide, optionally substituted carbocyclic aryl, optionally substituted (aryl)cycloalkyl, optionally substituted (aryl)heterocycloalkyl, optionally substituted heteroaryl, optionally substituted (heteroaryl)cycloalkyl, optionally substituted (heteroaryl)heterocycloalkyl, wherein each heteroaryl has from from 1 to 3 rings, 5 to 7 ring members in each ring and, in at least one of said rings, from 1 to about 3 heteroatoms selected from the group consisting of N, O, and S;
R₁″ is chosen from optionally substituted alkyl, optionally substituted alkenyl, optionally substituted alkynyl, optionally substituted heterocycloalkyl, optionally substituted (cycloalkyl)alkyl, optionally substituted (heterocycloalkyl)alkyl, optionally substituted mono or dialkylamino, optionally substituted alkanoyl, optionally substituted carbocyclic aryl, optionally substituted (aryl)cycloalkyl, optionally substituted (aryl)heterocycloalkyl, optionally substituted heteroaryl, optionally substituted (heteroaryl)cycloalkyl, optionally substituted (heteroaryl)heterocycloalkyl, wherein each heteroaryl has from 1 to 3 rings, 5 to 7 ring members in each ring and, in at least one of said rings, from 1 to about 3 heteroatoms selected from the group consisting of N, O, and S;
R₃ is chosen from hydrogen, halogen, hydroxy, amino, cyano, nitro, alkyl, haloalkyl, alkoxy, aminoalkyl, and mono- and di-alkylamino;
R₁″ and R₃′ are independently chosen from hydrogen, halogen, alkyl, haloalkyl, and aminoalkyl;
R₂″ and R₃″ are independently chosen from hydrogen, alkyl, haloalkyl, optionally substituted mono or dialkylamino, optionally substituted alkanoyl, and aminoalkyl;
Z₄ is selected from NR and CR₄;
Z₅ is selected from NR and CR₅ (wherein in certain preferred compounds Z₄ and Z₅ are not both nitrogen);
R₄ and R₅ are independently chosen from hydrogen, halogen, hydroxy, amino, cyano, nitro, optionally substituted alkyl, optionally substituted alkenyl, optionally substituted alkynyl, optionally substituted alkoxy, optionally substituted mono or dialkylamino, optionally substituted (cycloalkyl)alkyl, optionally substituted alkylthio, optionally substituted alkylsulfinyl, optionally substituted alkylsulfonyl, optionally substituted mono- or dialkylcarboxamide, optionally substituted carbocyclic aryl, and optionally substituted heteroaryl, said optionally substituted heteroaryl having from 1 to 3 rings, 5 to 7 ring members in each ring and, in at least one of said rings, from 1 to about 3 heteroatoms selected from the group consisting of N, O, and S;
R₁₀ and R₁₁ are independently hydrogen or C₁-C₄ alkyl; and
m is 0, 1, or 2.

In certain preferred compounds of Formula I, Ar is not 2-bromophenyl when R₅ is alkoxy.

The invention further comprises methods of treating patients suffering from certain disorders with a therapeutically effective amount of at least one compound of the invention. These disorders include CNS disorders, particularly affective disorders, anxiety disorders, stress-related disorders, eating disorders and substance abuse. The patient suffering from these disorders may be a human or other animal (preferably a mammal), such as a domesticated companion animal (pet) or a livestock animal. Preferred compounds of the invention for such therapeutic purposes are those that antagonize the binding of CRF to CRF receptors (preferably CRF1, or less preferably CRF2 receptors). The ability of compounds to act as antagonists can be measured as an IC₅₀ value as described below.

According to yet another aspect, the present invention provides pharmaceutical compositions comprising compounds of Formula I and Formula XXXIII or the pharmaceutically acceptable salts (by which term is also encompassed pharmaceutically acceptable solvates) thereof, which compositions are useful for the treatment of the above-recited disorders. The invention further provides methods of treating patients suffering from any of the above-recited disorders with an effective amount of a compound or composition of the invention.

Additionally this invention relates to the use of the compounds of the invention (particularly labeled compounds of this invention) as probes for the localization of receptors in cells and tissues and as standards and reagents for use in determining the receptor-binding characteristics of test compounds.

Preferred 5-aryl-pyrazolo[4,3-d]pyrimidines, 6-aryl-pyrazolo[3,4-d]pyrimidines and related compounds of the invention exhibit good activity, i.e., a half-maximal inhibitory concentration (IC₅₀) of less than 1 millimolar, in the standard in vitro CRF receptor binding assay of Example 24, which follows. Particularly preferred 5-aryl-Pyrazolo[4,3-d]pyrimidines, 6-aryl-Pyrazolo[3,4-d]pyrimidines and related compounds of the invention exhibit an IC₅₀of about 1 micromolar or less, still more preferably an IC₅₀ of about 100 nanomolar or less even more preferably an IC₅₀ of about 10 nanomolar or less. Certain particularly preferred compounds of the invention will exhibit an IC₅₀ of 1 nanomolar or less in such a defined standard in vitro CRF receptor binding assay.

### DETAILED DESCRIPTION OF THE INVENTION

In addition to compounds of Formula I, described above, the invention is further directed to compounds and pharmaceutically acceptable salts of Formula I wherein:
R is independently selected at each occurrence to be absent or oxygen;
E is a single bond, O, or S(O)ₘ;
m is 0, 1, or 2;
Ar is chosen from:
phenyl which is mono-, di-, or tri-substituted with R_{A}, or 1-naphthyl, 2-naphthyl, pyridyl, pyrimidinyl, pyrazinyl, pyridizinyl, imidazo-pyridyl, imidazo-pyrimidinyl, imidazo-pyrazinyl, imidazo-pyridizinyl, thienyl, thiazolyl, oxazolyl, isoxazolyl, pyrrolyl, furanyl, and triazolyl, each of which is optionally mono-, di-, or tri-substituted with R_{A};
wherein in Ar, at least one of the positions ortho to the point of attachment of Ar shown in Formula I is substituted with R_{A};
the group: 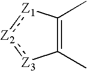
   represents a saturated, unsaturated or aromatic ring system comprising 2 or 3 adjacent nitrogen atoms, wherein:
Z₁ , is CR₁, CR₁R₁′, or NR₁″;
Z₂ is nitrogen or NR₂″;
Z₃ is CR₃, CR₃R₃′, nitrogen, NR₃″, oxygen, sulfur, sulfoxide or sulfone;
R₁ is chosen from
i) halogen, hydroxy, cyano, amino, C₁-C₁₀carbhydryl, —O(C₁-C₆carbhydryl), mono or di(C₁-C₆carbhydryl)amino, (C₃-C₇cyclocarbhydryl) C, -C₄carbhydryl, (C₃-C₇heterocycloalkyl)C₀-C₄carbhydryl, (benzoC₃-C₇cycloalkyl)C₀-C₄carbhydryl, (benzoC₃-C₇heterocycloalkyl)C₀-C₄carbhydryl, (C₁₋C₆)haloalkyl, and mono- and di-(C₁C₆)alkylamino, C₂-C₆alkanoyl; each of which is substituted with 0 or more substituents independently chosen from halogen, hydroxy, amino, oxo, cyano, C₁-C₆alkyl, C₁-C₆alkoxy, C₁-C₆hydroxyalkyl, C₁-C₆alkoxyC₁-C₆alkyl, C₁-C₆haloalkoxy, C₅-C₇heteroaryl, mono- and di-(C₁-C₆)alkylamino, and —XR_{C}, halo(C₁C₆)carbhydryl, —O(halo(C₁C₆)carbhydryl) and S(O)ₙ(C₁-C₆carbhydryl), -O(C₃-C₇cyclocarbhydryl)C₁-C₄carbhydryl, and S(O)ₙ(C₁-C₆carbhydryl), and
ii) phenyl which is mono-, di-, or tri-substituted with R_{A}, 1-naphthyl, 2-naphthyl, pyridyl, dihydropyridyl, tetrahydropyridyl, pyrimidinyl, pyrazinyl, pyridizinyl, thienyl, thiazolyl, oxazolyl, isoxazolyl, pyrrolyl, furanyl, and triazolyl, each of which is optionally mono-, di-, or tri-substituted with R_{A};
R₁″ is chosen from
i) C₁-C₁₀carbhydryl, (C₃-C₇cycloalkyl)C₁-C₄ carbhydryl, and halo(C₁C₆) carbhydryl, (C₃-C₇heterocycloalkyl)C₀-C₄carbhydryl, (benzoC₃-C₇cycloalkyl)C₀-C₄carbhydryl, (benzoC₃-C₇heterocycloalkyl)C₀-C₄carbhydryl, (C₁C₆)haloalkyl, and mono- and di-(C₁C₆)alkylamino, C₂-C₆alkanoyl; each of which is substituted with 0 or more substituents independently chosen from halogen, hydroxy, amino, oxo, cyano, C₁-C₆alkyl, C₁-C₆alkoxy, C₁-C₆hydroxyalkyl, C₁-C₆alkoxyC₁-C₆alkyl, C₁-C₆haloalkoxy, C₅-C₇heteroaryl, mono- and di-(C₁-C₆)alkylamino, and —XR_{C}, and
ii) phenyl which is mono-, di-, or tri-substituted with R_{A}, 1-naphthyl, 2-naphthyl, pyridyl, dihydropyridyl, tetrahydropyridyl, pyrimidinyl, pyrazinyl, pyridizinyl, thienyl, thiazolyl, oxazolyl, isoxazolyl, pyrrolyl, furanyl, and triazolyl, each of which is optionally mono-, di-, or tri-substituted with R_{A};
R₃ is chosen from hydrogen, halogen, hydroxy, amino, cyano, nitro, C₁-C₆alkyl, halo(C₁-C₆)alkyl, C₁-C₆alkoxy, amino(C₁-C₆)alkyl, and mono and di(C₁-C₆)alkylamino;
R₁′ and R₃′ are independently chosen from hydrogen, halogen, C₁-C₆alkyl, halo(C₁-C₆)alkyl, and amino(C₁-C₆)alkyl;
R₂″ and R₃″ are independently chosen from hydrogen, C₁-C₆alkyl, halo(C₁-C₆)alkyl, mono or di(C₁-C₆alkyl)amino, C₁-C₆alkanoyl and amino(C₁-C₆)alkyl;
Z₄ is selected from NR and CR₄;
Z₅ is selected from NR and CR₅ (wherein in certain preferred compounds Z₄ and Z₅ are not both nitrogen);
R₄ and R₅ are independently chosen from hydrogen, halogen, cyano, nitro, amino, mono or di(C₁-C₆ carbhydryl)amino, C₁-C₆ carbhydryl, (C₃-C₇cycloalkyl) C₁-C₄ carbhydryl,-O(C₃-C₇cycloalkyl) C₁-C₄ carbhydryl, halo(C₁-C₆) carbhydryl, -O(halo(C₁-C₆) carbhydryl), —O(C₁-C₆ carbhydryl), and S(O)ₙ(C₁-C₆ carbhydryl),
   where each carbhydrylis independently straight, branched, or cyclic, contains zero or 1 or more double or triple bonds, and is optionally substituted with one or more substituents independently chosen from halogen, hydroxy, amino, oxo, cyano, C₁-C₄alkoxy, and mono- or di(C₁-C₄)alkylamino, and
   where each C₃-C₇cycloalkyl is optionally substituted by one or more substituents independently chosen from halogen, amino, hydroxy, oxo, cyano, C₁-C₄alkoxy, and mono- or di(C₁-C₄)alkylamino;
R_{A} is independently selected at each occurrence from halogen, cyano, nitro, halo(C₁-C₆)alkyl, halo(C₁-C₆)alkoxy, hydroxy, amino, C₁-C₆alkyl substituted with 0-2 R_{B}, C₂-C₆alkenyl substituted with 0-2 R_{B}, C₂-C₆alkynyl substituted with 0-2 R_{B}, C₃-C₇Cycloalkyl substituted with 0-2 R_{B}, (C₃-C₇cycloalkyl)C₁-C₄alkyl substituted with 0-2 R_{B}, C₁-C₆alkoxy substituted with 0-2 R_{B}, —NH(C₁-C₆alkyl) substituted with 0-2 R_{B}, —N(C₁-C₆alkyl)(C₁-C₆alkyl) where each C₁-C₆alkyl is independently substituted with 0-2 R_{B}, —S(O)ₙ(C₁-C₆alkyl) substituted with 0-2 R_{B}, —XR_{C}, and Y;
R_{B} is independently selected at each occurrence from halogen, hydroxy, cyano, amino, C₁-C₄alkyl, —O(C₁-C₄alkyl), —NH(C₁-C₄alkyl), —N(C₁-C₄alkyl)( C₁-C₄alkyl), —S(O)ₙ(alkyl), halo(C₁-C₄)alkyl, halo(C₁-C₄)alkoxy, CO(C₁-C₄alkyl), CONH(C₁-C₄alkyl), CON(C₁-C₄alkyl)(C₁-C₄alkyl), —XR_{C}, and Y;
R_{C} and R_{D}, are the same or different, and are independently selected at each occurrence from: hydrogen, and
straight, branched, and cyclic alkyl groups, and (cycloalkyl)alkyl groups, said straight, branched, and cyclic alkyl groups, C₅-C₇heteroaryl(C₀-C₄alkyl), and (cycloalkyl)alkyl groups consist of 1 to 8 carbon atoms, and contain zero or one or more double or triple bonds, each of which 1 to 8 carbon atoms may be further substituted with one or more substituent(s) independently selected from oxo, hydroxy, halogen, cyano, amino, C₁-C₆alkoxy, —NH(C₁-C₆alkyl), —N(C₁-C₆alkyl)(C₁-C₆alkyl), —-NHC(═O)(C₁-C₆alkyl), —N(C₁-C₆alkyl)C(═O)(C₁-C₆alkyl), —NHS(O)ₙ(C₁-C₆alkyl), —S(O)ₙ(C₁-C₆alkyl), —S(O)ₙNH(C₁-C₆alkyl), —S(O)ₙ(C₁-C₆alkyl)(C₁-C₆alkyl), and Z;
X is independently selected at each occurrence from the group consisting of —CH₂—, —CHR_{D}—, —O—, —C(═O)—, —C(S)—, —C(═O)O—, —C(═S)O—, —S(O)ₙ—, —NH—, —NR_{D}—, —C(═O)NH—, —C(═O)NR_{D}—, —S(O)ₙNH—, —S(O)ₙNR_{D}—, —OC(═S)S—, —NHC(═O)—, —NR_{D}C(═O)—, —C(═S)NR_{D}—, —NHS(O)ₙ—, —OSiH₂—, —OSiH(C₁-C₄alkyl)-, —OSi(C₁-C₄alkyl)(C ₁-C₄alkyl)-, and —NR_{D}S(O)ₙ—;
Y and Z are independently selected at each occurrence from: 3- to 7-membered carbocyclic or heterocyclic groups which are saturated, unsaturated, or aromatic, which may be further substituted with one or more substituents independently selected from halogen, oxo, hydroxy, amino, cyano, C₁-C₄alkyl, —O(C₁-C₄alkyl), —NH(C₁-C₄alkyl), —N(C₁-C₄alkyl)(C₁-C₄alkyl),and —S(O)ₙ(alkyl),
   wherein said 3- to 7-memberered heterocyclic groups contain one or more heteroatom(s) independently selected from N, O, and S, with the point of attachment being either carbon or nitrogen; and
n is independently selected at each occurrence from 0, 1, and 2.

Such compounds and salts will be referred to as compounds and salts of Formula IA.

Particularly embodied by the invention are compounds and pharmaceutically acceptable salts of Formula II-Formula XXII shown in TABLE I.

TABLE I

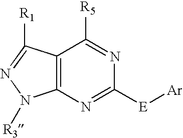

Formula II

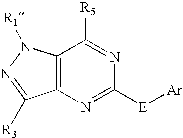

Formula III

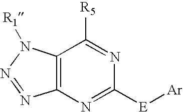

Formula IV

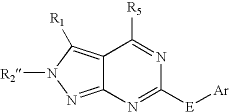

Formula V

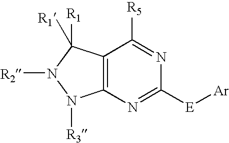

Formula VI

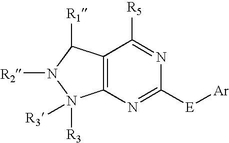

Formula VII

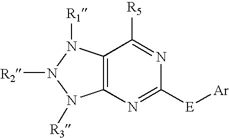

Formula VIII

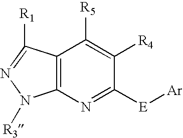

Formula IX

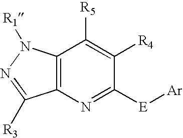

Formula X

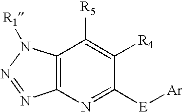

Formula XI

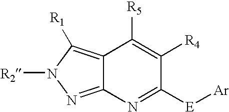

Formula XII

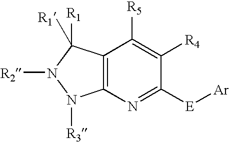

Formula XIII

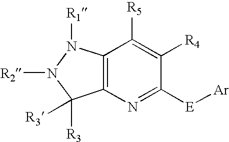

Formula XIV

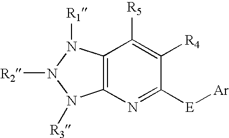

Formula XV

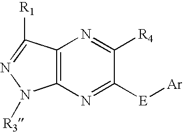

Formula XVI

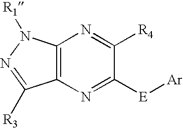

Formula XVII

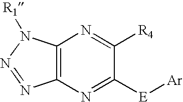

Formula XVIII

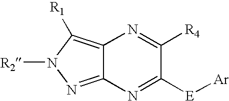

Formula XIX

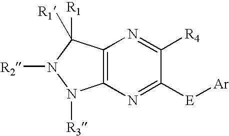

Formula XX

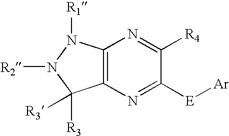

Formula XXI

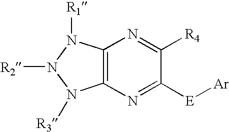

Formula XXII

For the compounds and pharmaceutically acceptable salts of Formula II-Formula XXII R₁, R₁′, R₁″, R₂″, R₃, R₃′, R₃″, R₄, R₅, and Ar are as defined for Formula I or more preferably as defined for Formula IA.

Preferred compounds and pharmaceutically acceptable salts of Formula II-Formula XXII are those wherein:
E is a single bond, O, or S(O)ₘ;
m is 0, 1, or 2;
R₁ and R₁″ are defined for Formula I or more preferably as defined for Formula IA;
R₁′ is hydrogen or C₁-C₆ alkyl;
R₂″ is selected from hydrogen, methyl, and ethyl;
R₃ and R₃′ are independently selected from hydrogen and C₁-C₆alkyl; and
R₃″ is selected from hydrogen and C₁-C₆alkyl;
R₄ and R₅ are independently selected from hydrogen, halogen, cyano, amino, C₁-C₆alkyl, C₁-C₆alkoxy, C₃-C₇cycloalkyl, (C₃-C₇cycloalkyl)C₁-C₄alkyl, (C₃-C₇cycloalkyl)C₁-C₄alkoxy, mono and di(C₁-C₆alkyl)amino, amino(C₁-C₆-)alkyl, mono and di(C₁-C₆alkyl)amino(C₁-C₆)alkyl, halo(C₁-C₆)alkyl, and halo(C₁-C₆)alkoxy; and
Ar is selected from the group consisting of phenyl, pyridyl and pyrimidinyl, imidazo-pyridyl, imidazo-pyrimidinyl, imidazo-pyrazinyl, imidazo-pyridizinyl, each of which is mono-di- or tri-substituted with substituents independently chosen from halogen, cyano, nitro, halo(C₁-C₆)alkyl, halo(C₁-C₆)alkoxy, hydroxy, amino, C₁-C₆alkyl substituted, C₂-C₆alkenyl, C₂-C₆alkynyl, C₃-C₇cycloalkyl, (C₃-C₇cycloalkyl)C₁-C₄alkyl, C₁-C₆alkoxy, mono- and di(C₁-C₆alkyl)amino, amino(C₁-C₆)alkyl, and mono- or di(C₁-C₆alkyl)amino, wherein, in Ar, at least one of the positions ortho to the point of attachment of Ar shown in Formula II-XXII is substituted.

Certain preferred compounds and of Formula II-Formula XXII are those wherein
R₁ or R₁″ is chosen from 2-ethylbutyl or 2-ethylpropyl and Ar is di- or tri-substituted phenyl or pyridyl.

Other preferred compounds of Formula II-Formula XXII, include those compounds in which R₁ or R₁″ is selected from C₁-C₁₀alkyl and (C₃-C₇cycloalkyl)C₀-C₄alkyl, each of which is substituted with 0 or more substituents independently chosen from halogen, hydroxy, amino, oxo, cyano, C₁-C₄alkoxy, and mono- and di-(C₁-C₄)alkylamino.

Certain other preferred compounds of Formula II-Formula XXII, include those compounds in which R₁ or R₁″ is selected from C₃₋₆heterocycloalkyl and (C₃₋₆heterocycloalkyl)C₁₋₄alkyl, each of which is substituted with 0-4 substitutents selected from halogen, amino, hydroxy, nitro, cyano, C₁-C₆alkyl, C₁-C₆alkoxy, C₁-C₆hydroxyalkyl, C₁-C₆alkoxyC₁-C₆alkyl, (C₁-C₆)haloalkyl, (C₁-C₆)haloalkoxy, mono- and di-(C₁-C₆)alkylamino, XR_{C}. In some preferred compounds of Formula II-Formula XXII, R₁ or R₁″ is chosen from tetrahydrofuranyl, tetrahydropyranyl, morpholinyl, pyrrolidinyl, piperidinyl, piperazinyl [2.2.1]-azabicyclic rings, [2.2.2]-azabicyclic rings, [3.3.1]-azabicyclic rings, quinuclidinyl, azetidinyl, azetidinonyl, oxindolyl, dihydroimidazolyl, and pyrrolidinonyl, each of which is substituted with from 0 to 2 substituents independently chosen from: (i) halogen, hydroxy, amino, cyano, or (ii) C₁-C₄alkyl, C₁-C₄alkoxy, and mono- and di-(C₁-C₄)alkylamino, each of which is substituted with 0 or 1 substituents selected from halogen, hydroxy, amino, C₁₋₂ alkoxy, or C₃₋₆heterocycloalkyl.

Certain other preferred compounds of Formula II-Formula XXII, include those compounds in which R₁ or R₁″ is selected from 3-pentyl, 2-butyl, 1-methoxy-but-2-yl, 1-dimethylamino-but-2-yl, 3-(thiazol-2-yl)-1H-pyrazol-1-yl, and groups of formula: 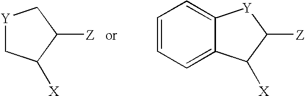
wherein X is the point of attachment to the nitrogen of the imidazo ring,
Y is selected from CH₂, O, S, S(O), SO₂, NC₁-C₈alkyl (including linear and branched alkyl groups), NC₁-C₆haloalkyl, NC₃-C₈cycloalkyl, NC(O)C₁-C₈alkyl (including linear and branched alkyl groups), NC(O)C₁-C₆haloalkyl, NC(O)C₃-C₈cycloalkyl, N-benzoyl, N-benzyl, NCOOC₁-C₈alkyl (including linear and branched alkyl groups), NCOOC₁-C₆haloalkyl, NCOOC₃-C₈cycloalkyl, and
Z is selected from hydrogen, hydroxy, amino, NC₁-C₈alkyl (including linear and branched alkyl groups), NHC₁-C₆haloalkyl, NHC₃-C₈cycloalkyl, NHC(O)C₁-C₈alkyl (including linear and branched alkyl groups), NHC(O)C₁-C₆haloalkyl, NHC(O)C₃-C₈cycloalkyl, NH-benzoyl, NH-benzyl, NHCOOC₁-C₈alkyl (including linear and branched alkyl groups), NHCOOC₁-C₆haloalkyl, NHCOOC₃-C₈cycloalkyl, C₁-C₈alkoxy (including linear and branched alkoxy groups), C₁-C₆haloalkoxy, C₃-C₈cycloalkoxy, OC(O)C₁-C₈alkyl (including linear and branched alkyl groups), OC(O)C₁-C₆haloalkyl, OC(O)C₃-C₈cycloalkyl, benzoyloxy, benzyloxy, OCONHC₁-C₈alkyl (including linear and branched alkyl groups), OCONHC₁-C₆haloalkyl, OCONHC₃-C₈cycloalkyl, C₁-C₈alkylthio (including linear and branched alkyl groups), C₁-C₆haloalkylthio, C₃-C₈cycloalkylthio, S(O)C₁-C₈alkyl(including linear and branched alkyl groups), S(O)C₁-C₆haloalkyl, S(O)C₃-C₈cycloalkyl, SO₂C₁-C₈alkyl (including linear and branched alkyl groups), SO₂C₁-C₆haloalkyl, SO₂C₃-C₈cycloalkyl.

In yet other aspects, preferred compounds of Formula II-Formula XXII and compounds include those compounds in which R₁ or R₁″ is selected from 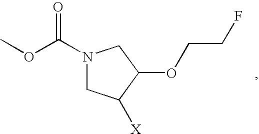
or more preferably a group of formula 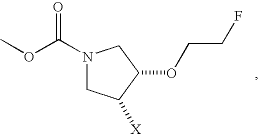
wherein X is the point of attachment to the nitrogen of the imidazo ring.

The invention further provides compounds of Formula XXIII 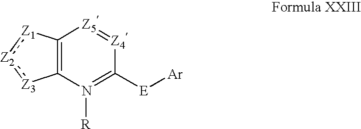
and the pharmaceutically acceptable salts thereof, wherein:
E is a single bond, O, or S(O)ₘ;
m is 0, 1, or 2;
Ar is chosen from:
   phenyl which is mono-, di-, or tri-substituted;
   1-naphthyl and 2-naphthyl, each of which is optionally mono-, di-, or tri-substituted; and
   optionally mono-, di-, or tri-substituted heteroaryl, said heteroaryl having from 1 to 3 rings, 5 to 7 ring members in each ring and, in at least one of said rings, from 1 to about 3 heteroatoms selected from the group consisting of N, O, and S;
   wherein in Ar, at least one of the positions ortho to the point of attachment of Ar shown in Formula XXIII is substituted;
R is independently selected at each occurrence to be absent or oxygen;
the group: 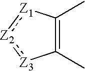
   represents a saturated, unsaturated or aromatic 5-membered ring system containing 2 or 3 nitrogen atoms, wherein:
Z₁ is CR₁, CR₁R₁′, or NR₁″;
Z₂ is nitrogen or NR₂″,
Z₃ is CR₃, CR₃R₃′, nitrogen, NR₃″, oxygen, sulfur, sulfoxide or sulfone;
R₁ is chosen from halogen, hydroxy, cyano, amino, optionally substituted alkyl, optionally substituted alkenyl, optionally substituted alkynyl, optionally substituted alkoxy, optionally substituted mono or dialkylamino, optionally substituted heterocycloalkyl, optionally substituted (cycloalkyl)alkyl, optionally substituted (heterocycloalkyl)alkyl, optionally substituted alkylthio, optionally substituted alkylsulfinyl, optionally substituted alkylsulfonyl, optionally substituted mono- or dialkylcarboxamide, optionally substituted carbocyclic aryl, optionally substituted (aryl)cycloalkyl, optionally substituted (aryl)heterocycloalkyl, and optionally substituted heteroaryl, optionally substituted (heteroaryl)cycloalkyl, optionally substituted (heteroaryl)heterocycloalkyl, wherein each heteroaryl has from said optionally substituted heteroaryl having from 1 to 3 rings, 5 to 7 ring members in each ring and, in at least one of said rings, from 1 to about 3 heteroatoms selected from the group consisting of N, O, and S;
R₁″ is chosen from optionally substituted alkyl, optionally substituted alkenyl, optionally substituted alkynyl, optionally substituted heterocycloalkyl, optionally substituted (cycloalkyl)alkyl, optionally substituted (heterocycloalkyl)alkyl, optionally substituted mono or dialkylamino, optionally substituted alkanoyl, optionally substituted carbocyclic aryl, optionally substituted (aryl)cycloalkyl, optionally substituted (aryl)heterocycloalkyl, and optionally substituted heteroaryl, optionally substituted (heteroaryl)cycloalkyl, optionally substituted (heteroaryl)heterocycloalkyl, wherein each heteroaryl has from said optionally substituted heteroaryl having from 1 to 3 rings, 5 to 7 ring members in each ring and, in at least one of said rings, from 1 to about 3 heteroatoms selected from the group consisting of N, O, and S;
R₃ is chosen from hydrogen, halogen, hydroxy, amino, cyano, nitro, alkyl, haloalkyl, alkoxy, aminoalkyl, and mono- and di-alkylamino;
R₁′ and R₃′ are independently chosen from hydrogen, halogen, alkyl, haloalkyl, and aminoalkyl;
R₂″ and R₃″ are independently chosen from hydrogen, alkyl, haloalkyl, optionally substituted mono or dialkylamino, optionally substituted alkanoyl, and aminoalkyl;
Z₄′ is NR₄″ or C═O;
Z₅′ is NR₅″ or C═O;
wherein one of Z₄′ or Z₅′ is C═O; and
R₄″ and R₅″ are independently chosen from hydrogen, alkyl, aminoalkyl, and haloalkyl.

Preferred compounds and pharmaceutically acceptable salts of Formula XXIII are those wherein R, Ar, Z₁, Z₂, and Z₃ are as defined for Formula IA;
Z₄′ is NR₄″ or C═O;
Z₅′ is NR₅″ or C═O;
wherein one of Z₄′ or Z₅′ is C═O; and
R₄″ and R₅″ are independently chosen from hydrogen, C₁-C₆alkyl, amino(C₁-C₆)alkyl, and halo(C₁-C₆)alkyl. Such compounds will be referred to as compounds of Formula XXIIIA.

Also particularly embodied by the invention are compounds of Formula XXIV-Formula XXXVII are shown in TABLE II.

TABLE II

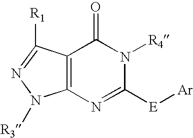

Formula XXIV

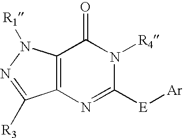

Formula XXV

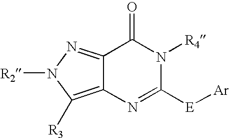

Formula XXVII

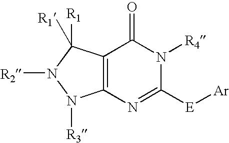

Formula XXVIII

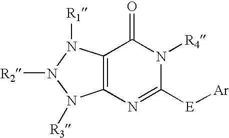

Formula XXX

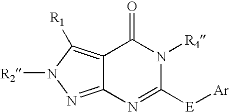

Formula XXXI

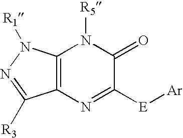

Formula XXXIII

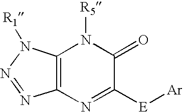

Formula XXXIV

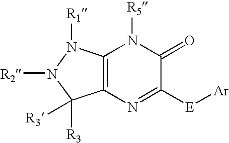

Formula XXXVI

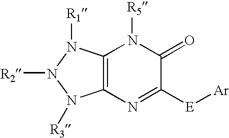

Formula XXXVI

In compounds of Formula XXIV-Formula XXXVII R₁, R₁′, R₁″, R₂″, R₃, R₃′, R₃″, R₄″, R₅″, E and Ar are as defined for compounds and salts of Formula XXIII or more preferably as defined for compounds of Formula XXIIIA.

Preferred compounds and pharmaceutically acceptable salts of Formuls XXIV-Formula Formula XXXVII are those wherein:
R₁ and R₁″ are as defined for Formula XXIII or more preferably as defined for compounds of Formula XXIIIA;
E is a single bond, O, or S(O)ₘ;
m is 0, 1, or 2;
R₁′ is hydrogen or C₁-C₆ alkyl;
R₃ and R₃′ are independently selected from hydrogen and C₁-C₆alkyl; and
R₂″ and R₃″ are independently selected from hydrogen and C₁-C₆alkyl;
R₄″ and R₅″ are selected from hydrogen, methyl, and ethyl;
Ar is selected from the group consisting of phenyl, pyridyl and pyrimidinyl, imidazo-pyridyl, imidazo-pyrimidinyl, imidazo-pyrazinyl, imidazo-pyridizinyl, which is mono- di- or trisubstituted with substituents independently chosen from halogen, cyano, nitro, halo(C₁-C₆)alkyl, halo(C₁-C₆)alkoxy, hydroxy, amino, C₁-C₆alkyl substituted, C₂-C₆alkenyl, C₂-C₆alkynyl, C₃-C₇cycloalkyl, (C₃-C₇cycloalkyl)C₁-C₄alkyl, C₁-C₆alkoxy, mono- and di(C₁-C₆alkyl)amino, amino(C₁-C₆)alkyl, and mono- and di(C₁-C₆alkyl)amino, wherein, in Ar, at least one of the positions ortho to the point of attachment of Ar shown in Formula XXIV-Formula XXXVII is substituted.

Certain preferred compounds and of Formula XXIV-Formula XXXVII are those wherein
R₁ or R₁″ is chosen from 2-ethylbutyl or 2-ethylpropyl and Ar is di- or tri-substituted phenyl or pyridyl.

Other preferred compounds of Formula XXIV-Formula XXXVII, include those compounds in which R₁ or R₁″ is selected from C₁-C₁₀alkyl and (C₃-C₇cycloalkyl)C₀-C₄alkyl, each of which is substituted with 0 or more substituents independently chosen from halogen, hydroxy, amino, oxo, cyano, C₁-C₄alkoxy, and mono- and di-(C₁-C₄)alkylamino.

Certain other preferred compounds of Formula XXIV-Formula XXXVII, include those compounds in which R₁ or R₁″ R₁″ is selected from C₃₋₆heterocycloalkyl and (C₃₋₆heterocycloalkyl)C₁₋₄alkyl, each of which is substituted with 0-4 substitutents selected from halogen, amino, hydroxy, nitro, cyano, C₁-C₆alkyl, C₁-C₆alkoxy, C₁-C₆hydroxyalkyl, C₁-C₆alkoxyC₁-C₆alkyl, (C₁-C₆)haloalkyl, (C₁-C₆)haloalkoxy, mono- and di-(C₁-C₆)alkylamino, XR_{C}. In some preferred compounds of Formula XXIV-Formula XXXVII, R₁ or R₁″ is chosen from tetrahydrofuranyl, tetrahydropyranyl, morpholinyl, pyrrolidinyl, piperidinyl, piperazinyl [2.2.1]-azabicyclic rings, [2.2.2]-azabicyclic rings, [3.3.1]-azabicyclic rings, quinuclidinyl, azetidinyl, azetidinonyl, oxindolyl, dihydroimidazolyl, and pyrrolidinonyl, each of which is substituted with from 0 to 2 substituents independently chosen from: (i) halogen, hydroxy, amino, cyano, or (ii) C₁-C₄alkyl, C₁-C₄alkoxy, and mono- and di-(C₁-C₄)alkylamino, each of which is substituted with 0 or 1 substituents selected from halogen, hydroxy, amino, C₁₋₂alkoxy, or C₃₋₆heterocycloalkyl.

Certain other preferred compounds of Formula XXIV-Formula XXXVII, include those compounds in which R₁ or R₁″ is selected from 3-pentyl, 2-butyl, 1-methoxy-but-2-yl, 1-dimethylamino-but-2-yl, 3-(thiazol-2-yl)-1H-pyrazol-1-yl, and groups of formula: 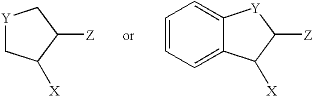
wherein X is the point of attachment to the nitrogen of the imidazo ring,
Y is selected from CH₂, O, S, S(O), SO₂, NC₁-C₈alkyl (including linear and branched alkyl groups), NC₁-C₆haloalkyl, NC₃-C₈cycloalkyl, NC(O)C₁-C₈alkyl (including linear and branched alkyl groups), NC(O)C₁-C₆haloalkyl, NC(O)C₃-C₈cycloalkyl, N-benzoyl, N-benzyl, NCOOC₁-C₈alkyl (including linear and branched alkyl groups), NCOOC₁-C₆haloalkyl, NCOOC₃-C₈cycloalkyl, and
Z is selected from hydrogen, hydroxy, amino, NC₁-C₈alkyl (including linear and branched alkyl groups), NHC₁-C₆haloalkyl, NHC₃-C₈cycloalkyl, NHC(O)C₁-C₈alkyl (including linear and branched alkyl groups), NHC(O)C₁-C₆haloalkyl, NHC(O)C₃-C₈cycloalkyl, NH-benzoyl, NH-benzyl, NHCOOC₁-C₈alkyl (including linear and branched alkyl groups), NHCOOC₁-C₆haloalkyl, NHCOOC₃-C₈cycloalkyl, C₁-C₈alkoxy (including linear and branched alkoxy groups), C₁-C₆haloalkoxy, C₃-C₈cycloalkoxy, OC(O)C₁-C₈alkyl (including linear and branched alkyl groups), OC(O)C₁-C₆haloalkyl, OC(O)C₃-C₈cycloalkyl, benzoyloxy, benzyloxy, OCONHC₁-C₈alkyl (including linear and branched alkyl groups), OCONHC₁-C₆haloalkyl, OCONHC₃-C₈cycloalkyl, C₁-C₈alkylthio (including linear and branched alkyl groups), C₁-C₆haloalkylthio, C₃-C₈cycloalkylthio, S(O)C₁-C₈alkyl(including linear and branched alkyl groups), S(O)C₁-C₆haloalkyl, S(O)C₃-C₈cycloalkyl, SO₂C₁-C₈alkyl (including linear and branched alkyl groups), SO₂C₁-C₆haloalkyl, SO₂C₃-C₈cycloalkyl.

In yet other aspects, preferred compounds of Formula XXIV-Formula XXXVII include those compounds in which R₁ or R₁″ is selected from 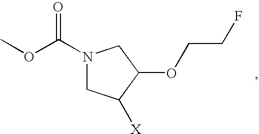
or more preferably a group of formula 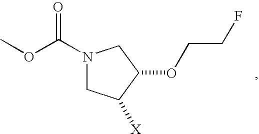
wherein X is the point of attachment to the nitrogen of the imidazo ring.

In yet another aspect, the invention provides compounds according to Formula XXXVIII: 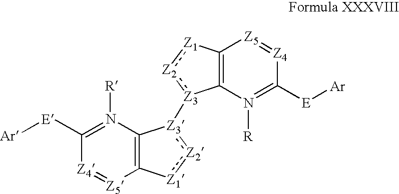
or a pharmaceutically acceptable salt thereof, wherein:
R is independently selected at each occurrence to be absent or oxygen;
E is a single bond, O, or S(O)ₘ;
m is 0, 1, or 2;
Ar and Ar′ are independently chosen from:
phenyl which is mono-, di-, or tri-substituted with R_{A}, or 1-naphthyl, 2-naphthyl, pyridyl, pyrimidinyl, pyrazinyl, pyridizinyl, imidazo-pyridyl, imidazo-pyrimidinyl, imidazo-pyrazinyl, imidazo-pyridizinyl, thienyl, thiazolyl, oxazolyl, isoxazolyl, pyrrolyl, furanyl, and triazolyl, each of which is optionally mono-, di-, or tri-substituted with R_{A};
wherein in Ar and Ar′, at least one of the positions ortho to the point of attachment of Ar and Ar′ shown in Formula XXXVIII are substituted with R_{A};
the groups: 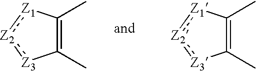
   represents a saturated, unsaturated or aromatic ring system comprising 2 or 3 adjacent nitrogen atoms, wherein:
Z₁ and Z₁′ are independently selected from CR₁, CR₁R₁′, or NR₁″;
Z₂ and Z₂′ are nitrogen or NR₂″;
Z₃ and Z₃′ are CR₃, CR₃R₃′, nitrogen, NR₃″, oxygen, sulfur, sulfoxide or sulfone;
R₁ is chosen from
i) halogen, hydroxy, cyano, amino, C₁-C₁₀carbhydryl, —O(C₁-C₆ carbhydryl), mono or di(C₁-C₆ carbhydryl)amino, (C₃-C₇cyclocarbhydryl) C₁-C₄ carbhydryl, (C₃₋₆heterocycloalkyl)C₀-C₄carbhydryl, (benzoC₃-C₇cycloalkyl)C₀-C₄carbhydryl, (benzoC₃-C₇heterocycloalkyl)C₀-C₄carbhydryl, (C₁-C₆)haloalkyl, and mono- and di-(C₁-C₆)alkylamino, C₂-C₆alkanoyl; each of which is substituted with 0 or more substituents independently chosen from halogen, hydroxy, amino, oxo, cyano, C₁-C₆alkyl, C₁-C₆alkoxy, C₁-C₆hydroxyalkyl, C₁-C₆alkoxyC₁-C₆alkyl, C₁-C₆haloalkoxy, C₅-C₇heteroaryl, mono- and di-(C₁-C₆)alkylamino, and —XR_{C}, halo(C₁C₆)carbhydryl, —O(halo(C₁C₆) carbhydryl) and S(O)ₙ(C₁-C₆ carbhydryl), —O(C₃-C₇cyclo carbhydryl)C₁-C₄ carbhydryl, and S(O)ₙ(Cₗ-C₆ carbhydryl), and
ii) phenyl which is mono-, di-, or tri-substituted with R_{A}, 1-naphthyl, 2-naphthyl, pyridyl, dihydropyridyl, tetrahydropyridyl, pyrimidinyl, pyrazinyl, pyridizinyl, thienyl, thiazolyl, oxazolyl, isoxazolyl, pyrrolyl, furanyl, and triazolyl, each of which is optionally mono-, di-, or tri-substituted with R_{A};
R₁″ is chosen from
i) C₁-C₁₀carbhydryl, (C₃-C₇cycloalkyl)C₁-C₄ carbhydryl, and halo(C₁C₆) carbhydryl, (C₃₋₆heterocycloalkyl)C₀-C₄carbhydryl, (benzoC₃-C₇cycloalkyl)C₀-C₄carbhydryl, (benzo C₃₋₆heterocycloalkyl)C₀-C₄carbhydryl, (C₁C₆)haloalkyl, and mono- and di-(C₁-C₆)alkylamino, C₂-C₆alkanoyl; each of which is substituted with 0 or more substituents independently chosen from halogen, hydroxy, amino, oxo, cyano, C₁-C₆alkyl, C₁-C₆alkoxy, C₁-C₆hydroxyalkyl, C₁-C₆alkoxyC₁-C₆alkyl, C₁-C₆haloalkoxy, C₅-C₇heteroaryl, mono- and di-(C₁-C₆)alkylamino, and —XR_{C}, and
ii) phenyl which is mono-, di-, or tri-substituted with R_{A}, benzyl, 1-naphthyl, 2-naphthyl, pyridyl, dihydropyridyl, tetrahydropyridyl, pyrimidinyl, pyrazinyl, pyridizinyl, thienyl, thiazolyl, oxazolyl, isoxazolyl, pyrrolyl, furanyl, and triazolyl, each of which is optionally mono-, di-, or tri-substituted with R_{A};
R₃ is chosen from hydrogen, halogen, hydroxy, amino, cyano, nitro, C₁-C₆alkyl, halo(C₁-C₆)alkyl, C₁-C₆alkoxy, amino(C₁-C₆)alkyl, and mono and di(C₁-C₆)alkylamino;
R₁′ and R₃′ are independently chosen from hydrogen, halogen, C₁-C₆alkyl, halo(C₁-C₆)alkyl, and amino(C₁-C₆)alkyl;
R₂″ and R₃″ are independently chosen from hydrogen, C₁-C₆alkyl, halo(C₁-C₆)alkyl, mono or di(C₁-C₆alkyl)amino, C₁-C₆alkanoyl and amino(C₁-C₆)alkyl;
Z₄ and Z₄′ are selected from NR and CR₄;
Z₅ and Z₅′ are selected from NR and CR₅ (in certain preferred compounds zero or one of Z₄ and Z₅ is NR and zero or one of Z₄′and Z₅′ is NR);
R₄ and R₅ are independently chosen from hydrogen, halogen, cyano, nitro, amino, mono or di(C₁-C₆ carbhydryl)amino, C₁-C₆ carbhydryl, (C₃-C₇cycloalkyl) C₁-C₄ carbhydryl,—O(C₃-C₇cycloalkyl) C₁-C₄ carbhydryl, halo(C₁-C₆) carbhydryl, —O(halo(C₁-C₆) carbhydryl), —O(C₁-C₆ carbhydryl), S(O)ₙ(C₁-C₆ carbhydryl), N(H)(S(O)ₙ(C₁-C₆ carbhydryl)), N(C₁-C₆ carbhydryl) (S(O)ₙ(C₁-C₆ carbhydryl)
   where each carbhydrylis independently straight, branched, or cyclic, contains zero or 1 or more double or triple bonds, and is optionally substituted with one or more substituents independently chosen from halogen, hydroxy, amino, oxo, cyano, C₁-C₄alkoxy, and mono- or di(C₁-C₄)alkylamino, and
   where each C₃-C₇cycloalkyl is optionally substituted by one or more substituents independently chosen from halogen, amino, hydroxy, oxo, cyano, C₁-C₄alkoxy, and mono- or di(C₁-C₄)alkylamino;
R_{A} is independently selected at each occurrence from halogen, cyano, nitro, halo(C₁-C₆)alkyl, halo(C₁-C₆)alkoxy, hydroxy, amino, C₁-C₆alkyl substituted with 0-2 R_{B}, C₂-C₆alkenyl substituted with 0-2 R_{B}, C₂-C₆alkynyl substituted with 0-2 R_{B}, C₃-C₇cycloalkyl substituted with 0-2 R_{B}, (C₃-C₇cycloalkyl)C₁-C₄alkyl substituted with 0-2 R_{B}, C₁-C₆alkoxy substituted with 0-2 R_{B}, —NH(C₁-C₆alkyl) substituted with 0-2 R_{B}, —N(C₁-C₆alkyl)(C₁-C₆alkyl) where each C₁-C₆alkyl is independently substituted with 0-2 R_{B}, —S(O)ₙ(C₁-C₆alkyl) substituted with 0-2 R_{B}, —XR_{C}, and Y;
R_{B} is independently selected at each occurrence from halogen, hydroxy, cyano, amino, C₁-C₄alkyl, —O(C₁-C₄alkyl), —NH(C₁-C₄alkyl), —N(C₁-C₄alkyl)(C₁-C₄alkyl), —S(O)ₙ(alkyl), halo(C₁-C₄)alkyl, halo(C₁-C₄)alkoxy, CO(C₁-C₄alkyl), CONH(C₁-C₄alkyl), CON(C₁-C₄alkyl)(C₁-C₄alkyl), —XR_{C}, and Y;
R_{C} and R_{D}, are the same or different, and are independently selected at each occurrence from:
   hydrogen, and straight, branched, and cyclic alkyl groups, and (cycloalkyl)alkyl groups, said straight, branched, and cyclic alkyl groups, C₅-C₇heteroaryl(C₀-C₄alkyl), and (cycloalkyl)alkyl groups consist of 1 to 8 carbon atoms, and contain zero or one or more double or triple bonds, each of which 1 to 8 carbon atoms may be further substituted with one or more substituent(s) independently selected from oxo, hydroxy, halogen, cyano, amino, C₁-C₆alkoxy, —NH(C₁-C₆alkyl), —N(C₁-C₆alkyl)(C₁-C₆alkyl), —NHC(═O)(C₁-C₆alkyl), —N(C₁-C₆alkyl)C(═O)(C₁-C₆alkyl), —NHS(O)ₙ(C₁-C₆alkyl), —S(O)ₙ(C₁-C₆alkyl), —S(O)ₙNH(C₁-C₆alkyl), —S(O)ₙN(C₁-C₆alkyl)(C₁-C₆alkyl), and Z;
X is independently selected at each occurrence from the group consisting of —CH₂—, —CHR_{D}—, —O—, —C(═O)—, —C(S)—, —C(═O)O—, —C(═S)O—, —S(O)ₙ—, —NH—, —NR_{D}—, —C(═O)NH—, —C(═O)NR_{D}—, —S(O)ₙNH—, —S(O)ₙNR_{D}—, —OC(═S)S—, —NHC(═O)—, —NR_{D}C(═O)—, —C(═S)NR_{D}—, —NHS(O)ₙ—, —OSiH₂—, —OSiH(C₁-C₄alkyl)—, —OSi(C₁-C₄alkyl)(C₁-C₄alkyl)-, and —NR_{D}S(O)ₙ—;
Y and Z are independently selected at each occurrence from: 3- to 7-membered carbocyclic or heterocyclic groups which are saturated, unsaturated, or aromatic, which may be further substituted with one or more substituents independently selected from halogen, oxo, hydroxy, amino, cyano, C₁-C₄alkyl, —O(C₁-C₄alkyl), —NH(C₁-C₄alkyl), —N(C₁-C₄alkyl)(C₁-C₄alkyl),and —S(O)ₙ(alkyl),
   wherein said 3- to 7-memberered heterocyclic groups contain one or more heteroatom(s) independently selected from N, O, and S, with the point of attachment being either carbon or nitrogen; and
n is independently selected at each occurrence from 0, 1, and 2.

Compounds of the invention are useful in treating a variety of conditions including affective disorders, anxiety disorders, stress disorders, eating disorders, and drug addiction.

Affective disorders include all types of depression, bipolar disorder, cyclothymia, and dysthymia.

Anxiety disorders include generalized anxiety disorder, panic, phobias and obsessive-compulsive disorder.

Stress-related disorders include post-traumatic stress disorder, hemorrhagic stress, stress-induced psychotic episodes, psychosocial dwarfism, stress headaches, stress-induced immune systems disorders such as stress-induced fever, and stress-related sleep disorders.

Eating disorders include anorexia nervosa, bulimia nervosa, and obesity.

Modulators of the CRF receptors are also useful in the treatment (e.g., symptomatic treatment)of a variety of neurological disorders including supranuclear palsy, AIDS related dementias, multiinfarct dementia, neurodegenerative disorders such as Alzheimer's disease, Parkinson's disease, and Huntington's disease, head trauma, spinal cord trauma, ischemic neuronal damage, amyotrophic lateral sclerosis, disorders of pain perception such as fibromyalgia and epilepsy.

Additionally compounds of Formula I are useful as modulators of the CRF receptor in the treatment (e.g., symptomatic treatment) of a number of gastrointestinal, cardiovascular, hormonal, autoimmune and inflammatory conditions. Such conditions include irritable bowel syndrome, ulcers, Crohn's disease, spastic colon, diarrhea, post operative ilius and colonic hypersensitivity associated with psychopathological disturbances or stress, hypertension, tachycardia, congestive heart failure, infertility, euthyroid sick syndrome, inflammatory conditions effected by rheumatoid arthritis and osteoarthritis, pain, asthma, psoriasis and allergies.

Compounds of Formula I are also useful as modulators of the CRF1 receptor in the treatment of animal disorders associated with aberrant CRF levels. These conditions include porcine stress syndrome, bovine shipping fever, equine paroxysmal fibrillation, and dysfunctions induced by confinement in chickens, sheering stress in sheep or human-animal interaction related stress in dogs, psychosocial dwarfism and hypoglycemia.

Typical subjects to which compounds of the invention may be administered will be mammals, particularly primates, especially humans. For veterinary applications, a wide variety of subjects will be suitable, e.g. livestock such as cattle, sheep, goats, cows, swine and the like; poultry such as chickens, ducks, geese, turkeys, and the like; and other domesticated animals particularly pets such as dogs and cats. For diagnostic or research applications, a wide variety of mammals will be suitable subjects including rodents (e.g. mice, rats, hamsters), rabbits, primates, and swine such as inbred pigs and the like. Additionally, for in vitro applications, such as in vitro diagnostic and research applications, body fluids (e.g., blood, plasma, serum, CSF, lymph, cellular interstitial fluid, aqueous humor, saliva, synovial fluid, feces, or urine) and cell and tissue samples of the above subjects will be suitable for use.

The CRF binding compounds provided by this invention and labeled derivatives thereof are also useful as standards and reagents in determining the ability of test compounds (e.g., a potential pharmaceutical) to bind to a CRF receptor.

Labeled derivatives the CRF antagonist compounds provided by this invention are also useful as radiotracers for positron emission tomography (PET) imaging or for single photon emission computerized tomography (SPECT).

More particularly compounds of the invention may be used for demonstrating the presence of CRF receptors in cell or tissue samples. This may be done by preparing a plurality of matched cell or tissue samples, at least one of which is prepared as an experiment sample and at least one of which is prepared as a control sample. The experimental sample is prepared by contacting (under conditions that permit binding of CRF to CRF receptors within cell and tissue samples) at least one of the matched cell or tissue samples that has not previously been contacted with any compound or salt of the invention with an experimental solution comprising the detectably-labeled preparation of the selected compound or salt at a first measured molar concentration. The control sample is prepared by in the same manner as the experimental sample and is incubated in a solution that contains the same ingredients as the experimental solution but that also contains an unlabelled preparation of the same compound or salt of the invention at a molar concentration that is greater than the first measured molar concentration.

The experimental and control samples are then washed to remove unbound detectably-labeled compound. The amount of detectably-labeled compound remaining bound to each sample is then measured and the amount of detectably-labeled compound in the experimental and control samples is compared. A comparison that indicates the detection of a greater amount of detectable label in the at least one washed experimental sample than is detected in any of the at least one washed control samples demonstrates the presence of CRF receptors in that experimental sample.

The detectably-labeled compound used in this procedure may be labeled with any detectable label, such as a radioactive label, a biological tag such as biotin (which can be detected by binding to detectably-labeled avidin), an enzyme (e.g., alkaline phosphatase, beta galactosidase, or a like enzyme that can be detected its activity in a colorimetric assay) or a directly or indirectly luminescent label. When tissue sections are used in this procedure and the detectably-labeled compound is radiolabeled, the bound, labeled compound may be detected autoradiographically to generate an autoradiogram. When autoradiography is used, the amount of detectable label in an experimental or control sample may be measured by viewing the autoradiograms and comparing the exposure density of the autoradiograms.

The present invention also pertains to methods of inhibiting the binding of CRF to CRF receptors (preferably CFR1 receptors) which methods involve contacting a solution containing a CRF antagonist compound of the invention with cells expressing CRF receptors, wherein the compound is present in the solution at a concentration sufficient to inhibit CRF binding to CRF receptors in vitro. This method includes inhibiting the binding of CRF to CRF receptors in vivo, e.g., in a patient given an amount of a compound of Formula I that would be sufficient to inhibit the binding of CRF to CRF receptors in vitro. In one embodiment, such methods are useful in treating physiological disorders associated with excess concentrations of CRF. The amount of a compound that would be sufficient to inhibit the binding of a CRF to the CRF receptor may be readily determined via a CRF receptor binding assay (see, e.g., Example 24), or from the EC₅₀ of a CRF receptor functional assay, such as a standard assay of CRF receptor mediated chemotaxis. The CRF receptors used to determine in vitro binding may be obtained from a variety of sources, for example from cells that naturally express CRF receptors, e.g. IMR32 cells or from cells expressing cloned human CRF receptors.

The present invention also pertains to methods for altering the activity of CRF receptors, said method comprising exposing cells expressing such receptors to an effective amount of a compound of the invention, wherein the compound is present in the solution at a concentration sufficient to specifically alter the signal transduction activity in response to CRF in cells expressing CRF receptors in vitro, preferred cells for this purpose are those that express high levels of CRF receptors (i.e., equal to or greater than the number of CRF1 receptors per cell found in differentiated IMR-32 human neuroblastoma cells), with IMR-32 cells being particularly preferred for testing the concentration of a compound required to alter the activity of CRF1 receptors. This method includes altering the signal transduction activity of CRF receptors in vivo, e.g., in a patient given an amount of a compound of Formula I that would be sufficient to alter the signal transduction activity in response to CRF in cells expressing CRF receptors in vitro. The amount of a compound that would be sufficient to alter the signal transduction activity in response to CRF of CRF receptors may also be determined via an assay of CRF receptor mediated signal transduction, such as an assay wherein the binding of CRF to a cell surface CRF receptor effects a changes in reporter gene expression.

The present invention also pertains to packaged pharmaceutical compositions for treating disorders responsive to CRF receptor modulation, e.g., eating disorders, depression or stress. The packaged pharmaceutical compositions include a container holding a therapeutically effective amount of at least one CRF₁ receptor modulator as described supra and instructions for using the treating disorder responsive to CRF1 receptor modulation in the patient.

Chemical Description and Terminology

The compounds herein described may have one or more asymmetric centers or planes. Compounds of the present invention containing an asymmetrically substituted atom may be isolated in optically active or racemic forms. It is well known in the art how to prepare optically active forms, such as by resolution of racemic forms (racemates), by asymmetric synthesis, or by synthesis from optically active starting materials. Resolution of the racemates can be accomplished, for example, by conventional methods such as crystallization in the presence of a resolving agent, or chromatography, using, for example a chiral HPLC column. Many geometric isomers of olefins, C═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms. All chiral (enantiomeric and diastereomeric), and racemic forms, as well as all geometric isomeric forms of a structure are intended, unless the specific stereochemistry or isomeric form is specifically indicated.

When any variable occurs more than one time in any constituent or formula for a compound, its definition at each occurrence is independent of its definition at every other occurrence. Thus, for example, if a group is shown to be substituted with 0-2 R*, then said group may optionally be substituted with up to two R* groups and R* at each occurrence is selected independently from the definition of R*. Also, combinations of substituents and/or variables are permissible only if such combinations result in stable compounds.

Formula I includes, but is not limited to, compounds of Formula IA-XXII. Formula XXIII includes, but is not limited to, compounds of Formula XXIIIA-Formula XXXVII As indicated above, various substituents of the various formulae (compounds of Formula I-Formula XXXVII) are “optionally substituted”, including Ar, Z₁, Z₂, Z₃, Z₄, Z₅, Z₄′, and Z₅′ of Formula I and Formula XXIII and subformulae thereof, and such substituents as recited in the sub-formulae such as Formula I and Formula XXIII and subformulae. The term “substituted,” as used herein, means that any one or more hydrogens on the designated atom or group is replaced with a selection from the indicated group of substituents, provided that the designated atom's normal valence is not exceeded, and that the substitution results in a stable compound. When a substituent is oxo (keto, i.e., ═O), then 2 hydrogens on an atom are replaced. The present invention is intended to include all isotopes (including radioisotopes) of atoms occurring in the present compounds.

When substituents such as Ar, Z₁, Z₂, Z₃, Z₄, Z₅, Z₄′, and Z₅′ are further substituted, they may be so substituted at one or more available positions, typically 1 to 3 or 4 positions, by one or more suitable groups such as those disclosed herein. Suitable groups that may be present on a “substituted” Ar, Z₁, Z₂, Z₃, Z₄, Z₅, Z₄′, and Z₅′or other group include e.g., halogen; cyano; hydroxyl; nitro; azido; alkanoyl (such as a C₁-C₆ alkanoyl group such as acyl or the like); carboxamido; alkyl groups (including cycloalkyl groups, having 1 to about 8 carbon atoms, preferably 1, 2, 3, 4, 5, or 6 carbon atoms); alkenyl and alkynyl groups (including groups having one or more unsaturated linkages and from 2 to about 8, preferably 2, 3, 4, 5 or 6, carbon atoms); alkoxy groups having one or more oxygen linkages and from 1 to about 8, preferably 1, 2, 3, 4, 5 or 6 carbon atoms; aryloxy such as phenoxy; alkylthio groups including those having one or more thioether linkages and from 1 to about 8 carbon atoms, preferably 1, 2, 3, 4, 5 or 6 carbon atoms; alkylsulfinyl groups including those having one or more sulfinyl linkages and from 1 to about 8 carbon atoms, preferably 1, 2, 3, 4, 5, or 6 carbon atoms; alkylsulfonyl groups including those having one or more sulfonyl linkages and from 1 to about 8 carbon atoms, preferably 1, 2, 3, 4, 5, or 6 carbon atoms; aminoalkyl groups including groups having one or more N atoms and from 1 to about 8, preferably 1, 2, 3, 4, 5 or 6, carbon atoms; carbocyclic aryl having 6 or more carbons and one or more rings, (e.g., phenyl, biphenyl, naphthyl, or the like, each ring either substituted or unsubstituted aromatic); arylalkyl having 1 to 3 separate or fused rings and from 6 to about 18 ring carbon atoms, with benzyl being a preferred arylalkyl group; arylalkoxy having 1 to 3 separate or fused rings and from 6 to about 18 ring carbon atoms, with O-benzyl being a preferred arylalkoxy group; or a saturated, unsaturated, or aromatic heterocyclic group having 1 to 3 separate or fused rings with 3 to about 8 members per ring and one or more N, O or S atoms, e.g. coumarinyl, quinolinyl, isoquinolinyl, quinazolinyl, pyridyl, pyrazinyl, pyrimidyl, furanyl, pyrrolyl, thienyl, thiazolyl, triazinyl, oxazolyl, isoxazolyl, imidazolyl, indolyl, benzofuranyl, benzothiazolyl, tetrahydrofuranyl, tetrahydropyranyl, piperidinyl, morpholinyl, piperazinyl, and pyrrolidinyl. Such heterocyclic groups may be further substituted, e.g. with hydroxy, alkyl, alkoxy, halogen and amino.

As used herein, “alkyl” is intended to include both branched and straight-chain saturated aliphatic hydrocarbon groups, having the specified number of carbon atoms. Examples of alkyl include, but are not limited to, methyl, ethyl, n-propyl, i-propyl, n-butyl, s-butyl, t-butyl, n-pentyl, and s-pentyl. Preferred alkyl groups are C₁-C₁₀ alkyl groups. Especially preferred alkyl groups are methyl, ethyl, propyl, butyl, and 3-pentyl. The term C₁₋₄ alkyl as used herein includes alkyl groups consisting of 1 to 4 carbon atoms, which may contain a cyclopropyl moiety. Suitable examples are methyl, ethyl, and cyclopropylmethyl.

The term “carbhydryl” refers to both branched and straight-chain hydrocarbon groups, which are saturated or unsaturated. In other words, a carbhydryl group may be alkyl, alkenyl or alkynyl. The number of carbon atoms may be specified as indicated above.

“Cycloalkyl” is intended to include saturated ring groups, having the specified number of carbon atoms, such as cyclopropyl, cyclobutyl, cyclopentyl, or cyclohexyl. Cycloalkyl groups typically will have 3 to about 8 ring members.

In the term “(C₃-C₇cycloalkyl)C₁-C₄alkyl”, cycloalkyl, and alkyl are as defined above, and the point of attachment is on the alkyl group. This term encompasses, but is not limited to, cyclopropylmethyl, cyclohexylmethyl, and cyclohexylmethyl.

“Alkenyl” is intended to include hydrocarbon chains of either a straight or branched configuration comprising one or more unsaturated carbon-carbon bonds, which may occur in any stable point along the chain, such as ethenyl and propenyl. Alkenyl groups typically will have 2 to about 8 carbon atoms, more typically 2 to about 6 carbon atoms.

“Alkynyl” is intended to include hydrocarbon chains of either a straight or branched configuration comprising one or more carbon-carbon triple bonds, which may occur in any stable point along the chain, such as ethynyl and propynyl. Alkynyl groups typically will have 2 to about 8 carbon atoms, more typically 2 to about 6 carbon atoms.

“Haloalkyl” is intended to include both branched and straight-chain saturated aliphatic hydrocarbon groups having the specified number of carbon atoms, substituted with 1 or more halogen atoms. Examples of haloalkyl include, but are not limited to, mono-, di-, or tri-fluoromethyl, mono-, di-, or tri-chloromethyl, mono-, di-, tri-, tetra-, or penta-fluoroethyl, and mono-, di-, tri-, tetra-, or penta-chloroethyl. Typical haloalkyl groups will have 1 to about 8 carbon atoms, more typically 1 to about 6 carbon atoms.

“Alkoxy” represents an alkyl group as defined above with the indicated number of carbon atoms attached through an oxygen bridge. Examples of alkoxy include, but are not limited to, methoxy, ethoxy, n-propoxy, i-propoxy, n-butoxy, 2-butoxy, t-butoxy, n-pentoxy, 2-pentoxy, 3-pentoxy, isopentoxy, neopentoxy, n-hexoxy, 2-hexoxy, 3-hexoxy, and 3-methylpentoxy. Alkoxy groups typically have 1 to about 8 carbon atoms, more typically 1 to about 6 carbon atoms.

“Halolkoxy” represents a haloalkyl group as defined above with the indicated number of carbon atoms attached through an oxygen bridge.

As used herein, the term “alkylthio” includes those groups having one or more thioether linkages and preferably from 1 to about 8 carbon atoms, more typically 1 to about 6 carbon atoms.

As used herein, the term “alkylsulfinyl” includes those groups having one or more sulfoxide (SO) linkage groups and typically from 1 to about 8 carbon atoms, more typically 1 to about 6 carbon atoms.

As used herein, the term “alkylsulfonyl” includes those groups having one or more sulfonyl (SO₂) linkage groups and typically from 1 to about 8 carbon atoms, more typically 1 to about 6 carbon atoms.

As used herein, the term “alkylamino” includes those groups having one or more primary, secondary and/or tertiary amine groups and typically from 1 to about 8 carbon atoms, more typically 1 to about 6 carbon atoms.

“Halo” or “halogen” as used herein refers to fluoro, chloro, bromo, or iodo; and “counter-ion” is used to represent a small, negatively charged species such as chloride, bromide, hydroxide, acetate, sulfate, and the like.

As used herein, “carbocyclic group” is intended to mean any stable 3- to 7-membered monocyclic or bicyclic or 7-to 13-membered bicyclic or tricyclic group, any of which may be saturated, partially unsaturated, or aromatic. In addition to those exemplified elsewhere herein, examples of such carbocycles include, but are not limited to, cyclopropyl, cyclobutyl, cyclopentyl, cyclohexyl, cycloheptyl, adamantyl, cyclooctyl, [3.3.0]bicyclooctanyl, [4.3.0]bicyclononanyl, [4.4.0]bicyclodecanyl, [2.2.2]bicyclooctanyl, fluorenyl, phenyl, naphthyl, indanyl, and tetrahydronaphthyl.

As used herein, the term “heterocyclic group” is intended to include saturated, partially unsaturated, or unsaturated (aromatic) groups having 1 to 3 (preferably fused) rings with 3 to about 8 members per ring at least one ring containing an atom selected from N, O or S. The nitrogen and sulfur heteroatoms may optionally be oxidized. The term or “heterocycloalkyl” is used to refer to saturated heterocyclic groups having one or more non-carbon ring atoms (e.g., N, O, S, P, Si, or the like) and a specified number of carbon atoms. Thus, a C₃₋₆heterocycloalkyl.

The heterocyclic ring may be attached to its pendant group at any heteroatom or carbon atom that results in a stable structure. The heterocyclic rings described herein may be substituted on carbon or on a nitrogen atom if the resulting compound is stable. A nitrogen in the heterocycle may optionally be quatemized. As used herein, the term “aromatic heterocyclic system” is intended to include any stable 5-to 7-membered monocyclic or 10- to 14-membered bicyclic heterocyclic aromatic ring system which comprises carbon atoms and from 1 to 4 heteroatoms independently selected from the group consisting of N, O and S. It is preferred that the total number of S and O atoms in the aromatic heterocycle is not more than 2, more preferably not more than 1.

Examples of heterocycles include, but are not limited to, those exemplified elsewhere herein and further include acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazolinyl, carbazolyl, NH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl;-1,2,5oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperazinyl, piperidinyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, and xanthenyl.

Preferred heterocyclic groups include, but are not limited to, pyridinyl, pyrimidinyl, furanyl, thienyl, pyrrolyl, pyrazolyl, pyrrolidinyl, morpholinyl, piperidinyl, piperazinyl, and imidazolyl. Also included are fused ring and spiro compounds containing, for example, the above heterocycles.

As used herein, the term “carbocyclic aryl” includes groups that contain 1 to 3 separate or fused rings and from 6 to about 18 ring atoms, without hetero atoms as ring members. Specifically preferred carbocyclic aryl groups include phenyl, and naphthyl including 1-napthyl and 2-naphthyl.

As used herein, “pharmaceutically acceptable salts” refer to derivatives of the disclosed compounds wherein the parent compound is modified by making non-toxic acid or base salts thereof, and further refers to pharmaceutically acceptable solvates of such compounds and such salts. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts and the quaternary ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, malefic, hydroxymaleic, phenylacetic, glutamic, benzoic, salicylic, mesylic, sulfanilic, 2-acetoxybenzoic, fumaric, toluenesulfonic, methanesulfonic, ethane disulfonic, oxalic, isethionic, HOOC—(CH₂)n-COOH where n is 0-4, and the like. The pharmaceutically acceptable salts of the present invention can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are preferred, where practicable. Lists of additional suitable salts may be found, e.g., in Remington's Pharmaceutical Sciences, 17th ed., Mack Publishing Company, Easton, Pa., p. 1418 (1985).

“Prodrugs” are intended to include any compounds that become compounds of Formula I when administered to a mammalian subject, e.g., upon metabolic processing of the prodrug. Examples of prodrugs include, but are not limited to, acetate, formate and benzoate and like derivatives of functional groups (such as alcohol or amine groups) in the compounds of Formula I.

Combinations of substituents and/or variables are permissible only if such combinations result in stable compounds or useful synthetic intermediates. A stable compound or stable structure is meant to imply a compound that is sufficiently robust to survive isolation from a reaction mixture, and subsequent formulation into an effective therapeutic agent. The term “therapeutically effective amount” of a compound of this invention means an amount effective, when administered to a human or non-human patient, to provide a therapeutic benefit such as an amelioration of symptoms, e.g., an amount effective to antagonize the effects of pathogenic levels of CRF or to treat the symptoms of stress disorders, affective disorder, anxiety or depression.

Pharmaceutical Preparations

The compounds of general Formula I may be administered orally, topically, transdermally, parenterally, by inhalation or spray or rectally or vaginally in dosage unit formulations containing conventional non-toxic pharmaceutically acceptable carriers, adjuvants and vehicles. The term parenteral as used herein includes subcutaneous, intravenous, intramuscular, intrathecal and like types of injection or infusion techniques. In addition, there is provided a pharmaceutical formulation comprising a compound of general Formula I and a pharmaceutically acceptable carrier. One or more compounds of general Formula I may be present in association with one or more non-toxic pharmaceutically acceptable carriers and/or diluents and/or adjuvants and if desired other active ingredients.

The pharmaceutical compositions containing compounds of general Formula I may be in a form suitable for oral use, for example, as tablets, troches, lozenges, aqueous or oily suspensions, dispersible powders or granules, emulsion, hard or soft capsules, or syrups or elixirs.

Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients that are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monosterate or glyceryl distearate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in admixture with excipients suitable for the manufacture of aqueous suspensions. Such excipients are suspending agents, for example sodium carboxymethylcellulose, methylcellulose, hydropropylmethylcellulose, sodium alginate, polyvinylpyrrolidone, gum tragacanth and gum acacia; dispersing or wetting agents may be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending the active ingredients in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide palatable oral preparations. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

Pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol, anhydrides, for example sorbitan monoleate, and condensation products of the said partial esters with ethylene oxide, for example polyoxyethylene sorbitan monoleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents. The pharmaceutical compositions may be in the form of a sterile injectable aqueous or oleaginous suspension. This suspension may be formulated according to the known art using those suitable dispersing or wetting agents and suspending agents that have been mentioned above. The sterile injectable preparation may also be sterile injectable solution or suspension in a non-toxic parentally acceptable dilutent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds of general Formula I may also be administered in the form of suppositories, e.g., for rectal administration of the drug. These compositions can be prepared by mixing the drug with a suitable non-irritating excipient that is solid at ordinary temperatures but liquid at body temperature and will therefore melt in the body to release the drug. Such materials include cocoa butter and polyethylene glycols.

Compounds of general Formula I and general Formula XXIII may be administered parenterally in a sterile medium. The drug, depending on the vehicle and concentration used, can either be suspended or dissolved in the vehicle. Advantageously, one or more adjuvants such as preservatives, buffering agents, or local anesthetics can also be present in the vehicle.

Dosage levels of the order of from about 0.05 mg to about 100 mg per kilogram of body weight per day are useful in the treatment of the above-indicated conditions, preferred dosages range from about 0.1 to about 30 mg per kg and more preferably from about 0.5 to about 5 mg per kg per subject per day. The amount of active ingredient that may be combined with the carrier materials to produce a single dosage form will vary depending upon the host treated and the particular mode of administration. Dosage unit forms will generally contain between from about 0.1 mg to about 750 mg of an active ingredient.

Frequency of dosage may also vary depending on the compound used and the particular disease treated. However, for treatment of most CNS and gastrointestinal disorders, a dosage regimen of four times daily, preferably three times daily, more preferably two times daily and most preferably once daily is contemplated. For the treatment of stress and depression a dosage regimen of 1 or 2 times daily is particularly preferred.

It will be understood, however, that the specific dose level for any particular patient will depend upon a variety of factors including the activity of the specific compound employed, the age, body weight, general health, sex, diet, time of administration, route of administration, and rate of excretion, drug combination (i.e. other drugs being used to treat the patient) and the severity of the particular disease undergoing therapy.

Preferred compounds of the invention will have certain pharmacological properties. Such properties include, but are not limited to oral bioavailability, such that the preferred oral dosage forms discussed above can provide therapeutically effective levels of the compound in vivo. Penetration of the blood brain barrier is necessary for most compounds used to treat CNS disorders, while low brain levels of compounds used to treat periphereal disorders are generally preferred.

Assays may be used to predict these desirable pharmacological properties. Assays used to predict bioavailability include transport across human intestinal cell monolayers, including Caco-2 cell monolayers. Toxicity to cultured hepatocyctes may be used to predict compound toxicity, with non-toxic compounds being preferred. Penetration of the blood brain barrier of a compound in humans may be predicted from the brain levels of the compound in laboratory animals given the compound, e.g., intravenously.

Percentage of serum protein binding may be predicted from albumin binding assays. Examples of such assays are described in a review by Oravcova, et al. (Journal of Chromatography B (1996) volume 677, pages 1-27). Preferred compounds exhibit reversible serum protein binding. Preferably this binding is less than 99%, more preferably less than 95%, even more preferably less than 90%, and most preferably less than 80%.

Frequency of administration is generally inversely proportional to the in vivo half-life of a compound. In vivo half-lives of compounds may be predicted from in vitro assays of microsomal half-life as described by Kuhnz and Gieschen (Drug Metabolism and Disposition, (1998) volume 26, pages 1120-1127). Preferred half lives are those allowing for a preferred frequency of administration.

As discussed above, preferred compounds of the invention exhibit good activity in standard in vitro CRF receptor binding assays, preferably the assay as specified in Example 24, which follows. References herein to “standard in vitro receptor binding assay” are intended to refer to protocols such as the protocol as defined in Example XXXX, which follows. Generally preferred compounds of the invention have an IC₅₀ (half-maximal inhibitory concentration) of about 1 micromolar or less, still more preferably and IC₅₀ of about 100 nanomolar or less even more preferably an IC₅₀ of about 10 nanomolar or less or even 1 nanomolar or less in such a defined standard in vitro CRF receptor binding assay.

### EXAMPLES

Preparation of Compounds

The compounds of the present invention can be prepared in a number of ways well known to one skilled in the art of organic synthesis. The compounds of the present invention can be synthesized using the methods described below, together with synthetic methods known in the art of synthetic organic chemistry, or variations thereon as appreciated by those skilled in the art. Preferred methods include but are not limited to those methods described below. Each of the references cited below are hereby incorporated herein by reference. Preferred methods for the preparation of compounds of the present invention include, but are not limited to, those described in Schemes 1 to 5. Those who are skilled in the art will recognize that the starting materials may be varied and additional steps employed to produce compounds encompassed by the present invention.

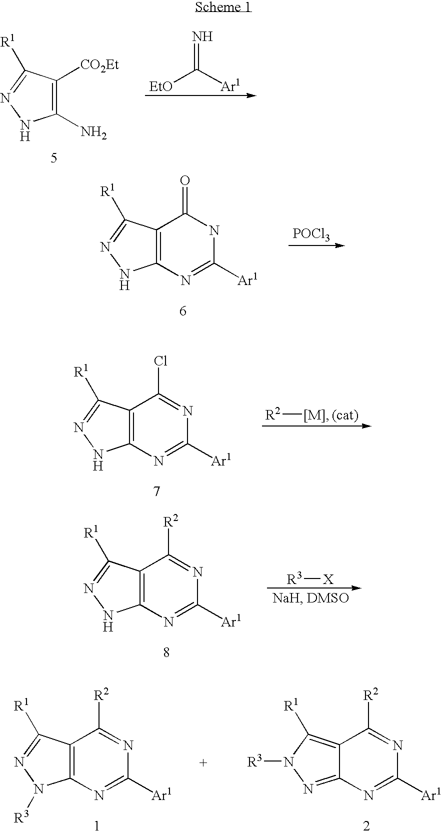

Compounds of formula 5 (Scheme 1) can be prepared according to a known literature procedure (Ref: Bull. Chem. Soc. Jap. 1969, 42, 1653-1659) and may be cyclized to pyrazolopyrimidones 6 by a number of methods known in the art, including but not limited to treatment with a suitable benzimidate in inert solvents such as but not limited to pyridine at temperatures ranging from 0° C. to 115° C. Conversion of the pyrazolopyrimidone 6 to the pyrazolopyrimidine 7 may be carried out by treatment with a chlorination agent such as but not limited to POCl₃ or SOCl₂ with or without the presence of an N,N-dialkyl aniline such as but not limited to N,N-dimethyl aniline or N,N-diethyl aniline at temperatures ranging from 0° C. to 105° C. Displacement of the chloride in pyrazolopyrimidine 7 to give 8 may be achieved by treatment with a variety of nucleophiles (R³-[M]) in the presence or absence of a transition metal catalyst. The nucleophiles may include sodium or potassium (thio)alkoxide, alkylamine, and organometallic reagent such as but not limited to alkyl Grignard reagents, alkyl or arylboronic acids or its ester, and alkyl or arylstannanes. More commonly employed reagent/catalyst pairs include alkyl or arylboronic acid/palladium(0) (Suzuki reaction; N. Miyaura and A. Suzuki, Chem. Rev. 1995, 95, 2457), aryl trialkylstannane/palladium(0) (Stille reaction; T. N. Mitchell, Synthesis 1992, 803), or arylzinc/palladium(0) and alkyl Grignard/nickel(II). Palladium(0) represents a catalytic system made of a various combination of metal/ligand pair which includes, but not limited to, tetrakis(triphenylphosphine)palladium(0), palladium(II) acetate/tri(o-tolyl)phosphine, tris(dibenzylideneacetone)dipalladium(0)/tri-tert-butylphosphine and dichloro[1,1′-bis(diphenylphosphine)ferrocene]palladium(0). Nickel(II) represents a nickel-containing catalyst such as [1,2-bis(diphenylphosphino)ethane] dichloronickel(II) and [1,3-bis(diphenylphosphino)propane]dichloronickel(II). N-alkylation of 8 to give 1 and 2 may be accomplished using a base such as but not limited to alkali metal hydride or alkali metal alkoxide in inert solvents such as but not limited to THF, DMF, or methyl sulfoxide. Alkylation may be conducted using alkyl halide, suitably bromide, iodide, tosylate or mesylate at temperatures ranging from −78° C. to 100° C. Compounds of the formula 1 and 2 may be separated by those skilled in the art by methods such as but not limited to flash chromatography, crystallization or distillation.

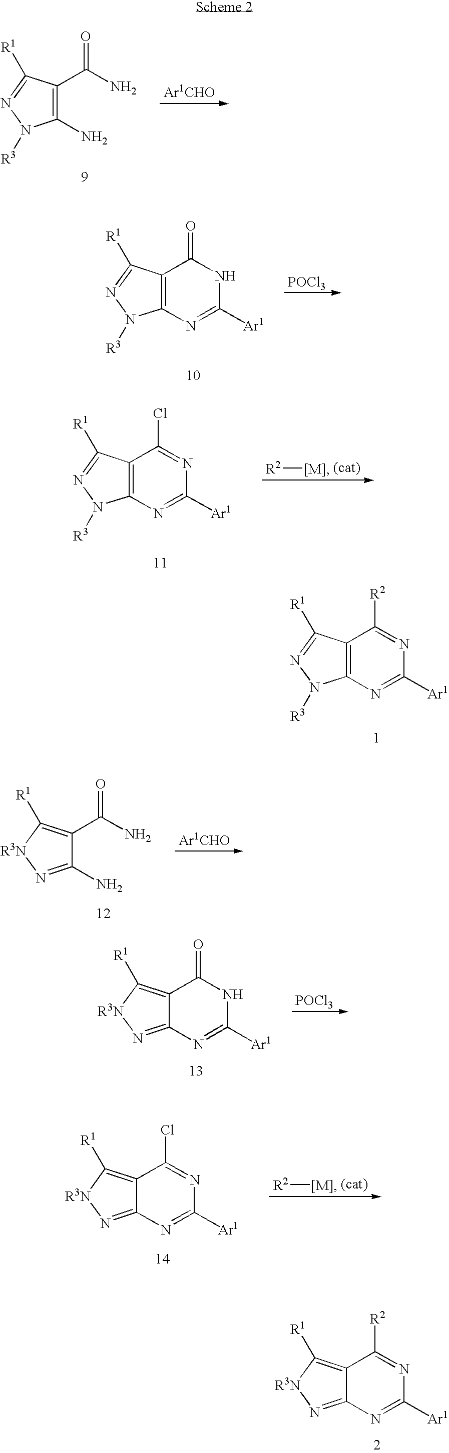

An alternative synthesis of compounds of the formula 1 and 2 is shown in Scheme 2. Compounds of the formula 9 and 12 are available commercially or can be prepared according to known literature procedures (Ref Bacon et al., WO 9628448 and Bacon et al., U.S. Pat. No. 5,294,612). Thus a suitably substituted 5-amino-pyrazolo-4-carboxamide 9 (or 12) is reacted with an excess of an appropriately substituted aldehyde in inert solvents such as but not limited to xylenes, toluene or benzene, with or without the use of an acid catalyst such as but not limited to p-toluenesulfonic acid or acetic acid at temperatures ranging from room temperature up to the boiling point of the reaction mixture to afford compounds of the formula 10 (or 13). Conversion of the pyrazolopyrimidone 10 (or 13) to the pyrazolopyrimidine 11 (or 14) may be carried out by treatment with a chlorination agent such as but not limited to POCl₃ or SOCl₂ with or without the presence of an N,N-dialkyl aniline such as but not limited to N,N-dimethyl aniline or N,N-diethyl aniline at temperatures ranging from 0° C. to 105° C. Displacement of the chloride in pyrazolopyrimidine 11 (or 14) to give 1 (or 2) may be achieved by treatment with a variety of nucleophiles (R³-[M]) in the presence or absence of a transition metal catalyst. The nucleophiles may include sodium or potassium (thio)alkoxide, alkylamine, and organometallic reagent such as but not limited to alkyl Grignard reagents, alkyl or arylboronic acids or its ester, and alkyl or arylstannanes. More commonly employed reagent/catalyst pairs include alkyl or arylboronic acid/palladium(0) (Suzuki reaction; N. Miyaura and A. Suzuki, Chem. Rev. 1995, 95, 2457), aryl trialkylstannane/palladium(0) (Stille reaction; T. N. Mitchell, Synthesis 1992, 803), or arylzinc/palladium(0) and alkyl Grignard/nickel(II). Palladium(0) represents a catalytic system made of a various combination of metal/ligand pair which includes, but not limited to, tetrakis(triphenylphosphine)palladium(0), palladium(II) acetate/tri(o-tolyl)phosphine, tris(dibenzylideneacetone)dipalladium(0)/tri-tert-butylphosphine and dichloro[1,1′-bis(diphenylphosphine)ferrocene]palladium(0). Nickel(II) represents a nickel-containing catalyst such as [1,2-bis(diphenylphosphino)ethane] dichloronickel(II) and [1,3-bis(diphenylphosphino)propane]dichloronickel(II).

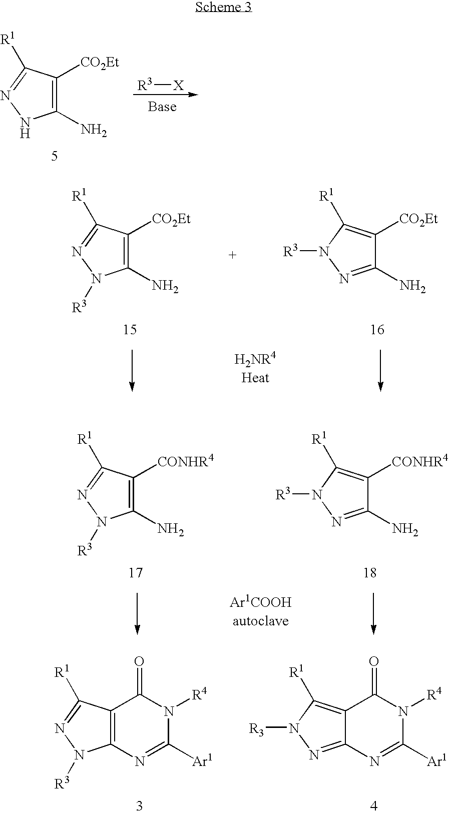

Compounds of the formula 3 or 4 may be prepared by the route outlined in Scheme 3. Pyrazoles V (Ref: Bull. Chem. Soc. Jap. 1969, 42, 1653-1659) are N-alkylated under a variety of different conditions to give mixtures of compounds of the formula 15 and 16. Alkylation may be conducted using alkyl halide, suitably bromide, iodide, tosylate or mesylate at temperatures ranging from −78° C. to 100° C. using bases such as but not limited to alkali metal carbonates or alkali metal hydroxides, alkali metal hydrides or alkali metal alkoxides in inert solvents such as but not limited to THF, DMF, or methyl sulfoxide. Alkylation may also be conducted under solid-liquid phase-transfer-catalyzed conditions such as but not limited to the use of alkyl halide, suitably bromide, iodide, tosylate or mesylate in inert solvents such as but not limited to xylenes, toluene or benzene using bases such as but not limited to alkali metal carbonates and phase transfer catalysts such as but not limited to Adogen 464. Compounds of the formula 15 and 16 may be separated by those skilled in the art by methods such as but not limited to flash chromatography, crystallization or distillation. Conversion of the esters 15 (or 16) to the amides 17 (or 18) may be carried out by treatment with a large excess of a primary amine at or above the refluxing temperature of the primary amine (the use of a suitable reaction vessel such as a sealed tube may be necessary). Cyclization of the amides 17 (or 18) may be carried out by treatment with a large excess of the appropriately substituted benzoic acid at temperatures ranging from room temperature to 250° C. in an autoclave.

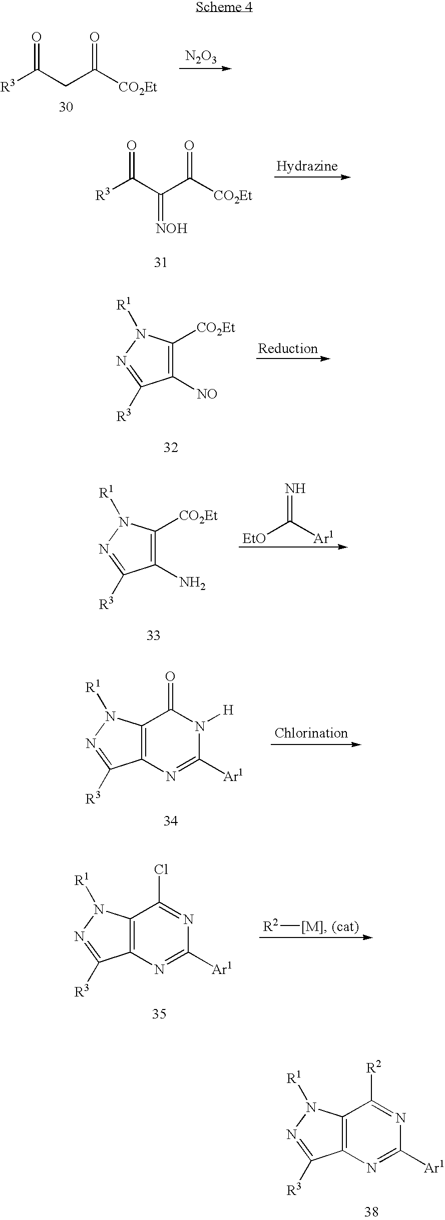

Conversion of the pyruvate 30 to the oxime 31 may be carried out with N₂O₃ generated by treatment of sodium nitrate with but not limited to hydrochloric acid or acetic acid (Scheme 4). Cyclization of the oxime 31 to the pyrazole 32 may be carried out by a number of methods known in the art, including the use of hydrazine or a monosubstituted hydrazine such as but not limited to hydrazine, alkylhydrazine or phenylhydrazine in solvents such as but not limited to methanol or ethanol. Reduction of the nitroso group in 32 may be accomplished by a variety of methods known in the art, including hydrogenation with hydrogen and transition metal catalysts or the use of sodium hydrosulfite in aqueous solutions to give the amine 33. Compounds of formula 33, which can also be prepared by known literature procedures (Ref: Journal of Organic Chemistry 1975, 40, 2825-2830 and Bull. Chem. Soc. Jpn. 1979, 52, 208-211) may be cyclized to pyrazolopyrimidone 34 by a number of methods known in the art, including but not limited to treatment with a suitable benzimidate in inert solvents such as but not limited to pyridine at temperatures ranging from 0° C. to 115° C. Conversion of the pyrazolopyrimidone 34 to the pyrazolopyrimidine 35 may be carried out by treatment of with a chlorination agent such as but not limited to POCl₃, in the presence of an N,N-dialkyl aniline such as but not limited to N,N-dimethyl aniline or NAN-diethyl aniline at temperatures ranging from 0° C. to 105° C. Displacement of the chloride in pyrazolopyrimidine 35 to give 38 may be achieved by treatment with a variety of nucleophiles (R₂-[M]) in the presence or absence of a transition metal catalyst. The nucleophiles may include sodium or potassium (thio)alkoxide, alkylamine, and organometallic reagent such as but not limited to alkyl Grignard reagents, alkyl or arylboronic acids or its ester, and alkyl or aryistannanes. More commonly employed reagent/catalyst pairs include alkyl or arylboronic acid/palladium(0) (Suzuki reaction; N. Miyaura and A. Suzuki, Chem. Rev. 1995, 95, 2457), aryl trialkylstannane/palladium(0) (Stille reaction; T. N. Mitchell, Synthesis 1992, 803), or arylzinc/palladium(0) and alkyl Grignard/nickel(II). Palladium(0) represents a catalytic system made of a various combination of metal/ligand pair which includes, but not limited to, tetrakis(triphenylphosphine)palladium(0), palladium(II) acetate/tri(o-tolyl)phosphine, tris(dibenzylideneacetone)dipalladium(0)/tri-tert-butylphosphine and dichloro[1,1′-bis(diphenylphosphine)ferrocene]palladium(0). Nickel(II) represents a nickel-containing catalyst such as [1,2-bis(diphenylphosphino)ethane] dichloronickel(II) and [1,3-bis(diphenylphosphino)propane]dichloronickel(II).

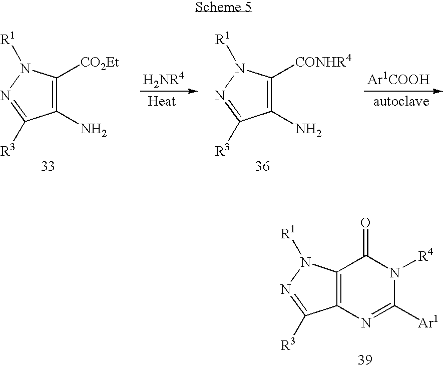

Compounds of formula 39 may be prepared by the route shown in Scheme 5. Treatment of pyrazole 33 with a large excess of a primary amine at or above the refluxing temperature of the primary amine (the use of a suitable reaction vessel such as a sealed tube may be necessary) gives compounds of formula 36. Cyclization of 36 to 39 may be carried out by treatment with a large excess of the appropriately substituted benzoic acid at temperatures ranging from room temperature to 250° C. in an autoclave.

### Example 1

The following compounds are prepared using the methods given in reaction Schemes 1, 2 and 3.

TABLE III

| | |
|---|---|
| | I |
| 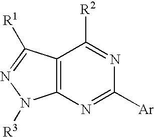 | |
| | II |
| 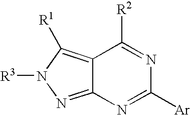 | |
| | III |
| 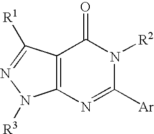 | |
| | III |
| 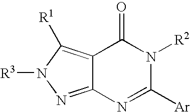 | |

| | Base | | | | |
|---|---|---|---|---|---|
| Ex # | Structure | R¹ | R² | R³ | Ar |
| 101 | I | 2-Ethylpropyl | CH₃ | H | 2,4,6- |
| | | | | | Trimethylphenyl |
| 102 | I | 2-Ethylpropyl | CH₃ | CH₃ | 2,4,6- |
| | | | | | Trimethylphenyl |
| 103 | I | 2-Ethylpropyl | CH₃ | H | 6-Fluoro-2,4- |
| | | | | | dimethoxyphenyl |
| 104 | I | 2-Ethylpropyl | CH₃ | CH₃ | 6-Fluoro-2,4- |
| | | | | | dimethoxyphenyl |
| 105 | I | 2-Ethylpropyl | CH₃ | H | 2,6-Dimethoxy-4- |
| | | | | | chlorophenyl |
| 106 | I | 2-Ethylpropyl | CH₃ | CH₃ | 2,6-Dimethoxy-4- |
| | | | | | chlorophenyl |
| 107 | I | 2-Ethylpropyl | CH₃ | H | 2,6-Dichloro-4- |
| | | | | | trifluoromethylphenyl |
| 108 | I | 2-Ethylpropyl | CH₃ | CH₃ | 2,6-Dichloro-4- |
| | | | | | trifluoromethylphenyl |
| 109 | I | 2-Ethylpropyl | CH₃ | H | 2-Methoxy-4,6- |
| | | | | | dimethylphenyl |
| 110 | I | 2-Ethylpropyl | CH₃ | CH₃ | 2-Methoxy-4,6- |
| | | | | | dimethylphenyl |
| 111 | I | 2-Ethylpropyl | CH₂CH₃ | CH₃ | 2-Methoxy-4,6- |
| | | | | | dimethylphenyl |
| 112 | I | 2-Ethylpropyl | OCH₃ | CH₃ | 2-Methoxy-4,6- |
| | | | | | dimethylphenyl |
| 113 | II | 2-Ethylpropyl | CH₃ | H | 2,4,6- |
| | | | | | Trimethylphenyl |
| 114 | II | 2-Ethylpropyl | CH₃ | CH₃ | 2,4,6- |
| | | | | | Trimethylphenyl |
| 115 | II | 2-Ethylpropyl | CH₃ | H | 6-Fluoro-2,4- |
| | | | | | dimethoxyphenyl |
| 116 | II | 2-Ethylpropyl | CH₃ | CH₃ | 6-Fluoro-2,4- |
| | | | | | dimethoxyphenyl |
| 117 | II | 2-Ethylpropyl | CH₃ | H | 2,6-Dimethoxy-4- |
| | | | | | chlorophenyl |
| 118 | II | 2-Ethylpropyl | CH₃ | CH₃ | 2,6-Dimethoxy-4- |
| | | | | | chlorophenyl |
| 119 | II | 2-Ethylpropyl | CH₃ | H | 2,6-Dimethoxy-4- |
| | | | | | trifluoromethylphenyl |
| 120 | II | 2-Ethylpropyl | CH₃ | CH₃ | 2,6-Dichloro-4- |
| | | | | | trifluoromethylphenyl |
| 121 | II | 2-Ethylpropyl | CH₂CH₃ | CH₃ | 2-Methoxy-4,6- |
| | | | | | dimethylphenyl |
| 122 | II | 2-Ethylpropyl | OCH₃ | CH₃ | 2-Methoxy-4,6- |
| | | | | | dimethylphenyl |
| 123 | III | 2-Ethylpropyl | CH₃ | H | 2,4,6- |
| | | | | | Trimethylphenyl |
| 124 | III | 2-Ethylpropyl | CH₃ | CH₃ | 2,4,6- |
| | | | | | Trimethylphenyl |
| 125 | III | 2-Ethylpropyl | CH₃ | H | 6-Fluoro-2,4- |
| | | | | | dimethoxyphenyl |
| 126 | III | 2-Ethylpropyl | CH₃ | CH₃ | 6-Fluoro-2,4- |
| | | | | | dimethoxyphenyl |
| 127 | III | 2-Ethylpropyl | CH₃ | H | 2,6-Dimethoxy-4- |
| | | | | | chlorophenyl |
| 128 | III | 2-Ethylpropyl | CH₃ | CH₃ | 2,6-Dimethoxy-4- |
| | | | | | chlorophenyl |
| 129 | III | 2-Ethylpropyl | CH₃ | H | 2,6-Dimethoxy-4- |
| | | | | | trifluoromethylphenyl |
| 130 | III | 2-Ethylpropyl | CH₃ | CH₃ | 2,6-Dichloro-4- |
| | | | | | trifluoromethylphenyl |
| 131 | III | 2-Ethylpropyl | CH₂CH₃ | CH₃ | 2-Methoxy-4,6- |
| | | | | | dimethylphenyl |
| 132 | IV | 2-Ethylpropyl | CH₃ | H | 2,4,6- |
| | | | | | Trimethylphenyl |
| 133 | IV | 2-Ethylpropyl | CH₃ | CH₃ | 2,4,6- |
| | | | | | Trimethylphenyl |
| 134 | IV | 2-Ethylpropyl | CH₃ | H | 6-Fluoro-2,4- |
| | | | | | dimethoxyphenyl |
| 135 | IV | 2-Ethylpropyl | CH₃ | CH₃ | 6-Fluoro-2,4- |
| | | | | | dimethoxyphenyl |
| 136 | IV | 2-Ethylpropyl | CH₃ | H | 2,6-Dimethoxy-4- |
| | | | | | chlorophenyl |
| 137 | IV | 2-Ethylpropyl | CH₃ | CH₃ | 2,6-Dimethoxy-4- |
| | | | | | chlorophenyl |
| 138 | IV | 2-Ethylpropyl | CH₃ | H | 2,6-Dichloro-4- |
| | | | | | trifluoromethylphenyl |
| 139 | IV | 2-Ethylpropyl | CH₃ | CH₃ | 2,6-Dichloro-4- |
| | | | | | trifluoromethylphenyl |
| 140 | IV | 2-Ethylpropyl | CH₂CH₃ | CH₃ | 2-Methoxy-4,6- |
| | | | | | dimethylphenyl |
| 141 | I | 2-Ethylbutyl | CH₃ | H | 2,4,6- |
| | | | | | Trimethylphenyl |
| 142 | I | 2-Ethylbutyl | CH₃ | CH₃ | 2,4,6- |
| | | | | | Trimethylphenyl |
| 143 | I | 2-Ethylbutyl | CH₃ | H | 6-Fluoro- |
| | | | | | 2,4dimethoxyphenyl |
| 144 | I | 2-Ethylbutyl | CH₃ | CH₃ | 6-Fluoro-2,4- |
| | | | | | dimethoxyphenyl |
| 145 | I | 2-Ethylbutyl | CH₃ | H | 2,6-Dimethoxy-4- |
| | | | | | chlorophenyl |
| 146 | I | 2-Ethylbutyl | CH₃ | CH₃ | 2,6-Dimethoxy-4- |
| | | | | | chlorophenyl |
| 147 | I | 2-Ethylbutyl | CH₃ | H | 2,6-Dichloro-4- |
| | | | | | trifluoromethylphenyl |
| 148 | I | 2-Ethylbutyl | CH₃ | CH₃ | 2,6-Dichloro-4- |
| | | | | | trifluoromethylphenyl |
| 149 | I | 2-Ethylbutyl | CH₃ | H | 2-Methoxy-4,6- |
| | | | | | dimethylphenyl |
| 150 | I | 2-Ethylbutyl | CH₃ | CH₃ | 2-Methoxy-4,6- |
| | | | | | dimethylphenyl |
| 151 | I | 2-Ethylbutyl | CH₂CH₃ | CH₃ | 2-Methoxy-4,6- |
| | | | | | dimethylphenyl |
| 152 | I | 2-Ethylbutyl | OCH₃ | CH₃ | 2-Methoxy-4,6- |
| | | | | | dimethylphenyl |
| 153 | II | 2-Ethylbutyl | CH₃ | H | 2,4,6- |
| | | | | | Trimethylphenyl |
| 154 | II | 2-Ethylbutyl | CH₃ | CH₃ | 2,4,6- |
| | | | | | Trimethylphenyl |
| 155 | II | 2-Ethylbutyl | CH₃ | H | 6-Fluoro-2,4- |
| | | | | | dimethoxyphenyl |
| 156 | II | 2-Ethylbutyl | CH₃ | CH₃ | 6-Fluoro-2,4- |
| | | | | | dimethoxyphenyl |
| 157 | II | 2-Ethylbutyl | CH₃ | H | 2,6-Dimethoxy-4- |
| | | | | | chlorophenyl |
| 158 | II | 2-Ethylbutyl | CH₃ | CH₃ | 2,6-Dimethoxy-4- |
| | | | | | chlorophenyl |
| 159 | II | 2-Ethylbutyl | CH₃ | H | 2,6-Dichloro-4- |
| | | | | | trifluoromethylphenyl |
| 160 | II | 2-Ethylbutyl | CH₃ | CH₃ | 2,6-Dichloro-4- |
| | | | | | trifluoromethylphenyl |
| 161 | II | 2-Ethylbutyl | CH₂CH₃ | CH₃ | 2-Methoxy-4,6- |
| | | | | | dimethylphenyl |
| 162 | II | 2-Ethylbutyl | OCH₃ | CH₃ | 2-Methoxy-4,6- |
| | | | | | dimethylphenyl |
| 163 | III | 2-Ethylbutyl | CH₃ | H | 2,4,6- |
| | | | | | Trimethylphenyl |
| 164 | III | 2-Ethylbutyl | CH₃ | CH₃ | 2,4,6- |
| | | | | | Trimethylphenyl |
| 165 | III | 2-Ethylbutyl | CH₃ | H | 6-Fluoro-2,4- |
| | | | | | dimethoxyphenyl |
| 166 | III | 2-Ethylbutyl | CH₃ | CH₃ | 6-Fluoro-2,4- |
| | | | | | dimethoxyphenyl |
| 167 | III | 2-Ethylbutyl | CH₃ | H | 2,6-Dimethoxy-4- |
| | | | | | chlorophenyl |
| 168 | III | 2-Ethylbutyl | CH₃ | CH₃ | 2,6-Dimethoxy-4- |
| | | | | | chlorophenyl |
| 169 | III | 2-Ethylbutyl | CH₃ | H | 2,6-Dichloro-4- |
| | | | | | trifluoromethylphenyl |
| 170 | III | 2-Ethylbutyl | CH₃ | CH₃ | 2,6-Dichloro-4- |
| | | | | | trifluoromethylphenyl |
| 171 | III | 2-Ethylbutyl | CH₂CH₃ | CH₃ | 2-Methoxy-4,6- |
| | | | | | dimethylphenyl |
| 172 | III | 2-Ethylbutyl | CH₂CH₃ | CH₃ | 2,,4- |
| | | | | | Dimethoxyphenyl |
| 173 | III | 2-Ethylbutyl | CH₂CH₃ | CH₃ | 2-Methoxy-4- |
| | | | | | trifluoromethoxy- |
| | | | | | phenyl |
| 174 | III | 2-Ethylbutyl | CH₂CH₃ | CH₃ | 2-Chloro-4- |
| | | | | | trifluoromethylphenyl |
| 175 | IV | 2-Ethylbutyl | CH₃ | H | 2,4,6- |
| | | | | | Trimethylphenyl |
| 176 | IV | 2-Ethylbutyl | CH₃ | CH₃ | 2,4,6- |
| | | | | | Trimethylphenyl |
| 177 | IV | 2-Ethylbutyl | CH₃ | H | 6-Fluoro-2,4- |
| | | | | | dimethoxyphenyl |
| 178 | IV | 2-Ethylbutyl | CH₃ | CH₃ | 6-Fluoro-2,4- |
| | | | | | dimethoxyphenyl |
| 179 | IV | 2-Ethylbutyl | CH₃ | H | 2,6-Dimethoxy-4- |
| | | | | | chlorophenyl |
| 180 | IV | 2-Ethylbutyl | CH₃ | CH₃ | 2,6-Dimethoxy-4- |
| | | | | | chlorophenyl |
| 181 | IV | 2-Ethylbutyl | CH₃ | H | 2,6-Dichloro-4- |
| | | | | | trifluoromethylphenyl |
| 182 | IV | 2-Ethylbutyl | CH₃ | CH₃ | 2,6-Dichloro-4- |
| | | | | | trifluoromethylphenyl |
| 183 | IV | 2-Ethylbutyl | CH₂CH₃ | CH₃ | 2-Methoxy-4,6- |
| | | | | | dimethylphenyl |
| 184 | IV | 2-Ethylbutyl | CH₂CH₃ | CH₃ | 2,4-Dimethoxyphenyl |
| 185 | IV | 2-Ethylbutyl | CH₂CH₃ | CH₃ | 2-Methoxy-4- |
| | | | | | trifluoromethoxy- |
| | | | | | phenyl |
| 186 | IV | 2-Ethylbutyl | CH₂CH₃ | CH₃ | 2-Chloro-4- |
| | | | | | trifluoromethylphenyl |
| 187 | IV | 2-Ethylbutyl | CH₂CH₃ | H | 2,4-Dimethoxyphenyl |
| 188 | IV | 2-Ethylbutyl | CH₂CH₃ | H | 2-Methoxy-4- |
| | | | | | trifluoromethoxy- |
| | | | | | phenyl |
| 189 | IV | 2-Ethylbutyl | CH₂CH₃ | H | 2-Chloro-4- |
| | | | | | trifluoromethylphenyl |

The following compounds can be prepared using the methods given in reaction Schemes 4 and 5.

TABLE IV

| | |
|---|---|
| 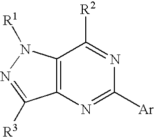 | 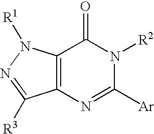 |

| | Base | | | | |
|---|---|---|---|---|---|
| Ex # | Structure | R¹ | R² | R³ | Ar |
| 201 | I | 2-Ethylpropyl | CH₃ | H | 2,4,6- |
| | | | | | Trimethylphenyl |
| 202 | I | 2-Ethylpropyl | CH₃ | CH₃ | 2,4,6- |
| | | | | | Trimethylphenyl |
| 203 | I | 2-Ethylpropyl | CH₃ | H | 6-Fluoro-2,4- |
| | | | | | dimethoxyphenyl |
| 204 | I | 2-Ethylpropyl | CH₃ | CH₃ | 6-Fluoro-2,4- |
| | | | | | dimethoxyphenyl |
| 205 | I | 2-Ethylpropyl | CH₃ | H | 2,6-Dimethoxy-4- |
| | | | | | chlorophenyl |
| 206 | I | 2-Ethylpropyl | CH₃ | CH₃ | 2,6-Dimethoxy-4- |
| | | | | | chlorophenyl |
| 207 | I | 2-Ethylpropyl | CH₃ | H | 2,6-Dimethoxy-4- |
| | | | | | trifluoromethylphenyl |
| 208 | I | 2-Ethylpropyl | CH₃ | CH₃ | 2,6-Dichloro-4- |
| | | | | | trifluoromethylphenyl |
| 209 | I | 2-Ethylpropyl | CH₃ | H | 2-Methoxy-4,6- |
| | | | | | dimethylphenyl |
| 210 | I | 2-Ethylpropyl | CH₃ | CH₃ | 2-Methoxy-4,6- |
| | | | | | dimethylphenyl |
| 211 | I | 2-Ethylpropyl | CH₂CH₃ | CH₃ | 2-Methoxy-4,6- |
| | | | | | dimethylphenyl |
| 212 | I | 2-Ethylpropyl | OCH₃ | CH₃ | 2-Methoxy-4,6- |
| | | | | | dimethylphenyl |
| 213 | II | 2-Ethylpropyl | CH₃ | H | 2,4,6- |
| | | | | | Trimethylphenyl |
| 214 | II | 2-Ethylpropyl | CH₃ | CH₃ | 2,4,6- |
| | | | | | Trimethylphenyl |
| 215 | II | 2-Ethylpropyl | CH₃ | H | 6-Fluoro-2,4- |
| | | | | | dimethoxyphenyl |
| 216 | II | 2-Ethylpropyl | CH₃ | CH₃ | 6-Fluoro-2,4- |
| | | | | | dimethoxyphenyl |
| 217 | II | 2-Ethylpropyl | CH₃ | H | 2,6-Dimethoxy-4- |
| | | | | | chlorophenyl |
| 218 | II | 2-Ethylpropyl | CH₃ | CH₃ | 2,6-Dimethoxy-4- |
| | | | | | chlorophenyl |
| 219 | II | 2-Ethylpropyl | CH₃ | H | 2,6-Dichloro-4- |
| | | | | | trifluoromethylphenyl |
| 220 | II | 2-Ethylpropyl | CH₃ | CH₃ | 2,6-Dichloro-4- |
| | | | | | trifluoromethylphenyl |
| 221 | II | 2-Ethylpropyl | CH₂CH₃ | CH₃ | 2-Methoxy-4,6- |
| | | | | | dimethylphenyl |
| 222 | II | 2-Ethylpropyl | OCH₃ | CH₃ | 2-Methoxy-4,6- |
| | | | | | dimethylphenyl |
| 223 | III | 2-Ethylpropyl | CH₃ | H | 2,4,6- |
| | | | | | Trimethylphenyl |
| 224 | III | 2-Ethylpropyl | CH₃ | CH₃ | 2,4,6- |
| | | | | | Trimethylphenyl |
| 225 | III | 2-Ethylpropyl | CH₃ | H | 6-Fluoro-2,4- |
| | | | | | dimethoxyphenyl |
| 226 | III | 2-Ethylpropyl | CH₃ | CH₃ | 6-Fluoro-2,4- |
| | | | | | dimethoxyphenyl |
| 227 | III | 2-Ethylpropyl | CH₃ | H | 2,6-Dimethoxy-4- |
| | | | | | chlorophenyl |
| 228 | III | 2-Ethylpropyl | CH₃ | CH₃ | 2,6-Dimethoxy-4- |
| | | | | | chlorophenyl |
| 229 | III | 2-Ethylpropyl | CH₃ | H | 2,6-Dichloro-4- |
| | | | | | trifluoromethylphenyl |
| 230 | III | 2-Ethylpropyl | CH₃ | CH₃ | 2,6-Dichloro-4- |
| | | | | | trifluoromethylphenyl |
| 231 | III | 2-Ethylpropyl | CH₂CH₃ | CH₃ | 2-Methoxy-4,6- |
| | | | | | dimethylphenyl |
| 232 | IV | 2-Ethylpropyl | CH₃ | H | 2,4,6- |
| | | | | | Trimethylphenyl |
| 233 | IV | 2-Ethylpropyl | CH₃ | CH₃ | 2,4,6- |
| | | | | | Trimethylphenyl |
| 234 | IV | 2-Ethylpropyl | CH₃ | H | 6-Fluoro-2,4- |
| | | | | | dimethoxyphenyl |
| 235 | IV | 2-Ethylpropyl | CH₃ | CH₃ | 6-Fluoro-2,4- |
| | | | | | dimethoxyphenyl |
| 236 | IV | 2-Ethylpropyl | CH₃ | H | 2,6-Dimethoxy-4- |
| | | | | | chlorophenyl |
| 237 | IV | 2-Ethylpropyl | CH₃ | CH₃ | 2,6-Dimethoxy-4- |
| | | | | | chlorophenyl |
| 238 | IV | 2-Ethylpropyl | CH₃ | H | 2,6-Dichloro-4- |
| | | | | | trifluoromethylphenyl |
| 239 | IV | 2-Ethylpropyl | CH₃ | CH₃ | 2,6-Dichloro-4- |
| | | | | | trifluoromethylphenyl |
| 240 | IV | 2-Ethylpropyl | CH₂CH₃ | CH₃ | 2-Methoxy-4,6- |
| | | | | | dimethylphenyl |
| 241 | I | 2-Ethylbutyl | CH₃ | H | 2,4,6- |
| | | | | | Trimethylphenyl |
| 242 | I | 2-Ethylbutyl | CH₃ | CH₃ | 2,4,6- |
| | | | | | Trimethylphenyl |
| 243 | I | 2-Ethylbutyl | CH₃ | H | 6-Fluoro-2,4- |
| | | | | | dimethoxyphenyl |
| 244 | I | 2-Ethylbutyl | CH₃ | CH₃ | 6-Fluoro-2,4- |
| | | | | | dimethoxyphenyl |
| 245 | I | 2-Ethylbutyl | CH₃ | H | 2,6-Dimethoxy-4- |
| | | | | | chlorophenyl |
| 246 | I | 2-Ethylbutyl | CH₃ | CH₃ | 2,6-Dimethoxy-4- |
| | | | | | chlorophenyl |
| 247 | I | 2-Ethylbutyl | CH₃ | H | 2,6-Dichloro-4- |
| | | | | | trifluoromethylphenyl |
| 248 | I | 2-Ethylbutyl | CH₃ | CH₃ | 2,6-Dichloro-4- |
| | | | | | trifluoromethylphenyl |
| 249 | I | 2-Ethylbutyl | CH₃ | H | 2-Methoxy-4,6- |
| | | | | | dimethylphenyl |
| 250 | I | 2-Ethylbutyl | CH₃ | CH₃ | 2-Methoxy-4,6- |
| | | | | | dimethylphenyl |
| 251 | I | 2-Ethylbutyl | CH₂CH₃ | CH₃ | 2-Methoxy-4,6- |
| | | | | | dimethylphenyl |
| 252 | I | 2-Ethylbutyl | OCH₃ | CH₃ | 2-Methoxy-4,6- |
| | | | | | dimethylphenyl |
| 253 | II | 2-Ethylbutyl | CH₃ | H | 2,4,6- |
| | | | | | Trimethylphenyl |
| 254 | II | 2-Ethylbutyl | CH₃ | CH₃ | 2,4,6- |
| | | | | | Trimethylphenyl |
| 255 | II | 2-Ethylbutyl | CH₃ | H | 6-Fluoro-2,4- |
| | | | | | dimethoxyphenyl |
| 256 | II | 2-Ethylbutyl | CH₃ | CH₃ | 6-Fluoro-2,4- |
| | | | | | dimethoxyphenyl |
| 257 | II | 2-Ethylbutyl | CH₃ | H | 2,6-Dimethoxy-4- |
| | | | | | chlorophenyl |
| 258 | II | 2-Ethylbutyl | CH₃ | CH₃ | 2,6-Dimethoxy-4- |
| | | | | | chlorophenyl |
| 259 | II | 2-Ethylbutyl | CH₃ | H | 2,6-Dichloro-4- |
| | | | | | trifluoromethylphenyl |
| 260 | II | 2-Ethylbutyl | CH₃ | CH₃ | 2,6-Dichloro-4- |
| | | | | | trifluoromethylphenyl |
| 261 | II | 2-Ethylbutyl | CH₂CH₃ | CH₃ | 2-Methoxy-4,6- |
| | | | | | dimethylphenyl |
| 262 | II | 2-Ethylbutyl | OCH₃ | CH₃ | 2-Methoxy-4,6- |
| | | | | | dimethylphenyl |

### Example 2

### Preparation of Boronic Acid Intermediates

### A. Synthesis of 2-(Dimethylamino)-4-methoxypyridin-5-boronic acid and 2-(Dimethylamino)-4-isopropoxypyridin-5-boronic acid

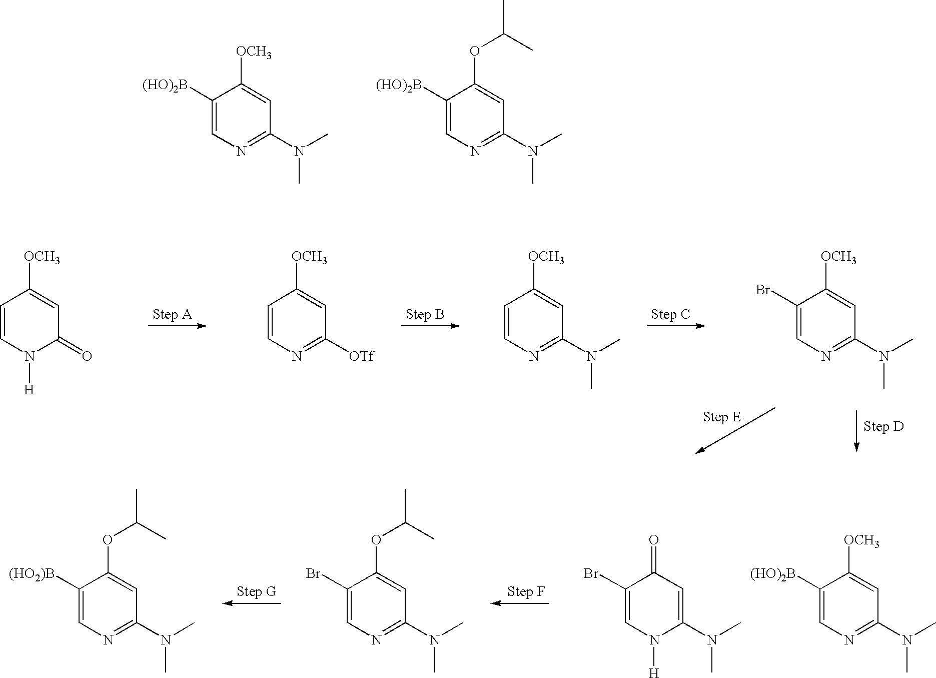 Step A

To a stirred solution of 4-methoxy-1H-pyridin-2-one (Walters and Shay, Tetrahedron Letters 36 (1995), 7575) in methylene chloride (30 mL) at 0° C. is added triflic anhydride (12.9 g) followed by triethylamine (8.4 g). The reaction mixture is stirred for 20 min and then allowed to warm to room temperature. The volatile components are evaporated under vacuum and then the residue is dissolved in EtOAc and washed consecutively with aqueous sodium bicarbonate, water and brine solution. The organic phase is separated, dried and evaporated under vacuum to give trifluoro-methanesulfonic acid 4-methoxy-pyridin-2-yl ester. It is used in the next step without further purification.

Step B

Trifluoro-methanesulfonic acid 4-methoxy-pyridin-2-yl ester (0.5 g) and dimethylamine (2.4 mL of 2M in THF) are dissolved in DMSO (7 mL) and warmed overnight at 40° C. EtOAc is added to the reaction mixture and it is washed with brine solution. The organic phase is separated, dried, and evaporated under vacuum. Silica gel purification gives (4-methoxypyridin-2-yl)dimethylamine. It is used in the next step without further purification.

Step C

N-bromosuccinimide (1.75 g) is added portionwise to a solution of (4-methoxy-pyridin-2-yl)dimethylamine (1.5 g) at 0° C. in chloroform (30 mL). After 30 min water (4 mL) is added to the reaction mixture and it is extracted three times with methylene chloride. The combined organic phase is separated, dried and evaporated under vacuum. Silica gel purification gives (5-bromo-4-methoxy-pyridin-2-yl)dimethylamine. LCMS: Rt 1.20 min m/z 231.03(M+H)⁺.

Step D

To a mixture of n-butyllithium (2.68 mL of 1.6M in hexanes) and toluene (7.4 mL) at -65° C. is added dropwise (5-bromo-4-methoxy-pyridin-2-yl)dimethylamine (0.9 g) in toluene (4 mL). The reaction mixture is stirred in the cold for 30 min and the THF (1.6 mL) is added and stirring is continued for a further 15 min. Triisopropylborate (1.5 g) is then added slowly and stirring is continued for 45 min. The reaction mixture is then allowed to warm to room temperature overnight and 1N HCl (10 mL) is added. The aqueous layer is separated and the organic phase is washed consecutively with 1N HCl and water. The combined aqueous phase was adjusted to pH7 with solid sodium bicarbonate and extracted with 1:1 EtOAc/THF. The organic phase is separated, dried and evaporated under vacuum to give 2-(dimethylamino)-4-methoxypyridin-5-boronic acid. LCMS: Rt 2.56 min m/z 197.12(M+H)⁺

Step E

(5-Bromo-4-methoxy-pyridin-2-yl)dimethylamine (2 g) and sodium thiomethoxide (3 g) in DMF (50 mL) are heated at 110° C. overnight in a sealed tube. This mixture containing 5-bromo-2-dimethylamino-1H-pyridin-4-one is taken to the next step without purification. LCMS: Rt 1.83 min m/z 216.9(M+H)⁺

Step F

To the mixture containing 5-bromo-2-dimethylamino-1H-pyridin-4-one is added isopropyl iodide (1 mL) and potassium carbonate (2.4 g). Heating is continued again at 70° C. overnight and then the reaction mixture is filtered through Celite. The Celite is washed well with EtOAc and then the combined filtrate is washed consecutively with water and brine. The organic phase is then separated, dried and evaporated under vacuum. Purification over silica gel gives 5-bromo-4-isopropoxy-pyridin-2-yl)dimethyl-amine. LCMS: Rt 1.92 min m/z 259.05(M+H)⁺

Step G

Analogous to the preparation of 2-(dimethylamino)-4-methoxypyridin-5-boronic acid in Step D, 5-bromo-4-isopropoxy-pyridin-2-yl)dimethyl-amine is treated successively with n-butyllithium and triisopropylborate to give 2-(dimethy-lamino)-4-isopropoxypyridin-5-boronic acid. LCMS: Rt 1.87 min m/z 225.1(M+H)⁺

### B. Synthesis of 2-(Diethylamino)-4-ethylpyridin-5-boronic acid

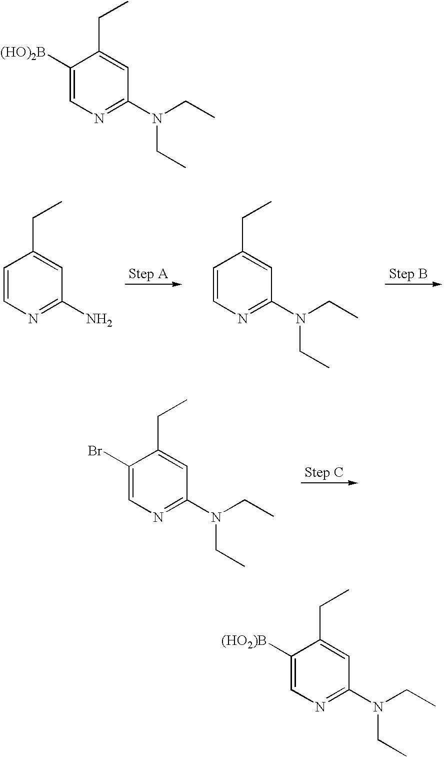 Step A

2-Amino-4-ethylpyridine (4.70 g) is dissolved in dichloromethane (80 mL). Addition of acetaldehyde (8.60 mL) and stirring for 10 min is followed by addition of sodium triacetoxyborohydride (24.6 g). After 1 h, the reaction is put into a mixture of water (300 mL) and sat. sodium bicarbonate (50 mL). Extraction with DCM (3×200 mL) and drying over magnesium sulfate yields a crude mixture that is used in step B without any further purification. LCMS: m/z 179.17 (M+H)⁺

Step B

The crude mixture from step A is dissolved in chloroform (150 mL) and cooled to 0° C. Addition of NBS (6.50 g, in three portions) is followed by stirring for 15 min. The light yellow solution is then put into a mixture of water (500 mL) and sat. sodium bicarbonate (100 mL). Extraction with DCM (3×150 mL) and drying over magnesium sulfate yields a crude mixture that is purified on silica gel. LCMS: m/z 257.10 (M+H)⁺

Step C

t-BuLi (50.1 mL, 1.7N in pentanes) is added to THF (200 mL) at −78 ° C. Slow addition of the purified material from step B (7.31 g, in 30 mL of THF) is followed by stirring for 15 min at −78° C. Upon LCMS check for unreacted bromide, triisopropyl borate (26.2 mL) is added and the reaction mixture is warmed to room temperature over night. The yellowish solution is then put into a mixture of water (1000 mL) and sat. sodium bicarbonate (100 mL). Extraction with DCM (3×300 mL) and drying over magnesium sulfate yields a crude material of good purity that can be used directly in palladium mediated couplings. LCMS: m/z 223.19 (M+H)⁺

### C. Synthesis of 2-isopropyl-6-methoxypyridine-3-boronic acid

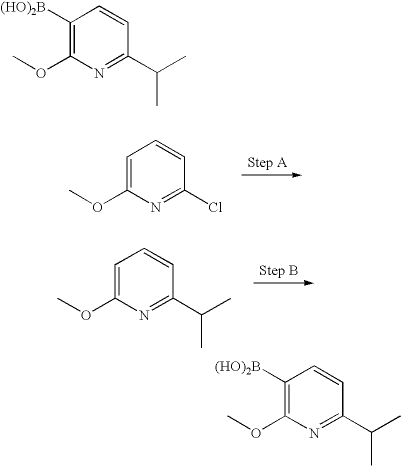 Step A

Following the procedure of Forstner et al. (JACS 124 (2002) 13856), 2-chloro-6-methoxypyridine (10 g) is stirred at −30° C. in a mixture of THF (2300 mL) and NMP (335 mL). Fe(acac)₃ (14.8 g) is added, followed by isopropyl magnesium chloride (490 mL of 2M in THF). The reaction mixture is allowed to warm to 0° C. over 1 hour and then saturated aqueous ammonium chloride (1000 mL) is added. The aqueous phase is separated and the organic layer is washed two times with water (1000 mL). The organic layer is distilled under reduced pressure to give 2-isopropyl-6-methoxypyridine. LCMS: Rt 1.95 min m/z 152.12(M+H)⁺

Step B

2-Isopropyl-6-methoxypyridine (191.4 g) and TMEDA (146.3 g) are dissolved in diethyl ether (1565 mL) and cooled to −60C. n-BuLi (760 mL of 2M) is added over 10 min. and the reaction mixture is allowed to warm to room temperature over 3.5 hours. The reaction mixture is chilled again to −60° C., triisopropylborate (476.2 g) is added and stirring is continued for 24 hours. 3M HCl is then added (510 mL), followed by water (2500 mL). The aqueous phase is separated and the organic layer is washed three times with 5% aqueous NaCl (1500 mL). The four aqueous phases are sequentially extracted with diethyl ether (2000 mL) and the combined ether extracts are concentrated under vacuum to give 2-isopropyl-6-methoxypyridine-3-boronic acid. LCMS: Rt 2.80 min m/z 196.11 (M+H)⁺

### D. Synthesis of 2-methoxy-4-trifluoromethoxyphenylboronic acid

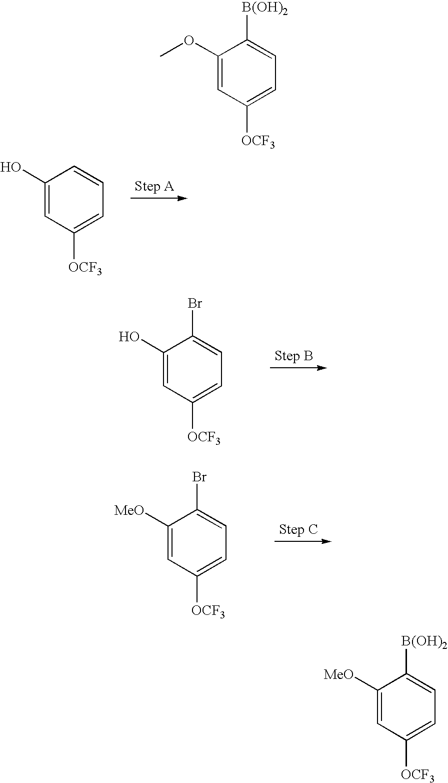 Step A

3-Trifluoromethoxyphenol (256.42 g) is dissolved in dichloromethane (2000 mL) and cooled to 5-10° C. under nitrogen. Bromine (241.6 g) is added dropwise over 2 hours, maintaining the temperature between 5-10° C. and then the cooling bath is removed. Water (1000 mL) is added and the mixtue is stirred for 10 minutes and separated. More water is added to the organic phase (500 mL) followed by powdered sodium carbonate (10-12 g) until the pH is 10-11. The organic layer is separated again, dried and concentrated under vacuum. Distillation affords 2-bromo-5-trifluoromethoxyphenol, which is used in the next step without further purification.

Step B

To 2-bromo-5-trifluoromethoxyphenol (479 g) dissolved in toluene (2600 mL) at 1-10° C. is added a solution of sodium hydroxide (80 g) in water (400 mL). The reaction mixture is stirred for 20 min and then tetra-n-butylammonium bromide (24 g) is added. Dimethyl sulfate (239.3 g) is divided into four portions and one portion is added to the mixture every 30 min, maintaining the internal temperature around 12-15° C. The reaction mixture is stirred overnight at this temperature and then water (1000 mL) is added and the organic layer is separated. It is washed consecutively with water (600 mL) and brine (600 mL) and then dried and evaporated to give 3-trifluoromethoxyanisole, which is used in the next step without further purification.

Step C

n-Butyllithium (156 mL of 2.5 M solution in hexanes) is added under nitrogen to THF (800 mL) over a period of 5 min while maintaining the temperature between −77 and −67° C. 2-Methoxy-4-trifluoromethoxy bromobenzene (100 g) is added over a 10-min period while maintaining the temperature between −76.0 and −62° C. Trimethylborate (53.8 g) is added over 10 min at a temperature of −76.3 to −63.2° C. After 1 hour, 200 ml of 2 N hydrochloric acid (200 mL) is added to pH 1. The mixture is allowed to warm to room temperature and the organic phase is separated and concentrated under vacuum to give crude 2-methoxy-4-trifluoromethoxyphenylboronic acid. The solid is treated with boiling n-heptane to give 2-methoxy-4-trifluoromethoxyphenylboronic acid. 1H—NMR (CDCl₃, 400 MHz) □ 7.89 (d, J=8.5 Hz, 1H), 6.90 (d, J=8.5 Hz, 1H), 6.75 (s, 1H), 6.13 (bs, 2H), 3.94 (s, 3H).

### Example 3

### Synthesis of 1-(1-Ethyl-propyl)-5-(2-methoxy-4-trifluoromethoxy-phenyl)-6-methyl-1H-[1,2,3]triazolo[4,5-b]pyrazine

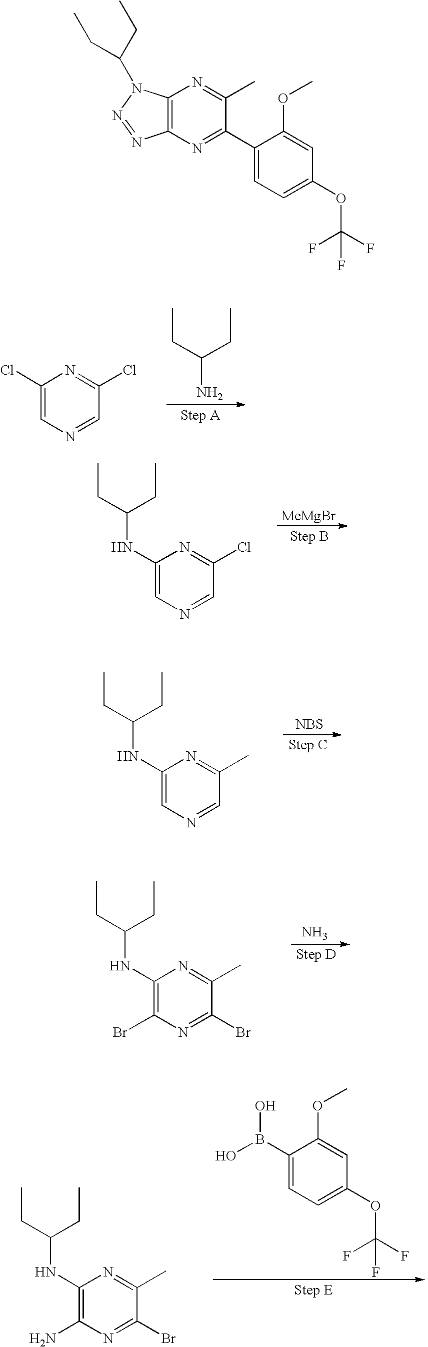 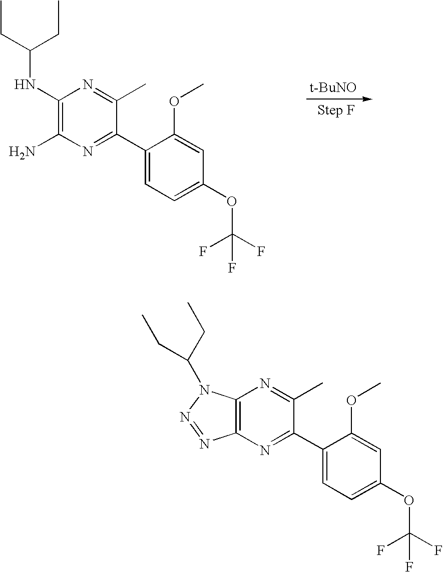 Step A

A solution of 2,6-dichloropyrazine (2.2 g) and 1-ethylpropylamine (5 mL) in EtOH (10 mL) is heated at 140° C. in a Teflon-sealed pressure tube for 14 hours. The resulting solution is concentrated in vacuo, diluted by water, and extracted twice with hexane-ethyl ether. Combined extracts are dried (sodium sulfate), filtered, concentrated in vacuo, and the residue filtered through a short pad of silica gel. The filtrate is concentrated to yield 2-(3-pentylamino)-6-chloropyrazine as a brown oil that solidified on standing.

Step B

[1,3-bis(diphenylphosphino)propane]dichloronickel(II) (540 mg) is added to a solution of 6-chloro-pyrazin-2-yl-(1-ethyl-propyl)-amine (4.26 g, 21.3 mmol) in THF (30 mL) at room temperature. After 10 minutes at room temperature, methylmagnesium bromide (3.0 M in diethyl ether, 15.7 mL, 47.1 mmol) is added dropwise at 0° C. The reaction mixture is stirred at room temperature for 1 hour. The resulting dark solution is poured into aqueous ammonium chloride and extracted twice with ether. Combined extracts are dried (sodium sulfate), filtered, concentrated, and submitted to flash chromatography to yield the desired product as a light brown oil.

Step C

A solution of the oil obtained from Step B (3.7 g, 20.9 mmol) in chloroform (60 mL) is cooled to 0° C. (ice-water bath) and N-bromosuccinimide (7.8 g, 44.0 mmol) is added in portions. After the addition is complete, the reaction mixture is stirred for 1 hour more while being allowed to warm to room temperature. The mixture is then concentrated to a small volume in vacuo, triturated with hexane, filtered, washed with hexane, and the filtrate concentrated and submitted to flash chromatography on silica gel (8% ethyl acetate in hexane) to yield 3,5-dibromo-6-methyl-pyrazin-2-yl)-(1-ethyl-propyl)-amine.

Step D

The product from step C (5.1 g, 15 mmol) is dissolved at room temperature in a solution of ammonia in ethanol (50 mL, 2M) in a pressure tube. Copper(0) (100 mg, 1.6 mmol) is added, and the mixture heated at 100 C for 16 hours. The reaction mixture is concentrated under reduced pressure, and the residue dissolved in ether and washed with brine (5×100 mL). The organic fractions are dried (magnesium sulfate), concentrated under reduced pressure, and the residue submitted to flash chromatography on silica gel eluting with ethyl acetate in hexanes, 5 to 15%). 5-Bromo-N²-(1-ethyl-propyl)-6-methyl-pyrazine-2,3-diamine is obtained as an oil. H-1 NMR: 4.2 (br, 2H), 3.94 (m, 1H), 3.83 (d, 1H), 2.39 (s, 3H), 1.63 (m, 2H), 1.49 (m, 2H), 0.91 (t, 6H).

Step E

The product from step D (1.4 g, 5.1 mmol), 2-methoxy-4-trifluoromethoxyphenylboronic acid (2.4 g, 10 mmol), and tetrakis(triphenylphosphine)palladium(0) (100 mg) are suspended in a mixture of toluene (40 mL) and K₂CO₃ solution (10 mL, 2M in water) in a pressure tube. The reaction mixture is heated at 80° C. (oil bath temperature) for 16 h. After cooling, the heterogeneous mixture is partitioned between ether and sodium bicarbonate solution, and the organic phase washed with brine, dried (MgSO₄) and concentrated under reduced pressure. Flash chromatography (ethyl acetate 25% in hexanes) produces the title compound as a light-yellow solid. MS: 385 (M+1). H-1 NMR: 7.25 (d, 1H), 6.88 (d, 1H), 6.78 (s, 1H), 3.9-4.1 (m, 4H), 3.79 (s, 3H), 2.14 (s, 3H), 1.65 (m, 2H), 1.55 (m, 2H), 0.94 (t, 6H). C-13 NMR: 157.72, 140.80, 140.31, 143.78, 140.04, 132.88, 131.90, 127.77, 112.60, 104.39, 55.63, 52.63, 26.65, 20.83, 10.05. F-19 NMR: -58.08.

Step F

The product of step E (50 mg) is dissolved in 2 mL of THF at room temperature. To the solution is added one drop of acetic acid and tBuNO (0.1 mL) and the mixture is refluxed for 50 min. After cooling, the mixture is partitioned between ether and sodium bicarbonate solution, and the organic phase washed with brine, dried (MgSO₄) and concentrated under reduced pressure. Flash chromatography (ethyl acetate 25% in hexanes) produces the title compound as amorphous. MS m/z 396.39 (M+H)⁺

### Example 4

### Synthesis of 5-(2-Methoxy-4-trifluoromethoxy-phenyl)-6-methyl-1H-pyrazolo[3.4-b]pyrazin-3-ol and 6-(2-Methoxy-4-trifluoromethoxy-phenyl)-5-methyl-1H-pyrazolo[3,4-b]pyrazin-3-ol

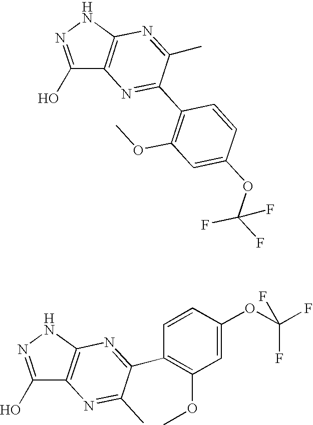 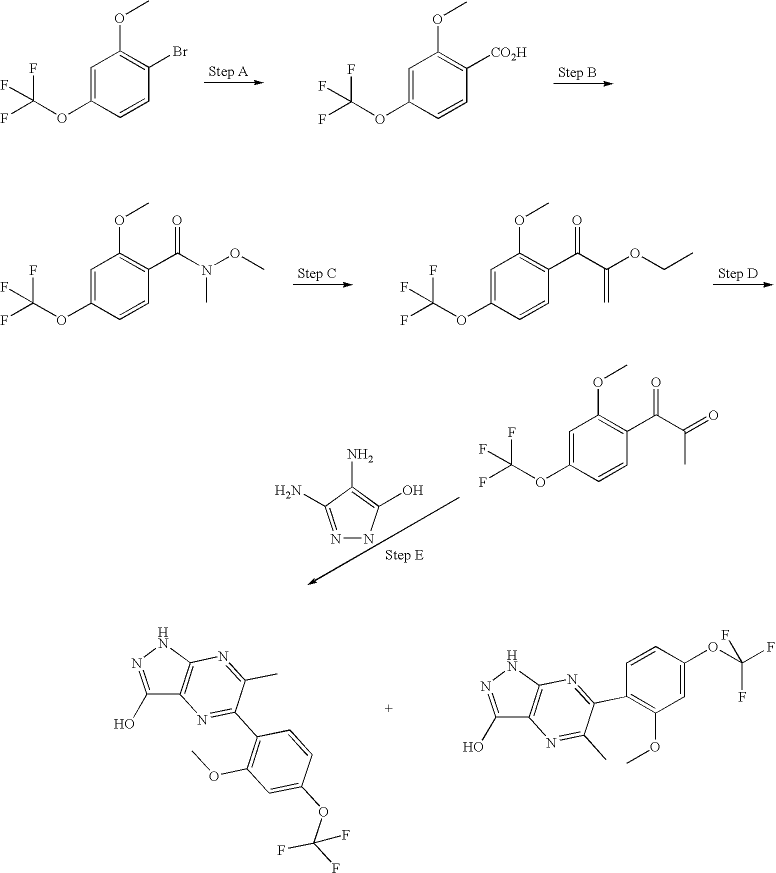 Step A

1-Bromo-2-methoxy-4-trifluoromethoxy-benzene (15 g) in anhydrous diethyl ether (120 mL) is cooled to −78° C. and subsequently treated with n-butyllithium in hexanes (23.2 mL, 2.5N). After stirring for 20 min, reaction mixture is added into freshly pulverized dry ice and is allowed to come to ambient temperature. Water (300 mL) is added and the mixture is extracted with diethyl ether. The organic phase is separated and dried over sodium sulfate to afford 2-methoxy-4-trifluoromethoxy-benzoic acid. LCMS: Rt 2.58 min m/z 219.04(M+H)⁺.

Step B

Similar to a procedure by Angelastro et al. (JOC, 1989, 3913), 2-methoxy-4-trifluoromethoxy-benzoic acid (9.9 g), N-Methylmorpholine (9.22 mL), isobutyl chloroformate and N,O-dimethylhydroxylamine hydrochloride (4.26 g) are used to synthesize 2,N-Dimethoxy-N-methyl-4-trifluoromethoxy-benzamide. LCMS: Rt 2.71 min m/z 280.05(M+H)⁺

Step C

Similar to a procedure by Angelastro et al. (JOC, 1989, 3913), 2,N-Dimethoxy-N-methyl-4-trifluoromethoxy-benzamide (10 g), ethyl vinyl ether (15.5 mL) and t-BuLi (105 mL, 1.5M in pentane) in THF (250 mL) are used to synthesize 2-Ethoxy-1-(2-methoxy-4-trifluoromethoxy-phenyl)-propenone. LCMS: Rt 3.27 min m/z 291.09(M+H)⁺

Step D

Similar to a procedure by Angelastro et al. (JOC, 1989, 3913), 2-ethoxy-1-(2-methoxy-4-trifluoromethoxy-phenyl)-propenone (11.68 g), concentrated HCl (80 mL) and 1,4-dioxane are used to synthesize 1-(2-Methoxy-4-trifluoromethoxy-phenyl)-propane-1,2-dione. LCMS: Rt 3.12 min m/z 263.06(M+H)⁺

Step E

1-(2-Methoxy-4-trifluoromethoxy-phenyl)-propane-1,2-dione (1.42 g) and 3,4-diamino-5-hydroxypyrazole sulfate (1.26 g) are stirred in MeOH (40 mL) over the weekend at ambient temperature. The precipitated product is filtered and dried to get 5-(2-methoxy-4-trifluoromethoxy-phenyl)-6-methyl-1H-pyrazolo[3,4-b]pyrazin-3-ol as a white powder (LCMS: Rt 2.85 min m/z 341.1(M+H)⁺. Water (75 mL) is added into the filtrate to precipitate out the other regio isomer. This is filtered and dried to afford 6-(2-methoxy-4-trifluoromethoxy-phenyl)-5-methyl-1H-pyrazolo[3,4-b]pyrazin-3-ol as a white powder. LCMS: Rt 2.87 min m/z 341.1(M+H)⁺

### Example 5

### Synthesis of 1-(1-Ethyl-propyl)-5-(2-methoxy-4-trifluoromethoxy-phenyl)-3,6-dimethyl-1H-pyrazolo[3,4-b]pyrazine, 1-(1-Ethyl-propyl)-5-(2-methoxy-4-trifluoromethoxy-phenyl)-6-methyl-1H-pyrazolo [3,4-b]pyrazine and 1,1′-Bis-(1-ethyl-propyl)-5,5′-bis-(2-methoxy-4-trifluoromethoxy-phenyl)-6,6′-dimethyl-1H,1′H-[3,3′]bi[pyrazolo[3.4-b]pyrazinyl]

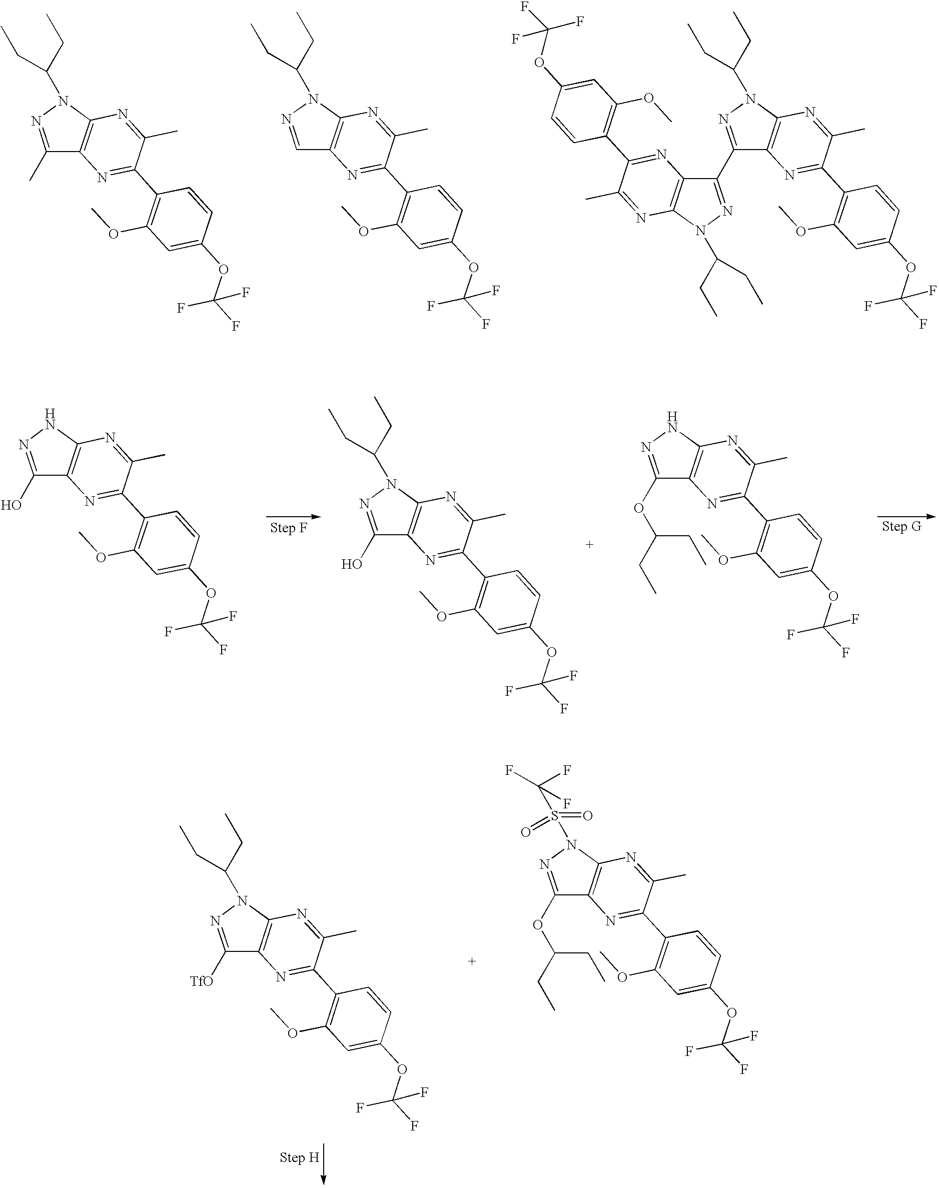 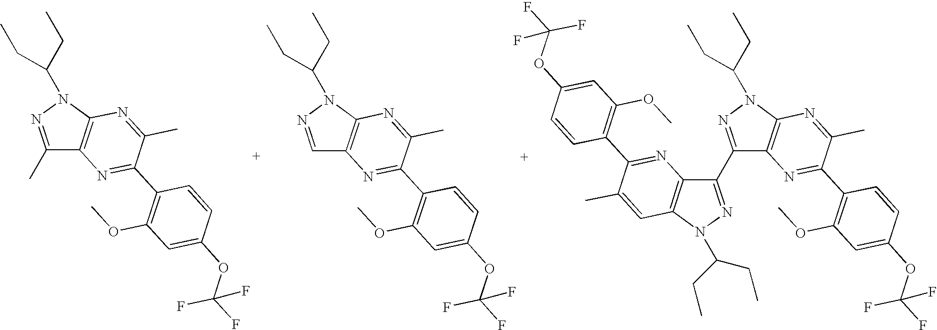 Step F

5-(2-Methoxy-4-trifluoromethoxy-phenyl)-6-methyl-1H-pyrazolo[3,4-b]pyrazin-3-ol (540 mg) and K₂CO₃ (220 mg) are dissolved in DMF (7 mL). 3-Bromopentane is slowly added and the mixture is heated to 60° C. After 1.5 h reaction is cooled to RT, filtered, concentrated and purified on silica gel to afford 1-(1-ethyl-propyl)-5-(2-methoxy-4-trifluoromethoxy-phenyl)-6-methyl-1H-pyrazolo[3,4-b]pyrazin-3-ol and 3-(1-ethyl-propoxy)-5-(2-methoxy-4-trifluoromethoxy-phenyl)-6-methyl-1H-pyrazolo[3,4-b]pyrazine as a mixture. LCMS: Rt 3.49 and 3.63 min m/z 41 1.13(M+H)⁺

Step G

The mixture of 1-(1-ethyl-propyl)-5-(2-methoxy-4-trifluoromethoxy-phenyl)-6-methyl-1H-pyrazolo[3,4-b]pyrazin-3-ol and 3-(1-ethyl-propoxy)-5-(2-methoxy-4-trifluoromethoxy-phenyl)-6-methyl-1H-pyrazolo[3,4-b]pyrazine (192 mg) and trifluoromethanesulfonic anhydride (90 uL) is dissolved in DCM (3.3 mL). Cooling to 0° C., triethyl amine is added dropwise and the cooling bath is removed. After 15 min, all the solvents are removed under vacuum and the residue is purified on silica gel to afford trifluoro-methanesulfonic acid1-(1-ethyl-propyl)-5-(2-methoxy-4-trifluoro-methoxy-phenyl)-6-methyl-1H-pyrazolo[3,4-b]pyrazin-3-yl ester. LCMS: Rt 4.42 min m/z 543.0(M+H)⁺ and 3-(1-ethyl-propoxy)-5-(2-methoxy-4-trifluoromethoxy-phenyl)-6-methyl-1-trifluoromethanesulfonyl-1H-pyrazolo[3,4-b]pyrazine. LCMS: Rt 4.10 min m/z 473.04(M+H)⁺

Step H

Trifluoro-methanesulfonicacid 1-(1-ethyl-propyl)-5-(2-methoxy-4-trifluoromethoxy-phenyl)-6-methyl-1H-pyrazolo[3,4-b]pyrazin-3-yl ester (142 mg) and methyl boronic acid (156 mg) are dissolved in toluene (5 mL). After 10 min of degassing, tetrakis(triphenylphosphine)palladium(0) (24 mg) is added, followed by 1 min of degassing. Upon addition of aqueous 1N sodium carbonate solution (1 mL) and lithium chloride (33 mg), the reaction mixture is heated to 100° C. for 16 h. Subsequently, the crude mixture is purified on silica gel to afford 1-(1-ethyl-propyl)-5-(2-methoxy-4-trifluoromethoxy-phenyl)-3,6-dimethyl-1 H-pyrazolo[3,4-b]pyrazine LCMS: Rt 4.07 min m/z 409.2(M+H)⁺, 1-(1-ethyl-propyl)-5-(2-methoxy-4-trifluoromethoxy-phenyl)-6-methyl-1H-pyrazolo[3,4-b]pyrazine, Rt 4.08 min m/z 395.16(M+H)⁺ and 1,1′-bis-(1-ethyl-propyl)-5,5′-bis-(2-methoxy-4-trifluoromethoxy-phenyl)-6,6′-dimethyl-1 H, 1′H-[3,3′]bi[pyrazolo[3,4-b]pyrazinyl]. Rt 4.79 min m/z 787.23(M+H)⁺

### Example 6

### Synthesis of 3-(1-Ethyl-propoxy)-6-(2-methoxy-4-trifluoromethoxy-phenyl)-1,5-dimethyl-1H-pyrazolo[3,4-b]pyrazine

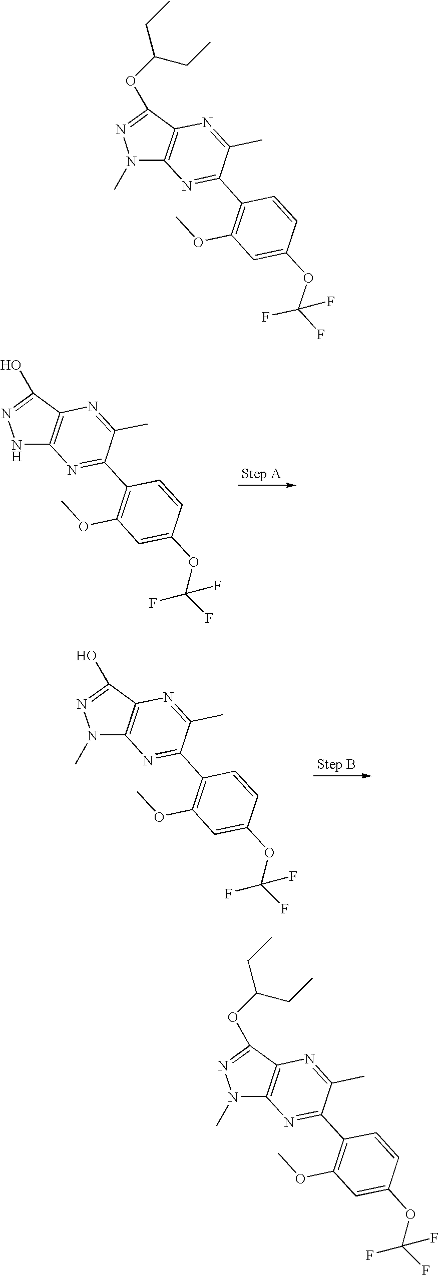 Step A 6-(2-Methoxy-4-trifluoromethoxy-phenyl)-5-methyl-1 H-pyrazolo[3,4-b]pyrazin-3-ol (740 mg) and K₂CO₃ (300 mg) are dissolved in DMF (7 mL). Methyl iodide (300 mg) is slowly added and the mixture heated to 60° C. After 1.5 h the reaction is cooled to RT, filtered, concentrated and purified on silica gel to afford 6-(2-methoxy-4-trifluoromethoxy-phenyl)-1,5-dimethyl-1H-pyrazolo[3,4-b]pyrazin-3-ol. Rt 3.15 min m/z 355.1(M+H)⁺
Step B

6-(2-Methoxy-4-trifluoromethoxy-phenyl)-1,5-dimethyl-1 H-pyrazolo[3,4-b]pyrazin-3-ol (30 mg) and K₂CO₃ (23 mg) are dissolved in DMF (0.5 mL). 3-Bromopentane (19 mg) is slowly added and heated to 60° C. After 1.5 h the reaction is cooled to RT, water is added (500 uL), and the mixture is extracted with EtOAc. The organic phase is separated and dried over sodium sulfate, concentrated and purified on silica gel to afford 3-(1-ethyl-propoxy)-6-(2-methoxy-4-trifluoromethoxy-phenyl)-1,5-dimethyl-1 H-pyrazolo[3,4-b]pyrazine. Rt 4.05 min m/z 425.16(M+H)⁺

### Example 7

### Synthesis of 5-(2,4-Dichloro-phenyl)-1-(1-ethyl-propyl)-3,6-dimethyl-1H-pyrazolo[3,4-b]pyrazine and 6-Ethyl-1-(1-ethyl-propyl)-5-(6-isopropyl-2-methoxy-pyridin-3-yl)-3-methyl-1H-pyrazolo[3,4-b]pyrazine

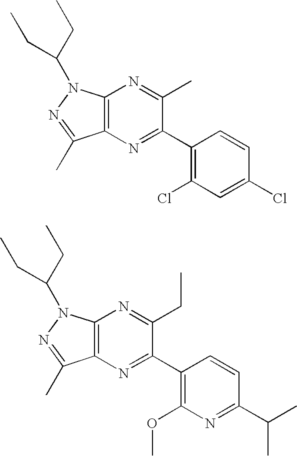 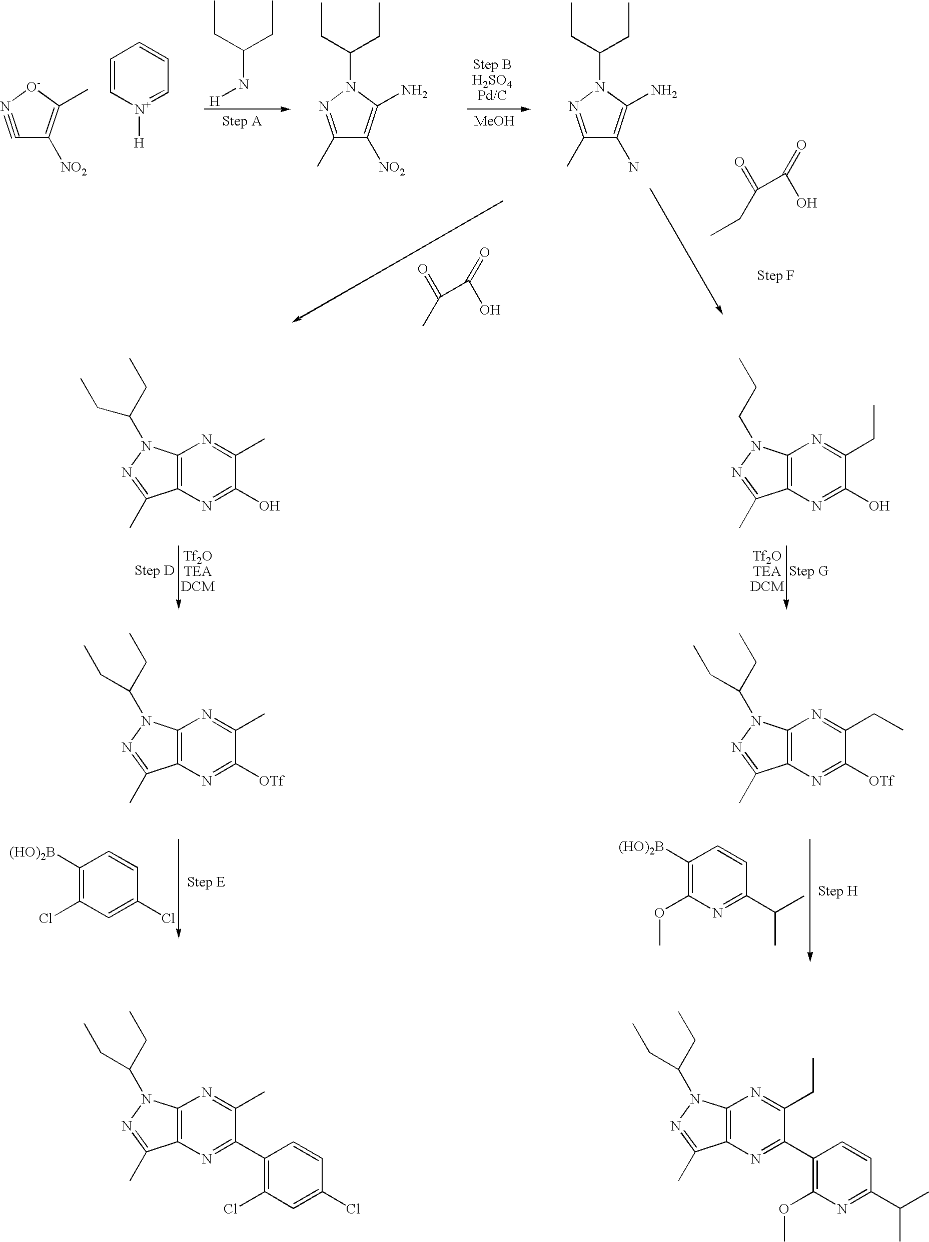 Step A

Analogous to the method described by Alberola et al. (J. Het Chem. 1986, 1035), 1-nitro-1-cyanoacetone pyridinium salt (20.5 g) (described by Alberola et al. J. Het Chem. 1982, 1073), 3-pentylhydrazine hydrochloride (20 g) (described by Arvanitis et al. WO9911643) and triethylamine (36 g) are dissolved in methanol (100 mL) and heated for 18 hours at 75° C. The solvents are then evaporated and the residue is distributed between ethyl acetate and aqueous hydrochloric acid. The organic phase is separated and dried over magnesium sulfate. Final purification over silica gel affords 2-(1-ethyl-propyl)-5-methyl-4-nitro-2H-pyrazol-3-ylamine. Rt 2.38 min m/z 213.1(M+H)⁺

Step B

2-(1-Ethyl-propyl)-5-methyl-4-nitro-2H-pyrazol-3-ylamine (512 mg), sulfuric acid (129 uL), 10% Pd/C (100 mg) and MeOH (10 mL) are shaken on a Parr shaker for 4 hrs under 55 psi hydrogen. Filtering through celite and concentration gives 2-(1-ethyl-propyl)-5-methyl-2H-pyrazole-3,4-diamine sulfate as a white powder. Rt 1.85 min m/z 183.2(M+H)⁺

Step C

2-(1-Ethyl-propyl)-5-methyl-2H-pyrazole-3,4-diamine sulfate from the previous step, pyruvic acid (255 mg) and EDAC.HCl (556 mg) are dissolved in a mixture of DCM (10 mL) and DMF (2 mL). After stirring overnight at RT, the solvents are removed under vacuum and the residue is purified on silica gel to afford 1-(1-ethyl-propyl)-3,6-dimethyl-1H-pyrazolo[3,4-b]pyrazin-5-ol. Rt 2.72 min m/z 235.2(M+H)⁺

Step D

1-(1-Ethyl-propyl)-3,6-dimethyl-1H-pyrazolo[3,4-b]pyrazin-5-ol (90 mg) and trifluoromethanesulfonic anhydride (74 uL) are dissolved in DCM (2 mL). After cooling to 0° C., triethyl amine (118 uL) is added dropwise and the cooling bath is removed. After 15 min, all the solvents are removed under vacuum and the residue is purified on silica gel to afford trifluoro-methanesulfonic acid 1-(1-ethyl-propyl)-3,6-dimethyl-1 H-pyrazolo[3,4-b]pyrazin-5-yl ester. Rt 4.15 min m/z 367.1(M+H)⁺

Step E

Trifluoro-methanesulfonic acid 1-(1-ethyl-propyl)-3,6-dimethyl-1H-pyrazolo[3,4-b]pyrazin-5-yl ester (130 mg) and 2,4-dichlorobenzene boronic acid (71 mg) are dissolved in toluene (2.5 mL). After 10 min of degassing, tetrakis(triphenylphosphine)palladium(0) (33 mg) is added, followed by 1 min of degassing. Upon addition of aqueous 1N sodium carbonate solution (710 uL) and lithium chloride (45 mg), the reaction mixture is heated to 100° C. for 16 h. Subsequently, the crude mixture is purified on silica gel to afford 5-(2,4-dichloro-phenyl)-1-(1-ethyl-propyl)-3,6-dimethyl-1H-pyrazolo[3,4-b]pyrazine. Rt 4.28 min m/z 363.1(M+H)⁺

Using the analogous boronic acids in step E, the following compounds are synthesized:

### 1-(1-Ethyl-1propyl)-5-(6-isopropyl-2-methoxy-pyridin-3-yl)-3,6-dimethyl-1H-pyrazolo[3,4-b]pyrazine. Rt 4.38 min m/z 368.3(M+H)⁺

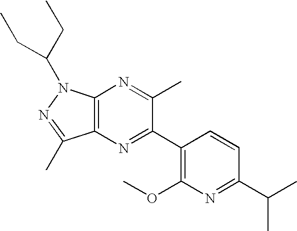

### {5-[1-(1-Ethyl-propyl)-3,6-dimethyl-1H-pyrazolo[3,4-b]pyrazin-5-yl]-4-methoxy-pyridin-2-yl}-dimethyl-amine. Rt 2.70 min m/z 369.2(M+H)⁺

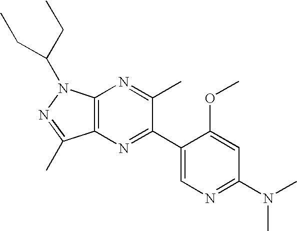

### {5-[1-(1-Ethyl-propyl)-3,6-dimethyl-1H-pyrazolo[3,4-b]pyrazin-5-yl]-4-isoproproxy-pyridin-2-yl}-dimethyl-amine. Rt 2.5 min m/z 397.2(M+H)⁺

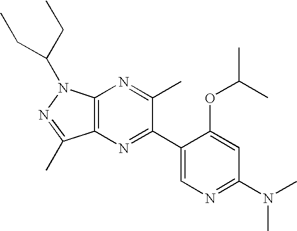 Step F

2-(1-Ethyl-propyl)-5-methyl-2H-pyrazole-3,4-diamine sulfate (5 g), 2-ketobutyric acid (1.83 g) and 4-(4,6-dimethoxy[1.3.5]triazin-2-yl)-4-methylmorpholinium chloride hydrate (5 g) are dissolved in DMF (80 mL). After stirring overnight at RT, water is added and the mixture is extracted with EtOAc. The organic phase is separated and dried over sodium sulfate and the residue is purified over silica gel to afford 6-ethyl-1-(1-ethyl-propyl)-3-methyl-1H-pyrazolo[3,4-b]pyrazin-5-ol. Rt 2.76 min m/z 249.17(M+H)⁺

Step G

In a manner analogous to step D, 6-ethyl-1-(1-ethyl-propyl)-3-methyl-1H-pyrazolo[3,4-b]pyrazin-5-ol (3.9 g) and trifluoromethanesulfonic anhydride (4.22 mL) afford trifluoro-methanesulfonic acid 6-ethyl-1-(1-ethyl-propyl)-3-methyl-1 H-pyrazolo[3,4-b]pyrazin-5-yl ester.

Rt 4.3 min m/z 381.1(M+H)⁺.

Step H

In a manner analogous to step E, trifluoro-methanesulfonic acid 6-ethyl-1-(1-ethyl-propyl)-3-methyl-1H-pyrazolo[3,4-b]pyrazin-5-yl ester (1.78 g) and 6-isopropyl-2-methoxy-3-pyridine boronic acid (1.08 g) afford 6-ethyl-1-(1-ethyl-propyl)-5-(6-isopropyl-2-methoxy-pyridin-3-yl)-3-methyl-1 H-pyrazolo[3,4-b]pyrazine.

Rt 4.37 min m/z 382.25(M+H)⁺

Using the analogous boronic acids in step H, the following compounds are synthesized:

### 6-Ethyl-1-(1-ethyl-propyl)-5-(2-methoxy-4-trifluoromethoxy-phenyl)-3-methyl-1H-pyrazolo[3,4-b]pyrazine. Rt 4.29 min m/z 423.2(M+H)⁺

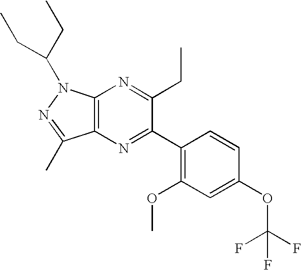

### {5-[6-Ethyl-1-(1-ethyl-propyl)-3-methyl-1H-pyrazolo[3,4-b]pyrazin-5-yl]-4-isopropoxy-pyridin-2-yl}-dimethyl-amine. Rt 2.99 min m/z 411.3(M+H)⁺

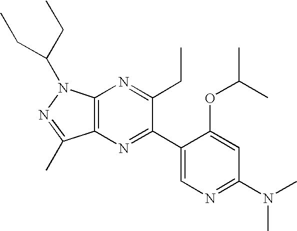

### 5-(2-Chloro-4-methoxy-phenyl)-6-ethyl-1-(1-ethyl-propyl)-3-methyl-1H-pyrazolo[13,4-biyrazine. Rt 4.24 min m/z 373.2(M+H)⁺

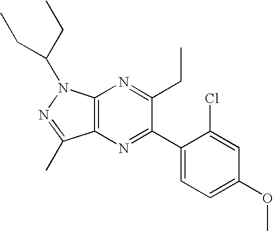

### Diethyl- {4-ethyl-5-[6-ethyl-1-(1-ethyl-propyl)-3-methyl-1H-pyrazolo[3,4-b]pyrazin-5-yl]-pyridin-2-yl}-amine. Rt 3.12 min m/z 409.3(M+H)⁺

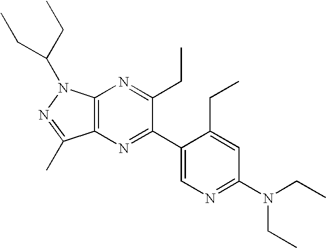

### Synthesis of 1-benzyl-5-(6-isopropyl-2-methoxy-pyridin-3-yl)-3-methyl-1H-pyrazolo[3,4-b]pyrazine

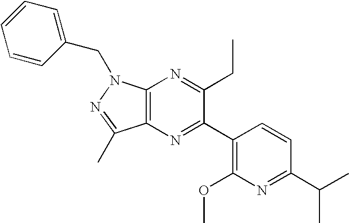

Substituting benzylhydrazine hydrochloride for 3-pentylhydrazine hydrochloride in step A and following step F affords, in analogous fashion, 1-benzyl-6-ethyl-5-(6-isopropyl-2-methoxy-pyridin-3-yl)-3-methyl-1H-pyrazolo[3,4-b]pyrazine. Rt 4.20 min m/z 402.2(M+H)⁺.

### Synthesis of 1-(2-Benzyloxy-1-benzyloxymethyl-ethyl)-6-ethyl-5-(6-isopropyl-2-methoxy-pyridin-3-yl)-3-methyl-1H-pyrazolo[3.4-b]pyrazine

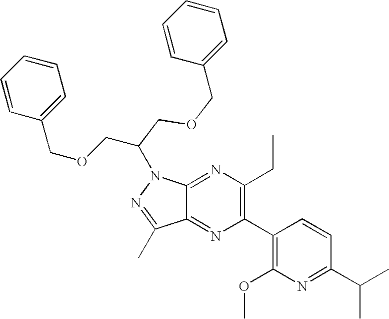

Substituting (2-benzyloxy-1-benzyloxymethyl-ethyl)-hydrazine hemioxalate (Tetrahedron 67 (2001) 8917-8923) for 3-pentylhydrazine hydrochloride in step A and following step F affords, in analogous fashion, 2-(2-benzyloxy-1-benzyloxymethyl-ethyl)-5-methyl-4-nitro-2H-pyrazol-3-ylamine. LCMS: m/z 397.19 (M+H)⁺, Rt 3.27 mins.

### Synthesis of 6-ethyl-1-(2-methoxy-1-methyl-ethyl)-5-(6-isopropyl-2-methoxy-pyridin-3-yl)-3-methyl-1H-pyrazolo[3,4-b]pyrazine

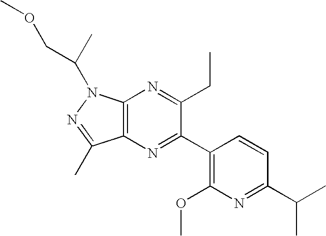

Substituting (2-Methoxy-1-methyl-ethyl)-hydrazine hydrochloride for 3-pentylhydrazine hydrochloride in step A affords, in analogous fashion, 6-ethyl-1-(2-methoxy-1-methyl-ethyl)-5-(6-isopropyl-2-methoxy-pyridin-3-yl)-3-methyl-1 H-pyrazolo[3,4-b]pyrazine. Rt 3.92 min m/z 384.21(M+H)^{+.}

### Example 8

### Synthesis of (2-Methoxy-1-methyl-ethyl)-hydrazine hydrochloride

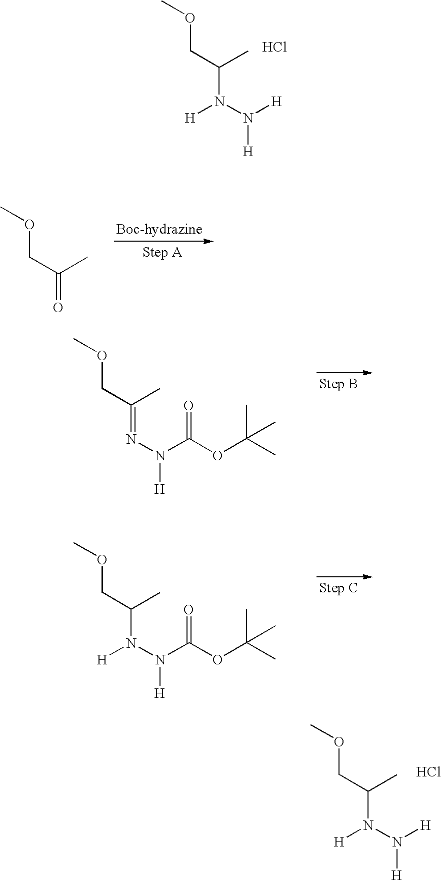 Step A

1-Methoxy-propan-2-one (10 g) in heptane (400 mL) is warmed to 50° C. and Boc-hydrazine (l 9.5g) in toluene (30 mL) is added. After the addition, the reaction is heated to 70° C. for 2 h and stirred overnight at RT. The precipitate formed is collected, washed with heptane and dried to afford N′-(2-Methoxy-1-methyl-ethylidene)-hydrazine-carboxylic acid tert-butyl ester. Rt 1.93 min m/z 203.13(M+H)⁺.

Step B

N′-(2-Methoxy-1-methyl-ethylidene)-hydrazinecarboxylic acid tert-butyl ester (18.6 g), PtO₂ (1 g) and glacial acetic acid (92 mL) are shaken on a Parr shaker for 1.5 hrs under 55 psi hydrogen. After filtering the mixture through celite and concentrating under vacuum, half-saturated aqueous sodium bicarbonate is added and the mixture is extracted with ether. The organic phase is separated, dried over sodium sulfate and concentrated under vacuum to afford N′-(2-Methoxy-1-methyl-ethyl)-hydrazinecarboxylic acid tert-butyl ester. Rt 1.67 min m/z 205.16(M+H)⁺.

Step C

N′-(2-Methoxy-1-methyl-ethyl)-hydrazinecarboxylic acid tert-butyl ester (4.26 g) and 1M HCl in ether (50 mL) are refluxed for 1 hr. Removal of the solvent under vacuum affords (2-Methoxy-1-methyl-ethyl)-hydrazine hydrochloride. Rt 0.47 min m/z 105.11(M+H)⁺.

### Example 9

### Synthesis of {3-[6-Ethyl-1-(1-ethyl-propyl)-3-methyl-1H-pyrazolo[3,4-b]pyrazin-5-yl]-6-isopropyl-pyridin-2-yl}-dimethyl-amine

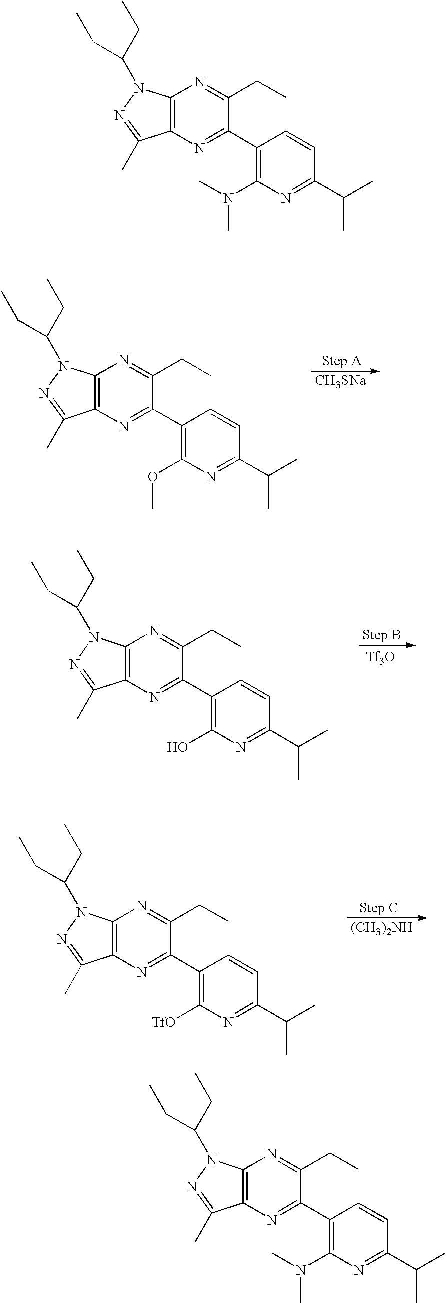 Step A

6-Ethyl-1-(1-ethyl-propyl)-5-(6-isopropyl-2-methoxy-pyridin-3-yl)-3-methyl-1H-pyrazolo[3,4-b]pyrazine (1.57 g) and sodium methanethiolate (2.88 g) are dissolved in DMF (40 mL) and heated to 110° C for 1 hr. After cooling the mixture to RT, EtOAc (40 mL) is added and the mixture is washed with WATER (2×30 mL) and brine. The organic phase is separated, dried over sodium sulfate and concentrated to afford 3-[6-ethyl-1-(1-ethyl-propyl)-3-methyl-1H-pyrazolo[3,4-b]pyrazin-5-yl]-6-isopropyl-pyridin-2-ol. Rt 3.18 min m/z 368.34(M+H)⁺.

Step B

3-[6-Ethyl-1-(1-ethyl-propyl)-3-methyl-1H-pyrazolo[3,4-b]pyrazin-5-yl]-6-isopropyl-pyridin-2-ol (1.11 g) and trifluoromethanesulfonic anhydride (610 uL) are dissolved in DCM (30 mL). After cooling to 0° C., triethyl amine (926 uL) is added dropwise and the cooling bath is removed. After 15 min, all the solvents are removed under vacuum and the remaining residue is purified on silica gel to afford trifluoro-methanesulfonic acid 3-[6-ethyl-1-(1-ethyl-propyl)-3-methyl-1H-pyrazolo[3,4-b]pyrazin-5-yl]-6-isopropyl-pyridin-2-ylester. Rt 4.25 min m/z 500.18(M+H)⁺.

Step C

Trifluoromethanesulfonic acid 3-[6-ethyl-1-(1-ethyl-propyl)-3-methyl-1H-pyrazolo[3,4-b]pyrazin-5-yl]-6-isopropyl-pyridin-2-ylester (50 mg) and dimethyl amine (2M in THF, 100 uL) are dissolved in DMSO (500 uL). After microwaving at 130° C. for 15 min; Water (500 uL) is added and the mixture is extracted with EtOAc. The organic phase is separated, dried over sodium sulfate and concentrated under vacuum to afford a residue that is purified over silica gel to afford {3-[6-ethyl-1-(1-ethyl-propyl)-3-methyl-1H-pyrazolo[3,4-b]pyrazin-5-yl]-6-isopropyl-pyridin-2-yl}-dimethyl-amine. Rt 3.58 min m/z 395.27(M+H)⁺.

Using analogous amines in step C, the following compounds are synthesized:

### 2-({3-[6-Ethyl-1-(1-ethyl-propyl)-3-methyl-1H-pyrazolo[3,4-b]pyrazin-5-yl]-6-isopropyl-pyridin-2-yl}-methyl-amino)-ethanol. Rt 2.8 min m/z 425.28(M+H)⁺

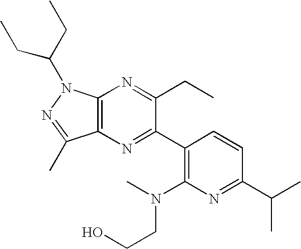

### 1-{3-[6-Ethyl-1-(1-ethyl-propyl)-3-methyl-1H-pyrazolo[3,4-b]pyrazin-5-yl]-6-isopropyl-pyridin-2-yl}-pyrrolidin-3-ol. Rt 2.63 min m/z 437.28(M+H)⁺.

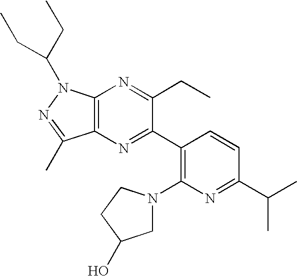

### {3-[6-Ethyl-1-(1-ethyl-propyl)-3-methyl-1H-pyrazolo[3,4-b]pyrazin-5-yl]-6-isopropyl-pyridin-2-yl}-(2-methoxy-ethyl)-amine. Rt 2.9 min m/z 425.28(M+H)⁺.

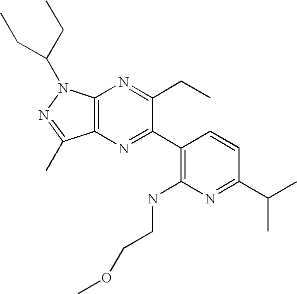

### 3′-[6-Ethyl-1-(1-ethyl-propyl)-3-methyl-1H-pyrazolo[3,4-b]pyrazin-5-yl]-6′-isopropyl-3,4,5,6-tetrahydro-2H-[1,2′]bipyridinyl. Rt 4.58 min m/z 435.30(M+H)⁺.

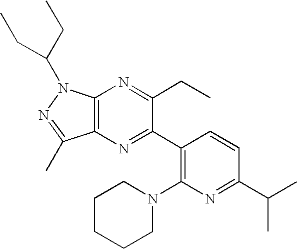

### {3-[6-Ethyl-1-(1-ethyl-propyl)-3-methyl-1H-pyrazolo[3,4-b]pyrazin-5-yl]-6-isopropyl-pyridin-2-yl}-methyl-amine. Rt 2.92 min m/z 381.3(M+H)⁺.

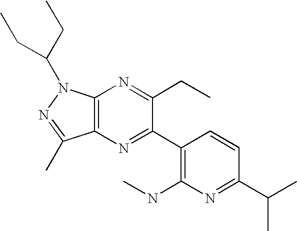

### {3-[6-Ethyl-1-(1-ethyl-propyl)-3-methyl-1H-pyrazolo[3,4-b]pyrazin-5-yl]-6-isopropyl-pyridin-2-yl}-(3-piperidin-1-yl-propyl)-amine. Rt 2.84 min m/z 492.4(M+H)⁺.

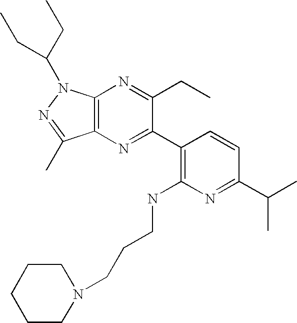

### (1-{3-[6-Ethyl-1-(1-ethyl-propyl)-3-methyl-1H-pyrazolo[3,4-b]pyrazin-5-yl]-6-isopropyl-pyridin-2-yl}-pyrrolidin-3-yl)-dimethyl-amine. Rt 2.85 min m/z 464.34(M+H)⁺.

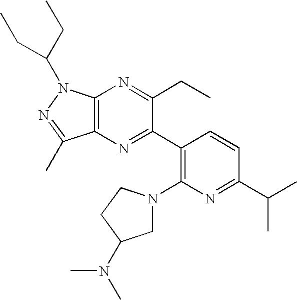

### {3-[6-Ethyl-1-(1-ethyl-propyl)-3-methyl-1H-pyrazolo[3,4-b]pyrazin-5-yl]-6-isopropyl-pyridin-2-yl}-(tetrahydro-furan-2-ylmethyl)-amine. Rt 2.77 min m/z 451.28(M+H)⁺.

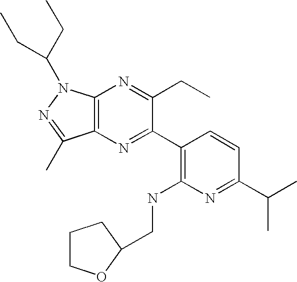

### Synthesis of {3-[1-(1-ethyl-propyl)-3,6-dimethyl-1H-pyrazolo[3,4-b]pyrazin-5-yl]-6-isopropyl-pyridin-2-yl}-(2-methoxy-ethyl)-amine

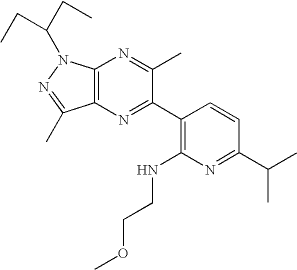

Analogously, substituting 1-(1-ethyl-propyl)-5-(6-isopropyl-2-methoxy-pyridin-3-yl)-3,6-dimethyl-1H-pyrazolo[3,4-b]pyrazine in step A and methoxyethylamine in step C affords {3-[1-(1-ethyl-propyl)-3,6-dimethyl-1H-pyrazolo[3,4-b]pyrazin-5-yl]-6-isopropyl-pyridin-2-yl}-(2-methoxy-ethyl)-amine. Rt=2.65 min, m/z 411.29 (M+H)⁺

Using analogous amines in step C, the following compounds are synthesized:

### {3-[1-(1-Ethyl-propyl)-3,6-dimethyl-1H-pyrazolo[3,4-b]pyrazin-5-yl]-6-isopropyl-pyridin-2-yl}-[2-(1H-imidazol-4-yl)-ethyl]-amine. Rt=2.18 min, m/z 447.30 (M+H)⁺

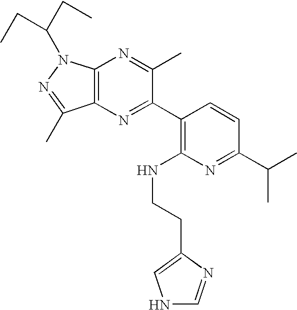

### 1-(1-Ethyl-propyl)-5-(6-isopropyl-2-morpholin-4-yl-pyridin-3-yl)-3,6-dimethyl-1H-pyrazolo[3,4-b]pyrazine. Rt=3.92 min. m/z 423.27 (M+H)⁺.

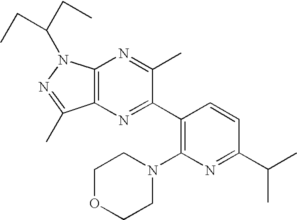

### N-(2-{3-[1-(1-Ethyl-propyl)-3,6-dimethyl-1H-pyrazolo[3,4-b]pyrazin-5-yl]-6-isopropyl-pyridin-2-ylamino}-ethyl)-acetamide. m/z 438.29 (M+H)⁺, Rt 2.37 min.

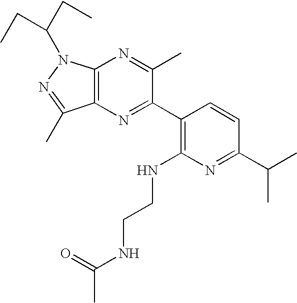

### N′-{3-[1-(1-Ethyl-propyl)-3,6-dimethyl-1H-pyrazolo[3,4-b]pyrazin-5-yl]-6-isopropyl-pyridin-2-yl}-N,N-dimethyl-pentane-1,5-diamine. m/z 466.38 (M+H)⁺, Rt 2.07 min.

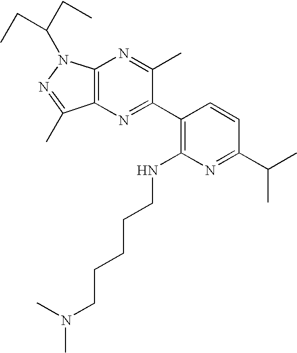

### {3-[1-(1-Ethyl-propyl)-3,6-dimethyl-1H-pyrazolo[3,4-b]pyrazin-5-yl]-6-isopropyl-pyridin-2-yl}-methyl-amine. m/z 367.3 (M+H)⁺, Rt 2.72 min.

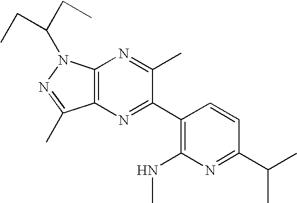

### {3-[1-(1-Ethyl-propyl)-3,6-dimethyl-1H-pyrazolo[3,4-b]pyrazin-5-yl]-6-isopropyl-pyridin-2-yl}-dimethyl-amine. m/z 381.26 (M+H)⁺, Rt 3.18 min.

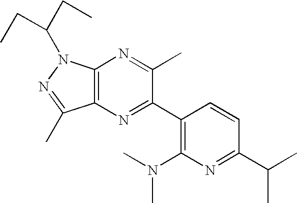

### 5-(2-Azetidin-1-yl-6-isopropyl-pyridin-3-yl)-1-(1-ethyl-propyl)-3,6-dimethyl-1H-pyrazolo[3,4-b]pyrazine. m/z 393.3 (M+H)⁺, Rt 2.95 min.

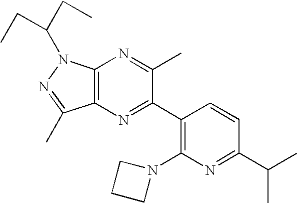

### N′-{3-[1-(1-Ethyl-propyl)-3,6-dimethyl-1H-pyrazolo[3,4-b]pyrazin-5-yl]-6-isopropyl-pyridin-2-yl}-N,N-dimethyl-ethane-1,2-diamine. m/z 424.32 (M+H)⁺, Rt 2.70 mins.

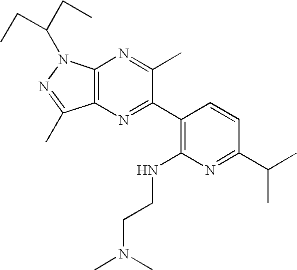

### Synthesis of {3-[6-Ethyl-1-(2-methoxy-1-methyl-ethyl)-3-methyl-1H-pyrazolo[3,4-b]pyrazin-5-yl]-6-isopropyl-pyridin-2-yl}-methylamine

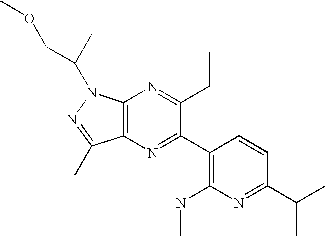

Substituting 6-ethyl-1-(2-methoxy-1-methyl-ethyl)-5-(6-isopropyl-2-methoxy-pyridin-3-yl)-3-methyl-1H-pyrazolo[3,4-b]pyrazine for 6-ethyl-1-(1-ethyl-propyl)-5-(6-isopropyl-2-methoxy-pyridin-3-yl)-3-methyl-1H-pyrazolo[3,4-b]pyrazine in step A and methylamine for dimethylamine in step C gives, in an analogous fashion, {3-[6-Ethyl-1-(2-methoxy-1-methyl-ethyl)-3-methyl-1H-pyrazolo[3,4-b]pyrazin-5-yl]-6-isopropyl-pyridin-2-yl}-methylamine. Rt 2.32 min m/z 383.24(M+H)⁺

### Synthesis of 6-Ethyl-5-(2-ethyl-6-isopropyl-pyridin-3-yl)-1-(2-methoxy-1-methyl-ethyl)-3-methyl-1H-pyrazolo[3,4-b]pyrazine

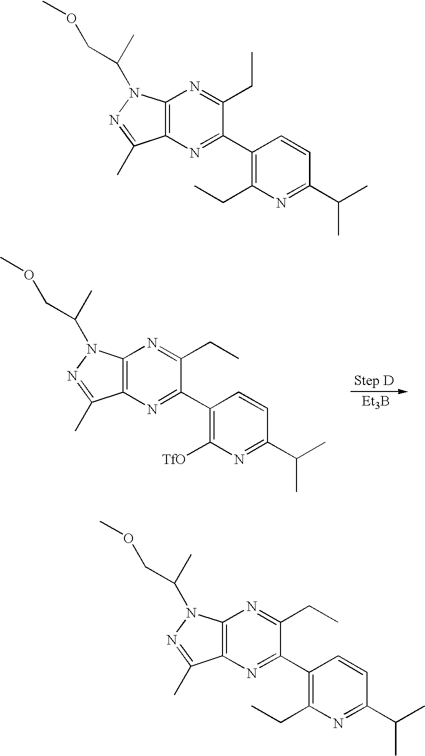 Step D

6-Ethyl-1-(2-methoxy-1-methyl-ethyl)-5-(6-isopropyl-2-methoxy-pyridin-3-yl)-3-methyl-1H-pyrazolo[3,4-b]pyrazine is substituted for 6-ethyl-1-(1-ethyl-propyl)-5-(6-isopropyl-2-methoxy-pyridin-3-yl)-3-methyl-1H-pyrazolo[3,4-b]pyrazine in step A and step D is carried out in the following fashion: trifluoro-methanesulfonic acid 3-[6-ethyl-1-(2-methoxy-1-methyl-ethyl)-3-methyl-1H-pyrazolo[3,4-b]pyrazin-5-yl]-6-isopropyl-pyridin-2-yl ester (57 mg) and triethyl borane (1M in hexane, 341 uL) are dissolved in toluene (1.5 mL). After 10 min of degassing, tetrakis(triphenylphosphine)palladium(0) (10.5 mg) is added, followed by 1 min of degassing. Upon addition of aqueous 1N sodium carbonate solution (228 uL) and lithium chloride (14.5 mg), the reaction mixture is heated to 100° C. for 2 h. The mixture is then cooled to RT, water is added, and the mixture is extracted with EtOAc. The organic phase is separated, dried over sodium sulfate and evaporated under vacuum. Silica gel purification affords 6-ethyl-5-(2-ethyl-6-isopropyl-pyridin-3-yl)-1-(2-methoxy-1-methyl-ethyl)-3-methyl-1H-pyrazolo[3,4-b]pyrazine. Rt 2.42 min m/z 382.26(M+H)⁺

### Example 10

### Synthesis of {3-[1-benzyl-6-ethyl-3-methyl-1H-pyrazolo[3,4-b]pyrazin-5-yl]-6-isopropyl-pyridin-2-yl}-methylamine

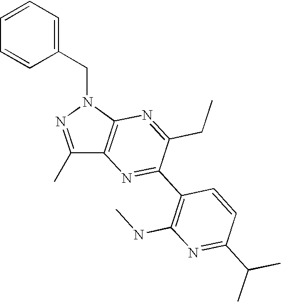 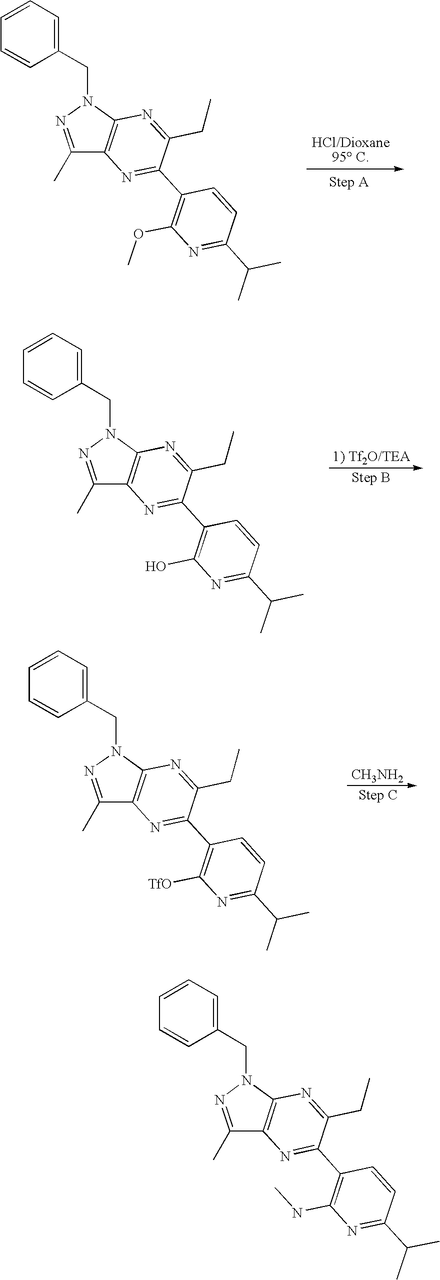 Step A

1-Benzyl-6-ethyl-5-(6-isopropyl-2-methoxy-pyridin-3-yl)-3-methyl-1H-pyrazolo[3,4-b]pyrazine (1.9 g) is dissolved in 4M HCl in 1,4-dioxane (25 mL) and heated to 95° C. for 40 min. All the solvent is removed under vacuum and EtOAc (30 mL) and Water (20 mL) are added. The precipitated solid is collected and dried to afford 3-(1-benzyl-6-ethyl-3-methyl-1H-pyrazolo[3,4-b]pyrazin-5-yl)-6-isopropyl-pyridin-2-ol. Rt 3.059 min m/z 388.2(M+H)⁺

Step B

Analogous to the preparation of trifluoromethanesulfonic acid 3-[6-ethyl-1-(1-ethyl-propyl)-3-methyl-1H-pyrazolo[3,4-b]pyrazin-5-yl]-6-isopropyl-pyridin-2-ylester, 3-(1-benzyl-6-ethyl-3-methyl-1H-pyrazolo[3,4-b]pyrazin-5-yl)-6-isopropyl-pyridin-2-ol (1.6 g) and trifluoromethanesulfonic anhydride (873 uL) afford trifluoro-methanesulfonic acid 3-(1-benzyl-6-ethyl-3-methyl-1H-pyrazolo[3,4-b]pyrazin-5-yl)-6-isopropyl-pyridin-2-yl ester. Rt 4.1 min m/z 520.2(M+H)⁺

Step C

Trifluoromethanesulfonic acid 3-(1-benzyl-6-ethyl-3-methyl-1H-pyrazolo[3,4-b]pyrazin-5-yl)-6-isopropyl-pyridin-2-yl ester (1.4 g) and methyl amine (2M in NMP, 14 mL) are heated at 80° C. for 2 h. After cooling to RT, Water (20 mL) is added and the mixture is extracted with EtOAc. The organic phase is separated, dried over sodium sulfate and evaporated under vacuum. Silica gel purification of the residue affords [3-(1-Benzyl-6-ethyl-3-methyl-1H-pyrazolo[3,4-b]pyrazin-5-yl)-6-isopropyl-pyridin-2-yl]-methyl-amine. Rt 2.65 min m/z 401.3(M+H)⁺

### Example 11

### Synthesis of {3-[1-(1-Diethoxymethyl-propyl)-6-ethyl-3-methyl-1H-pyrazolo[3,4-b]pyrazin-5-yl]-6-isopropyl-pyridin-2-yl}-methyl-amine and [3-{6-Ethyl-3-methyl-1-(1-morpholin-4-ylmethyl-propyl)-1H-pyrazolo[3,4-b]pyrazin-5-yl]-6-isopropyl-pyridin-2-yl}-methyl-amine

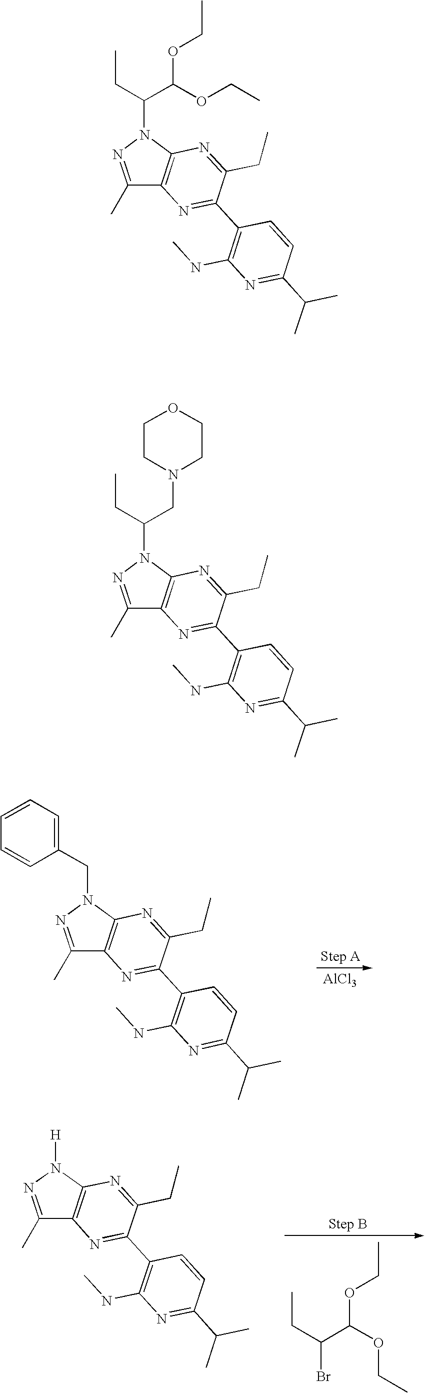 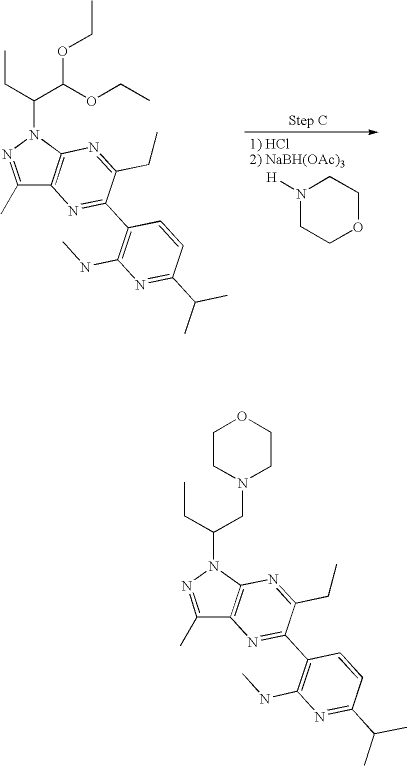 Step A

[3-(1-Benzyl-6-ethyl-3-methyl-1H-pyrazolo[3,4-b]pyrazin-5-yl)-6-isopropyl-pyridin-2-yl]-methyl-amine (130 mg) is dissolved in anhydrous toluene (8 mL) and aluminum chloride (173 mg) is added. The mixture is heated to 50° C. for 16 h and then all the solvent is removed under vacuum. The remaining residue is redissolved in EtOAc and is added into iced saturated ammonium chloride slurry. The mixture is extracted with EtOAc and the organic phase is separated, dried over sodium sulfate evaporated under vacuum. Silica gel purification affords [3-(6-Ethyl-3-methyl-1H-pyrazolo[3,4-b]pyrazin-5-yl)-6-isopropyl-pyridin-2-yl]-methyl-amine. Rt 1.90 min m/z 311.2(M+H)⁺

Step B

[3-(6-Ethyl-3-methyl-1H-pyrazolo[3,4-b]pyrazin-5-yl)-6-isopropyl-pyridin-2-yl]-methyl-amine (66 mg), alpha-bromobutyraldehyde diethyl acetal (72 mg) and K2CO3 (74 mg) are dissolved in DMF (1 mL) and heated to 60° C. After 18 h the reaction is cooled to RT, filtered, concentrated and purified on silica gel to afford {3-[1-(1-diethoxymethyl-propyl)-6-ethyl-3-methyl-1H-pyrazolo[3,4-b]pyrazin-5-yl]-6-isopropyl-pyridin-2-yl}-methyl-amine. Rt 2.58 min m/z 455.3(M+H)⁺

Step C

{3-[1-(1-Diethoxymethyl-propyl)-6-ethyl-3-methyl-1H-pyrazolo[3,4-b]pyrazin-5-yl]-6-isopropyl-pyridin-2-yl}-methyl-amine (20 mg) and 1N aqueous HCl (100 uL) are dissolved in acetone (500 uL) and heated to 60° C. After 24 h the reaction is cooled to RT, concentrated and purified on silica gel to afford the intermediate aldehyde which is then treated with morpholine and sodium triacetoxyborohydride in DCM. After overnight stirring, saturated aqueous sodium bicarbonate is added and the mixture is extracted with EtOAc. The organic phase is separated, dried over sodium sulfate and evaporated under vacuum. Silica gel purification affords {3-[6-Ethyl-3-methyl-1-(1-morpholin-4-ylmethyl-propyl)-1H-pyrazolo[3,4-b]pyrazin-5-yl]-6-isopropyl-pyridin-2-yl}-methyl-amine. Rt 2.62 min m/z 452.3(M+H)⁺

Using the analogous alkylating agents in step B, the following compounds are synthesized:

### {3-[6-Ethyl-1-(1-methoxymethyl-propyl)-3-methyl-1H-pyrazolo[3,4-b]pyrazin-5-yl]-6-isopropyl-pyridin-2-yl}-methyl-amine. Rt 2.82 min m/z 397.2(M+H)⁺.

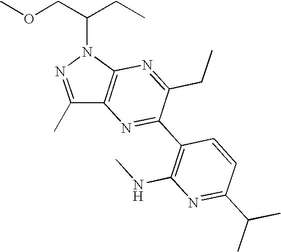

### [3-(1-sec-Butyl-6-ethyl-3-methyl-1H-pyrazolo[3,4-b]pyrazin-5-yl)-6-isopropyl-pyridin-2-yl]-methyl-amine. Rt 2.60 min m/z 367.3(M+H)⁺.

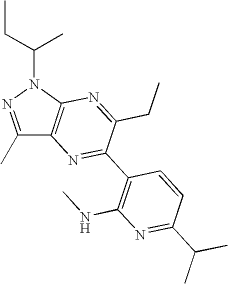

### Example 12

### Synthesis of 1-Benzyl-5-(6-isopropyl-2-methoxy-pyridin-3-yl)-3,6-dimethyl-1H-pyrazolo [3,4-b]pyrazine

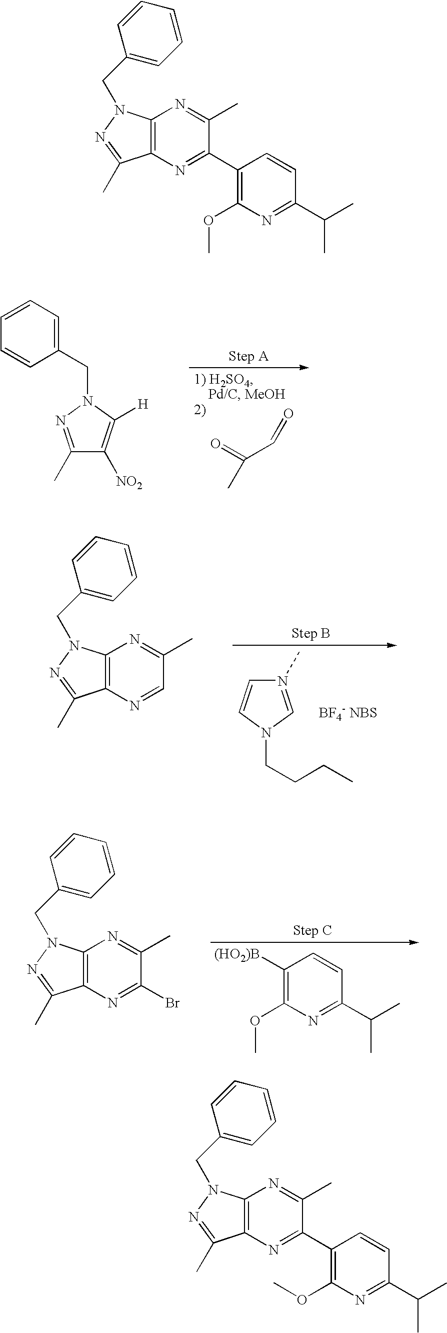 Step A

2-Benzyl-5-methyl-4-nitro-2H-pyrazol-3-ylamine (1.68 g), sulfuric acid (405 uL), 10% Pd/C (425 mg) and MeOH (26 mL) are shaken on a parr shaker for 4 hrs under 55 psi hydrogen. After filtering through celite, pyruvic aldehyde (40% in Water, 1.8 g) is added and the reaction is stirred over the weekend. Removal of solvents under vacuum and silica gel purification affords 1-benzyl-3,6-dimethyl-1H-pyrazolo[3,4-b]pyrazine. Rt 2.85 min m/z 239.14(M+H)⁺

Step B

1-Benzyl-3,6-dimethyl-1H-pyrazolo[3,4-b]pyrazine (955 mg) and 1-butyl-3-methylimidazolium tetrafluoroborate (2 g) are heated to 110° C. and NBS (2.85 g) is added in several portions. After stirring for 10 min, diethyl ether is added and the ether layer is decanted (repeat three times). The combined ether layer is washed with Water, dried over sodium sulfate and concentrated. Final purification over silica gel affords 1-benzyl-5-bromo-3,6-dimethyl-1H-pyrazolo[3,4-b]pyrazine. Rt 3.42 min m/z 317.06(M+H)⁺

Step C

1-Benzyl-5-bromo-3,6-dimethyl-1H-pyrazolo[3,4-b]pyrazine (11 mg) and 6-isopropyl-2-methoxy-3-pyridine boronic acid (10 mg) are dissolved in toluene (600 uL). After 10 min of degassing, tetrakis(triphenylphosphine)palladium(0) (5 mg) is added, followed by 1 min of degassing. Upon addition of an aqueous 1N sodium carbonate solution (1 mL), the reaction mixture is microwaved 140° C. for 5 min. Subsequently, the crude mixture is purified on silica gel to afford 1-benzyl-5-(6-isopropyl-2-methoxy-pyridin-3-yl)-3,6-dimethyl-1H-pyrazolo[3,4-b]pyrazine. Rt 3.97 min m/z 388.20(M+H)⁺

### Example 13

### Synthesis of 1-Isopropyl-5-(6-isopropyl-2-methoxy-pyridin-3-yl)-3,6-dimethyl-1H-pyrazolo[3,4-b]pyrazine

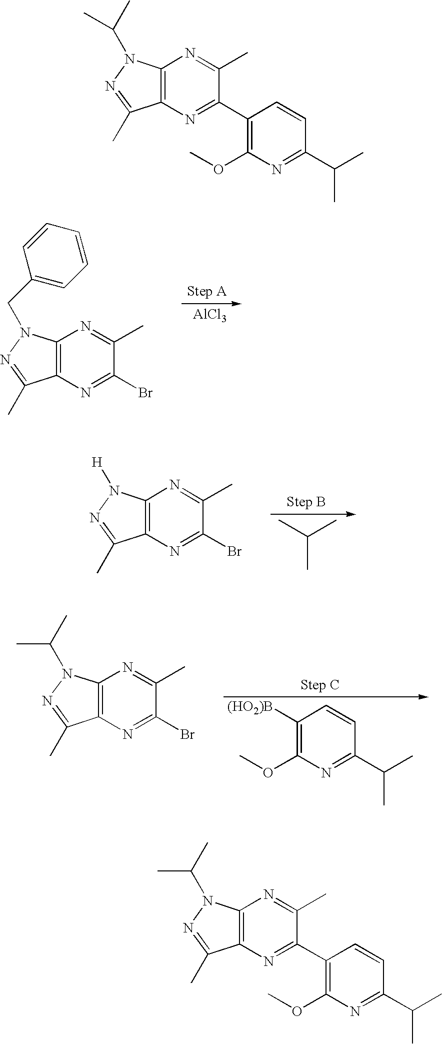 Step A

1-Benzyl-5-bromo-3,6-dimethyl-1H-pyrazolo[3,4-b]pyrazine (105 mg) is dissolved in anhydrous toluene (8 mL), aluminum chloride (176 mg) is added and the mixture is warmed to 50° C. for 1 h. All the solvent is removed under vacuum and the redidue is redissolved in EtOAc and is added into iced saturated NH₄Cl slurry. The mixture is extracted with EtOAc and the organic phase is separated and dried over sodium sulfate. Evaporation and silica gel purification afford 5-bromo-3,6-dimethyl-1H-pyrazolo[3,4-b]pyrazine. Rt 2.27 min m/z 227.00(M+H)⁺.

Step B

5-Bromo-3,6-dimethyl-1H-pyrazolo[3,4-b]pyrazine (19.6 mg) and K₂CO₃ (24 mg) are dissolved in DMF (1 mL). 2-iodopropane is added and the mixture is warmed to 60° C. After 1.5 h the reaction is cooled to RT, filtered, concentrated and purified on silica gel to afford 5-bromo-1-isopropyl-3,6-dimethyl-1H-pyrazolo[3,4-b]pyrazine. This compound was used without further purification in the next step.

Step C

5-Bromo-1-isopropyl-3,6-dimethyl-1H-pyrazolo[3,4-b]pyrazine (20 mg) and 6-isopropyl-2-methoxy-3-pyridine boronic acid (20 mg) are dissolved in toluene (600 uL). After 10 min of degassing, tetrakis(triphenylphosphine)palladium(0) (8 mg) is added, followed by 1 min of degassing. Upon addition of aqueous IN sodium carbonate solution (258 uL), the reaction mixture is heated to 90° C. for 3.5 h. The mixture is then cooled to RT, water is added, and it is extracted with EtOAc. The organic phase is separated, dried over sodium sulfate and evaporated under vacuum. Silica gel purification afford 1-Isopropyl-5-(6-isopropyl-2-methoxy-pyridin-3-yl)-3,6-dimethyl-1H-pyrazolo[3,4-b]pyrazine. Rt 3.98 min m/z 340.22(M+H)⁺

### Example 14

### Synthesis of Diethyl-{4-ethyl-5-[1-(1-ethyl-propyl)-3,6-dimethyl-1H-pyrazolo[3,4-b]pyrazin-5-yl]-pyridin-2-yl}-amine

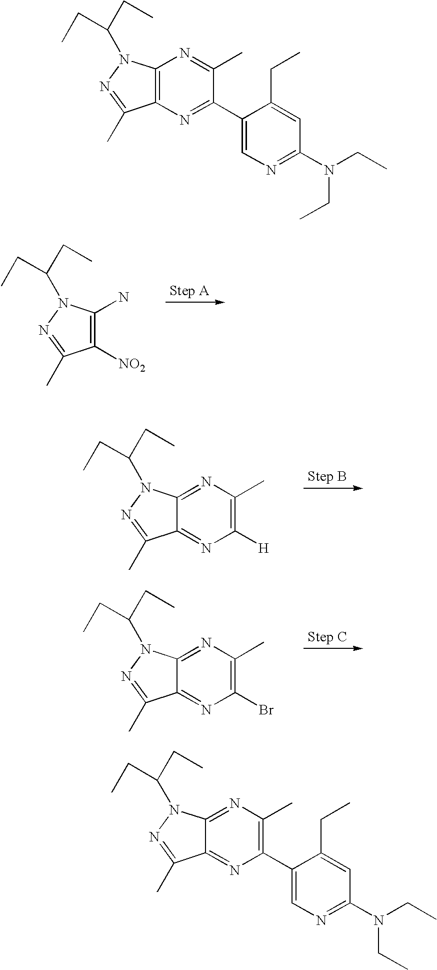 Step A

Analogous to the preparation of 1-benzyl-3,6-dimethyl-1H-pyrazolo[3,4-b]pyrazine, 2-(1-ethyl-propyl)-5-methyl-4-nitro-2H-pyrazol-3-ylamine is reduced and reacted with pyruvic aldehyde to give 1-(1-ethyl-propyl)-3,6-dimethyl-1H-pyrazolo[3,4-b]pyrazine. LCMS: m/z 219.14 (M+H)⁺, Rt=2.97 mins.

Step B

Analogous to the preparation of 1-benzyl-5-bromo-3,6-dimethyl-1H-pyrazolo[3,4-b]pyrazine, 1-(1-ethyl-propyl)-3,6-dimethyl-1H-pyrazolo[3,4-b]pyrazine is brominated to give 5-bromo-1-(1-ethyl-propyl)-3,6-dimethyl-1H-pyrazolo[3,4-b]pyrazine. LCMS: m/z 297.05 (M+H)⁺, Rt=3.65 mins.

Step C

Analogous to the preparation of 1-benzyl-5-(6-isopropyl-2-methoxy-pyridin-3-yl)-3,6-dimethyl-1H-pyrazolo[3,4-b]pyrazine, the palladium-mediated coupling of 5-bromo-1-(1-ethyl-propyl)-3,6-dimethyl-1H-pyrazolo[3,4-b]pyrazine (50 mg) with 2-dimethylamino-4-ethyl-5-pyridineboronic acid (45 mg) followed by purification on silica gel affords diethyl-{4-ethyl-5-[1-(1-ethyl-propyl)-3,6-dimethyl-1H-pyrazolo[3,4-b]pyrazin-5-yl]-pyridin-2-yl}-amine. LCMS: m/z 395.28 (M+H)⁺, Rt=2.57 mins.

### Example 15

### Synthesis of 5-(6-Diethylamino-4-ethyl-pyridin-3-yl)-1-(1-ethyl-propyl)-3-methyl-1H-pyrazolo[3,4-b]pyrazin-6-yl]-methyl-amine

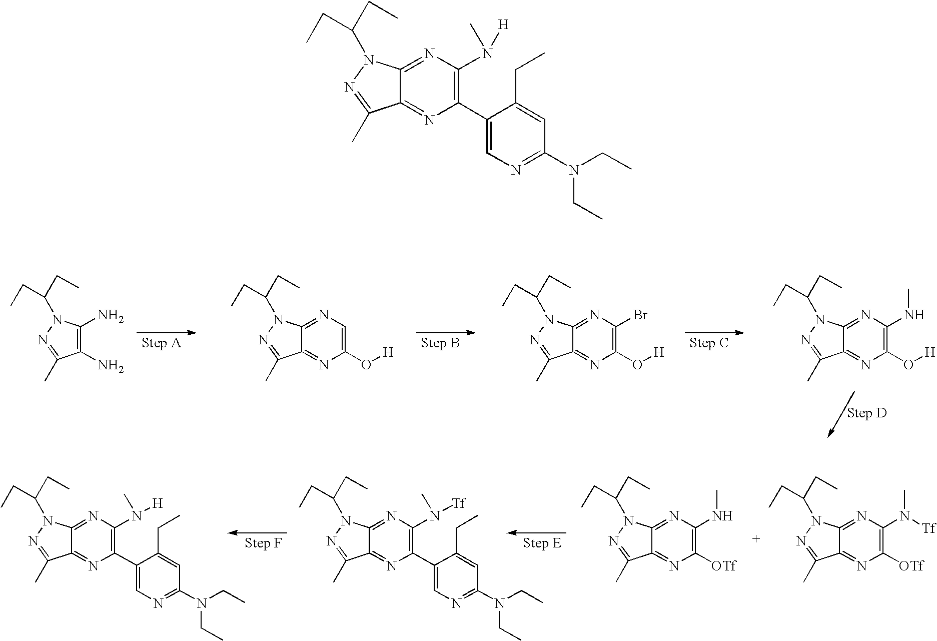 Step A

2-(]-Ethyl-propyl)-5-methyl-2H-pyrazole-3,4-diamine (2.5 g) and glyoxylic acid hydrate (1.5 g) are dissolved in methanol (40 mL). After cooling in an ice bath, glacial acetic acid is added (20 mL). The resulting solution is allowed to warm slowly to room temperature. After stirring for 9 hours, further glyoxylic acid hydrate (1.0 g) is added and allowed to stir at room temperature for a further 12 hours. The reaction is evaporated and treated with saturated sodium bicarbonate solution until any effervescence ceased. Extraction with DCM (4×50 mL) and drying over magnesium sulfate yields a crude product. Trituration of the crude product with ethyl ether gives 1-(1-ethyl-propyl)-3-methyl-1H-pyrazolo[3,4-b]pyrazin-5-ol. LCMS: m/z 221.2 (M+H)⁺, Rt 2.59 mins.

Step B

1-(1-Ethyl-propyl)-3-methyl-1H-pyrazolo[3,4-b]pyrazin-5-ol (922 mg) and N-bromosuccinimide (783 mg) are dissolved in chloroform (25 mL) and the resulting solution stirred at room temperature for 5 hours. Further N-bromosuccinimide (90 mg) is added and the mixture is stirred for 3 days. The reaction is diluted with DCM, washed with water (3×30 mL) and dried over magnesium sulfate. Evaporation directly gives 6-bromo-1-(1-ethyl-propyl)-3-methyl-1H-pyrazolo[3,4-b]pyrazin-5-ol. LCMS: m/z 299.1 (M+H)⁺, Rt 2.92 mins.

Step C

6-Bromo-1-(1-ethyl-propyl)-3-methyl-1H-pyrazolo[3,4-b]pyrazin-5-ol (550 mg) is dissolved in methylamine solution in THF (10 mL, 2.0 M) and the resulting solution is heated to 50° C. for 12 hours. The reaction mixture is evaporated to dryness and the residue is treated with saturated sodium bicarbonate solution. Extraction with EtOAc (2×40 mL), drying over magnesium sulfate and evaporation directly gives 1-(1-ethyl-propyl)-3-methyl-6-methylamino-1H-pyrazolo[3,4-b]pyrazin-5-ol. LCMS: m/z 250.2 (M+H)⁺, Rt 2.67 mins.

Step D

Analogous to the preparation of trifluoro-methanesulfonic acid 1-(1-ethyl-propyl)-3,6-dimethyl-1H-pyrazolo[3,4-b]pyrazin-5-yl ester, 1-(1-ethyl-propyl)-3-methyl-6-methylamino-1H-pyrazolo[3,4-b]pyrazin-5-ol (250 mg) is reacted with triflic anhydride (0.24 mL) in the presence of triethyl amine (0.35 mL). Purification on silica gel gives trifluoro-methanesulfonic acid 1-(1-ethyl-propyl)-3-methyl-6-methylamino-1H-pyrazolo[3,4-b]pyrazin-5-yl ester and trifluoro-methanesulfonic acid 1-(1-ethyl-propyl)-3-methyl-6-(methyl-trifluoromethanesulfonyl-amino)-1H-pyrazolo[3,4-b]pyrazin-5-yl ester as a mixture that is taken onto step E without further purification.

Step E

Analogous to the preparation of 5-(2,4-dichloro-phenyl)-1-(1-ethyl-propyl)-3,6-dimethyl-1H-pyrazolo[3,4-b]pyrazine, the palladium-mediated coupling of a mixture of trifluoro-methanesulfonic acid 1-(1-ethyl-propyl)-3-methyl-6-methylamino-1H-pyrazolo[3,4-b]pyrazin-5-yl ester and trifluoro-methanesulfonic acid 1-(1-ethyl-propyl)-3-methyl-6-(methyl-trifluoromethanesulfonyl-amino)-1H-pyrazolo[3,4-b]pyrazin-5-yl ester (100 mg) with 2-dimethylamino-4-ethyl-5-pyridineboronic acid (76 mg) gives N-[5-(6-diethylamino-4-ethyl-pyridin-3-yl)-1-(1-ethyl-propyl)-3-methyl-1H-pyrazolo[3,4-b]pyrazin-6-yl]-C,C,C-trifluoro-N-methyl-methanesulfonamide. LCMS: m/z 542.25 (M+H)⁺, Rt 3.05 mins.

Step F

A solution of N-[5-(6-diethylamino-4-ethyl-pyridin-3-yl)-1-(1-ethyl-propyl)-3-methyl-1H-pyrazolo[3,4-b]pyrazin-6-yl]-C,C,C-trifluoro-N-methyl-methanesufonamide (25 mg) in THF (1 mL) is treated with lithium aluminium hydride solution in THF (0.3mL, 1.0 M). After stirring at room temperature for 30 mins, the reaction is quenched with saturated sodium sulfate solution and then concentrated to low volume. The residue is extracted with DCM and the combined extracts evaporated. Purification on silica gel gives 5-(6-diethylamino-4-ethyl-pyridin-3-yl)-1-(1-ethyl-propyl)-3-methyl-1H-pyrazolo[3,4-b]pyrazin-6-yl]-methyl-amine. LCMS: m/z 410.31 (M+H)⁺, Rt 2.63 mins.

Using the analogous boronic acids in step E, the following compounds are synthesized:

### [1-(1-Ethyl-propyl)-5-(2-methoxy-4-trifluoromethoxy-phenyl)-3-methyl-1H-pyrazolo[3,4-b]pyrazin-6-yl]-methyl-amine. LCMS: m/z 424.19 (M+H)⁺, Rt 3.58 mins.

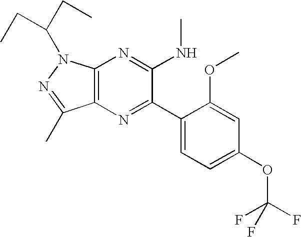

### [1-(1-Ethyl-propyl)-5-(6-isopropyl-2-methoxy-pyridin-3-yl)-3-methyl-1H-pyrazolo[3,4-b]pyrazin-6-yl]-methyl-amine. LCMS: m/z 383.25 (M+H)+, Rt 3.88 mins.

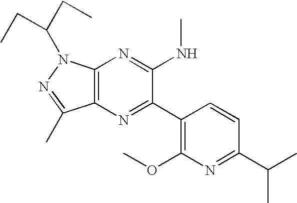

### Example 16

### Synthesis of 1-(1-Ethyl-propyl)-6-methoxy-5-(2-methoxy-4-trifluoromethoxy-phenyl)-3-methyl-1H-pyrazolo[3,4-b]pyrazine and 1-(1-Ethyl-propyl)-6-hydroxy-5-(2-methoxy-4-trifluoromethoxy-phenyl)-3-methyl-1H-pyrazolo[3,4-b]pyrazine

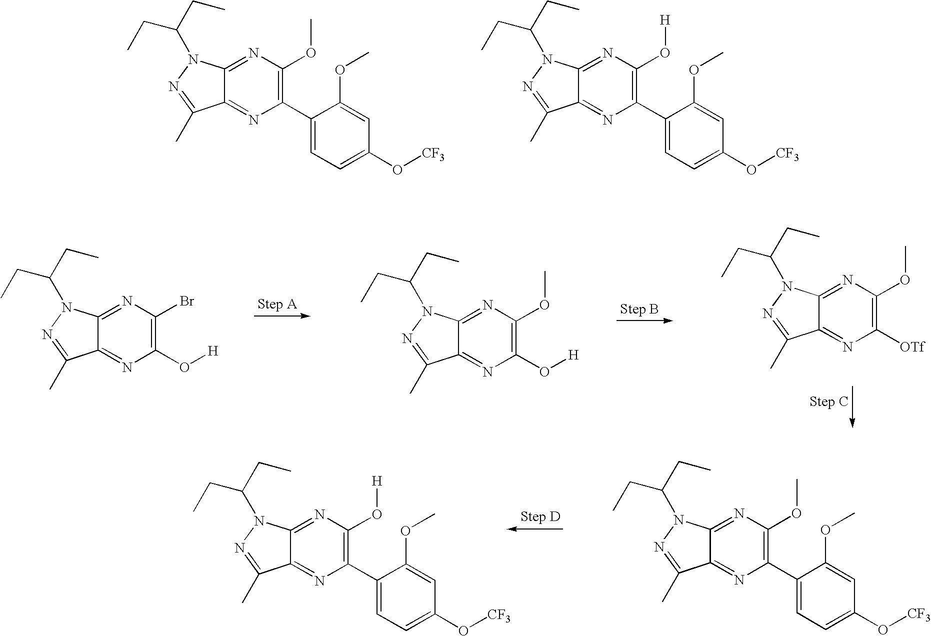 Step A

A solution of 6-bromo-1-(1-ethyl-propyl)-3-methyl-1H-pyrazolo[3,4-b]pyrazin-5-ol (550 mg) in methanol (20 mL) is treated with sodium methoxide solution in methanol (10 mL, 25% wt. solution). After stirring at room temperature for 14 hours, the reaction mixture is concentrated to low volume. The residue is diluted with water and the pH adjusted to 7 with hydrochloric acid solution. Extraction with EtOAc (5×30 mL), drying over magnesium sulfate and evaporation directly gives 1-(1-ethyl-propyl)-6-methoxy-3-methyl-1H-pyrazolo[3,4-b]pyrazin-5-ol. LCMS: m/z 251.15 (M+H)⁺, Rt 2.33 mins.

Step B

Analogous to the preparation of trifluoro-methanesulfonic acid 1-(1-ethyl-propyl)-3,6-dimethyl-1H-pyrazolo[3,4-b]pyrazin-5-yl ester, 1-(1-ethyl-propyl)-6-methoxy-3-methyl-1H-pyrazolo[3,4-b]pyrazin-5-ol (940 mg) is reacted with triflic anhydride (0.88 mL) in the presence of triethyl amine (1.5 mL). Purification on silica gel gives trifluoro-methanesulfonic acid 1-(1-ethyl-propyl)-6-methoxy-3-methyl-1H-pyrazolo[3,4-b]pyrazin-5-yl ester. LCMS: m/z 383.10 (M+H)⁺, Rt 4.02 mins.

Step C

Analogous to the preparation of 5-(2,4-dichloro-phenyl)-1-(1-ethyl-propyl)-3,6-dimethyl-1H-pyrazolo[3,4-b]pyrazine, the palladium mediated coupling of trifluoro-methanesulfonic acid 1-(1-ethyl-propyl)-6-methoxy-3-methyl-1H-pyrazolo[3,4-b]pyrazin-5-yl ester (450 mg) with 2-methoxy-4-trifluromethoxybenzeneboronic acid (361 mg) gives 1-(1-ethyl-propyl)-6-methoxy-5-(2-methoxy-4-trifluoromethoxy-phenyl)-3-methyl-1H-pyrazolo[3,4-b]pyrazine. LCMS: m/z 425.14 (M+H)⁺, Rt 3.82 mins.

Step D

Analogous to the preparation of 3-[6-ethyl-1-(1-ethyl-propyl)-3-methyl-1H-pyrazolo[3,4-b]pyrazin-5-yl]-6-isopropyl-pyridin-2-ol, the reaction of diethyl- {4-ethyl-5-[1-(1-ethyl-propyl)-6-methoxy-3-methyl-1H-pyrazolo[3,4-b]pyrazin-5-yl]-pyridin-2-yl}-amine (153 mg) with sodium thiomethoxide (261 mg) gives 5-(6-diethylamino-4-ethyl-pyridin-3-yl)-1-(1-ethyl-propyl)-3-methyl-1H-pyrazolo[3,4-b]pyrazin-6-ol. LCMS: m/z 397.2 (M+H)⁺, Rt 2.17 mins.

Using the analogous boronic acids in step C, the following compounds are synthesized:

### Diethyl-{4-ethyl-5-[1-(1-ethyl-propyl)-6-methoxy-3-methyl-1H-pyrazolo[3,4-b[pyrazin-5-yl]-pyridin-2-yl}-amine. LCMS: m/z 411.25 (M+H)⁺, Rt 2.68 mins.

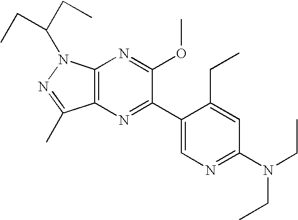

### 5-(6-Diethylamino-4-ethyl-pyridin-3-yl)-1-(1-ethyl-propyl)-3-methyl-1H-pyrazolo[3,4-b]pyrazin-6-ol. LCMS: m/z 397.2 (M+H)⁺, Rt 2.17 mins.

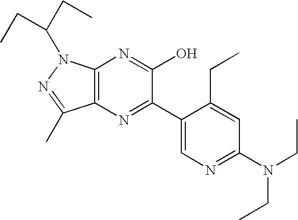

### 1-(1-Ethyl-propyl)-5-(6-isopropyl-2-methoxy-pyridin-3-yl)-6-methoxy-3-methyl-1H-pyrazolo[3,4-b]pyrazine. LCMS: m/z 384.21 (M+H)⁺, Rt 4.22 mins.

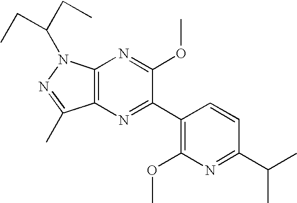

### Example 17

### Synthesis of 6-Ethyl-5-(6-isopropyl-2-methoxy-pyridin-3-yl)-1-(2-methoxy-1-methoxymethyl-ethyl)-3-methyl-1H-pyrazolo[3,4-b]pyrazine

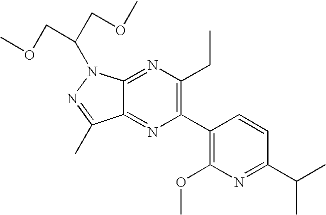 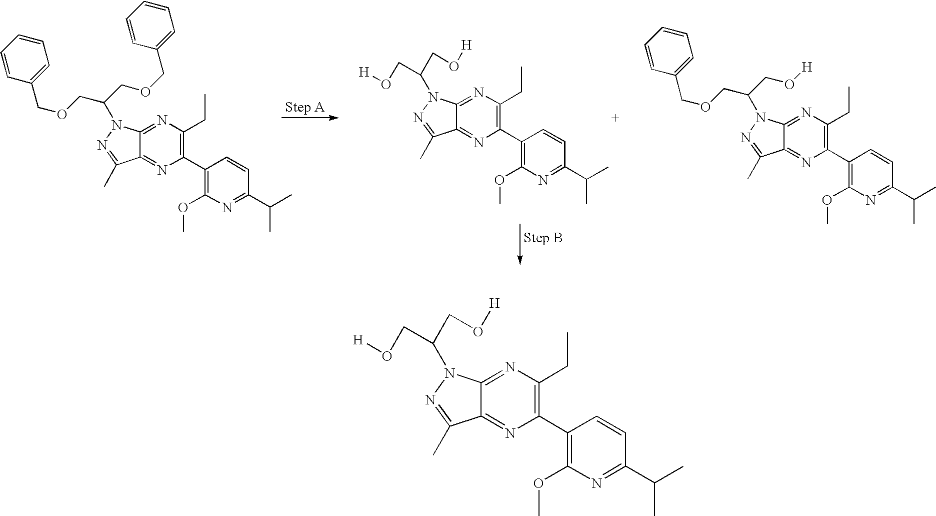 Step A

A suspension of 1-(2-benzyloxy-1-benzyloxymethyl-ethyl)-6-ethyl-5-(6-isopropyl-2-methoxy-pyridin-3-yl)-3-methyl-1H-pyrazolo[3,4-b]pyrazine (793 mg) and palladium on activated charcoal (60 mg, 10% wt.) in ethanol (30 mL) is stirred under a hydrogen atmosphere for 16 hours. Further palladium on activated charcoal (60 mg, 10% wt.) is added and the reaction mixture stirred under a hydrogen atmosphere for further 6 hours. The reaction mixture is filtered and the filtrate is evaporated. Purification on silica gel gives 3-benzyloxy-2-[6-ethyl-5-(6-isopropyl-2-methoxy-pyridin-3-yl)-3-methyl-pyrazolo[3,4-b]pyrazin-1-yl]-propan-1-ol and 2-[6-ethyl-5-(6-isopropyl-2-methoxy-pyridin-3-yl)-3-methyl-pyrazolo[3,4-b]pyrazin-1-yl]-propane-1,3-diol. LCMS (monobenzyl): m/z 476.2 (M+H)⁺, Rt 3.70 mins. LCMS (diol): m/z 386.2 (M+H)⁺, Rt 2.87 mins.

Step B

A solution of 2-[6-ethyl-5-(6-isopropyl-2-methoxy-pyridin-3-yl)-3-methyl-pyrazolo[3,4-b]pyrazin-1-yl]-propane-1,3-diol (65 mg) in DMF (2 mL) is cooled to −8° C. and treated with sodium hydride (10 mg, 95%). The resulting mixture is treated with methyl iodide (0.029 mL) and the reaction mixture allowed to warm to room temperature. After stirring at room temperature for an hour, the mixture is diluted with saturated brine and extracted with ethyl ether (2×30 mL). The combined extracts are evaporated. Purification on silica gel gives 6-ethyl-5-(6-isopropyl-2-methoxy-pyridin-3-yl)-1-(2-methoxy-1-methoxymethyl-ethyl)-3-methyl-1H-pyrazolo[3,4-b]pyrazine. LCMS: m/z 414.3 (M+H)⁺, Rt 3.73 mins.

### Synthesis of {3-[6-Ethyl-1-(2-methoxy-1-methoxymethyl-ethyl)-3-methyl-1H-pyrazolo[3,4-b]pyrazin-5-yl]-6-isopropyl-pyridin-2-yl}-methyl-amine

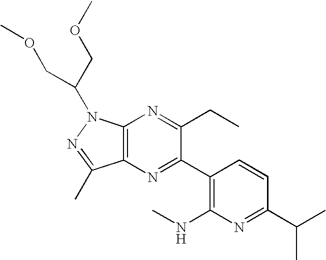

Starting with 6-ethyl-5-(6-isopropyl-2-methoxy-pyridin-3-yl)-1-(2-methoxy-1-methoxymethyl-ethyl)-3-methyl-1H-pyrazolo[3,4-b]pyrazine, {3-[6-ethyl-1-(2-methoxy-1-methoxymethyl-ethyl)-3-methyl-1H-pyrazolo[3,4-b]pyrazin-5-yl]-6-isopropyl-pyridin-2-yl}-methyl-amine is obtained in analogous fashion to {3-[6-ethyl-1-(1-ethyl-propyl)-3-methyl-1H-pyrazolo[3,4-b]pyrazin-5-yl]-6-isopropyl-pyridin-2-yl}-methyl-amine. LCMS: m/z 413.3 (M+H)⁺, Rt=1.95 mins.

### Synthesis of {3-[6-Ethyl-1-(2-methoxy-1-methoxymethyl-ethyl)-3-methyl-1H-pyrazolo[3,4-b]pyrazin-5-yl]-6-isopropyl-pyridin-2-yl}-dimethyl-amine

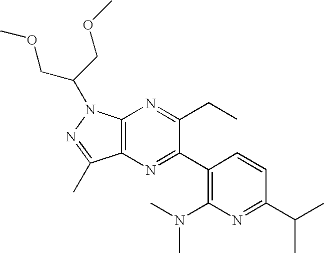

Substituting dimethylamine for methylamine gives, in analogous fashion, {3-[6-ethyl-1-(2-methoxy-1-methoxymethyl-ethyl)-3-methyl-1H-pyrazolo[3,4-b]pyrazin-5-yl]-6-isopropyl-pyridin-2-yl}-dimethyl-amine. LCMS: m/z 427.3 (M+H)⁺, Rt=2.72 mins.

### Synthesis of 6-Ethyl-5-(2-ethyl-6-isopropyl-pyridin-3-yl)-1-(2-methoxy-1-methoxymethyl-ethyl)-3-methyl-1H-pyrazolo[3,4-b]pyrazine

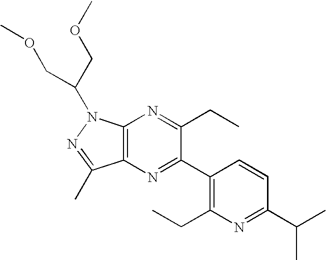

In like manner, 6-ethyl-5-(2-ethyl-6-isopropyl-pyridin-3-yl)-1-(2-methoxy-1-methoxymethyl-ethyl)-3-methyl-1H-pyrazolo[3,4-b]pyrazine is obtained analogously to 6-ethyl-5-(2-ethyl-6-isopropyl-pyridin-3-yl)-1-(2-methoxy-1-methyl-ethyl)-3-methyl-1H-pyrazolo[3,4-b]pyrazine. LCMS: m/z 412.3 (M+H)⁺, Rt=2.17 mins.

### Synthesis of 5-(6-Isopropyl-2-methoxy-pyridin-3-yl)-1-(2-methoxy-1-methoxymethyl-ethyl)-3,6-dimethyl-1H-pyrazolo[3,4-b]pyrazine

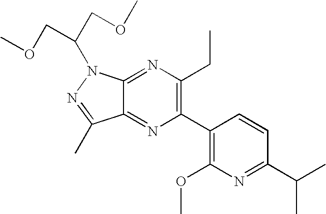

Analogously, 2-[5-(6-isopropyl-2-methoxy-pyridin-3-yl)-3,6-dimethyl-pyrazolo[3,4-b]pyrazin-1-yl]-propane-1,3-diol (168 mg) is reacted with methyl iodide (0.079 mL). Purification on silica gel gives 5-(6-isopropyl-2-methoxy-pyridin-3-yl)-1-(2-methoxy-1-methoxymethyl-ethyl)-3,6-dimethyl-1H-pyrazolo[3,4-b]pyrazine. LCMS: m/z 400.1 (M+H)⁺, Rt 3.68 mins.

### Example 18

### Synthesis of Cyclobutyl-{2-[6-ethyl-5-(6-isopropyl-2-methoxy-pyridin-3-yl)-3-methyl-pyrazolo[3,4-b]pyrazin-1-yl]-3-methoxy-propyl}-amine

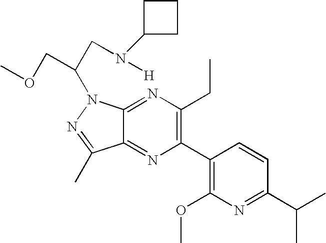 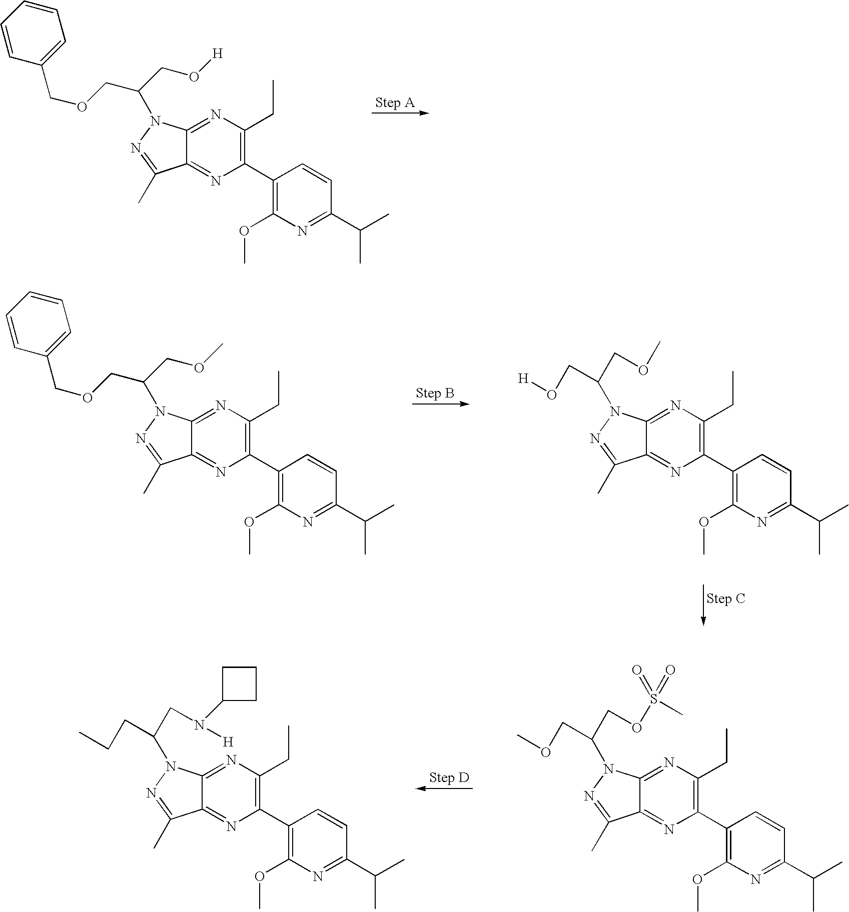 Step A

Analogous to the preparation of 6-ethyl-5-(6-isopropyl-2-methoxy-pyridin-3-yl)-1-(2-methoxy-1-methoxymethyl-ethyl)-3-methyl-1H-pyrazolo[3,4-b]pyrazine, 3-benzyloxy-2-[6-ethyl-5-(6-isopropyl-2-methoxy-pyridin-3-yl)-3-methyl-pyrazolo[3,4-b]pyrazin-1-yl]-propan-1-ol (325 mg) is reacted sodium iodidewith methyl iodide (0.060 mL). Purification on silica gel gives 1-(2-benzyloxy-1-methoxymethyl-ethyl)-6-ethyl-5-(6-isopropyl-2-methoxy-pyridin-3-yl)-3-methyl-1H-pyrazolo[3,4-b]pyrazine. LCMS: m/z 490.2 (M+H)⁺, Rt 4.37 mins.

Step B

Analogous to the preparation of 3-benzyloxy-2-[6-ethyl-5-(6-isopropyl-2-methoxy-pyridin-3-yl)-3-methyl-pyrazolo[3,4-b]pyrazin-1-yl]-propan-1-ol, 1-(2-benzyloxy-1-methoxymethyl-ethyl)-6-ethyl-5-(6-isopropyl-2-methoxy-pyridin-3-yl)-3-methyl-1H-pyrazolo[3,4-b]pyrazine (282 mg) is reacted with hydrogen in the presence of palladium on activated charcoal. Evaporation of the filtrate directly gave 2-[6-ethyl-5-(6-isopropyl-2-methoxy-pyridin-3-yl)-3-methyl-pyrazolo[3,4-b]pyrazin-1-yl]-3-methoxy-propan-1-ol. LCMS: m/z 400.2 (M+H)⁺, Rt 3.30 mins.

Step C

Analogous to the preparation of trifluoro-methanesulfonic acid 1-(1-ethyl-propyl)-3,6-dimethyl-1H-pyrazolo[3,4-b]pyrazin-5-yl ester, 2-[6-ethyl-5-(6-isopropyl-2-methoxy-pyridin-3-yl)-3-methyl-pyrazolo[3,4-b]pyrazin-1-yl]-3-methoxy-propan-1-ol (207 mg) is reacted with mesyl chloride (0.044 mL) in the presence of triethylamine. Evaporation of the solvent extracts directly gives methanesulfonic acid 2-[6-ethyl-5-(6-isopropyl-2-methoxy-pyridin-3-yl)-3-methyl-pyrazolo[3,4-b]pyrazin-1-yl]-3-methoxy-propyl ester. LCMS: m/z 478.2 (M+H)⁺, Rt=3.55 mins.

Step D

Analogous to the preparation of {3-[6-ethyl-1-(1-ethyl-propyl)-3-methyl-1H-pyrazolo[3,4-b]pyrazin-5-yl]-6-isopropyl-pyridin-2-yl}-dimethyl-amine, methanesulfonic acid 2-[6-ethyl-5-(6-isopropyl-2-methoxy-pyridin-3-yl)-3-methyl-pyrazolo[3,4-b]pyrazin-1-yl]-3-methoxy-propyl ester (55 mg) is reacated with cyclobutyl amine (0.098 mL). Purification on silica gel gives cyclobutyl-{2-[6-ethyl-5-(6-isopropyl-2-methoxy-pyridin-3-yl)-3-methyl-pyrazolo[3,4-b]pyrazin-1-yl]-3-methoxy-propyl}-amine. LCMS: m/z 453.3 (M+H)⁺, Rt=2.37 mins.

Using analogous amines in step D, the following compounds are synthesized:

### 6-Ethyl-5-(6-isopropyl-2-methoxy-pyridin-3-yl)-1-(1-methoxymethyl-2-pyrrolidin-1-yl-ethyl)-3-methyl-1H-pyrazolo[3,4-b]pyrazine. LCMS: m/z 453.3 (M+H)⁺, Rt=2.27 mins.

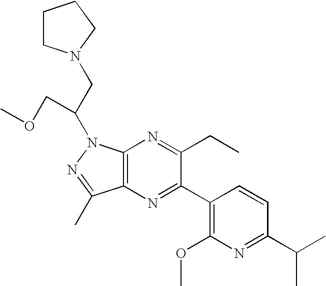

### Ethyl-{2-[6-ethyl-5-(6-isopropyl-2-methoxy-pyridin-3-yl)-3-methyl-pyrazolo [3,4-b]pyrazin-1-yl]-3-methoxy-propyl}-methyl-amine. LCMS: m/z 441.3 (M+H)⁺. Rt=2.27 mins.

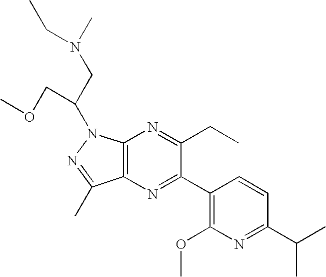

### 6-Ethyl-5-(6-isopropyl-2-methoxy-pyridin-3-yl)-1-(1-methoxymethyl-2-morpholin-4-yl-ethyl)-3-methyl-1H-pyrazolo[3,4-b]pyrazine. LCMS: m/z 469.3 (M+H)⁺. Rt=2.35 mins.

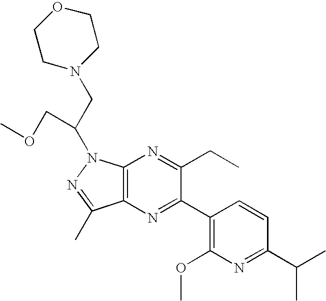

### Example 19

### Synthesis of Cyclobutyl-{2-[6-ethyl-5-(6-isopropyl-2-methoxy-pyridin-3-yl)-3-methyl-pyrazolo[3,4-b]pyrazin-1-yl]-propyl}-amine and 6-Ethyl-1-isopropyl-5-(6-isopropyl-2-methoxy-pyridin-3-yl)-3-methyl-1H-pyrazolo[3,4-b]pyrazine

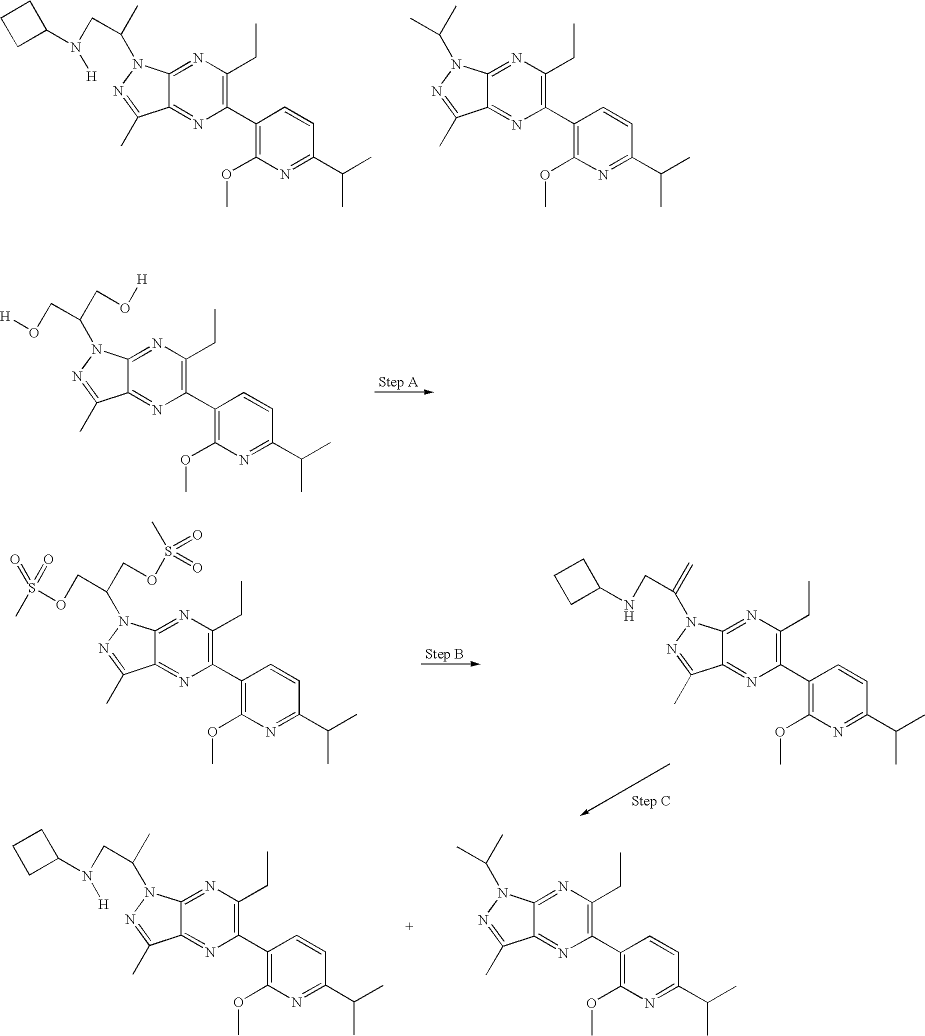 Step A

Analogous to the preparation of trifluoro-methanesulfonic acid 1-(1-ethyl-propyl)-3,6-dimethyl-1H-pyrazolo[3,4-b]pyrazin-5-yl ester, 2-[6-ethyl-5-(6-isopropyl-2-methoxy-pyridin-3-yl)-3-methyl-pyrazolo[3,4-b]pyrazin-1-yl]-propane-1,3-diol (177 mg) is reacted with mesyl chloride (0.078 mL). Evaporation of the solvent extracts directly gives methanesulfonic acid 2-[6-ethyl-5-(6-isopropyl-2-methoxy-pyridin-3-yl)-3-methyl-pyrazolo[3,4-b]pyrazin-1-yl]-3-methanesulfonyloxy-propyl ester. LCMS: m/z 542.1 (M+H)⁺, Rt=3.37 mins.

Step B

Analogous to the preparation of {3-[6-ethyl-1-(1-ethyl-propyl)-3-methyl-1H-pyrazolo[3,4-b]pyrazin-5-yl]-6-isopropyl-pyridin-2-yl}-dimethyl-amine, methanesulfonic acid 2-[6-ethyl-5-(6-isopropyl-2-methoxy-pyridin-3-yl)-3-methyl-pyrazolo[3,4-b]pyrazin-1-yl]-3-methanesulfonyloxy-propyl ester (55 mg) is reacted with cyclobutyl amine (0.174 mL). Purification on silica gel gives cyclobutyl-{2-[6-ethyl-5-(6-isopropyl-2-methoxy-pyridin-3-yl)-3-methyl-pyrazolo[3,4-b]pyrazin-1-yl]-allyl}-amine. LCMS: m/z 421.3 (M+H)⁺, Rt=2.50 mins.

Step C

A suspension of cyclobutyl-{2-[6-ethyl-5-(6-isopropyl-2-methoxy-pyridin-3-yl)-3-methyl-pyrazolo[3,4-b]pyrazin-1-yl]-allyl}-amine (14 mg) and palladium on activated charcoal (3 mg, 10% wt.) in ethanol (3 mL) is shaken under 20 PSI hydrogen atmosphere for 2 hours. Purification on silica gel gives cyclobutyl-{2-[6-ethyl-5-(6-isopropyl-2-methoxy-pyridin-3-yl)-3-methyl-pyrazolo[3,4-b]pyrazin-1-yl]-propyl}-amine and 6-ethyl-1-isopropyl-5-(6-isopropyl-2-methoxy-pyridin-3-yl)-3-methyl-1H-pyrazolo[3,4-b]pyrazine. LCMS (cyclobutylamino): m/z 423.3 (M+H)⁺, Rt 3.02 mins. LCMS (isopropyl): m/z 354.2 (M+H)⁺, Rt 4.59 mins.

Using analogous amines in step C, the following compounds are synthesized:

### 6-Ethyl-5-(6-isopropyl-2-methoxy-pyridin-3-yl)-3-methyl-1-(1-pyrrolidin-1-ylmethyl-vinyl)-1H-pyrazolo[3,4-b]pyrazine LCMS: m/z 421.3 (M+H)⁺, Rt=2.47 mins.

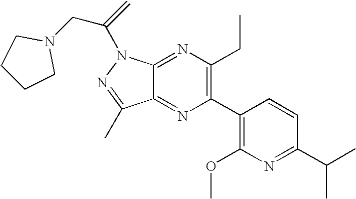

### 6-Ethyl-5-(6-isopropyl-2-methoxy-pyridin-3-yl)-3-methyl-1-(1-methyl-2-pyrrolidin-1-yl-ethyl)-1H-pyrazolo[3,4-b]pyrazine. LCMS: m/z 423.3 (M+H)⁺Rt 3.02 mins.

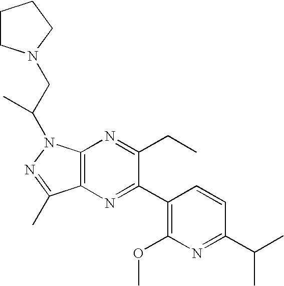

### Ethyl-{2-[6-ethyl-5-(6-isopropyl-2-methoxy-pyridin-3-yl)-3-methyl-pyrazolo [3,4-b]pyrazin-1-yl]-allyl}-methyl-amine. LCMS: m/z 409.3 (M+H)⁺, Rt=2.45 mins.

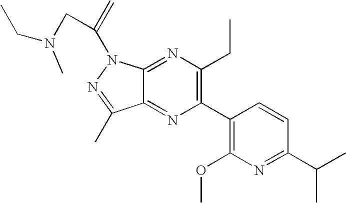

### Ethyl-{2-[6-ethyl-5-(6-isopropyl-2-methoxy-pyridin-3-yl)-3-methyl-pyrazolo[3,4-b]pyrazin-1-yl]-propyl}-methyl-amine. LCMS: m/z 411.3 (M+H)⁺, Rt 2.90 mins.

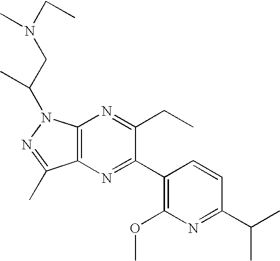

### 6-Ethyl-5-(6-isopropyl-2-methoxy-pyridin-3-yl)-3-methyl-1-(1-morpholin-4-ylmethyl-vinyl)-1H-pyrazolo[3,4-b]pyrazine. LCMS: m/z 437.3 (M+H)⁺, Rt=2.42 mins.

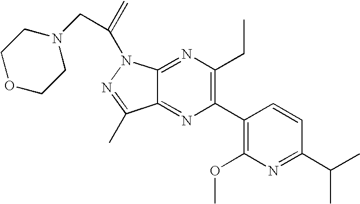

### 6-Ethyl-5-(6-isopropyl-2-methoxy-pyridin-3-yl)-3-methyl-1-(1-methyl-2-morpholin-4-yl-ethyl)-1H-pyrazolo[3,4-b]pyrazine. LCMS: m/z 439.3 (M+H)⁺, Rt 2.99 mins.

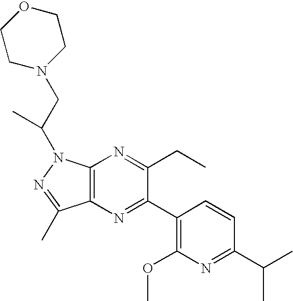

### Example 20

### Synthesis of Diethyl-{4-ethyl-5-[3-(1-ethyl-propyl)-1,5-dimethyl-1H-pyrazolo[3,4-b]pyridin-6-yl]-pyridin-2-yl}-amine

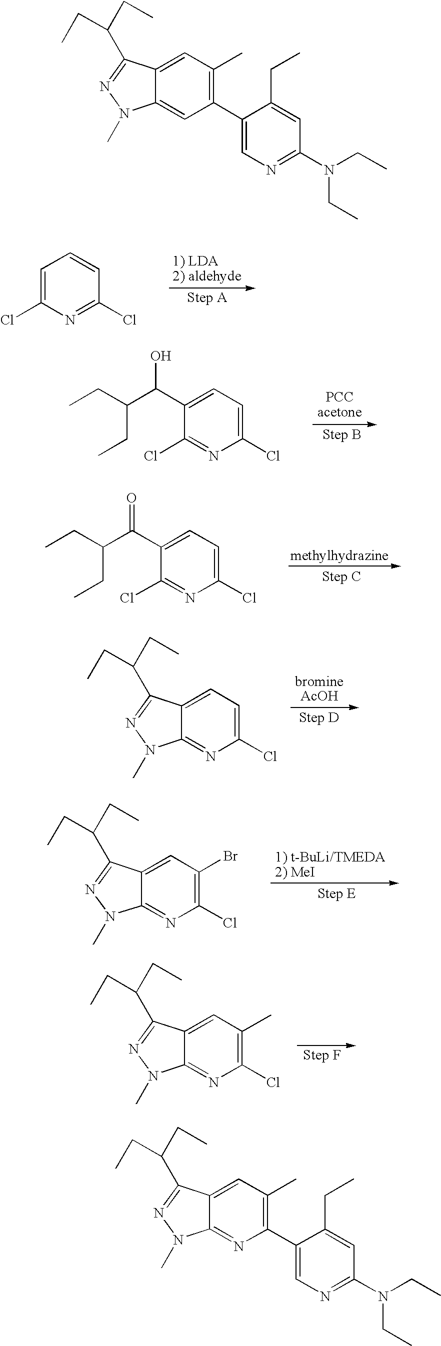 Step A

Diisopropylamine (14.3 mL) in THF (125 mL) is cooled to −78° C. and subsequently treated with n-butyllithium in hexanes (62.5 mL, 1.6N). After stirring for 1 h, 2,6-dichloropyridine (14.8 g) in THF (50 mL) is added slowly. Stirring for 1 h is followed by slow addition of 2-ethylbutyraldehyde (13.5 mL) in THF (50 mL). After stirring for 1½ h the reaction mixture is put into saturated ammonium chloride solution (500 mL). Extraction with DCM (3×300 mL) and drying over magnesium sulfate yields a crude product. Purification on silica gel affords 1-(2,6-dichloro-pyridin-3-yl)-2-ethyl-butan-1-ol. LCMS: m/z 248.12 (M+H)⁺

Step B

1-(2,6-Dichloro-pyridin-3-yl)-2-ethyl-butan-1-ol (17.09 g) is dissolved in dry acetone (700 mL). Dry powdered molecular sieves (53 g, 4A) and PCC (52 g) are added and the mixture is stirred over night. Filtering through celite (200 g) and purification on silica gel affords 1-(2,6-dichloro-pyridin-3-yl)-2-ethyl-butan-1-one. Rf (CH₂Cl₂/hexane=3:1)=0.38

Step C

1-(2,6-Dichloro-pyridin-3-yl)-2-ethyl-butan-1-one is dissolved in ethanol (300 mL), treated with methylhydrazine (8.25 g), and heated to 60° C. for 2 h. The reaction mixture is put into water (500 mL), extracted with DCM (3×200 mL) and dried over magnesium sulfate. Purification on silica gel affords 6-chloro-3-(1-ethyl-propyl)-1-methyl-1H-pyrazolo[3,4-b]pyridine. LCMS: m/z 238.17 (M+H)⁺

Step D

6-Chloro-3-(1-ethyl-propyl)-1-methyl-1H-pyrazolo[3,4-b]pyridine (3.0 g) is dissolved in glacial acetic acid (100 mL). Addition of bromine (2.59 mL) and heating to 60° C. for 16 h shows traces of the starting material still remaining. Addition of bromine (0.5 mL) and heating to 60° C. for 1 h is followed by addition of saturated sodium carbonate (500 mL) and 1N sodium sulfite (200 mL). Extraction with DCM (4×200 mL) and drying over magnesium sulfate leads to a crude mixture which is purified on silica gel to afford 5-bromo-6-chloro-3-(1-ethyl-propyl)-1-methyl-1H-pyrazolo[3,4-b]pyridine. LCMS: m/z 316.07 (M+H)⁺

Step E

TMEDA (4.29 mL) in THF (100 mL) is cooled to −78° C. and then treated with t-butyllithium in pentane (13.9 mL, 1.7N). Stirring for 5 min is followed by slow addition of 5-bromo-6-chloro-3-(1-ethyl-propyl)-1-methyl-1H-pyrazolo[3,4-b]pyridine (3 g) in THF (15 mL). The resulting orange/red solution is treated after 20 min with iodomethane (2.37 mL) and subsequently stirred for 1 h. Being put into a mixture of water (300 ml) and saturated sodium bicarbonate (100 mL), the aqueous layer is extracted with DCM (3×200 mL). The combined organic layers are dried over sodium sulfate. Purification on silica gel affords 6-chloro-3-(1-ethyl-propyl)-1,5-dimethyl-1H-pyrazolo[3,4-b]pyridine. LCMS: m/z 252.18 (M+H)⁺

Step F

6-Chloro-3-(1-ethyl-propyl)-1,5-dimethyl-1H-pyrazolo[3,4-b]pyridine (100 mg) and 2-dimethylamino-4-ethyl-5-pyridineboronic acid are dissolved in DME (5 mL). After 10 min of degassing, tetrakis(triphenylphosphine)palladium(0) (46 mg) is added, followed by 1 min of degassing. Upon addition of an aqueous 1N sodium carbonate solution (1 mL), the reaction mixture is heated to 80° C. for 16 h. Subsequently, the crude mixture is put into water (100 mL), extracted with DCM (3×100 mL), and dried over sodium carbonate. Purification on silica gel affords Diethyl-{4-ethyl-5-[3-(1-ethyl-propyl)-1,5-dimethyl-1H-pyrazolo[3,4-b]pyridin-6-yl]-pyridin-2-yl}-amine. LCMS: m/z 394.32 (M+H)⁺

Using analogous boronic acids, the following compounds are prepared.

### 3-(1-Ethyl-propyl)-6-(2-methoxy-4-trifluoromethoxy-phenyl)-1,5-dimethyl-1H-pyrazolo[3,4-b]pyridine. MS m/z 408.21 (M+H)⁺

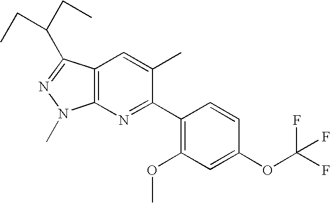

### 6-(2-Chloro-4-methoxy-phenyl)-3-(1-ethyl-propyl)-1,5-dimethyl-1H-pyrazolo [3,4-b]pyridine. MS m/z 358.19 (M+H)⁺

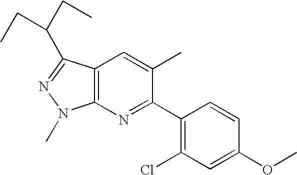

### Example 21

### Synthesis of 5-Chloro-3-(1-ethyl-propyl)-6-(2-methoxy-4-trifluoromethoxy-phenyl)-1-methyl-1H-pyrazolo[3,4-b]pyridine and 5-Chloro-6-(5-chloro-2-methoxy-4-trifluoromethoxy-phenyl)-3-(1-ethyl-propyl)-1-methyl-1H-pyrazolo[3,4-b]pyridine

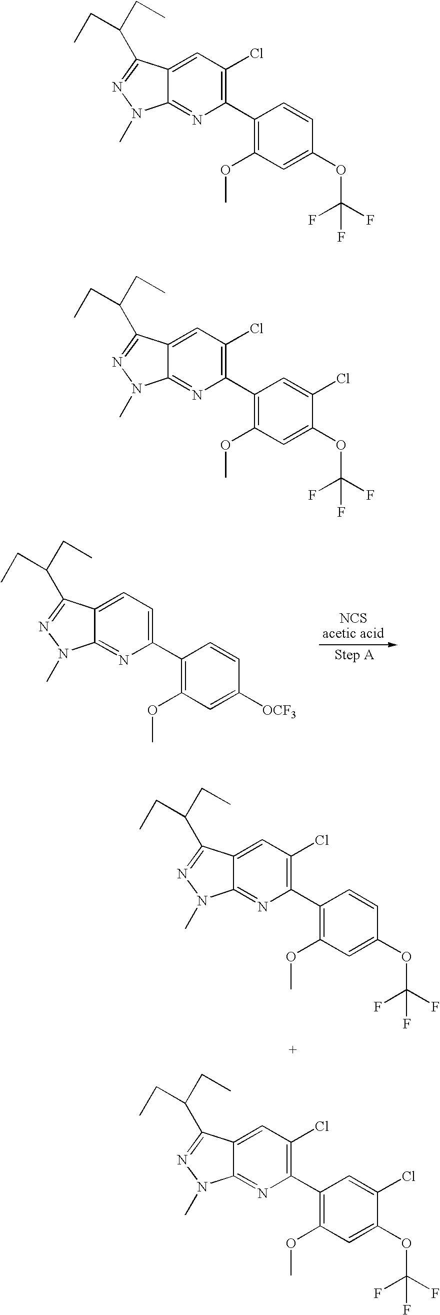 Step A

3-(1-Ethyl-propyl)-6-(2-methoxy-4-methyl-phenyl)-1-methyl-1H-pyrazolo[3,4-b]pyridine (100 mg) and NCS (102 mg) are dissolved in glacial acetic acid (5 mL). The clear mixture is heated to 60° C. for 3½ h or until LCMS control shows the disappearance of all starting material. Prolonged reaction time leads to increased formation of the dichloro-compound. The resulting mixture is put into water (100 mL), extracted with DCM (3×100 mL), and dried over magnesium sulfate. Final purification via preparative TLC yields the two title compounds in an approximate 1/1 ratio.

LCMS (monochloride): m/z 428.17 (M+H)⁺

LCMS (dichloride): LCMS: m/z 462.11 (M+H)⁺

### Example 22

### Synthesis of 5-Ethyl-3-(1-ethyl-propyl)-6-(2-methoxy-4-trifluoromethoxy-phenyl)-1-methyl-1H-pyrazolo[3,4-b]pyridine

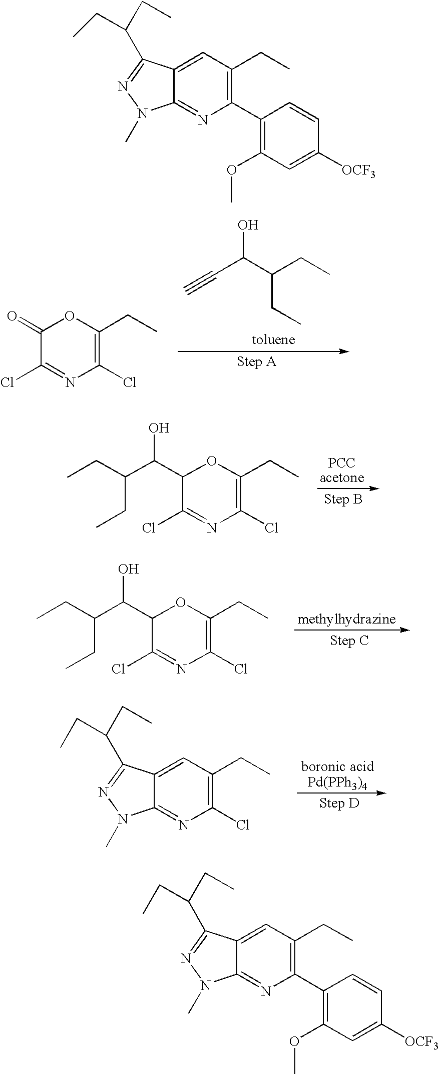 Step A

Similar to a procedure by Hoornaert et al. (Synthesis, 1991, 765), 1-(2,6-dichloro-5-ethyl-pyridin-3-yl)-2-ethyl-butan-1-ol is prepared by Diels-Alder reaction of 3,5-dichloro-6-ethyl-[1,4]oxazin-2-one and 4-ethyl-3-hydroxy-1-hexyne. Rf (CH₂Cl₂)=0.52

Step B

1-(2,6-Dichloro-5-ethyl-pyridin-3-yl)-2-ethyl-butan-1-one is analogously synthesized by PCC (1.12 g) oxidation of 1-(2,6-dichloro-5-ethyl-pyridin-3-yl)-2-ethyl-butan-1-ol (144 mg) in acetone. Purification on silica gel affords 1-(2,6-dichloro-5-ethyl-pyridin-3-yl)-2-ethyl-butan-1-ol. LCMS: m/z 274.12 (M+H)⁺

Step C

6-Chloro-5-ethyl-3-(1-ethyl-propyl)-1-methyl-1H-pyrazolo[3,4-b]pyridine is synthesized by condensation of 1-(2,6-dichloro-5-ethyl-pyridin-3-yl)-2-ethyl-butan-1-one (133 mg) with methylhydrazine (53 □L). Purification on silica gel affords the compound. LCMS: m/z 266.20 (M+H)⁺

Step D

Analogously, 5-Ethyl-3-(1-ethyl-propyl)-6-(2-methoxy-4-trifluoromethoxy-phenyl)-1-methyl-1H-pyrazolo[3,4-b]pyridine is synthesized by palladium mediated coupling of 6-chloro-5-ethyl-3-(1-ethyl-propyl)-1-methyl-1H-pyrazolo[3,4-b]pyridine (91 mg) with 2-methoxy-4-trifluromethoxybenzeneboronic acid (87 mg). Purification on silica gel affords the title compound. LCMS: m/z 422.22 (M+H)⁺

### Example 23

### Synthesis of (1-Ethyl-propyl)-{5-[3-(1-ethyl-propyl)-1,5-dimethyl-1H-pyrazolo[3,4-b]pyridin-6-yl]-3-methoxy-6-methyl-pyrazin-2-yl}-amine

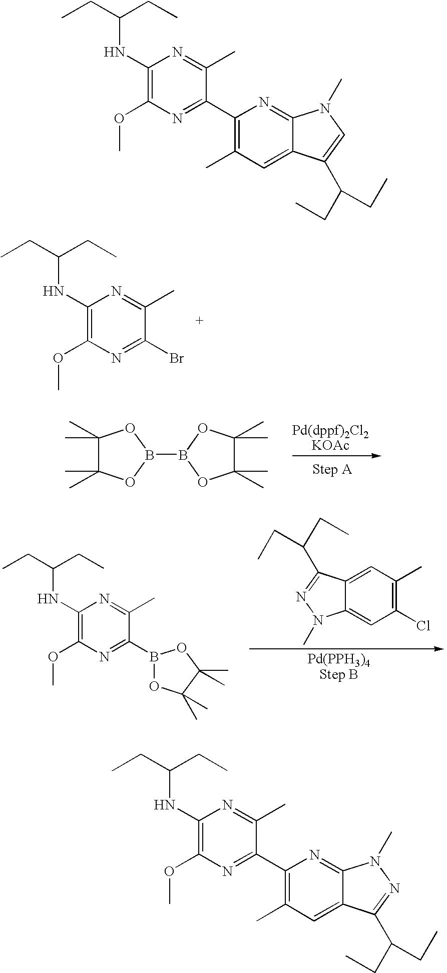 Step A

(5-Bromo-3-methoxy-6-methyl-pyrazin-2-yl)-(1-ethyl-propyl)-amine (229 mg), bis(pinacolato)diboran (242 mg), potassium acetate (233 mg), and (1,1′-bis(diphenylphosphino)ferrocene)dichloropalladium(II) (130 mg, complex with DCM) are dissolved in DMSO (5 mL) and then heated to 80° C. for 2 days. The resulting crude mixture is taken onto step B once LCMS confirms all starting material is consumed.

Step B

6-Chloro-3-(1-ethyl-propyl)-1,5-dimethyl-1H-pyrazolo[3,4-b]pyridine (100 mg), tetrakis(triphenylphosphine)palladium(0) (92 mg), and cesium carbonate (259 mg) are added into the crude mixture from step A. The resulting black suspension is heated to 80° C. for 2 days, until LCMS confirms almost complete conversion. Subsequently, the mixture is put into water (100 mL), extracted with DCM (3×100 mL), and dried over magnesium sulfate. Final purification on silica gel affords (1-ethyl-propyl)-{5-[3-(I-ethyl-propyl)-1,5-dimethyl-1H-pyrazolo[3,4-b]pyridin-6-yl]-3-methoxy-6-methyl-pyrazin-2-yl}-amine. LCMS: m/z 425.34 (M+H)⁺

TABLE V

| Cmpd | | | MS, | |
|---|---|---|---|---|
| # | STRUCTURE | COMPOUND NAME | m/z | Rt(min) |
| 301 | 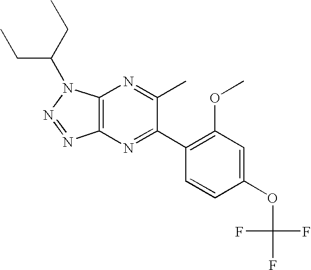 | 1-(1-Ethyl-propyl)-5-(2-methoxy- 4-trifluoromethoxy-phenyl)-6- methyl-1H-[1,2,3]triazolo[4,5- b] pyrazine | 396.29 | |
| 302 | 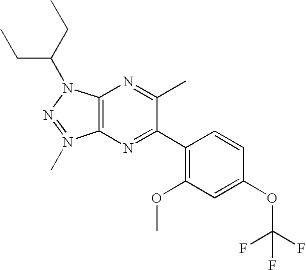 | 1-(1-Ethyl-propyl)-5-(2-methoxy- 4-trifluoromethoxy-phenyl)-3,6- dimethyl-1H-pyrazolo[3,4- b]pyrazine | 409.18 | 3.98 |
| 303 | 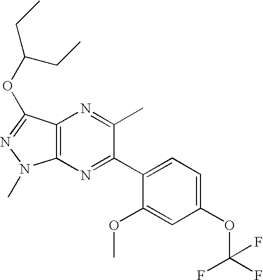 | 3-(1-Ethyl-propoxy)-6-(2-methoxy- 4-trifluoromethoxy-phenyl)-1,5- dimethyl-1H-pyrazolo[3,4- b]pyrazine | 425.16 | 4.03 |
| 304 | 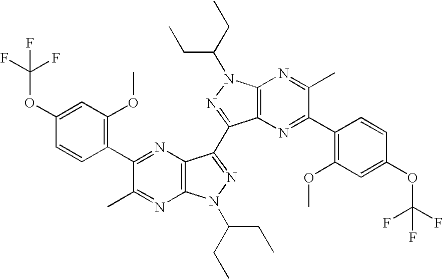 | 1,1′-Bis-(1-ethyl-propyl)-5,5′- bis-(2-methoxy-4-trifluoro- methoxy-phenyl)-6,6′-dimethyl- 1H,1′H-[3,3]bi[pyrazolo[3,4-b]pyrazinyl] | 787.24 | 4.80 |
| 305 | 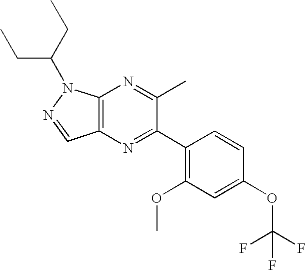 | 1-(1-Ethyl-propyl)-5-(2-methoxy-4- trifluoromethoxy-phenyl)-6-methyl- 1H-pyrazolo[3,4-b]pyrazine | 395.16 | 4.08 |
| 306 | 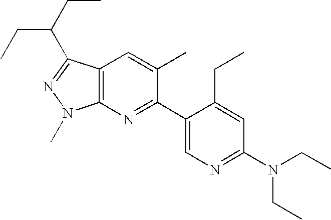 | Diethyl-{4-ethyl-5-[3-(1-ethyl-propyl)- 1,5-dimethyl-1H-pyrazolo[3,4- b]pyridin-6-yl]-pyridin-yl}-amine | 394.32 | |
| 307 | 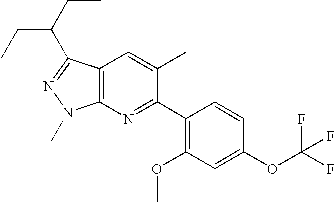 | 3-(1-Ethyl-propyl)-6-(2-methoxy-4- trifluoromethoxy-phenyl)-1,5- diethyl-1H-pyrazolo[3,4-b]pyridine | 408.21 | |
| 308 | 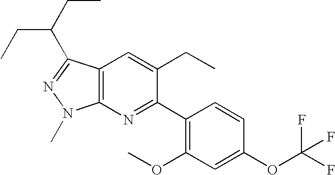 | 5-Ethyl-3-(1-ethyl-propyl)-6-(2- methoxy-4-trifluoromethoxy- phenyl)-1-methyl-1H-pyrazolo[3,4- b]pyridine | 422.22 | |
| 309 | 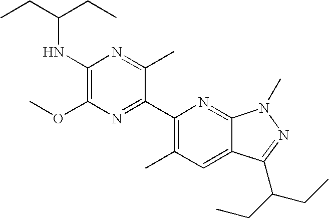 | (1-Ethyl-propyl)-{5-[3-(1-ethyl- propyl)-1,5-dimethyl-1H- pyrazolo[3,4-b]pyridin-6-yl]-3- methoxy-6-methyl-pyrazin-2-yl}- amine | 425.34 | |
| 310 | 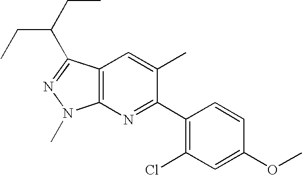 | 6-(2-Chloro-4-methoxy-phenyl)-3- (1-ethyl-propyl)-1,5-dimethyl-1H- pyrazolo[3,4-b]pyridine | 358.19 | |
| 311 | 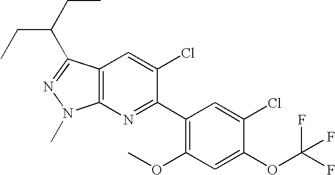 | 5-Chloro-6-(5-chloro-2-methoxy-4- trifluoromethoxy-phenyl)-3-(1-ethyl- propyl)-1-methyl-1H-pyrazolo[3,4- b]pyridine | 462.11 | |
| 312 | 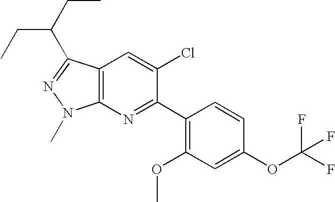 | 5-Chloro-3-(1-ethyl-propyl)-6-(2- methoxy-4-trifluoromethoxy- phenyl)-1-methyl-1H-pyrazolo[3,4- b]pyridine | 428.17 | |
| 313 | 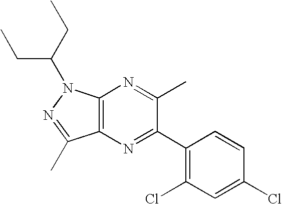 | 5-(2,4-Dichloro-phenyl)-1-(1-ethyl- propyl)-3,6-dimethyl-1H-pyrazolo[3,4- b]pyrazine | 363.11 | 4.27 |
| 314 | 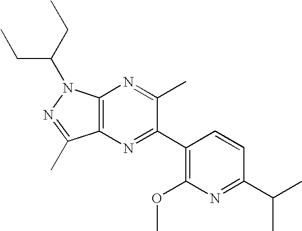 | 1-(1-Ethyl-propyl)-5-(6-isopropyl-2- methoxy-pyridin-3-yl)-3,6-dimethyl- 1H-pyrazolo[3,4-b]pyrazine | 368.3 | 4.38 |
| 315 | 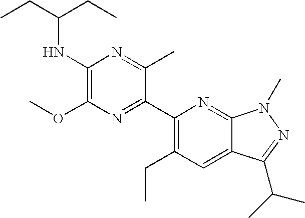 | [5-(5-Ethyl-3-isopropyl-1-methyl-1H- pyrazolo[3,4-b]pyridin-6-yl)-3- methoxy-6-methyl-pyrazin-2-yl]-(1-ethyl- propyl)-amine | 411.21 | |
| 316 | 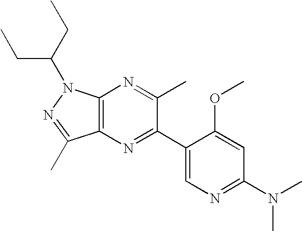 | {5-[1-(1-Ethyl-propyl)-3,6-dimethyl- 1H-pyrazolo[3,4-b]pyrazin-5-yl]-4- methoxy-pyridin-2-yl}-dimethyl-amine | 369.2 | 2.25 |
| 317 | 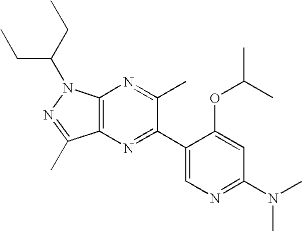 | {5-[1-(1-Ethyl-propyl)-3,6-dimethyl- 1H-pyrazolo[3,4-b]pyrazin-5-yl]-4- isopropoxy-pyridin-2-yl}-dimethyl-amine | 397.2 | 2.53 |
| 318 | 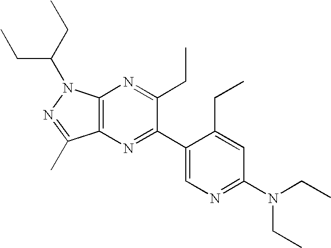 | Diethyl-{4-ethyl-5-[6-ethyl-1-(1- ethyl-propyl)-3-methyl-1H- pyrazolo[3,4-b]pyrazin-5-yl]-pyridin-2- yl}-amine | 409.3 | 3.12 |
| 319 | 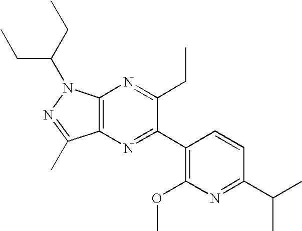 | 6-Ethyl-1-(1-ethyl-propyl)-5-(6- isopropyl-2-methoxy-pyridin-3-yl)-3- methyl-1H-pyrazolo[3,4-b]pyrazine | 382.3 | 4.69 |
| 320 | 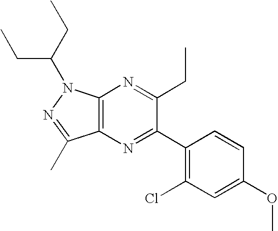 | 5-(2-Chloro-4-methoxy-phenyl)-6- ethyl-1-(1-ethyl-propyl)-3-methyl- 1H-pyrazolo[3,4-b]pyrazine | 373.2 | 4.24 |
| 321 | 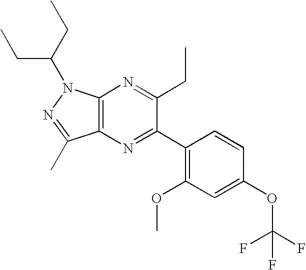 | 6-Ethyl-1-(1-ethyl-propyl)-5-(2- methoxy-4-trifluoromethoxy- phenyl)-3-methyl-1H-pyrazolo[3,4- b]pyrazine | 423.2 | 4.29 |
| 322 | 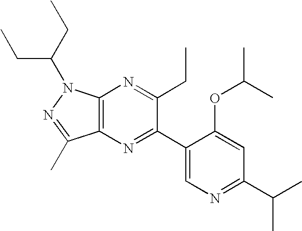 | {5-[6-Ethyl-1-(1-ethyl-propyl)-3- methyl-1H-pyrazolo[3,4-b]pyrazin- 5-yl]-4-isopropoxy-pyridin-2-yl}- dimethyl-amine | 411.3 | 2.99 |
| 323 | 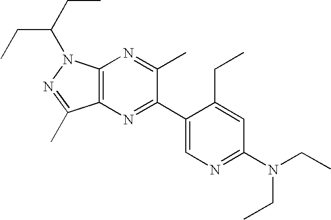 | Diethyl-{4-ethyl-5-[1-(1-ethyl-propyl)- 3,6-dimethyl-1H-pyrazolo[3,4-b]pyrazin-5-yl]-pyridin-2-yl}-amine | 395.28 | 2.57 |
| 324 | 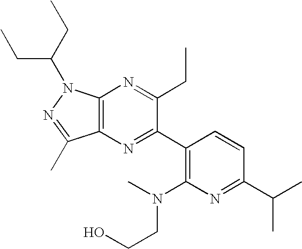 | 2-({3-[6-Ethyl-1-(1-ethyl-propyl)-3- methyl-1H-pyrazolo[3,4-b]pyrazin- 5-yl]-6-isopropyl-pyridin-2-yl}- methyl-amino)-ethanol | 425.28 | 2.80 |
| 325 | 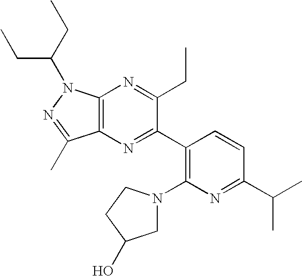 | 1-{3-[6-Ethyl-1-(1-ethyl-propyl)-3- methyl-1H-pyrazolo[3,4-b]pyrazin- 5-yl]-6-isopropyl-pyridin-2-yl}- pyrrolidin-3-ol | 437.28 | 2.63 |
| 326 | 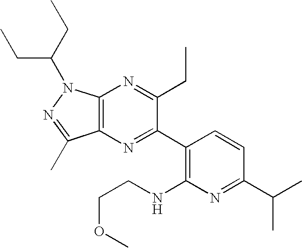 | {3-[6-Ethyl-1-(1-ethyl-propyl)-3- methyl-1H-pyrazolo[3,4-b]pyrazin- 5-yl]-6-isopropyl-pyndin-2-yl}-(2- methoxy-ethyl)-amine | 425.28 | 2.90 |
| 327 | 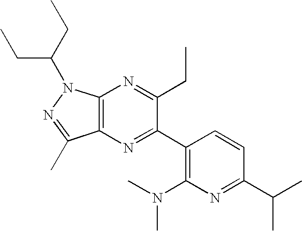 | {3-(6-Ethyl-1-(1-ethyl-propyl)-3- methyl-1H-pyrazolo[3,4-b]pyrazin- 5-yl]-6-isopropyl-pyndin-2-yl}- methyl-amine | 395.27 | 3.58 |
| 328 | 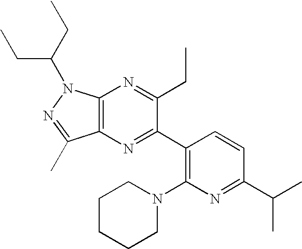 | 3′-[6-Ethyl-1-(1-ethyl-propyl)-3- methyl-1H-pyrazolo[3,4-b]pyrazin- 5-yl]-6-isopropyl-3,4,5,6-tetrahydro- 2H-[1,2′]bipyridinyl | 435.30 | 4.58 |
| 329 | 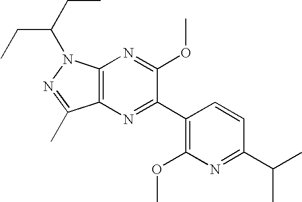 | 1-(1-Ethyl-propyl)-5-(6-isopropyl-2- methoxy-pyridin-3-yl)-6-methoxy-3- methyl-1H-pyrazolo[3,4-b]pyrazine | 384.21 | 4.22 |
| 330 | 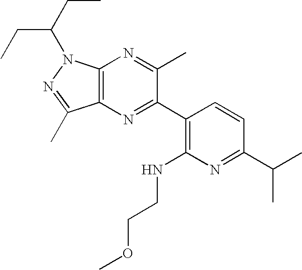 | {3-[1-(1-Ethyl-propyl)-3,6-dimethyl- 1H-pyrazolo[3,4-b]pyrazin-5-yl]-6- isopropyl-pyridin-2-yl}-(2-methoxy- ethyl)-amine | 411.29 | 2.65 |
| 331 | 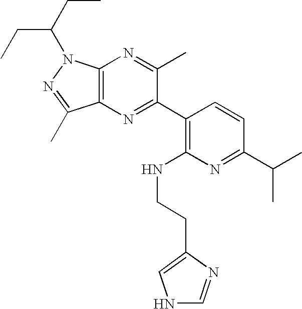 | {3-[1-(1-Ethyl-propyl)-3,6-dimethyl- 1H-pyrazolo[3,4-b]pyrazin-5-yl]-6- isopropyl-pyridin-2-yl}-[2-(1H- imidazol-4-yl)-ethyl]-amine | 447.30 | 2.18 |
| 332 | 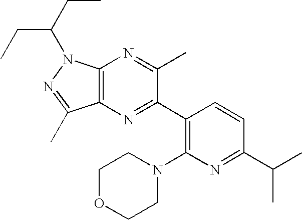 | 1-(1-Ethyl-propyl)-5-(6-isopropyl-2- morpholin-4-yl-pyridin-3-yl)-3,6- dimethyl-1H-pyrazolo[3,4-b]pyrazine | 423.27 | 3.92 |
| 333 | 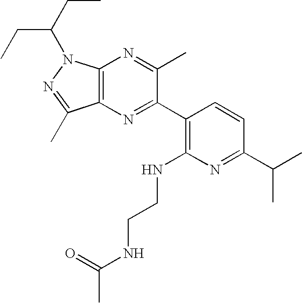 | N-(2-{3-[1-(1-Ethyl-propyl)-3,6- dimethyl-1H-pyrazolo[3,4-b]pyrazin- 5-yl]-6-isopropyl-pyridin-2-ylamino}- ethyl)-acetamide | 438.29 | 2.37 |
| 334 | 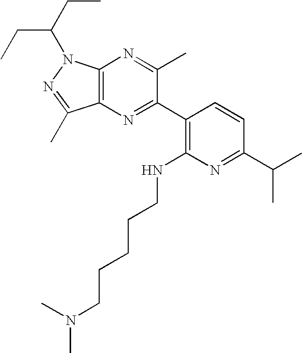 | N′-{3-[1-(1-Ethyl-propyl)-3,6- dimethyl-1H-pyrazolo[3,4-b]pyrazin- 5-yl]-6-isopropyl-pyridin-2-yl}-N,N- dimethyl-pentane-1,5-diamine | 466.38 | 2.07 |
| 335 | 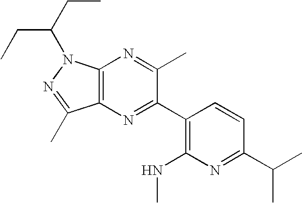 | {3-[1-(1-Ethyl-propyl)-3,6-dimethyl- 1H-pyrazolo[3,4-b]pyrazin-5-yl]-6- isopropyl-pyridin-2-yl}-methyl- amine | 367.3 | 2.72 |
| 336 | 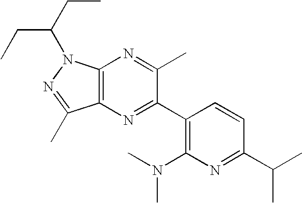 | {3-[1-(1-Ethyl-propyl)-3,6-dimethyl- 1H-pyrazolo[3,4-b]pyrazin-5-yl]-6- isopropyl-pyridin-2-yl}-dimethyl- amine | 381.26 | 3.18 |
| 337 | 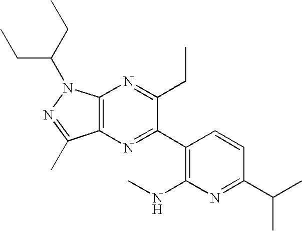 | {3-[6-Ethyl-1-(1-ethyl-propyl)-3- methyl-1H-pyrazolo[3,4-b]pyrazin- 5-yl]-6-isopropyl-pyridin-2-yl}- methyl-amine | 381.3 | 2.92 |
| 338 | 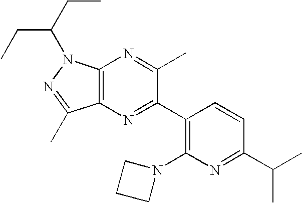 | 5-(2-Azetidin-1-yl-6-isopropyl- pyridin-3-yl)-1-(1-ethyl-propyl)-3,6- dimethyl-1H-pyrazolo[3,4-b]pyrazine | 393.3 | 2.95 |
| 339 | 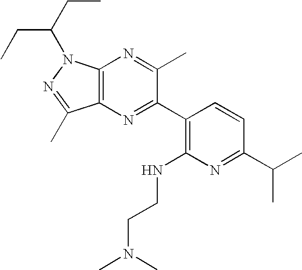 | N′-{3-[1-(1-Ethyl-propyl)-3,6- dimethyl-1H-pyrazolo[3,4-b]pyrazin- 5-yl]-6-isopropyl-pyridin-2-yl}-N,N- dimethyl-ethane-1,2-diamine | 424.32 | 2.70 |
| 340 | 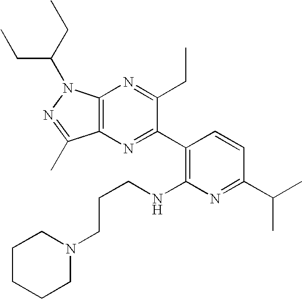 | {3-[6-Ethyl-1-(1-ethyl-propyl)-3- methyl-1H-pyrazolo[3,4-b]pyrazin- 5-yl]-6-isopropyl-pyridin-2-yl}-(3- piperidin-1-yl-propyl)-amine | 492.4 | 2.84 |
| 341 | 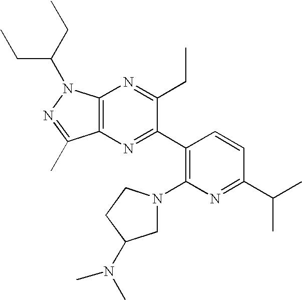 | (1-{3-[6-Ethyl-1-(1-ethyl-propyl)-3- methyl-1H-pyrazolo[3,4-b]pyrazin- 5-yl]-6-isopropyl-pyridin-2-yl}- pyrrolidin-3-yl)-dimethyl-amine | 464.36 | 2.98 |
| 342 | 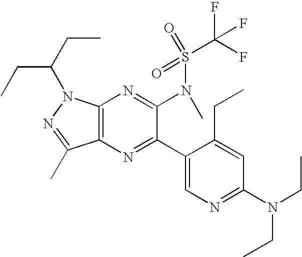 | N-[5-(6-Diethylamino-4-ethyl- pyridin-3-yl)-1-(1-ethyl-propyl)-3- methyl-1H-pyrazolo[3,4-b]pyrazin- 6-yl]-C,C,C-trifluoro-N-methyl- methanesulfonamide | 542.25 | 3.05 |
| 343 | 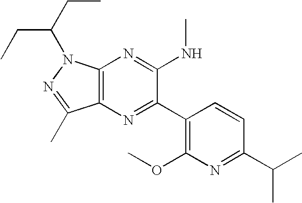 | [1-(1-Ethyl-propyl)-5-(6-isopropyl-2- methoxy-pyridin-3-yl)-3-methyl-1H- pyrazolo[3,4-b]pyrazin-6-yl]-methyl- amine | 383.25 | 3.88 |
| 344 | 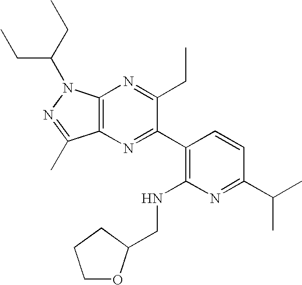 | {3-[6-Ethyl-1-(1-ethyl-propyl)-3- methyl-1H-pyrazolo[3,4-b]pyrazin- 5-yl]-6-isopropyl-pyridin-2-yl}- (tetrahydro-furan-2-ylmethyl)-amine | 451.28 | 2.77 |
| 345 | 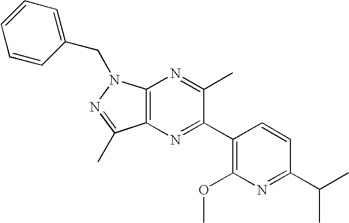 | 1-Benzyl-5-(6-isopropyl-2-methoxy- pyridin-3-yl)-3,6-dimethyl-1H- pyrazolo[3,4-b]pyrazine | 388.20 | 3.97 |
| 346 | 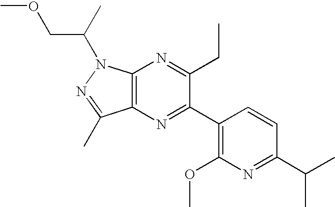 | 6-Ethyl-5-(6-isopropyl-2-methoxy- pyridin-3-yl)-1-(2-methoxy-1- methyl-ethyl)-3-methyl-1H- pyrazolo[3,4-b]pyrazine | 384.21 | 3.92 |
| 347 | 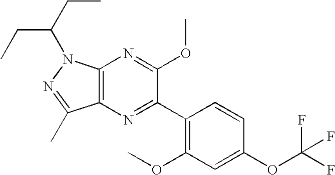 | 1-(1-Ethyl-propyl)-6-methoxy-5-(2- methoxy-4-trifluoromethoxy- phenyl)-3-methyl-1H-pyrazolo[3,4- b]pyrazine | 425.14 | 3.82 |
| 348 | 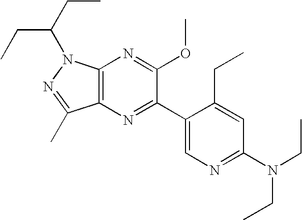 | Diethyl-{4-ethyl-5-[1-(1-ethyl- propyl)-6-methoxy-3-methyl-1H- pyrazolo[3,4-b]pyrazin-5-yl]-pyridin- yl}-amine | 411.25 | 2.68 |
| 349 | 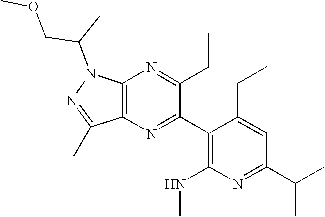 | {3-[6-Ethyl-1-(2-methoxy-1-methyl- ethyl)-3-methyl-1H-pyrazolo[3,4- b]pyrazin-5-yl]-6-isopropyl-pyridin- 2-yl}-methyl-amine | 383.24 | 2.32 |
| 350 | 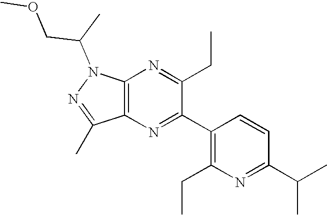 | 6-Ethyl-5-(2-ethyl-6-isopropyl- pyridin-3-yl)-1-(2-methoxy-1- methyl-ethyl)-3-methyl-1H- pyrazolo[3,4-b]pyrazine | 382.26 | 2.42 |
| 351 | 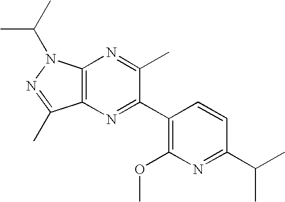 | 1-Isopropyl-5-(6-isopropyl-2- methoxy-pyridin-3-yl)-3,6-dimethyl- 1H-pyrazolo[3,4-b]pyrazine | 340.22 | 3.98 |
| 352 | 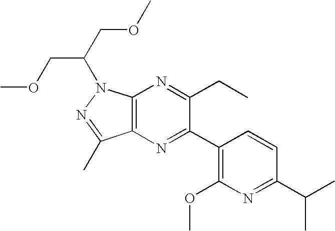 | 6-Ethyl-5-(6-isopropyl-2-methoxy- pyridin-3-yl)-1-(2-methoxy-1- methoxymethyl-ethyl)-3-methyl-1H- pyrazolo[3,4-b]pyrazine | 414.3 | 3.73 |
| 353 | 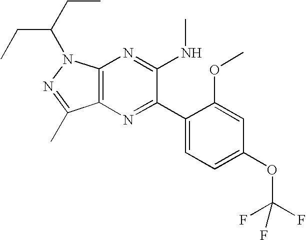 | [1-(1-Ethyl-propyl)-5-(2-methoxy-4- trifluoromethoxy-phenyl)-3-methyl- 1H-pyrazolo[3,4-b]pyrazin-6-yl]- methyl-amine | 424.19 | 3.58 |
| 354 | 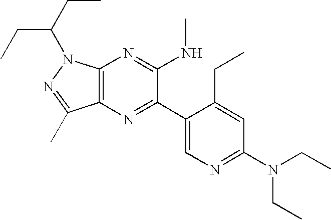 | [5-(6-Diethylamino-4-ethyl-pyridin- 3-yl)-1-(1-ethyl-propyl)-3-methyl- 1H-pyrazolo[3,4-b]pyrazin-6-yl]- methyl-amine | 410.31 | 2.63 |
| 355 | 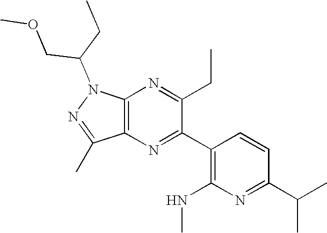 | {3-[6-Ethyl-1-(1-methoxymethyl- propyl)-3-methyl-1H-pyrazolo[3,4- b]pyrazin-5-yl]-6-isopropyl-pyridin- 2-yl}-methyl-amine | 397.2 | 2.82 |
| 356 | 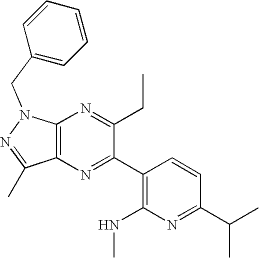 | [3-(1-Benzyl-6-ethyl-3-methyl-1H- pyrazolo[3,4-b]pyrazin-5-yl)-6- isopropyl-pyridin-2-yl]-methyl-amine | 401.3 | 2.65 |
| 357 | 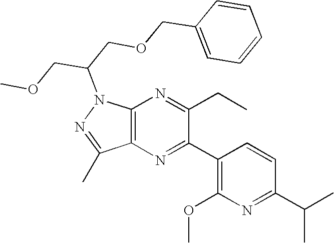 | 1-(2-Benzyloxy-1-methoxymethyl- ethyl)-6-ethyl-5-(6-isopropyl-2- methoxy-pyridin-3-yl)-3-methyl-1H- pyrazolo[3,4-b]pyrazine | 490.2 | 4.37 |
| 358 | 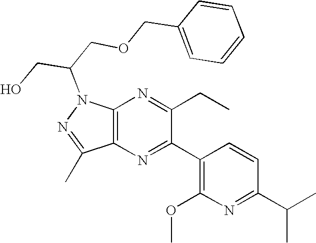 | 3-Benzyloxy-2-[6-ethyl-5-(6- isopropyl-2-methoxy-pyridin-3-yl)-3- methyl-pyrazolo[3,4-b]pyrazin-1-yl]- propan-1-ol | 476.2 | 3.70 |
| 359 | 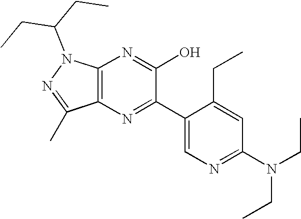 | 5-(6-Diethylamino-4-ethyl-pyridin-3- yl)-1-(1-ethyl-propyl)-3-methyl-1H- pyrazolo[3,4-b]pyrazin-6-ol | 397.2 | 2.17 |
| 360 | 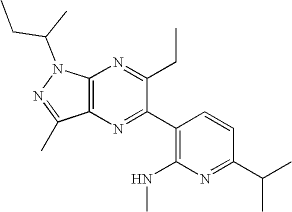 | [3-(1-sec-Butyl-6-ethyl-3-methyl- 1H-pyrazolo[3,4-b]pyrazin-5-yl)-6- isopropyl-pyridin-2-yl]-methyl-amine | 367.3 | 2.60 |
| 361 | 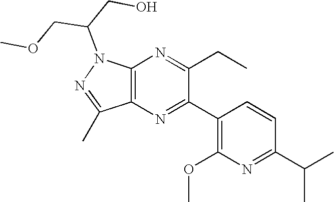 | 2-[6-Ethyl-5-(6-isopropyl-2- methoxy-pyridin-3-yl)-3-methyl- pyrazolo[3,4-b]pyrazin-1-yl]-3- methoxy-propan-1-oI | 400.2 | 3.30 |
| 362 | 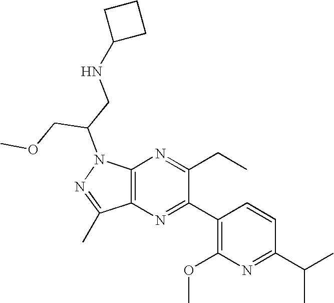 | Cyclobutyl-{2-[6-ethyl-5-(6- isopropyl-2-methoxy-pyridin-3-yl)-3- methyl-pyrazolo[3,4-b]pyrazin-1-yl]- 3-methoxy-propyl}-amine | 453.3 | 2.37 |
| 363 | 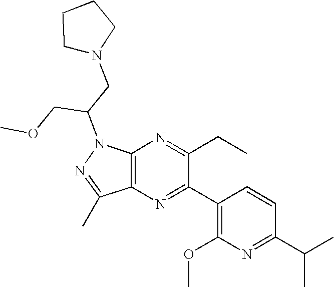 | 6-Ethyl-5-(6-isopropyl-2-methoxy- pyridin-3-yl)-1-(1-methoxymethyl-2- pyrrolidin-1-yl-ethyl)-3-methyl-1H- pyrazolo[3,4-b]pyrazine | 453.3 | 2.27 |
| 364 | 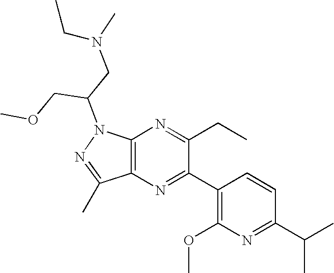 | Ethyl-{2-[6-ethyl-5-(6-isopropyl-2- methoxy-pyridin-3-yl)-3-methyl- pyrazolo[3,4-b]pyrazin-1-yl]-3- methoxy-propyl}-methyl-amine | 441.3 | 2.27 |
| 365 | 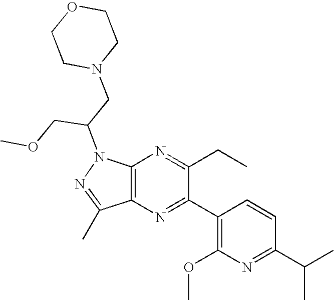 | 6-Ethyl-5-(6-Isopropyl-2-methoxy- pyridin-3-yl)-1-(1-methoxymethyl-2- morpholin-4-yl-ethyl)-3-methyl-1H- pyrazolo[3,4-b]pyrazine | 469.3 | 2.35 |
| 366 | 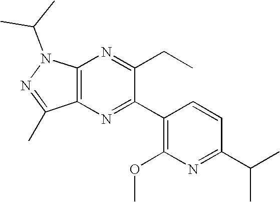 | 6-Ethyl-1-isopropyl-5-(6-isopropyl- 2-methoxy-pyridin-3-yl)-3-methyl- 1H-pyrazolo[3,4-b]pyrazine | 354.2 | 4.59 |
| 367 | 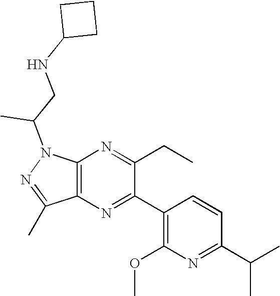 | Cyclobutyl-{2-[6-ethyl-5-(6- isopropyl-2-methoxy-pyridin-3-yl)-3- methyl-pyrazolo[3,4-b]pyrazin-1-yl]- propyl}-amine | 423.3 | 3.02 |
| 368 | 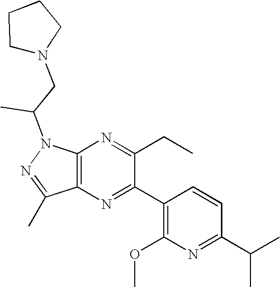 | 6-Ethyl-5-(6-isopropyl-2-methoxy- pyridin-3-yl)-3-methyl-1-(1-methyl- 2-pyrrolidin-1-yl-ethyl)-1H- pyrazolo[3,4-b]pyrazine | 423.3 | 3.02 |
| 369 | 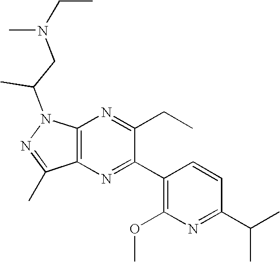 | Ethyl-{2-[6-ethyl-5-(6-isopropyl-2- methoxy-pyridin-3-yl)-3-methyl- pyrazolo[3,4-b]pyrazin-1-yl]-propyl}- methyl-amine | 411.3 | 2.90 |
| 370 | 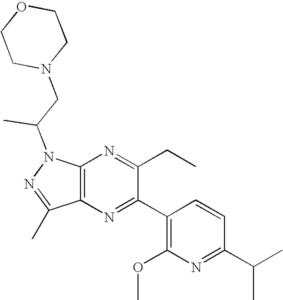 | 6-Ethyl-5-(6-isopropyl-2-methoxy- pyridin-3-yl)-3-methyl-1-(1-methyl- 2-morpholin-4-yl-ethyl)-1H- pyrazolo[3,4-b]pyrazine | 439.3 | 2.99 |
| 371 | 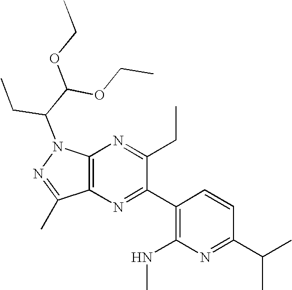 | {3-[1-(1-Diethoxymethyl-propyl)-6- ethyl-3-methyl-1H-pyrazolo[3,4- b]pyrazin-5-yl]-6-isopropyl-pyridin- 2-yl}-methyl-amine | 455.3 | 2.58 |
| 372 | 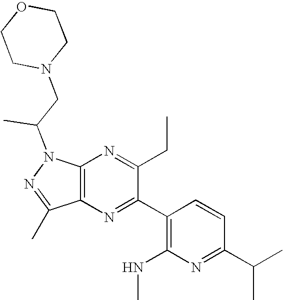 | {3-[6-Ethyl-3-methyl-1-(1- morpholin-4-ylmethyl-propyl)-1H- pyrazolo[3,4-b]pyrazin-5-yl]-6- isopropyl-pyridin-2-yl}-methyl- amine | 452.3 | 2.62 |
| 373 | 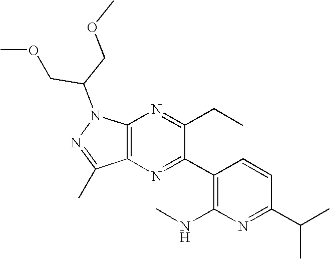 | {3-[6-Ethyl-1-(2-methoxy-1- methoxymethyl-ethyl)-3-methyl-1H- pyrazolo[3,4-b]pyrazin-5-yl]-6- isopropyl-pyridin-2-yl}-methyl- amine | 413.3 | 1.95 |
| 374 | 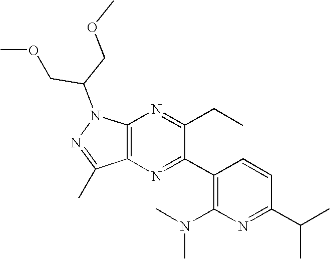 | {3-[6-Ethyl-1-(2-methoxy-1- methoxymethyl-ethyl)-3-methyl-1H- pyrazolo[3,4-b]pyrazin-5-yl]-6- isopropyl-pyridin-2-yl}-dimethyl- amine | 427.3 | 2.72 |
| 375 | 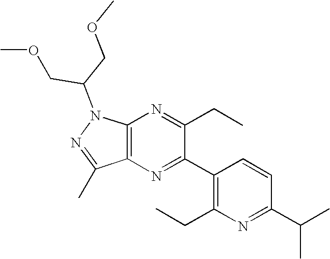 | 6-Ethyl-5-(2-ethyl-6-isopropyl- pyridin-3-yl)-1-(2-methoxy-1- methoxymethyl-ethyl)-3-methyl-1H- pyrazolo[3,4-b]pyrazine | 412.3 | 2.17 |
| 376 | 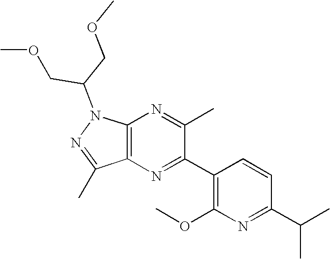 | 5-(6-Isopropyl-2-methoxy-pyridin-3- yl)-1-(2-methoxy-1-methoxymethyl- ethyl)-3,6-dimethyl-1H-pyrazolo[3,4- b]pyrazine | 400.1 | 3.68 |

### Example 24

Assay for CRF Receptor Binding Activity

As discussed above, the following assay is defined herein as a standard in vitro CRF receptor binding assay.

The pharmaceutical utility of compounds of this invention is indicated by the following assay for CRF1 receptor activity. The CRF receptor binding is performed using a modified version of the assay described by Grigoriadis and De Souza (Methods in Neurosciences, Vol. 5, 1991). IMR-32 human neuroblastoma cells, a cell-line that naturally expresses the CRF1 receptor, are grown in IMR-32 Medium, which consists of EMEM w/Earle's BSS (JRH Biosciences, Cat# 51411) plus, as supplements, 2 mM L-Glutamine, 10% Fetal Bovine Serum, 25 mM HEPES (pH 7.2), 1 mM Sodium Pyruvate and Non-Essential Amino Acids (JRH Biosciences, Cat# 58572). The cells are grown to confluence and split three times (all splits and harvest are carried out using NO-ZYME—JRH Biosciences, Cat# 59226). The cells are first split 1:2, incubated for 3 days and split 1:3, and finally incubated for 4 days and split 1:5. The cells are then incubated for an additional 4 days before being differentiated by treatment with 5-bromo-2′deoxyuridine (BrdU, Sigma, Cat# B9285). The medium is replaced every 3-4 days with IMR-32 medium w/2.5 uM BrdU and the cells are harvested after 10 days of BrdU treatment and washed with calcium and magnesium-free PBS.

To prepare receptor containing membranes cells are homogenized in wash buffer (50 mM Tris HCl, 10 mM MgCl₂, 2 mM EGTA, pH 7.4) and centrifuged at 48,000×g for 10 minutes at 4° C. The pellet is re-suspended in wash buffer and the homogenization and centrifugation steps are performed two additional times.

Membrane pellets (containing CRF receptors) are re-suspended in 50 mM Tris buffer pH 7.7 containing 10 mM MgCl₂ and 2 mM EDTA and centrifuged for 10 minutes at 48,000 g. Membranes are washed again and brought to a final concentration of 1500 ug/ml in binding buffer (Tris buffer above with 0.1% BSA, 15 mM bacitracin and 0.01 mg/ml aprotinin.). For the binding assay, 100 ul of the membrane preparation are added to 96 well microtube plates containing 100 ul of ¹²⁵I-CRF (SA 2200 Ci/mmol, final concentration of 100 pM) and 50 ul of test compound. Binding is carried out at room temperature for 2 hours. Plates are then harvested on a BRANDEL 96 well cell harvester and filters are counted for gamma emissions on a Wallac 1205 BETAPLATE liquid scintillation counter. Non-specific binding is defined by 1 mM cold CRF. IC₅₀ values are calculated with the non-linear curve fitting program RS/1 (BBN Software Products Corp., Cambridge, Mass.). The binding affinity for the compounds of Formula I and Formula XXXIII expressed as IC₅₀ value, generally ranges from about 0.5 nanomolar to about 10 micromolar. Preferred compounds of Formula I and Formula XXXIII exhibit IC₅₀ values of less than or equal to 1.5 micromolar, more preferred compounds of Formula I and Formula XXXIII exhibit IC₅₀ values of less than 500 nanomolar, still more preferred compounds of Formula I and Formula XXXIII exhibit IC₅₀ values of less than 100 nanomolar, and most preferred compound of Formula I and Formula XXXIII exhibit IC₅₀ values of less than 10 nanomolar. The compounds shown in Examples 1-33 have been tested in this assay and found to exhibit IC₅₀ values of less than or equal to 4 micromolar.

### Example 25

Preparation of Radiolabeled Probe Compounds of the Invention

The compounds of the invention are prepared as radiolabeled probes by carrying out their synthesis using precursors comprising at least one atom that is a radioisotope. The radioisotope is preferably selected from of at least one of carbon (preferably ¹⁴C), hydrogen (preferably ³H), sulfur (preferably ³⁵S), or iodine (preferably ¹²⁵I). Such radiolabeled probes are conveniently synthesized by a radioisotope supplier specializing in custom synthesis of radiolabeled probe compounds. Such suppliers include Amersham Corporation, Arlington Heights, Ill.; Cambridge Isotope Laboratories, Inc. Andover, Mass.; SRI International, Menlo Park, Calif.; Wizard Laboratories, West Sacramento, Calif.; ChemSyn Laboratories, Lexena, Kans.; American Radiolabeled Chemicals, Inc., St. Louis, Mo.; and Moravek Biochemicals Inc., Brea, Calif.

Tritium labeled probe compounds are also conveniently prepared catalytically via platinum-catalyzed exchange in tritiated acetic acid, acid-catalyzed exchange in tritiated trifluoroacetic acid, or heterogeneous-catalyzed exchange with tritium gas. Such preparations are also conveniently carried out as a custom radiolabeling by any of the suppliers listed in the preceding paragraph using the compound of the invention as substrate. In addition, certain precursors may be subjected to tritium-halogen exchange with tritium gas, tritium gas reduction of unsaturated bonds, or reduction using sodium borotritide, as appropriate.

### Example 26

Receptor Autoradiography

Receptor autoradiography (receptor mapping) is carried out in vitro as described by Kuhar in sections 8.1.1 to 8.1.9 of Current Protocols in Pharmacology (1998) John Wiley & Sons, New York, using radiolabeled compounds of the invention prepared as described in the preceding Examples.

### Example 26

Additional Aspects of Preferred Compounds of the Invention

The most preferred compounds of the invention are suitable for pharmaceutical use in treating human patients. Accordingly, such preferred compounds are non-toxic. They do not exhibit single or multiple dose acute or long-term toxicity, mutagenicity (e.g., as determined in a bacterial reverse mutation assay such as an Ames test), teratogenicity, tumorogenicity, or the like, and rarely trigger adverse effects (side effects) when administered at therapeutically effective dosages.

Preferably, administration of such preferred compounds of the invention at certain doses (i.e., doses yielding therapeutically effective in vivo concentrations or preferably doses of 10, 50, 100, 150, or 200 mg/kg administered parenterally or prefrerably orally) does not result in prolongation of heart QT intervals (i.e., as determined by electrocardiography, e.g., in guinea pigs, minipigs or dogs). When administered daily for 5 or preferably ten days, such doses of such preferred compounds also do not cause liver enlargement resulting in an increase of liver to body weight ratio of more than 100%, preferably not more than 75% and more preferably not more than 50% over matched controls in laboratory rodents (e.g., mice or rats). In another aspect such doses of such preferred compounds also preferably do not cause liver enlargement resulting in an increase of liver to body weight ratio of more than 50%, preferably preferably not more than 25%, and more preferably not more than 10% over matched untreated controls in dogs or other non-rodent mammals.

In yet another aspect such doses of such preferred compounds also preferably do not promote the release of liver enzymes (e.g., ALT, LDH, or AST) from hepatocytes in vivo. Preferably such doses do not elevate serum levels of such enzymes by more than 100%, preferably not by more than 75% and more preferably not by more than 50% over matched untreated controls in laboratory rodents. Similarly, concentrations (in culture media or other such solutions that are contacted and incubated with cells in vitro) equivalent to two, fold, preferably five-fold, and most preferably ten-fold the minimum in vivo therapeutic concentration do not cause release of any of such liver enzymes from hepatocytes into culture medium in vitro above baseline levels seen in media from untreated cells.

Because side effects are often due to undesirable receptor activation or antagonism, preferred compounds of the invention exert their receptor-modulatory effects with high selectivity. This means that they do not bind to certain other receptors (other than CRF receptors) with high affinity, but rather only bind to, activate, or inhibit the activity of such other receptors with affinity constants of greater than 100 nanomolar, preferably greater than I micromolar, more preferably greater than 10 micromolar and most preferably greater than 100 micromolar. Such receptors preferably are selected from the group including ion channel receptors, including sodium ion channel receptors, neurotransmitter receptors such as alpha- and beta-adrenergic receptors, muscarinic receptors (particularly m1, m2, and m3 receptors), dopamine receptors, and metabotropic glutamate receptors; and also include histamine receptors and cytokine receptors, e.g., interleukin receptors, particularly IL-8 receptors. The group of other receptors to which preferred compounds do not bind with high affinity also includes GABA_{A} receptors, bioactive peptide receptors (including NPY and VIP receptors), neurokinin receptors, bradykinin receptors (e.g., BK1 receptors and BK2 receptors), and hormone receptors (including thyrotropin releasing hormone receptors and melanocyte-concentrating hormone receptors).

### Example 26a

Absence of Sodium Ion Channel Activity

Preferred compounds of the invention do not exhibit activity as sodium ion channel blockers. Sodium channel activity may be measured a standard in vitro sodium channel binding assays such as the assay given by Brown et al. (J. Aeurosci. 1986, 265, 17995-18004). Preferred compounds of the invention exhibit less than 15 percent inhibition, and more preferably less than 10 percent inhibition, of sodium channel specific ligand binding when present at a concentration of 4 uM. The sodium ion channel specific ligand used may be labeled batrachotoxinin, tetrodotoxin, or saxitoxin. Such assays, including the assay of Brown referred to above, are performed as a commercial service by CEREP, Inc., Redmond, Wash.

Alternatively, sodium ion channel activity may be measured in vivo in an assay of anti-epileptic activity. Anti-epileptic activity of compounds may be measured by the ability of the compounds to inhibit hind limb extension in the supra maximal electro shock model. Male Han Wistar rats (150-200 mg) are dosed i.p. with a suspension of 1 to 20 mg of test compound in 0.25% methylcellulose 2 hr. prior to test. A visual observation is carried out just prior to testing for the presence of ataxia. Using auricular electrodes a current of 200 mA, duration 200 millisec, is applied and the presence or absence of hind limb extension is noted. Preferred compounds of the invention do not exhibit significant anti-epileptic activity at the p<0.1 level of significance or more preferably at the p<0.05 level of significance as measured using a standard parametric assay of statistical significance such as a student's T test.

### Example 26b

Microsomal in vitro Half-Life

Compound half-life values (t_{1/2} values) may be determined via the following standard liver microsomal half-life assay. Pooled Human liver microsomes are obtained from XenoTech LLC, 3800 Cambridge St. Kansas's City, Kans., 66103 (catalog # H0610). Such liver microsomes may also be obtained from In Vitro Technologies, 1450 South Rolling Road, Baltamore, Md. 21227, or from Tissue Transformation Technologies, Edison Corporate Center, 175 May Street, Suite 600, Edison, N.J. 08837. Reactions are preformed as follows:

Reagents:

Phosphate buffer: 19 mL 0.1 M NaH₂PO₄, 81 mL 0.1 Na₂HPO₄, adjusted to pH 7.4 with H₃PO₄.

CoFactor Mixture: 16.2 mg NADP, 45.4 mg Glucose-6-phosphate in 4 mL 100 mM MgCl₂.

Glucose-6-phosphate dehydrogenase: 214.3 ul glucose-6-phosphate dehydrogenase suspension (Boehringer-Manheim catalog no. 0737224, distributed by Roche Molecular Biochemicals, 9115 Hague Road, P.O. Box 50414, Indianapolis, Ind. 46250) is diluted into 1285.7 ul distilled water.

Starting Reaction Mixture: 3 mL CoFactor Mixture, 1.2 mL Glucose-6-phosphate dehydrogenase.

Reaction:

6 test reactions are prepared, each containing 25 ul microsomes, 5 ul of a 100 uM solution of test compound, and 399 ul 0.1 M phosphate buffer. A seventh reaction is prepared as a positive control containing 25 ul microsomes, 399 ul 0.1 M phosphate buffer, and 5 ul of a 100 uM solution of a compound with known metabolic properties (e.g. DIAZEPAM or CLOZEPINE). Reactions are preincubated at 39° C. for 10 minutes. 71 ul Starting Reaction Mixture is added to 5 of the 6 test reactions and to the positive control, 71 ul 100 mM MgCl₂ is added to the sixth test reaction, which is used as a negative control. At each time point (0, 1, 3, 5, and 10 minutes) 75 ul of each reaction mix is pipetted into a well of a 96-well deep-well plate containing 75 ul ice-cold acetonitrile. Samples are vortexed and centrifuged 10 minutes at 3500 rpm (Sorval T 6000D centrifuge, H1000B rotor). 75 ul of supernatant from each reaction is transferred to a well of a 96-well plate containing 150 ul of a 0.5 uM solution of a compound with a known LCMS profile (internal standard) per well. LCMS analysis of each sample is carried out and the amount of unmetabolized test compound is measured as AUC, compound concentration vs time is plotted, and the t_{1/2} value of the test compound is extrapolated.

Preferred compounds of the invention exhibit in vitro t_{1/2} values of greater than 10 minutes and less than 4 hours. Most preferred compounds of the invention exhibit in vitro t_{1/2} values of between 30 minutes and 1 hour in human liver microsomes.

### Example 26c

MDCK Toxicity Assay

Compounds causing acute cytotoxicity will decrease ATP production by Madin Darby canine kidney (MDCK) cells in the following assay.

MDCK cells, ATCC no. CCL-34 (American Type Culture Collection, Manassas, Va.) are maintained in sterile conditions following the instructions in the ATCC production information sheet. The PACKARD, (Meriden, Conn.) ATP-LITE-M Luminescent ATP detection kit, product no. 6016941, allows measurement ATP production in MDCK cells.

Prior to assay 1 ul of test compound or control sample is pipetted into PACKARD (Meriden, Conn.) clear bottom 96-well plates. Test compounds and control samples are diluted in DMSO to give final concentration in the assay of 10 micromolar, 100 micromolar, or 200 micromolar. Control samples are drug or other compounds having known toxicity properties.

Confluent MDCK cells are trypsinized, harvested, and diluted to a concentration of 0.1×10⁶ cells/ ml with warm (37° C.) VITACELL Minimum Essential Medium Eagle (ATCC catalog # 30-2003). 100 ul of cells in medium is pipetted into each of all but five wells of each 96-well plate. Warm medium without cells (100 ul) is pipetted in the remaining five wells of each plate to provide standard curve control wells. These wells, to which no cells are added, are used to determine the standard curve. The plates are then incubated at 37° C. under 95% O₂, 5% CO₂ for 2 hours with constant shaking. After incubation, 50 ul of mammalian cell lysis solution is added per well, the wells are covered with PACKARD TOPSEAL stickers, and plates are shaken at approximately 700 rpm on a suitable shaker for 2 minutes.

During the incubation, PACKARD ATP LITE-M reagents are allowed to equilibrate to room temperature. Once equilibrated the lyophilized substrate solution is reconstituted in 5.5 mls of substrate buffer solution (from kit). Lyophilized ATP standard solution is reconstituted in deionized water to give a 10 mM stock. For the five control wells, 10 ul of serially diluted PACKARD standard is added to each of the five standard curve control wells to yield a final concentration in each subsequent well of 200 nM, 100 nM, 50 nM, 25 nM, and 12.5 nM.

PACKARD substrate solution (50 ul) is added to all wells. Wells are covered with PACKARD TOPSEAL stickers, and plates are shaken at approximately 700 rpm on a suitable shaker for 2 minutes. A white PACKARD sticker is attached to the bottom of each plate and samples are dark adapted by wrapping plates in foil and placing in the dark for 10 minutes. Luminescence is then measured at 22° C. using a luminescence counter, e.g. PACKARD TOPCOUNT Microplate Scintillation and Luminescense Counter or TECAN SPECTRAFLUOR PLUS.

Luminescence values at each drug concentration are compared to the values computed from the standard curve for that concentration. Preferred test compounds exhibit luminescence values 80% or more of the standard, or preferably 90% or more of the standard, when a 10 micromolar (uM) concentration of the test compound is used. When a 100 uM concentration of the test compound is used, preferred test compounds exhibit luminescence values 50% or more of the standard, or more preferably 80% or more of the standard.

1. A compound of the Formula I: 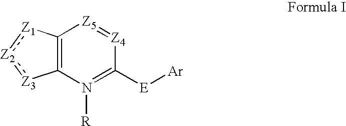
or a pharmaceutically acceptable salt thereof, wherein:
E is a single bond, O, S(O)ₘ, NR₁₀ or CR₁₀R₁₁;
Ar is chosen from:
phenyl which is mono-, di-, or tri-substituted;
1-naphthyl and 2-naphthyl, each of which is optionally mono-, di-, or tri-substituted; and
optionally mono-, di-, or tri-substituted heteroaryl, said heteroaryl having from 1 to 3 rings, 5 to 7 ring members in each ring and, in at least one of said rings, from 1 to about 3 heteroatoms selected from the group consisting of N, O, and S;
wherein in Ar, at least one of the positions ortho to the point of attachment of Ar shown in Formula I is substituted;
R is independently selected at each occurrence to be absent or oxygen;
the group: 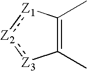
represents a saturated, unsaturated or aromatic 5-membered ring system containing 2 or 3 nitrogen atoms, wherein:
Z₁ is CR₁, CR₁R₁′, or NR₁″;
Z₂ is nitrogen, or NR₂″,
Z₃ is CR₃, CR₃R₃′, nitrogen, NR₃″, oxygen, sulfur, sulfoxide or sulfone;
R¹ is chosen from halogen, hydroxy, cyano, amino, optionally substituted alkyl, optionally substituted alkenyl, optionally substituted alkynyl optionally substituted alkoxy, optionally substituted mono or dialkylamino, optionally substituted heterocycloalkyl, optionally substituted (cycloalkyl)alkyl, optionally substituted (heterocycloalkyl)alkyl, optionally substituted alkylthio, optionally substituted alkylsulfinyl, optionally substituted alkylsulfonyl, optionally substituted mono or dialkylcarboxamide, optionally substituted carbocyclic aryl, optionally substituted (aryl)cycloalkyl, optionally substituted (aryl)heterocycloalkyl,optionally substituted heteroaryl, optionally substituted (heteroaryl)cycloalkyl, optionally substituted (heteroaryl)heterocycloalkyl, wherein each heteroaryl has from from 1 to 3 rings, 5 to 7 ring members in each ring and, in at least one of said rings, from 1 to about 3 heteroatoms selected from the group consisting of N, O, and S;
R₁″ is chosen from optionally substituted alkyl, optionally substituted alkenyl, optionally substituted alkynyl, optionally substituted heterocycloalkyl, optionally substituted (cycloalkyl)alkyl, optionally substituted (heterocycloalkyl)alkyl, optionally substituted mono or dialkylamino, optionally substituted alkanoyl, optionally substituted carbocyclic aryl, optionally substituted (aryl)cycloalkyl, optionally substituted (aryl)heterocycloalkyl, optionally substituted heteroaryl, optionally substituted (heteroaryl)cycloalkyl, optionally substituted (heteroaryl)heterocycloalkyl, wherein each heteroaryl has from 1 to 3 rings, 5 to 7 ring members in each ring and, in at least one of said rings, from 1 to about 3 heteroatoms selected from the group consisting of N, O, and S;
R₃ is chosen from hydrogen, halogen, hydroxy, amino, cyano, nitro, alkyl, haloalkyl, alkoxy, aminoalkyl, and mono- and di-alkylamino;
R₁′ and R₃′ are independently chosen from hydrogen, halogen, alkyl, haloalkyl, and aminoalkyl;
R₂″ and R₃″ are independently chosen from hydrogen, alkyl, haloalkyl, optionally substituted mono or dialkylamino, optionally substituted alkanoyl, and aminoalkyl;
Z₄ is selected from NR and CR₄;
Z₅ is selected from NR and CR₅;
R₄ and R₅ are independently chosen from hydrogen, halogen, hydroxy, amino, cyano, nitro, optionally substituted alkyl, optionally substituted alkenyl, optionally substituted alkynyl, optionally substituted alkoxy, optionally substituted mono or dialkylamino, optionally substituted (cycloalkyl)alkyl, optionally substituted alkylthio, optionally substituted alkylsulfinyl, optionally substituted alkylsulfonyl, optionally substituted mono- or dialkylcarboxamide, optionally substituted carbocyclic aryl, and optionally substituted heteroaryl, said optionally substituted heteroaryl having from 1 to 3 rings, 5 to 7 ring members in each ring and, in at least one of said rings, from 1 to about 3 heteroatoms selected from the group consisting of N, O, and S;
R₄″ and R₅″ are independently chosen from hydrogen, alkyl, haloalkyl, and aminoalkyl;
R₁₀ and R₁₁ are independently hydrogen or C₁-C₄ alkyl; and
m is 0, 1, or 2.

2. A compound of the Formula I: 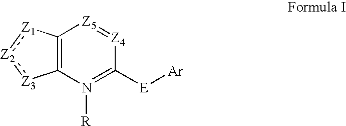
or a pharmaceutically acceptable salt thereof, wherein:
R is independently selected at each occurrence to be absent or oxygen;
E is a single bond, O, or S(O)ₘ;
m is 0, 1, or 2;
Ar is chosen from:
phenyl which is mono-, di-, or tri-substituted with R_{A}, or 1-naphthyl, 2-naphthyl, pyridyl, pyrimidinyl, pyrazinyl, pyridizinyl, imidazo-pyridyl, imidazo-pyrimidinyl, imidazo-pyrazinyl, imidazo-pyridizinyl, thienyl, thiazolyl, oxazolyl, isoxazolyl, pyrrolyl, furanyl, and triazolyl, each of which is optionally mono-, di-, or tri-substituted with R_{A};
wherein in Ar, at least one of the positions ortho to the point of attachment of Ar shown in Formula I is substituted with R_{A};
the group: 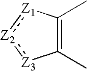
represents a saturated, unsaturated or aromatic ring system comprising 2 or 3 adjacent nitrogen atoms, wherein:
Z₁ is CR₁, CR₁R₁′, or NR₁″;
Z₂ is nitrogen or NR₂″;
Z₃ is CR₃, CR₃R₃′, nitrogen, NR₃″, oxygen, sulfur, sulfoxide or sulfone;
R₁ is chosen from
i) halogen, hydroxy, cyano, amino, C₁-C₁₀carbyhydryl, —O(C₁-C₆ carbyhydryl), mono or di(C₁-C₆ carbyhydryl)amino, (C₃-C₇cyclocarbyhydryl) C₁-C₄ carbyhydryl, (C₃-C₇heterocycloalkyl)C₀-C₄carbhydryl, (benzoC₃-C₇cycloalkyl)C₀-C₄carbhydryl, (benzoC₃-C₇heterocycloalkyl)C₀-C₄carbhydryl, (C₁C₆)haloalkyl, and mono- and di-(C₁C₆)alkylamino, C₂-C₆alkanoyl; each of which is substituted with 0 or more substituents independently chosen from halogen, hydroxy, amino, oxo, cyano, C₁-C₆alkyl, C₁-C₆alkoxy, C₁-C₆hydroxyalkyl, C₁-C₆alkoxyC₁-C₆alkyl, C₁-C₆haloalkoxy, C₅-C₇heteroaryl, mono- and di-(C₁-C₆)alkylamino, and —XR_{C}, halo(C₁C₆)carbhydryl, —O(halo(C₁C₆)carbhydryl) and S(O)ₙ(C₁-C₆carbhydryl), —O(C₃-C₇cyclocarbhydryl)C₁-C₄carbhydryl, and S(O)ₙ(C₁-C₆carbhydryl), and
ii) phenyl which is mono-, di-, or tri-substituted with R_{A}, 1-naphthyl, 2-naphthyl, pyridyl, dihydropyridyl, tetrahydropyridyl, pyrimidinyl, pyrazinyl, pyridizinyl, thienyl, thiazolyl, oxazolyl, isoxazolyl, pyrrolyl, furanyl, and triazolyl, each of which is optionally mono-, di-, or tri-substituted with R_{A};
R₁″ is chosen from
i) C₁-C₁₀carbhydryl, (C₃-C₇cycloalkyl)C₁-C₄ carbhydryl, and halo(C₁C₆) carbhydryl, (C₃₋₆heterocycloalkyl)C₀-C₄carbhydryl, (benzoC₃-C₇cycloalkyl)C₀-C₄carbhydryl, (benzoC₃-C₇heterocycloalkyl)C₀-C₄carbhydryl, (C₁C₆)haloalkyl, and mono- and di-(C₁C₆)alkylamino, C₂-C₆alkanoyl; each of which is substituted with 0 or more substituents independently chosen from halogen, hydroxy, amino, oxo, cyano, C₁-C₆alkyl, C₁-C₆alkoxy, C₁-C₆hydroxyalkyl, C₁-C₆alkoxyC₁-C₆alkyl, C₁-C₆haloalkoxy, C₅-C₇heteroaryl, mono- and di-(C₁-C₆)alkylamino, and —XR_{C}, and
ii) phenyl which is mono-, di-, or tri-substituted with R_{A}, benzyl, 1-naphthyl, 2-naphthyl, pyridyl, dihydropyridyl, tetrahydropyridyl, pyrimidinyl, pyrazinyl, pyridizinyl, thienyl, thiazolyl, oxazolyl, isoxazolyl, pyrrolyl, furanyl, and triazolyl, each of which is optionally mono-, di-, or tri-substituted with R_{A};
R₃ is chosen from hydrogen, halogen, hydroxy, amino, cyano, nitro, C₁-C₆alkyl, halo(C₁-C₆)alkyl, C₁-C₆alkoxy, amino(C₁-C₆)alkyl, and mono and di(C₁-C₆)alkylamino;
R₁′ and R₃′ are independently chosen from hydrogen, halogen, C₁-C₆alkyl, halo(C₁-C₆)alkyl, and amino(C₁-C₆)alkyl;
R₂″ and R₃″ are independently chosen from hydrogen, C₁-C₆alkyl, halo(C₁-C₆)alkyl, mono or di(C₁-C₆alkyl)amino, C₁-C₆alkanoyl and amino(C₁-C₆)alkyl;
Z₄ is selected from NR and CR₄;
Z₅ is selected from NR and CR₅;
R₄ and R₅ are independently chosen from hydrogen, halogen, cyano, nitro, amino, mono or di(C₁-C₆ carbhydryl)amino, C₁-C₆ carbhydryl, (C₃-C₇cycloalkyl) C₁-C₄ carbhydryl,—O(C₃-C₇cycloalkyl) C₁-C₄ carbhydryl, halo(C₁-C₆) carbhydryl, —O(halo(C₁-C₆) carbhydryl), —O(C₁-C₆ carbhydryl), S(O)ₙ(C₁-C₆ carbhydryl), N(H)(S(O)ₙ(C₁-C₆ carbhydryl)), N(C₁-C₆ carbhydryl) (S(O)ₙ(C₁-C₆ carbhydryl)
where each carbhydrylis independently straight, branched, or cyclic, contains zero or 1 or more double or triple bonds, and is optionally substituted with one or more substituents independently chosen from halogen, hydroxy, amino, oxo, cyano, C₁-C₄alkoxy, and mono- or di(C₁-C₄)alkylamino,
and
where each C₃-C₇cycloalkyl is optionally substituted by one or more substituents independently chosen from halogen, amino, hydroxy, oxo, cyano, C₁-C₄alkoxy, and mono- or di(C₁-C₄)alkylamino;
R₄″ and NR₅″ are independently selected from hydrogen,
R_{A} is independently selected at each occurrence from halogen, cyano, nitro, halo(C₁-C₆)alkyl, halo(C₁-C₆)alkoxy, hydroxy, amino, C₁-C₆alkyl substituted with 0-2 R_{B}, C₂-C₆alkenyl substituted with 0-2 R_{B}, C₂-C₆alkynyl substituted with 0-2 R_{B}, C₃-C₇cycloalkyl substituted with 0-2 R_{B}, (C₃-C₇cycloalkyl)C₁-C₄alkyl substituted with 0-2 R_{B},
C₁-C₆alkoxy substituted with 0-2 R_{B}, —NH(C₁-C₆alkyl) substituted with 0-2 R_{B}, —N(C₁-C₆alkyl)(C₁-C₆alkyl) where each C₁-C₆alkyl is independently substituted with 0-2 R_{B}, —S(O)ₙ(C₁-C₆alkyl) substituted with 0-2 R_{B}, —XR_{C}, and Y;
R_{B} is independently selected at each occurrence from halogen, hydroxy, cyano, amino, C₁-C₄alkyl, —O(C₁-C₄alkyl), —NH(C₁-C₄alkyl), —N(C₁-C₄alkyl)( C₁-C₄alkyl), —S(O)ₙ(alkyl), halo(C₁-C₄)alkyl, halo(C₁-C₄)alkoxy, CO(C₁-C₄alkyl), CONH(C₁-C₄alkyl), CON(C₁-C₄alkyl)( C₁-C₄alkyl), —XR_{C}, and Y;
R_{C} and R_{D}, are the same or different, and are independently selected at each occurrence from: hydrogen, and straight, branched, and cyclic alkyl groups, and (cycloalkyl)alkyl groups, said straight, branched, and cyclic alkyl groups, C₅-C₇heteroaryl(C₀-C₄alkyl), and (cycloalkyl)alkyl groups consist of 1 to 8 carbon atoms, and contain zero or one or more double or triple bonds, each of which 1 to 8 carbon atoms may be further substituted with one or more substituent(s) independently selected from oxo, hydroxy, halogen, cyano, amino, C₁-C₆alkoxy, —NH(C₁-C₆alkyl), —N(C₁-C₆alkyl)(C₁-C₆alkyl), —NHC(═O)(C₁-C₆alkyl), —N(C₁-C₆alkyl)C(═O)(C₁-C₆alkyl), —NHS(O)ₙ(C₁-C₆alkyl), —S(O)ₙ(C₁-C₆alkyl), —S(O)ₙNH(C₁-C₆alkyl), —S(O)ₙN(C₁-C₆alkyl)(C₁-C₆alkyl), and Z;
X is independently selected at each occurrence from the group consisting of —CH₂—, —CHR_{D}—, —O—, —C(═O)—, —C(S)—, —C(═O)O—, —C(═S)O—, —S(O)ₙ—, —NH—, —NR_{D}—, —C(═O)NH—, —C(═O)NR_{D}—, —S(O)ₙNH—, —S(O)ₙNR_{D}—, —OC(═S)S—, —NHC(═O)—, —NR_{D}C(═O)—, —C(═S)NR_{D}—, —NHS(O)ₙ—, —OSiH₂—, —OSiH(C₁-C₄alkyl)-, —OSi(C₁-C₄alkyl)(C₁-C₄alkyl)-, and —NR_{D}S(O)ₙ—;
Y and Z are independently selected at each occurrence from: 3- to 7-membered carbocyclic or heterocyclic groups which are saturated, unsaturated, or aromatic, which may be further substituted with one or more substituents independently selected from halogen, oxo, hydroxy, amino, cyano, C₁-C₄alkyl, —O(C₁-C₄alkyl), —NH(C₁-C₄alkyl), —N(C₁-C₄alkyl)(C₁-C₄alkyl), and —S(O)ₙ(alkyl),
wherein said 3- to 7-memberered heterocyclic groups contain one or more heteroatom(s) independently selected from N, O, and S, with the point of attachment being either carbon or nitrogen; and
n is independently selected at each occurrence from 0, 1, and 2.

3. A compound or salt according to claim 2 of Formula II 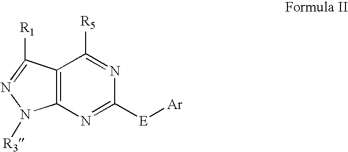
wherein R₁, R₃″, R₅, E, and Ar are as defined in claim 2.

4. A compound or salt according to claim 3, wherein:
R₁ is as defined for claim 3;
R₃″ is selected from hydrogen and C₁-C₆alkyl;
R₅ is selected from hydrogen, halogen, cyano, amino, C₁-C₆alkyl, C₁-C₆alkoxy, C₃-C₇cycloalkyl, (C₃-C₇cycloalkyl)C₁-C₄alkyl, (C₃-C₇cycloalkyl)C₁-C₄alkoxy, mono and di(C₁-C₆alkyl)amino, amino(C₁-C₆)alkyl, mono and di(C₁-C₆alkyl)amino(C₁-C₆)alkyl, halo(C₁-C₆)alkyl, and halo(C₁-C₆)alkoxy; and
Ar is selected from the group consisting of phenyl, pyridyl and pyrimidinyl, each of which is mono- di- or tri-substituted with substituents independently chosen from halogen, cyano, nitro, halo(C₁-C₆)alkyl, halo(C₁-C₆)alkoxy, hydroxy, amino, C₁-C₆alkyl substituted, C₂-C₆alkenyl, C₂-C₆alkynyl, C₃-C₇cycloalkyl, (C₃-C₇cycloalkyl)C₁-C₄alkyl, C₁-C₆alkoxy, mono- and di(C₁-C₆alkyl)amino, amino(C₁-C₆)alkyl, and mono- or di(C₁-C₆alkyl)amino, wherein, in Ar, at least one of the positions ortho to the point of attachment of Ar shown in Formula II is substituted.

5-7. (Cancelled).

8. A compound or salt according to claim 2 of Formula III: 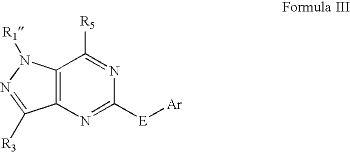
wherein R₁″, R₃, R₅, E, and Ar are as defined in claim 2.

9. A compound or salt according to claim 8, wherein
R₁″ is as defined for claim 8;
R₃ is selected from hydrogen and C₁-C₆alkyl;
R₅ is selected from hydrogen, halogen, cyano, amino, C₁-C₆alkyl, C₁-C₆alkoxy, C₃-C₇cycloalkyl, (C₃-C₇cycloalkyl)C₁-C₄alkyl, (C₃-C₇cycloalkyl)C₁-C₄alkoxy, mono and di(C₁-C₆alkyl)amino, amino(C₁-C₆)alkyl, mono and di(C₁-C₆alkyl)amino(C₁-C₆)alkyl, halo(C₁-C₆)alkyl, and halo(C₁-C₆)alkoxy; and
Ar is selected from the group consisting of phenyl, pyridyl and pyrimidinyl, each of which is mono- di- or trisubstituted with substituents independently chosen from halogen, cyano, nitro, halo(C₁-C₆)alkyl, halo(C₁-C₆)alkoxy, hydroxy, amino, C₁-C₆alkyl substituted, C₂-C₆alkenyl, C₂-C₆alkynyl, C₃-C₇cycloalkyl, (C₃-C₇cycloalkyl)C₁-C₄alkyl, C₁-C₆alkoxy, mono- and di(C₁-C₆alkyl)amino, amino(C₁-C₆)alkyl, and mono- or di(C₁-C₆alkyl)amino, wherein, in Ar, at least one of the positions ortho to the point of attachment of Ar shown in Formula III is substituted.

10-12. (Cancelled).

13. A compound or salt according to claim 2 of Formula IV 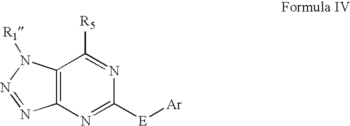
wherein R₁″, R₅, E, and Ar are as defined in claim 2.

14. A compound or salt according to claim 13, wherein:
R₁″ is as defined in claim 13;
R₅ is selected from hydrogen, halogen, cyano, amino, C₁-C₆alkyl, C₁-C₆alkoxy, C₃-C₇cycloalkyl, (C₃-C₇cycloalkyl)C₁-C₄alkyl, (C₃-C₇cycloalkyl)C₁-C₄alkoxy, mono and di(C₁-C₆alkyl)amino, amino(C₁-C₆)alkyl, mono and di(C₁-C₆alkyl)amino(C₁-C₆)alkyl, halo(C₁-C₆)alkyl, and halo(C₁-C₆)alkoxy; and
Ar is selected from the group consisting of phenyl, pyridyl and pyrimidinyl, each of which is mono- di- or trisubstituted with substituents independently chosen from halogen, cyano, nitro, halo(C₁-C₆)alkyl, halo(C₁-C₆)alkoxy, hydroxy, amino, C₁-C₆alkyl substituted,
C₂-C₆alkenyl, C₂-C₆alkynyl, C₃-C₇cycloalkyl, (C₃-C₇cycloalkyl)C₁-C₄alkyl, C₁-C₆alkoxy, mono- and di(C₁-C₆alkyl)amino, amino(C₁-C₆)alkyl, and mono- and di(C₁-C₆alkyl)amino, wherein, in Ar, at least one of the positions ortho to the point of attachment of Ar shown in Formula IV is substituted.

15-17. (Cancelled).

18. A compound or salt according to claim 2 of Formula V 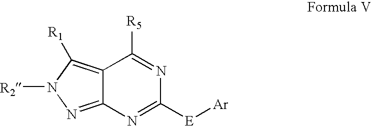
wherein R₁, R₂″, R₅, E, and Ar are as defined in claim 2.

19. A compound or salt according to claim 18, wherein
R₁ is as defined for claim 18;
R₂″ is selected from hydrogen, methyl, and ethyl;
R₅ is selected from hydrogen, halogen, cyano, amino, C₁-C₆alkyl, C₁-C₆alkoxy, C₃-C₇cycloalkyl, (C₃-C₇cycloalkyl)C₁-C₄alkyl, (C₃-C₇cycloalkyl)C₁-C₄alkoxy, mono and di(C₁-C₆alkyl)amino, amino(C₁-C₆)alkyl, mono and di(C₁-C₆alkyl)amino(C₁-C₆)alkyl, halo(C₁-C₆)alkyl, and halo(C₁-C₆)alkoxy; and
Ar is selected from the group consisting of phenyl, pyridyl and pyrimidinyl, each of which is mono- di- or trisubstituted with substituents independently chosen from halogen, cyano, nitro, halo(C₁-C₆)alkyl, halo(C₁-C₆)alkoxy, hydroxy, amino, C₁-C₆alkyl substituted, C₂-C₆alkenyl, C₂-C₆alkynyl, C₃-C₇cycloalkyl, (C₃-C₇cycloalkyl)C₁-C₄alkyl, C₁-C₆alkoxy, mono- and di(C₁-C₆alkyl)amino, amino(C₁-C₆)alkyl, and mono- and
di(C₁-C₆alkyl)amino, wherein, in Ar, at least one of the positions ortho to the point of attachment of Ar shown in Formula V is substituted.

20-22. (Cancelled).

23. A compound or salt according to claim 2 of Formula IX 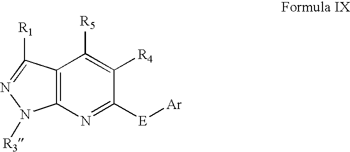
wherein R₁, R₃″, R₄, R₅, E, and Ar are as defined in claim 2.

24. A compound or salt according to claim 23, wherein:
R₁ is as defined for claim 2;
R₃″ is selected from hydrogen and C₁-C₆alkyl;
R₄ and R₅ are independently selected from hydrogen, halogen, cyano, amino, C₁-C₆alkyl, C₁-C₆alkoxy, C₃-C₇cycloalkyl, (C₃-C₇cycloalkyl)C₁-C₄alkyl, (C₃-C₇cycloalkyl)C₁-C₄alkoxy, mono and di(C₁-C₆alkyl)amino, amino(C₁-C₆)alkyl, mono and di(C₁-C₆alkyl)amino(C₁-C₆)alkyl, halo(C₁-C₆)alkyl, and halo(C₁-C₆)alkoxy; and
Ar is selected from the group consisting of phenyl, pyridyl and pyrimidinyl, each of which is mono- di- or tri-substituted with substituents independently chosen from halogen, cyano, nitro, halo(C₁-C₆)alkyl, halo(C₁-C₆)alkoxy, hydroxy, amino, C₁-C₆alkyl substituted, C₂-C₆alkenyl, C₂-C₆alkynyl, C₃-C₇cycloalkyl, (C₃-C₇cycloalkyl)C₁-C₄alkyl, C₁-C₆alkoxy, mono- and di(C₁-C₆alkyl)amino, amino(C₁-C₆)alkyl, and mono- or di(C₁-C₆alkyl)amino, wherein, in Ar, at least one of the positions ortho to the point of attachment of Ar shown in Formula IX is substituted.

25-27. (Cancelled).

28. A compound or salt according to claim 2 of Formula X: 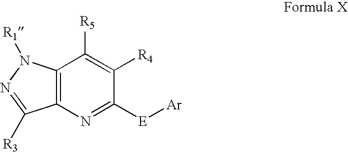
wherein R₁″, R₃, R₄, R₅, E, and Ar are as defined in claim 2.

29. A compound or salt according to claim 28, wherein
R₁″ is as defined for claim 2;
R₄ and R₅ are selected from hydrogen, halogen, cyano, amino, C₁-C₆alkyl, C₁-C₆alkoxy, C₃-C₇cycloalkyl, (C₃-C₇cycloalkyl)C₁-C₄alkyl, (C₃-C₇cycloalkyl)C₁-C₄alkoxy, mono and di(C₁-C₆alkyl)amino, amino(C₁-C₆)alkyl, mono and di(C₁-C₆alkyl)amino(C₁-C₆)alkyl, halo(C₁-C₆)alkyl, and halo(C₁-C₆)alkoxy; and
R₃ is selected from hydrogen and C₁-C₆alkyl; and
Ar is selected from the group consisting of phenyl, pyridyl and pyrimidinyl, each of which is mono- di- or trisubstituted with substituents independently chosen from halogen, cyano, nitro, halo(C₁-C₆)alkyl, halo(C₁-C₆)alkoxy, hydroxy, amino, C₁-C₆alkyl substituted,
C₂-C₆alkenyl, C₂-C₆alkynyl, C₃-C₇cycloalkyl, (C₃-C₇cycloalkyl)C₁-C₄alkyl, C₁-C₆alkoxy, mono- and di(C₁-C₆alkyl)amino, amino(C₁-C₆)alkyl, and mono- or di(C₁-C₆alkyl)amino, wherein, in Ar, at least one of the positions ortho to the point of attachment of Ar shown in Formula X is substituted.

30-32. (Cancelled).

33. A compound or salt according to claim 2 of Formula XI 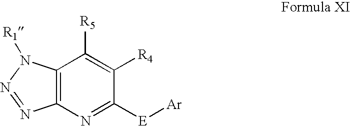
wherein R₁″, R₄, R₅, E, and Ar are as defined in claim 2.

34. A compound or salt according to claim 33, wherein:
R₁″ is as defined in claim 2;
R₄ and R₅ are selected from hydrogen, halogen, cyano, amino, C₁-C₆alkyl, C₁-C₆alkoxy, C₃-C₇cycloalkyl, (C₃-C₇cycloalkyl)C₁-C₄alkyl, (C₃-C₇cycloalkyl)C₁-C₄alkoxy, mono and di(C₁-C₆alkyl)amino, amino(C₁-C₆)alkyl, mono and di(C₁-C₆alkyl)amino(C₁-C₆)alkyl, halo(C₁-C₆)alkyl, and halo(C₁-C₆)alkoxy; and
Ar is selected from the group consisting of phenyl, pyridyl and pyrimidinyl, each of which is mono- di- or trisubstituted with substituents independently chosen from halogen, cyano, nitro, halo(C₁-C₆)alkyl, halo(C₁-C₆)alkoxy, hydroxy, amino, C₁-C₆alkyl substituted, C₂-C₆alkenyl, C₂-C₆alkynyl, C₃-C₇cycloalkyl, (C₃-C₇cycloalkyl)C₁-C₄alkyl, C₁-C₆alkoxy, mono- and di(C₁-C₆alkyl)amino, amino(C₁-C₆)alkyl, and mono- and di(C₁-C₆alkyl)amino, wherein, in Ar, at least one of the positions ortho to the point of attachment of Ar shown in Formula XI is substituted.

35-37. (Cancelled).

38. A compound or salt according to claim 2 of Formula XII 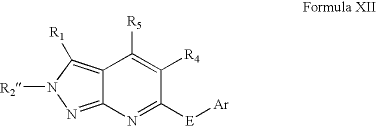
wherein R₁, R₂″, R₄, R₅, E, and Ar are as defined in claim 2.

39. A compound or salt according to claim 38, wherein
R₁ is as defined for claim 2;
R₂″ is selected from hydrogen, methyl, and ethyl;
R₄ and R₅ are selected from hydrogen, halogen, cyano, amino, C₁-C₆alkyl, C₁-C₆alkoxy, C₃-C₇cycloalkyl, (C₃-C₇cycloalkyl)C₁-C₄alkyl, (C₃-C₇cycloalkyl)C₁-C₄alkoxy, mono and di(C₁-C₆alkyl)amino, amino(C₁-C₆)alkyl, mono and di(C₁-C₆alkyl)amino(C₁-C₆)alkyl, halo(C₁-C₆)alkyl, and halo(C₁-C₆)alkoxy; and
Ar is selected from the group consisting of phenyl, pyridyl and pyrimidinyl, each of which is mono- di- or trisubstituted with substituents independently chosen from halogen, cyano, nitro, halo(C₁-C₆)alkyl, halo(C₁-C₆)alkoxy, hydroxy, amino, C₁-C₆alkyl substituted, C₂-C₆alkenyl, C₂-C₆alkynyl, C₃-C₇cycloalkyl, (C₃-C₇cycloalkyl)C₁-C₄alkyl, C₁-C₆alkoxy, mono- and di(C₁-C₆alkyl)amino, amino(C₁-C₆)alkyl, and mono- and di(C₁-C₆alkyl)amino, wherein, in Ar, at least one of the positions ortho to the point of attachment of Ar shown in Formula XII is substituted.

40-42. (Cancelled).

43. A compound or salt according to claim 2 of Formula XVI: 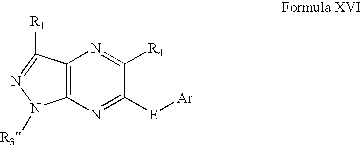
wherein R₁, R₃″, R₄, E, and Ar are as defined in claim 2.

44. A compound or salt according to claim 43, wherein:
R₁ is as defined for claim 43;
R₃″ is selected from hydrogen and C₁-C₆alkyl;
R₄ is selected from hydrogen, halogen, cyano, amino, C₁-C₆alkyl, C₁-C₆alkoxy, C₃-C₇cycloalkyl, (C₃-C₇cycloalkyl)C₁-C₄alkyl, (C₃-C₇cycloalkyl)C₁-C₄alkoxy, mono- and di-(C₁-C₆alkyl)amino, amino(C₁-C₆)alkyl, mono and di(C₁-C₆alkyl)amino(C₁-C₆)alkyl, halo(C₁-C₆)alkyl, and halo(C₁-C₆)alkoxy; and
Ar is selected from the group consisting of phenyl, pyridyl and pyrimidinyl, each of which is mono- di- or trisubstituted with substituents independently chosen from halogen, cyano, nitro, halo(C₁-C₆)alkyl, halo(C₁-C₆)alkoxy, hydroxy, amino, C₁-C₆alkyl substituted, C₂-C₆alkenyl, C₂-C₆alkynyl, C₃-C₇cycloalkyl, (C₃-C₇cycloalkyl)C₁-C₄alkyl, C₁-C₆alkoxy, mono- and di(C₁-C₆alkyl)amino, amino(C₁-C₆)alkyl, and mono- and
di(C₁-C₆alkyl)amino, wherein, in Ar, at least one of the positions ortho to the point of attachment of Ar shown in Formula XVI is substituted.

45-47. (Cancelled).

48. A compound or salt according to claim 2 of Formula XVII: 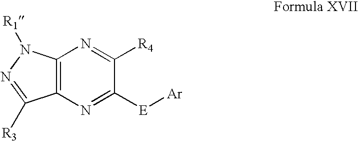
wherein R₁″, R₃, R₄, E, and Ar are as defined in claim 2.

49. A compound or salt according to claim 48, wherein
R₁″ is as defined for claim 48;
R₃ is selected from hydrogen and C₁-C₆alkyl;
R₄ is selected from hydrogen, halogen, cyano, amino, hydroxy, C₁-C₆alkyl, C₁-C₆alkoxy, C₃-C₇cycloalkyl, (C₃-C₇cycloalkyl)C₁-C₄alkyl, (C₃-C₇cycloalkyl)C₁-C₄alkoxy, mono- and di-(C₁-C₆alkyl)amino, amino(C₁-C₆)alkyl, mono and di(C₁-C₆alkyl)amino(C₁-C₆)alkyl, halo(C₁-C₆)alkyl, and halo(C₁-C₆)alkoxy; and
Ar is selected from the group consisting of phenyl, pyridyl and pyrimidinyl, each of which is mono- di- or trisubstituted with substituents independently chosen from halogen, cyano, nitro, halo(C₁-C₆)alkyl, halo(C₁-C₆)alkoxy, hydroxy, amino, C₁-C₆alkyl substituted, C₂-C₆alkenyl, C₂-C₆alkynyl, C₃-C₇cycloalkyl, (C₃-C₇cycloalkyl)C₁-C₄alkyl, C₁-C₆alkoxy, mono- and di(C₁-C₆alkyl)amino, amino(C₁-C₆)alkyl, and mono- and di(C₁-C₆alkyl)amino, wherein, in Ar, at least one of the positions ortho to the point of attachment of Ar shown in Formula XVII is substituted.

50-52. (Cancelled).

53. A compound or salt according to claim 2 of Formula 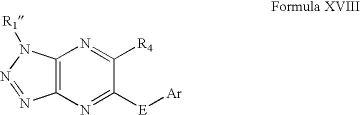
wherein R₁″, R₄, E, and Ar are as defined in claim 2.

54. A compound or salt according to claim 53, wherein:
R₁″ is as defined for claim 53;
R₄ is selected from hydrogen, halogen, cyano, amino, C₁-C₆alkyl, C₁-C₆alkoxy, C₃-C₇cycloalkyl, (C₃-C₇cycloalkyl)C₁-C₄alkyl, (C₃-C₇cycloalkyl)C₁-C₄alkoxy, mono and di(C₁-C₆alkyl)amino, amino(C₁-C₆)alkyl, mono and di(C₁-C₆alkyl)amino(C₁-C₆)alkyl, halo(C₁-C₆)alkyl, and halo(C₁-C₆)alkoxy; and
Ar is selected from the group consisting of phenyl, pyridyl and pyrimidinyl, each of which is mono- di- or trisubstituted with substituents independently chosen from halogen, cyano, nitro, halo(C₁-C₆)alkyl, halo(C₁-C₆)alkoxy, hydroxy, amino, C₁-C₆alkyl substituted, C₂-C₆alkenyl, C₂-C₆alkynyl, C₃-C₇cycloalkyl, (C₃-C₇cycloalkyl)C₁-C₄alkyl, C₁-C₆alkoxy, mono- and di(C₁-C₆alkyl)amino, amino(C₁-C₆)alkyl, and mono- and
di(C₁-C₆alkyl)amino, wherein, in Ar, at least one of the positions ortho to the point of attachment of Ar shown in Formula XVIII is substituted.

55-57. (Cancelled).

58. A compound or salt according to claim 2 of Formula XIX 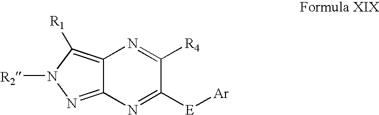
wherein R₁, R₂″, R₄, E, and Ar are as defined in claim 2.

59. A compound or salt according to claim 58, wherein
R₁ is as defined for claim 58;
R₂″ is selected from hydrogen, methyl, and ethyl;
R₄ is selected from hydrogen, halogen, cyano, amino, C₁-C₆alkyl, C₁-C₆alkoxy, C₃-C₇cycloalkyl, (C₃-C₇cycloalkyl)C₁-C₄alkyl, (C₃-C₇cycloalkyl)C₁-C₄alkoxy, mono and di(C₁-C₆alkyl)amino, amino(C₁-C₆)alkyl, mono and di(C₁-C₆alkyl)amino(C₁-C₆)alkyl, halo(C₁-C₆)alkyl, and halo(C₁-C₆)alkoxy; and
Ar is selected from the group consisting of phenyl, pyridyl and pyrimidinyl, each of which is mono- di- or trisubstituted with substituents independently chosen from halogen, cyano, nitro, halo(C₁-C₆)alkyl, halo(C₁-C₆)alkoxy, hydroxy, amino, C₁-C₆alkyl substituted, C₂-C₆alkenyl, C₂-C₆alkynyl, C₃-C₇cycloalkyl, (C₃-C₇cycloalkyl)C₁-C₄alkyl, C₁-C₆alkoxy, mono- and di(C₁-C₆alkyl)amino, amino(C₁-C₆)alkyl, and mono- and di(C₁-C₆alkyl)amino, wherein, in Ar, at least one of the positions ortho to the point of attachment of Ar shown in Formula XIX is substituted.

60-62. (Cancelled).

63. A compound of the Formula XXIII 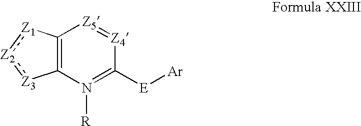
or a pharmaceutically acceptable salt thereof, wherein:
Ar is chosen from:
phenyl which is mono-, di-, or tri-substituted;
1-naphthyl and 2-naphthyl, each of which is optionally mono-, di-, or tri-substituted; and
optionally mono-, di-, or tri-substituted heteroaryl, said heteroaryl having from 1 to 3 rings, 5 to 7 ring members in each ring and, in at least one of said rings, from 1 to about 3 heteroatoms selected from the group consisting of N, O, and S;
wherein in Ar, at least one of the positions ortho to the point of attachment of Ar shown in Formula XXIII is substituted;
R is independently selected at each occurrence to be absent or oxygen;
the group: 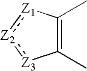
represents a saturated, unsaturated or aromatic 5-membered ring system containing 2 or 3 nitrogen atoms, wherein:
Z₁ is CR₁, CR₁R₁′, or NR₁″;
Z₂ is nitrogen or NR₂″,
Z₃ is CR₃, CR₃R₃′, nitrogen, NR₃″, oxygen, sulfur, sulfoxide or sulfone;
R₁ is chosen from halogen, hydroxy, cyano, amino, optionally substituted alkyl, optionally substituted alkenyl, optionally substituted alkynyl, optionally substituted alkoxy, optionally substituted mono or dialkylamino, optionally substituted heterocycloalkyl, optionally substituted (cycloalkyl)alkyl, optionally substituted (heterocycloalkyl)alkyl, optionally substituted alkyltlio, optionally substituted alkylsulfinyl, optionally substituted alkylsulfonyl, optionally substituted mono- or dialkylcarboxamide, optionally substituted carbocyclic aryl, optionally substituted (aryl)cycloalkyl, optionally substituted (aryl)heterocycloalkyl, optionally substituted heteroaryl, optionally substituted (heteroaryl)cycloalkyl, optionally substituted (heteroaryl)heterocycloalkyl, wherein each heteroaryl has from 1 to 3 rings, 5 to 7 ring members in each ring and, in at least one of said rings, from 1 to about 3 heteroatoms selected from the group consisting of N, O, and S;
R₁″ is chosen from optionally substituted alkyl, optionally substituted alkenyl, optionally substituted alkynyl, optionally substituted heterocycloalkyl, optionally substituted (cycloalkyl)alkyl, optionally substituted (heterocycloalkyl)alkyl, optionally substituted mono or dialkylamino, optionally substituted alkanoyl, optionally substituted carbocyclic aryl, optionally substituted (aryl)cycloalkyl, optionally substituted (aryl)heterocycloalkyl, optionally substituted heteroaryl, optionally substituted (heteroaryl)cycloalkyl, optionally substituted (heteroaryl)heterocycloalkyl, wherein each heteroaryl has from 1 to 3 rings, 5 to 7 ring members in each ring and, in at least one of said rings, from 1 to about 3 heteroatoms selected from the group consisting of N, O, and S;
R₃ is chosen from hydrogen, halogen, hydroxy, amino, cyano, nitro, alkyl, haloalkyl, alkoxy, aminoalkyl, and mono- and di-alkylamino;
R₁′ and R₃′ are independently chosen from hydrogen, halogen, alkyl, haloalkyl, and aminoalkyl;
R₂″ and R₃″ are independently chosen from hydrogen, alkyl, haloalkyl, optionally substituted mono or dialkylamino, optionally substituted alkanoyl, and aminoalkyl;
Z₄′ is NR₄″ or C═O;
Z₅′ is NR₅″ or C═O;
wherein one of Z₄′ or Z₅′ is C═O; and
R₄″ and R₅″ are independently chosen from hydrogen, alkyl, aminoalkyl, and haloalkyl.

64. A compound of the Formula XXIII: 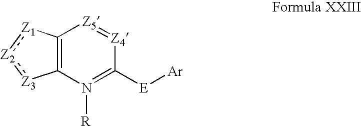
or a pharmaceutically acceptable salt thereof, wherein:
R is independently selected at each occurrence to be absent or oxygen;
E is a single bond, O, or S(O)ₘ;
m is 0, 1, or 2;
Ar is chosen from:
phenyl which is mono-, di-, or tri-substituted with R_{A}, or 1- naphthyl, 2-naphthyl, pyridyl, pyrimidinyl, pyrazinyl, pyridizinyl, imidazo-pyridyl, imidazo-pyrimidinyl, imidazo-pyrazinyl, imidazo-pyridizinyl, thienyl, thiazolyl, oxazolyl, isoxazolyl, pyrrolyl, furanyl, and triazolyl, each of which is optionally mono-, di-, or tri-substituted with R_{A};
wherein in Ar, at least one of the positions ortho to the point of attachment of Ar shown in Formula XXIII is substituted with R_{A};
the group: 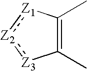
represents a saturated, unsaturated or aromatic ring system comprising 2 or 3 nitrogen atoms, wherein:
Z, is CR₁, CR₁R₁′, or NR₁″;
Z₂ is nitrogen or NR₂″,
Z₃ is CR₃, CR₃R₃′, nitrogen, NR₃″, oxygen, sulfur, sulfoxide or sulfone;
R¹ is chosen from
i) halogen, hydroxy, cyano, amino, C₁-C₁₀carbhydryl, —O(C₁-C₆ carbhydryl), mono or di(C₁-C₆ carbhydryl)amino, (C₃-C₇cycloalkyl) C₁-C₄ carbhydryl, (C₃₋₆heterocycloalkyl)C₀-C₄carbhydryl, (benzoC₃-C₇cycloalkyl)C₀-C₄carbhydryl, (benzo C₃₋₆heterocycloalkyl)C₀-C₄carbhydryl, (C₁C₆)haloalkyl, and mono- and di-(C₁C₆)alkylamino, C₂-C₆alkanoyl; each of which is substituted with 0 or more substituents independently chosen from halogen, hydroxy, amino, oxo, cyano, C₁-C₆alkyl, C₁-C₆alkoxy, C₁-C₆hydroxyalkyl, C₁-C₆alkoxyC₁-C₆alkyl, C₁-C₆haloalkoxy, C₅-C₇heteroaryl, mono- and di-(C₁-C₆)alkylamino, and —XR_{C}, halo(C₁C₆) carbhydryl, —O(halo(C₁C₆) carbhydryl) and S(O)ₙ(C₁-C₆ carbhydryl), —O(C₃-C₇cycloalkyl)C₁-C₄ carbhydryl, and S(O)ₙ(C₁-C₆ carbhydryl),
and
ii) phenyl which is mono-, di-, or tri-substituted with R_{A}, 1-naphthyl, 2-naphthyl, pyridyl, dihydropyridyl, tetrahydropyridyl, pyrimidinyl, pyrazinyl, pyridizinyl, thienyl, thiazolyl, oxazolyl, isoxazolyl, pyrrolyl, furanyl, and triazolyl, each of which is optionally mono-, di-, or tri-substituted with R_{A};
R₁″ is chosen from
i) C₁-C₁ carbhydryl, (C₃-C₇cycloalkyl)C₁-C₄ carbhydryl, and halo(C₁C₆) carbhydryl, (C₃₋₆heterocycloalkyl)C₀-C₄carbhydryl, (benzoC₃-C₇cycloalkyl)C₀-C₄carbhydryl, (benzo C₃₋₆heterocycloalkyl)C₀-C₄carbhydryl, (C₁C₆)haloalkyl, and mono- and di-(C₁C₆)alkylamino, C₂-C₆alkanoyl; each of which is substituted with 0 or more substituents independently chosen from halogen, hydroxy, amino, oxo, cyano, C₁-C₆alkyl, C₁-C₆alkoxy, C₁-C₆hydroxyalkyl, C₁-C₆alkoxyC₁-C₆alkyl, C₁-C₆haloalkoxy, C₅-C₇heteroaryl, mono- and di-(C₁-C₆)alkylamino, and —XR_{C} and
ii) phenyl which is mono-, di-, or tri-substituted with R_{A}, 1-naphthyl, 2-naphthyl, pyridyl, dihydropyridyl, tetrahydropyridyl, pyrimidinyl, pyrazinyl, pyridizinyl, thienyl, thiazolyl, oxazolyl, isoxazolyl, pyrrolyl, furany, and triazolyl, each of which is optionally mono-, di-, or tri-substituted with R_{A};
R₃ is chosen from hydrogen, halogen, hydroxy, amino, cyano, nitro, C₁-C₆alkyl, halo(C₁-C₆)alkyl, C₁-C₆alkoxy, amino(C₁-C₆)alkyl, and mono and di(C₁-C₆)alkylamino;
R₁′ and R₃′ are independently chosen from hydrogen, halogen, C₁-C₆alkyl, halo(C₁-C₆)alkyl, and amino(C₁-C₆)alkyl;
R₂″ and R₃″ are independently chosen from hydrogen, C₁-C₆alkyl, halo(C₁-C₆)alkyl, mono or di(C₁-C₆alkyl)amino, C₁-C₆alkanoyl and amino(C₁-C₆)alkyl;
Z₄′ is NR₄″ or C═O;
Z₅′ is NR₅″ or C═O;
wherein one of Z₄′ or Z₅′ is C═O;
R₄″ and R₅″ are independently chosen from hydrogen, C₁-C₆alkyl, amino(C₁-C₆)alkyl, and halo(C₁-C₆)alkyl;
R_{A} is independently selected at each occurrence from halogen, cyano, nitro, halo(C₁-C₆)alkyl, halo(C₁-C₆)alkoxy, hydroxy, amino, C₁-C₆alkyl substituted with 0-2 R_{B}, C₂-C₆alkenyl substituted with 0-2 R_{B}, C₂-C₆alkynyl substituted with 0-2 R_{B}, C₃-C₇cycloalkyl substituted with 0-2 R_{B}, (C₃-C₇cycloalkyl)C₁-C₄alkyl substituted with 0-2 R_{B}, C₁-C₆alkoxy substituted with 0-2 R_{B}, —NH(C₁-C₆alkyl) substituted with 0-2 R_{B}, —N(C₁-C₆alkyl)(C₁-C₆alkyl) where each C₁-C₆alkyl is independently substituted with 0-2 R_{B}, —S(O)ₙ(C₁-C₆alkyl) substituted with 0-2 R_{B}, —XR_{C}, and Y;
R_{B} is independently selected at each occurrence from halogen, hydroxy, cyano, amino, C₁-C₄alkyl, —O(C₁-C₄alkyl), —NH(C₁-C₄alkyl), —N(C₁-C₄alkyl)(C₁-C₄alkyl), —S(O)ₙ(alkyl), halo(C₁-C₄)alkyl, halo(C₁-C₄)alkoxy, CO(C₁-C₄alkyl), CONH(C₁-C₄alkyl), CON(C₁-C₄alkyl)(C₁-C₄alkyl), —XR_{C}, and Y;
R_{C} and R_{D}, are the same or different, and are independently selected at each occurrence from:
hydrogen, and
straight, branched, and cyclic alkyl groups, and (cycloalkyl)alkyl groups, said straight, branched, and cyclic alkyl groups, C₅-C₇heteroaryl(C₀-C₄alkyl), and (cycloalkyl)alkyl groups consisting of 1 to 8 carbon atoms, and containing zero or one or more double or triple bonds, each of which 1 to 8 carbon atoms may be further substituted with one or more substituent(s) independently selected from oxo, hydroxy, halogen, cyano, amino, C₁-C₆alkoxy, —NH(C₁-C₆alkyl), —N(C₁-C₆alkyl)(C₁-C₆alkyl), —NHC(═O)(C₁-C₆alkyl), —N(C₁-C₆alkyl)C(═O(C₁-C₆alkyl), —NHS(O)ₙ(C₁-C₆alkyl), —S(O)ₙ(C₁-C₆alkyl), —S(O)ₙNH(C₁-C₆alkyl), —S(O)ₙN(C₁-C₆alkyl)(C₁-C₆alkyl), and Z;
X is independently selected at each occurrence from the group consisting of —CH₂—, —CHR_{D}—, —O—, —C(═O)—, —C(S)—, —C(═O)O—, —C(═S)O—, —S(O)ₙ—, —NH—, —NR_{D}—, —C(═O)NH—, —C(═O)NR_{D}—, —S(O)ₙNH—, —S(O)ₙNR_{D}—, —OC(═S)S—, —NHC(═O)—, —NR_{D}C(═O)—, —C(═S)NR_{D}—, —NHS(O)ₙ—, —OSiH₂—, —OSiH(C₁-C₄alkyl)-, —OSi(C₁-C₄alkyl)(C₁-C₄alkyl)-, and —NR_{D}S(O)ₙ—;
Y and Z are independently selected at each occurrence from: 3- to 7-membered carbocyclic or heterocyclic groups which are saturated, unsaturated, or aromatic, which may be further substituted with one or more substituents independently selected from halogen, oxo, hydroxy, amino, cyano, C₁-C₄alkyl, —O(C₁-C₄alkyl), —NH(C₁-C₄alkyl), —N(C₁-C₄alkyl)(C₁-C₄alkyl),and —S(O)ₙ(alkyl),
wherein said 3- to 7-memberered heterocyclic groups contain one or more heteroatom(s) independently selected from N, O, and S, with the point of attachment being either carbon or nitrogen; and
n is independently selected at each occurrence from 0, 1, and 2.

65. A compound or salt according to claim 64 of Formula XIV: 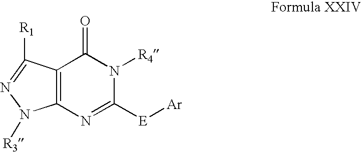
wherein R₁, R₃″, R₄″, E, and Ar are as defined in claim 64.

66. A compound or salt according to claim 65, wherein:
R₁ is as defined for claim 65;
R₃″ is selected from hydrogen and C₁-C₆alkyl;
R₄″ is selected from hydrogen, methyl, and ethyl;
Ar is selected from the group consisting of phenyl, pyridyl and pyrimidinyl which is mono- di- or trisubstituted with substituents independently chosen from halogen, cyano, nitro, halo(C₁-C₆)alkyl, halo(C₁-C₆)alkoxy, hydroxy, amino, C₁-C₆alkyl substituted, C₂-C₆alkenyl, C₂-C₆alkynyl, C₃-C₇cycloalkyl, (C₃-C₇cycloalkyl)C₁-C₄alkyl, C₁-C₆alkoxy, mono- and di(C₁-C₆alkyl)amino, amino(C₁-C₆)alkyl, and mono- and di(C₁-C₆alkyl)amino, wherein, in Ar, at least one of the positions ortho to the point of attachment of Ar shown in Formula XXIV is substituted.

67-69. (Cancelled).

70. A compound or salt according to claim 64 of Formula XXV: 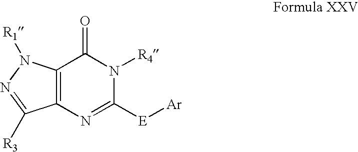
wherein R₁″, R₃, R₄″, E, and Ar are as defined in claim 64.

71. A compound or salt according to claim 70, wherein
R₁″ is as defined for claim 70;
R₃ is selected from hydrogen and C₁-C₆alkyl;
R₄″ is selected from hydrogen, methyl, and ethyl; and
Ar is selected from the group consisting of phenyl, pyridyl and pyrimidinyl, each of which is mono- di- or trisubstituted with substituents independently chosen from halogen, cyano, nitro, halo(C₁-C₆)alkyl, halo(C₁-C₆)alkoxy, hydroxy, amino, C₁-C₆alkyl substituted, C₂-C₆alkenyl, C₂-C₆alkynyl, C₃-C₇cycloalkyl, (C₃-C₇cycloalkyl)C₁-C₄alkyl, C₁-C₆alkoxy, mono- and di(C₁-C₆alkyl)amino, amino(C₁-C₆)alkyl, and mono- and di(C₁-C₆alkyl)amino, wherein, in Ar, at least one of the positions ortho to the point of attachment of Ar shown in Formula XXV is substituted.

72-74. (Cancelled).

75. A compound or salt according to claim 64 of Formula XXVI: 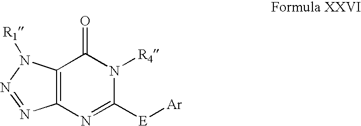
wherein R₁″, R₄″, E, and Ar are as defined in claim 64.

76. A compound or salt according to claim 75, wherein:
R₁″ is as defined for claim 75;
R₄″ is selected from hydrogen, methyl, and ethyl; and
Ar is selected from the group consisting of phenyl, pyridyl and pyrimidinyl which is mono- di- or trisubstituted with substituents independently chosen from halogen, cyano, nitro, halo(C₁-C₆)alkyl, halo(C₁-C₆)alkoxy, hydroxy, amino, C₁-C₆alkyl substituted, C₂-C₆alkenyl, C₂-C₆alkynyl, C₃-C₇cycloalkyl, (C₃-C₇cycloalkyl)C₁-C₄alkyl, C₁-C₆alkoxy, mono- and di(C₁-C₆alkyl)amino, amino(C₁-C₆)alkyl, and mono- and di(C₁-C₆alkyl)amino, wherein, in Ar, at least one of the positions ortho to the point of attachment of Ar shown in Formula XXVI is substituted.

77-79. (Cancelled).

80. A compound or salt according to claim 64 of Formula XXVII 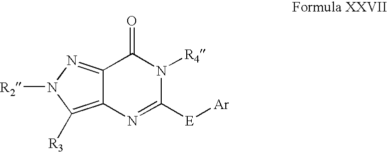
wherein R₂″, R₃, R₄″, E, and Ar are as defined in claim 64.

81. A compound or salt according to claim 80, wherein:
R₂″ is as defined for claim 80;
R₃ is selected from hydrogen, methyl, and ethyl;
R₄″ is selected from hydrogen, methyl, and ethyl; and
Ar is selected from the group consisting of phenyl, pyridyl and pyrimidinyl, each of which is mono- di- or tri-substituted with substituents independently chosen from halogen, cyano, nitro, halo(C₁-C₆)alkyl, halo(C₁-C₆)alkoxy, hydroxy, amino, C₁-C₆alkyl substituted, C₂-C₆alkenyl, C₂-C₆alkynyl, C₃-C₇cycloalkyl, (C₃-C₇cycloalkyl)C₁-C₄alkyl, C₁-C₆alkoxy, mono- and di(C₁-C₆alkyl)amino, amino(C₁-C₆)alkyl, and mono- and di(C₁-C₆alkyl)amino, wherein, in Ar, at least one of the positions ortho to the point of attachment of Ar shown in Formula XXVII is substituted.

82. A compound or salt according to claim 64 of Formula XXXI 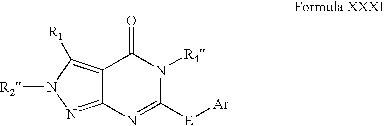
wherein R₁, R₂″, R₄″, E, and Ar are as defined in claim 64.

83. A compound or salt according to claim 82, wherein
R₁ is as defined for claim 82;
R₂″ is selected from hydrogen, methyl, and ethyl;
R₄″ is selected from hydrogen, methyl, and ethyl; and
Ar is selected from the group consisting of phenyl, pyridyl and pyrimidinyl, each of which is mono- di- or trisubstituted with substituents independently chosen from halogen, cyano, nitro, halo(C₁-C₆)alkyl, halo(C₁-C₆)alkoxy, hydroxy, amino, C₁-C₆alkyl substituted,
C₂-C₆alkenyl, C₂-C₆alkynyl, C₃-C₇cycloalkyl, (C₃-C₇cycloalkyl)C₁-C₄alkyl, C₁-C₆alkoxy, mono- and di(C₁-C₆alkyl)amino, amino(C₁-C₆)alkyl, and mono- and di(C₁-C₆alkyl)amino, wherein, in Ar, at least one of the positions ortho to the point of attachment of Ar shown in Formula XXXI is substituted.

84-86. (Cancelled).

87. A compound or salt according to claim 64 of Formula XXXII: 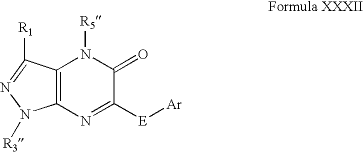
wherein R₁, R₃″, R₅″, E, and Ar are as defined in claim 64.

88. A compound or salt according to claim 87, wherein:
R₁ is as defined for claim 87;
R₃″ is selected from hydrogen and C₁-C₆alkyl;
R₅″ is selected from hydrogen, methyl, and ethyl; and
Ar is selected from the group consisting of phenyl, pyridyl and pyrimidinyl, each of which is mono- di- or trisubstituted with substituents independently chosen from halogen, cyano, nitro, halo(C₁-C₆)alkyl, halo(C₁-C₆)alkoxy, hydroxy, amino, C₁-C₆alkyl substituted, C₂-C₆alkenyl, C₂-C₆alkynyl, C₃-C₇cycloalkyl, (C₃-C₇cycloalkyl)C₁-C₄alkyl, C₁-C₆alkoxy, mono- and di(C₁-C₆alkyl)amino, amino(C₁-C₆)alkyl, and mono- and di(C₁-C₆alkyl)amino, wherein, in Ar, at least one of the positions ortho to the point of attachment of Ar shown in Formula XXXII is substituted.

89-91. (Cancelled).

92. A compound or salt according to claim 64 of Formula XXXIII: 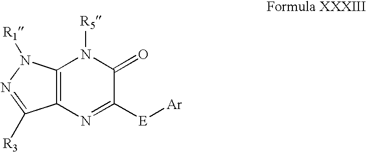
wherein R₁″, R₃, R₅″, E, and Ar are as defined in claim 64.

93. A compound or salt according to claim 92, wherein
R₁″ is as defined for claim 92;
R₅″ is selected from hydrogen, methyl, and ethyl;
R₃ is selected from hydrogen and C₁-C₆alkyl; and
Ar is selected from the group consisting of phenyl, pyridyl and pyrimidinyl, each of which is mono- di- or tri-substituted with substituents independently chosen from halogen, cyano, nitro, halo(C₁-C₆)alkyl, halo(C₁-C₆)alkoxy, hydroxy, amino, C₁-C₆alkyl substituted, C₂-C₆alkenyl, C₂-C₆alkynyl, C₃-C₇cycloalkyl, (C₃-C₇cycloalkyl)C₁-C₄alkyl, C₁-C₆alkoxy, mono- and di(C₁-C₆alkyl)amino, amino(C₁-C₆)alkyl, and mono- and di(C₁-C₆alkyl)amino, wherein, in Ar, at least one of the positions ortho to the point of attachment of Ar shown in Formula XXXIII is substituted.

94-96. (Cancelled).

97. A compound or salt according to claim 64 of Formula XXXIV: 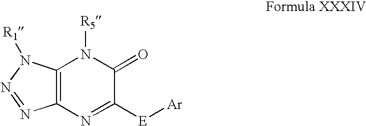
wherein R₁″, R₅″, E, and Ar are as defined in claim 64.

98. A compound or salt according to claim 97, wherein:
R₁″ is as defined for claim 97;
R₅″ is selected from hydrogen, methyl, and ethyl; and
Ar is selected from the group consisting of phenyl, pyridyl and pyrimidinyl, each of which is mono- di- or trisubstituted with substituents independently chosen from halogen, cyano, nitro, halo(C₁-C₆)alkyl, halo(C₁-C₆)alkoxy, hydroxy, amino, C₁-C₆alkyl substituted, C₂-C₆alkenyl, C₂-C₆alkynyl, C₃-C₇cycloalkyl, (C₃-C₇cycloalkyl)C₁-C₄alkyl, C₁-C₆alkoxy, mono- and di(C₁-C₆alkyl)amino, amino(C₁-C₆)alkyl, and mono- and di(C₁-C₆alkyl)amino, wherein, in Ar, at least one of the positions ortho to the point of attachment of Ar shown in Formula XXXIV is substituted.

99-101. (Cancelled).

102. A compound of the Formula XXXVIII: 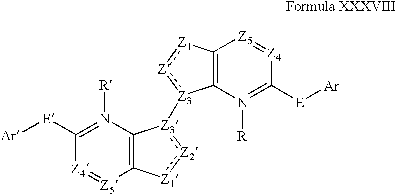
or a pharmaceutically acceptable salt thereof, wherein:
R and R′ are independently selected to be absent or oxygen;
E is a single bond, O, or S(O)ₘ;
m is 0, 1, or 2;
Ar and Ar′ are independently chosen from:
phenyl which is mono-, di-, or tri-substituted with R_{A}, or 1-naphthyl, 2-naphthyl, pyridyl, pyrimidinyl, pyrazinyl, pyridizinyl, imidazo-pyridyl, imidazo-pyrimidinyl, imidazo-pyrazinyl, imidazo-pyridizinyl, thienyl, thiazolyl, oxazolyl, isoxazolyl, pyrrolyl, furanyl, and triazolyl, each of which is optionally mono-, di-, or tri-substituted with R_{A};
wherein in Ar and Ar′, at least one of the positions ortho to the point of attachment of Ar and Ar′ shown in Formula XXXVIII are substituted with R_{A};
the groups: 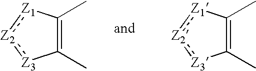
represents a saturated, unsaturated or aromatic ring system comprising 2 or 3 adjacent nitrogen atoms, wherein:
Z₁ and Z₁′ are independently selected from CR₁, CR₁R₁′, or NR₁″;
Z₂ and Z₂′ are nitrogen or NR₂″;
Z₃ and Z₃′ are CR₃, CR₃R₃′, nitrogen, NR₃″, oxygen, sulfur, sulfoxide or sulfone;
R₁ is chosen from
i) halogen, hydroxy, cyano, amino, C₁-C₁₀carbhydryl, —O(C₁-C₆ carbhydryl), mono or di(C₁-C₆ carbhydryl)amino, (C₃-C₇cyclocarbhydryl) C₁-C₄ carbhydryl, (C₃₋₆heterocycloalkyl)C₀-C₄carbhydryl, (benzoC₃-C₇cycloalkyl)C₀-C₄carbhydryl, (benzoC₃-C₇heterocycloalkyl)C₀-C₄carbhydryl, (C₁C₆)haloalkyl, and mono- and di-(C₁C₆)alkylamino, C₂-C₆alkanoyl; each of which is substituted with 0 or more substituents independently chosen from halogen, hydroxy, amino, oxo, cyano, C₁-C₆alkyl, C₁-C₆alkoxy, C₁-C₆hydroxyalkyl, C₁-C₆alkoxyC₁-C₆alkyl, C₁-C₆haloalkoxy, C₅-C₇heteroaryl, mono- and di-(C₁-C₆)alkylamino, and —XR_{C}, halo(C₁C₆)carbhydryl, —O(halo(C₁C₆)carbhydryl) and S(O)ₙ(C₁-C₆ carbhydryl), —O(C₃-C₇cyclo carbhydryl)C₁-C₄ carbhydryl, and S(O)ₙ(C₁-C₆ carbhydryl), and
ii) phenyl which is mono-, di-, or tri-substituted with R_{A}, 1-naphthyl, 2-naphthyl, pyridyl, dihydropyridyl, tetrahydropyridyl, pyrimidinyl, pyrazinyl, pyridizinyl, thienyl, thiazolyl, oxazolyl, isoxazolyl, pyrrolyl, furanyl, and triazolyl, each of which is optionally mono-, di-, or tri-substituted with R_{A};
R₁″ is chosen from
i) C₁-C₁₀carbhydryl, (C₃-C₇cycloalkyl)C₁-C₄ carbhydryl, and halo(C₁C₆) carbhydryl, (C₃₋₆heterocycloalkyl)C₀-C₄carbhydryl, (benzoC₃-C₇cycloalkyl)C₀-C₄carbhydryl, (benzo C₃₋₆heterocycloalkyl)C₀-C₄carbhydryl, (C₁C₆)haloalkyl, and mono- and di-(C₁C₆)alkylamino, C₂-C₆alkanoyl; each of which is substituted with 0 or more substituents independently chosen from halogen, hydroxy, amino, oxo, cyano, C₁-C₆alkyl, C₁-C₆alkoxy, C₁-C₆hydroxyalkyl, C₁-C₆alkoxyC₁-C₆alkyl, C₁-C₆haloalkoxy, C₅-C₇heteroaryl, mono- and di-(C₁-C₆)alkylamino, and —XR_{C}, and
ii) phenyl which is mono-, di-, or tri-substituted with R_{A}, benzyl, 1-naphthyl, 2-naphthyl, pyridyl, dihydropyridyl, tetrahydropyridyl, pyrimidinyl, pyrazinyl, pyridizinyl, thienyl, thiazolyl, oxazolyl, isoxazolyl, pyrrolyl, furanyl, and triazolyl, each of which is optionally mono-, di-, or tri-substituted with R_{A};
R₃ is chosen from hydrogen, halogen, hydroxy, amino, cyano, nitro, C₁-C₆alkyl, halo(C₁-C₆)alkyl, C₁-C₆alkoxy, amino(C₁-C₆)alkyl, and mono and di(C₁-C₆)alkylamino;
R₁′ and R₃′ are independently chosen from hydrogen, halogen, C₁-C₆alkyl, halo(C₁-C₆)alkyl, and amino(C₁-C₆)alkyl;
R₂″ and R₃″ are independently chosen from hydrogen, C₁-C₆alkyl, halo(C₁-C₆)alkyl, mono or di(C₁-C₆alkyl)amino, C₁-C₆alkanoyl and amino(C₁-C₆)alkyl;
Z₄ and Z₄′ are selected from NR and CR₄;
Z₅ and Z₅′ are selected from NR and CR₅;
R₄ and R₅ are independently chosen from hydrogen, halogen, cyano, nitro, amino, mono or di(C₁-C₆ carbhydryl)amino, C₁-C₆ carbhydryl, (C₃-C₇cycloalkyl) C₁-C₄ carbhydryl,—O(C₃-C₇cycloalkyl) C₁-C₄ carbhydryl, halo(C₁-C₆) carbhydryl, —O(halo(C₁-C₆) carbhydryl), —O(C₁-C₆ carbhydryl), S(O)ₙ(C₁-C₆ carbhydryl), N(H)(S(O)ₙ(C₁-C₆ carbhydryl)), N(C₁-C₆ carbhydryl) (S(O)ₙ(C₁-C₆ carbhydryl)
where each carbhydrylis independently straight, branched, or cyclic, contains zero or 1 or more double or triple bonds, and is optionally substituted with one or more substituents independently chosen from halogen, hydroxy, amino, oxo, cyano, C₁-C₄alkoxy, and mono- or di(C₁-C₄)alkylamino,
and
where each C₃-C₇cycloalkyl is optionally substituted by one or more substituents independently chosen from halogen, amino, hydroxy, oxo, cyano, C₁-C₄alkoxy, and mono- or di(C₁-C₄)alkylamino;
R_{A} is independently selected at each occurrence from halogen, cyano, nitro, halo(C₁-C₆)alkyl, halo(C₁-C₆)alkoxy, hydroxy, amino, C₁-C₆alkyl substituted with 0-2 R_{B}, C₂-C₆alkenyl substituted with 0-2 R_{B}, C₂-C₆alkynyl substituted with 0-2 R_{B}, C₃-C₇cycloalkyl substituted with 0-2 R_{B}, (C₃-C₇cycloalkyl)C₁-C₄alkyl substituted with 0-2 R_{B}, C₁-C₆alkoxy substituted with 0-2 R_{B}, —NH(C₁-C₆alkyl) substituted with 0-2 R_{B}, N(C₁-C₆alkyl)(C₁-C₆alkyl) where each C₁-C₆alkyl is independently substituted with 0-2 R_{B}, —S(O)ₙ(C₁-C₆alkyl) substituted with 0-2 R_{B}, —XR_{C}, and Y;
R_{B} is independently selected at each occurrence from halogen, hydroxy, cyano, amino, C₁-C₄alkyl, —O(C₁-C₄alkyl), —NH(C₁-C₄alkyl), —N(C₁-C₄alkyl)(C₁-C₄alkyl), —S(O)ₙ(alkyl), halo(C₁-C₄)alkyl, halo(C₁-C₄)alkoxy, CO(C₁-C₄alkyl), CONH(C₁-C₄alkyl), CON(C₁-C₄alkyl)(C₁-C₄alkyl), —XR_{C}, and Y;
R_{C} and R_{D}, are the same or different, and are independently selected at each occurrence from:
hydrogen, and straight, branched, and cyclic alkyl groups, and (cycloalkyl)alkyl groups, said straight, branched, and cyclic alkyl groups, C₅-C₇heteroaryl(C₀-C₄alkyl), and (cycloalkyl)alkyl groups consist of 1 to 8 carbon atoms, and contain zero or one or more double or triple bonds, each of which 1 to 8 carbon atoms may be further substituted with one or more substituent(s) independently selected from oxo, hydroxy, halogen, cyano, amino, C₁-C₆alkoxy, —NH(C₁-C₆alkyl), —N(C₁-C₆alkyl)(C₁-C₆alkyl), —NHC(═O)(C₁-C₆alkyl), —N(C₁-C₆alkyl)C(═O)(C₁-C₆alkyl), —NHS(O)ₙ(C₁-C₆alkyl), —S(O)ₙ(C₁-C₆alkyl), —S(O)ₙNH(C₁-C₆alkyl), —S(O)ₙN(C₁-C₆alkyl)(C₁-C₆alkyl), and Z;
X is independently selected at each occurrence from the group consisting of —CH₂—, —CHR_{D}—, —O—, —C(═O)—, —C(S)—, —C(═O)O—, —C(═S)O—, —S(O)ₙ—, —NH—, —NR_{D}—, —C(═O)NH—, —C(═O)NR_{D}—, —S(O)ₙNH—, —S(O)ₙNR_{D}—, —OC(═S)S—, —NHC(═O)—, —NR_{D}C(═O)—, —C(═S)NR_{D}—, —NHS(O)ₙ—, —OSiH₂—, —OSiH(C₁-C₄alkyl)-, —OSi(C₁-C₄alkyl)(C₁-C₄alkyl)-, and —NR_{D}S(O)ₙ—;
Y and Z are independently selected at each occurrence from: 3- to 7-membered carbocyclic or heterocyclic groups which are saturated, unsaturated, or aromatic, which may be further substituted with one or more substituents independently selected from halogen, oxo, hydroxy, amino, cyano, C₁-C₄alkyl, —O(C₁-C₄alkyl), —NH(C₁-C₄alkyl), —N(C₁-C₄alkyl)(C₁-C₄alkyl),and —S(O)ₙ(alkyl),
wherein said 3- to 7-memberered heterocyclic groups contain one or more heteroatom(s) independently selected from N, O, and S, with the point of attachment being either carbon or nitrogen; and
n is independently selected at each occurrence from 0, 1, and 2.

103. A compound or pharmaceutically acceptable salt thereof, wherein the compound is selected from the group consisting of:
1-(1-Ethyl-propyl)-5-(2-methoxy-4-trifluoromethoxy-phenyl)-6-methyl-1H-[1,2,3]triazolo[4,5-b]pyrazine;
1-(1-Ethyl-propyl)-5-(2-methoxy-4-trifluoromethoxy-phenyl)-3,6-dimethyl-1H-pyrazolo[3,4-b]pyrazine;
3-(1-Ethyl-propoxy)-6-(2-methoxy-4-trifluoromethoxy-phenyl)-1,5-dimethyl-1H-pyrazolo[3,4-b]pyrazine;
1,1′-Bis-(1-ethyl-propyl)-5,5′-bis-(2-methoxy-4-trifluoromethoxy-phenyl)-6,6′-dimethyl-1H, 1′H-[3,3′]bi[pyrazolo[3,4-b]pyrazinyl];
1-(1-Ethyl-propyl)-5-(2-methoxy-4-trifluoromethoxy-phenyl)-6-methyl-1H-pyrazolo[3,4-b]pyrazine;
Diethyl-{4-ethyl-5-[3-(1-ethyl-propyl)-1,5-dimethyl-1H-pyrazolo[3,4-b]pyridin-6-yl]-pyridin-2-yl}-amine;
3-(1-Ethyl-propyl)-6-(2-methoxy-4-trifluoromethoxy-phenyl)-1,5-dimethyl-1H-pyrazolo[3,4-b]pyridine;
5-Ethyl-3-(1-ethyl-propyl)-6-(2-methoxy-4-trifluoromethoxy-phenyl)-1-methyl-1H-pyrazolo[3,4-b]pyridine;
(1-Ethyl-propyl)-{5-[3-(1-ethyl-propyl)-1,5-dimethyl-1H-pyrazolo[3,4-b]pyridin-6-yl]-3-methoxy-6-methyl-pyrazin-2-yl}-amine;
6-(2-Chloro-4-methoxy-phenyl)-3-(1-ethyl-propyl)-1,5-dimethyl-1H-pyrazolo[3,4-b]pyridine;
5-Chloro-6-(5-chloro-2-methoxy-4-trifluoromethoxy-phenyl)-3-(1-ethyl-propyl)-1-methyl-1H-pyrazolo[3,4-b]pyridine;
5-Chloro-3-(1-ethyl-propyl)-6-(2-methoxy-4-trifluoromethoxy-phenyl)-1-methyl-1H-pyrazolo[3,4-b]pyridine;
5-(2,4-Dichloro-phenyl)-1-(1-ethyl-propyl)-3,6-dimethyl-1H-pyrazolo[3,4-b]pyrazine;
1-(1-Ethyl-propyl)-5-(6-isopropyl-2-methoxy-pyridin-3-yl)-3,6-dimethyl-1H-pyrazolo[3,4-b]pyrazine;
[5-(5-Ethyl-3-isopropyl-1-methyl-1H-pyrazolo [3,4-b]pyridin-6-yl)-3-methoxy-6-methyl-pyrazin-2-yl]-(1-ethyl-propyl)-amine;
{5-[1-(1-Ethyl-propyl)-3,6-dimethyl-1H-pyrazolo[3,4-b]pyrazin-5-yl]-4-methyoxy-pyridin-2-yl}-dimethyl-amine;
{5-[1-(1-Ethyl-propyl)-3,6-dimethyl-1H-pyrazolo[3,4-b]pyrazin-5-yl]-4-isopropoxy-pyridin-2-yl}-dimethyl-amine;
Diethyl-{4-ethyl-5-[6-ethyl-1-(1-ethyl-propyl)-3-methyl-1H-pyrazolo[3,4-b]pyrazin-5-yl]-pyridin-2-yl}-amine;
6-Ethyl-1-(1-ethyl-propyl)-5-(6-isopropyl-2-methoxy-pyridin-3-yl)-3-methyl-1H-pyrazolo[3,4-b]pyrazine;
5-(2-Chloro-4-methoxy-phenyl)-6-ethyl-1-(1-ethyl-propyl)-3-methyl-1H-pyrazolo[3,4-b]pyrazine;
6-Ethyl-1-(1-ethyl-propyl)-5-(2-methoxy-4-trifluoromethoxy-phenyl)-3-methyl-1H-pyrazolo[3,4-b]pyrazine;
{5-[6-Ethyl-1-(1-ethyl-propyl)-3-methyl-1H-pyrazolo[3,4-b]pyrazin-5-yl]-4-isopropoxy-pyridin-2-yl}-dimethyl-amine;
Diethyl-{4-ethyl-5-[1-(1-ethyl-propyl)-3,6-dimethyl-1H-pyrazolo[3,4-b]pyrazin-5-yl]-pyridin-2-yl}-amine;
2-({3-[6-Ethyl-1-(1-ethyl-propyl)-3-methyl-1H-pyrazolo[3,4-b]pyrazin-5-yl-6-isopropyl-pyridin-2-yl}-methyl-amino)-ethanol;
1-{3-[6-Ethyl-1-(1-ethyl-propyl)-3-methyl-1H-pyrazolo[3,4-b]pyrazin-5-yl]-6-isopropyl-pyridin-2-yl}-pyrrolidin-3-ol;
{3-[6-Ethyl-1-(1-ethyl-propyl)-3-methyl-1H-pyrazolo[3,4-b]pyrazin-5-yl]-6-isopropyl-pyridin-2-yl}-(2-methoxy-ethyl)-amine;
{3-[6-Ethyl-1-(1-ethyl-propyl)-3-methyl-1H-pyrazolo[3,4-b]pyrazin-5-yl]-6-isopropyl-pyridin-2-yl}-dimethyl-amine;
3′-[6-Ethyl-1-(1-ethyl-propyl)-3-methyl-1H-pyrazolo[3,4-b]pyrazin-5-yl]-6′-isopropyl-3,4,5,6-tetrahydro-2H-[1,2′]bipyridinyl;
1-(1-Ethyl-propyl)-5-(6-isopropyl-2-methoxy-pyridin-3-yl)-6-methoxy-3-methyl-1H-pyrazolo[3,4-b]pyrazine;
{3-[1-(1-Ethyl-propyl)-3,6-dimethyl-1H-pyrazolo[3,4-b]pyrazin-5-yl]-6-isopropyl-pyridin-2-yl}-(2-methoxy-ethyl)-amine;
{3-[1-(1-Ethyl-propyl)-3,6-dimethyl-1H-pyrazolo[3,4-b]pyrazin-5-yl]-6-isopropyl-pyridin-2-yl}-[2-(1H-imidazol-4-yl)-ethyl]-amine;
1-(1-Ethyl-propyl)-5-(6-isopropyl-2-morpholin-4-yl-pyridin-3-yl)-3,6-dimethyl-1H-pyrazolo[3,4-b]pyrazine;
N-(2-{3-[1-(1-Ethyl-propyl)-3,6-dimethyl-1H-pyrazolo[3,4-b]pyrazin-5-yl]-6-isopropyl-pyridin-2-ylamino}-ethyl)-acetamide;
N′-{3-[1-(1-Ethyl-propyl)-3,6-dimethyl-1H-pyrazolo[3,4-b]pyrazin-5-yl]-6-isopropyl-pyridin-2-yl}-N,N-dimethyl-pentane-1,5-diamine;
{3-[1-(1-Ethyl-propyl)-3,6-dimethyl-1H-pyrazolo[3,4-b]pyrazin-5-yl]-6-isopropyl-pyridin-2-yl}-methyl-amine;
{3-[1-(1-Ethyl-propyl)-3,6-dimethyl-1H-pyrazolo[3,4-b]pyrazin-5-yl]-6-isopropyl-pyridin-2-yl}-dimethyl-amine;
{3-[6-Ethyl-1-(1-ethyl-propyl)-3-methyl-1H-pyrazolo[3,4-b]pyrazin-5-yl]-6-isopropyl-pyridin-2-yl}-methyl-amine;
5-(2-Azetidin-1-yl-6-isopropyl-pyridin-3-yl)-1-(1-ethyl-propyl)-3,6-dimethyl-1H-pyrazolo[3,4-b]pyrazine;
N′-{3-[1-(1-Ethyl-propyl)-3,6-dimethyl-1H-pyrazolo[3,4-b]pyrazin-5-yl]-6-isopropyl-pyridin-2-yl}-N,N-dimethyl-ethane-1,2-diamine;
{3-[6-Ethyl-1-(1-ethyl-propyl)-3-methyl-1H-pyrazolo[3,4-b]pyrazin-5-yl]-6-isopropyl-pyridin-2-yl}-(3-piperidin-1-yl-propyl)-amine;
(1-{3-[6-Ethyl-1-(1-ethyl-propyl)-3-methyl-1H-pyrazolo[3,4-b]pyrazin-5-yl]-6-isopropyl-pyridin-2-yl}-pyrrolidin-3-yl)-dimethyl-amine;
N-[5-(6-Diethylamino-4-ethyl-pyridin-3-yl)-1-(1-ethyl-propyl)-3-methyl-1H-pyrazolo[3,4-b]pyrazin-6-yl]-C,C,C-trifluoro-N-methyl-methanesulfonamide;
[1-(1-Ethyl-propyl)-5-(6-isopropyl-2-methoxy-pyridin-3-yl)-3-methyl-1H-pyrazolo[3,4-b]pyrazin-6-yl]-methyl-amine;
{3-[6-Ethyl-1-(1-ethyl-propyl)-3-methyl-1H-pyrazolo[3,4-b]pyrazin-5-yl]-6-isopropyl-pyridin-2-yl}-(tetrahydro-furan-2-ylmethylamine;
1-Benzyl-5-(6-isopropyl-2-methoxy-pyridin-3-yl)-3,6-dimethyl-1H-pyrazolo[3,4-b]pyrazine;
6-Ethyl-5-(6-isopropyl-2-methoxy-pyridin-3-yl)-1-(2-methoxy-1-methyl-ethyl)-3-methyl-1H-pyrazolo[3,4-b]pyrazine;
1-(1-Ethyl-propyl)-6-methoxy-5-(2-methoxy-4-trifluoromethoxy-phenyl)-3-methyl-1H-pyrazolo[3,4-b]pyrazine;
Diethyl-{4-ethyl-5-[1-(1-ethyl-propyl)-6-methoxy-3-methyl-1H-pyrazolo[3,4-b]pyrazin-5-yl]-pyridin-2-yl}-amine;
{3-[6-Ethyl-1-(2-methoxy-1-methyl-ethyl)-3-methyl-1H-pyrazolo[3,4-b]pyrazin-5-yl]-6-isopropyl-pyridin-2-yl}-methyl-amine;
6-Ethyl-5-(2-ethyl-6-isopropyl-pyridin-3-yl)-1-(2-methoxy-1-methyl-ethyl)-3-methyl-1H-pyrazolo[3,4-b]pyrazine;
1-Isopropyl-5-(6-isopropyl-2-methoxy-pyridin-3-yl)-3,6-dimethyl-1H-pyrazolo[3,4-b]pyrazine;
6-Ethyl-5-(6-isopropyl-2-methoxy-pyridin-3-yl)-1-(2-methoxy-1-methoxymethyl-ethyl)-3-methyl-1H-pyrazolo[3,4-b]pyrazine;
[1-(1-Ethyl-propyl)-5-(2-methoxy-4-trifluoromethoxy-phenyl)-3-methyl-1H-pyrazolo[3,4-b]pyrazin-6-yl]-methyl-amine;
[5-(6-Diethylamino-4-ethyl-pyridin-3-yl)-1-(1-ethyl-propyl)-3-methyl-1H-pyrazolo[3,4-b]pyrazin-6-yl]-methyl-amine;
{3-[6-Ethyl-1-(1-methoxymethyl-propyl)-3-methyl-1H-pyrazolo[3,4-b]pyrazin-5-yl]-6-isopropyl-pyridin-2-yl}-methyl-amine;
[3-(1-Benzyl-6-ethyl-3-methyl-1H-pyrazolo[3,4-b]pyrazin-5-yl)-6-isopropyl-pyridin-2-yl]-methyl-amine;
1-(2-Benzyloxy-1-methoxymethyl-ethyl)-6-ethyl-5-(6-isopropyl-2-methoxy-pyridin-3-yl)-3-methyl-1H-pyrazolo[3,4-b]pyrazine;
3-Benzyloxy-2-[6-ethyl-5-(6-isopropyl-2-methoxy-pyridin-3-yl)-3-methyl-pyrazolo[3,4-b]pyrazin-1-yl]-propan-1-ol;
5-(6-Diethylamino-4-ethyl-pyridin-3-yl)-1-(1-ethyl-propyl)-3-methyl-1H-pyrazolo[3,4-b]pyrazin-6-ol;
[3-(1-sec-Butyl-6-ethyl-3-methyl-1H-pyrazolo[3,4-b]pyrazin-5-yl)-6-isopropyl-pyridin-2-yl]-methyl-amine;
2-[6-Ethyl-5-(6-isopropyl-2-methoxy-pyridin-3-yl)-3-methyl-pyrazolo[3,4-b]pyrazin-1-yl]-3-methoxy-propan-1-ol;
Cyclobutyl-{2-[6-ethyl-5-(6-isopropyl-2-methoxy-pyridin-3-yl)-3-methyl-pyrazolo[3,4-b]pyrazin-1-yl]-3-methoxy-propyl}-amine;
6-Ethyl-5-(6-isopropyl-2-methoxy-pyridin-3-yl)-1-(1-methoxymethyl-2-pyrrolidin-1-yl-ethyl)-3-methyl-1H-pyrazolo[3,4-b]pyrazine;
Ethyl-{2-[6-ethyl-5-(6-isopropyl-2-methoxy-pyridin-3-yl)-3-methyl-pyrazolo[3,4-b]pyrazin-1-yl]-3-methoxy-propyl}-methyl-amine;
6-Ethyl-5-(6-isopropyl-2-methoxy-pyridin-3-yl)-1-(1-methoxymethyl-2-morpholin-4-yl-ethyl)-3-methyl-1H-pyrazolo[3,4-b]pyrazine;
6-Ethyl-1-isopropyl-5-(6-isopropyl-2-methoxy-pyridin-3-yl)-3-methyl-1H-pyrazolo[3,4-b]pyrazine;
Cyclobutyl-{2-[6-ethyl-5-(6-isopropyl-2-methoxy-pyridin-3-yl)-3-methyl-pyrazolo[3,4-b]pyrazin-1-yl]-propyl}-amine;
6-Ethyl-5-(6-isopropyl-2-methoxy-pyridin-3-yl)-3-methyl-1-(1-methyl-2-pyrrolidin-1-yl-ethyl)-1H-pyrazolo[3,4-b]pyrazine;
Ethyl-{2-[6-ethyl-5-(6-isopropyl-2-methoxy-pyridin-3-yl)-3-methyl-pyrazolo [3,4-b]pyrazin-1-yl]-propyl}-methyl-amine;
6-Ethyl-5-(6-isopropyl-2-methoxy-pyridin-3-yl)-3-methyl-1-(1-methyl-2-morpholin-4-yl-ethyl)-1H-pyrazolo[3,4-b]pyrazine;
{3-[1-(1-Diethoxymethyl-propyl)-6-ethyl-3-methyl-1H-pyrazolo[3,4-b]pyrazin-5-yl]-6-isopropyl-pyridin-2-yl}-methyl-amine;
{3-[6-Ethyl-3-methyl-1-(1-morpholin-4-ylmethyl-propyl)-1H-pyrazolo[3,4-b]pyrazin-5-yl]-6-isopropyl-pyridin-2-yl}-methyl-amine;
{3-[6-Ethyl-1-(2-methoxy-1-methoxymethyl-ethyl)-3-methyl-1H-pyrazolo[3,4-b]pyrazin-5-yl]-6-isopropyl-pyridin-2-yl}-methyl-amine;
{3-[6-Ethyl-1-(2-methoxy-1-methoxymethyl-ethyl)-3-methyl-1H-pyrazolo [3,4-b]pyrazin-5-yl]-6-isopropyl-pyridin-2-yl}-dimethyl-amine;
6-Ethyl-5-(2-ethyl-6-isopropyl-pyridin-3-yl)-1-(2-methoxy-1-methoxymethyl-ethyl)-3-methyl-1H-pyrazolo[3,4-b]pyrazine; and
5-(6-Isopropyl-2-methoxy-pyridin-3-yl)-1-(2-methoxy-1-methoxymethyl-ethyl)-3,6-dimethyl-1H-pyrazolo[3,4-b]pyrazine.

104. A compound or salt according to claim 2 wherein, in a standard in vitro CRF receptor binding assay the compound exhibits an IC₅₀ value for CRF receptors of less than or equal to 1 micromolar.

105. A compound or salt according to claim 2 wherein, in a standard in vitro CRF receptor binding assay the compound exhibits an IC₅₀ value for CRF receptors of less than or equal to 100 nanomolar.

106. A compound or salt according to claim 2 wherein, in a standard in vitro CRF receptor binding assay, the compound exhibits an IC₅₀ value for CRF receptors of less than or equal to 10 nanomolar.

107. A method for treating an anxiety disorder, a stress-related disorder, or an eating disorder, comprising administering to a patient in need of such treatment a therapeutically effective amount of a compound or salt according to claim 2.

108. A method for treating an depression or bipolar disorder, comprising administering to a patient in need of such treatment a therapeutically effective amount of a compound or salt according to claim 2.

109. A method for treating anorexia nervosa, bulimia nervosa, or obesity, comprising administering to a patient in need of such treatment a therapeutically effective amount of a compound or salt according to claim 2.

110. A compound or salt according to claim 2, wherein in a standard in vitro Na channel functional assay the compound does not show any statistically significant detectable Na channel modulatory activity at the p<0.05 level of significance in a standard parametric test of statistical significance.

111. A method for demonstrating the presence of CRF receptors in cell or tissue samples, said method comprising:
preparing a plurality of matched cell or tissue samples,
preparing at least one control sample by contacting (under conditions that permit binding of CRF to CRF receptors within cell and tissue samples) at least one of the matched cell or tissue samples (that has not previously been contacted with any compound or salt of claim 2) with a control solution comprising a detectably-labeled preparation of a selected compound or salt of claim 2 at a first measured molar concentration, said control solution further comprising an unlabelled preparation of the selected compound or salt at a second measured molar concentration, which second measured concentration is greater than said first measured concentration,
preparing at least one experimental sample by contacting (under conditions that permit binding of CRF to CRF receptors within cell and tissue samples) at least one of the matched cell or tissue samples (that has not previously been contacted with any compound or salt of claim 2) with an experimental solution comprising the detectably-labeled preparation of the selected compound or salt at the first measured molar concentration, said experimental solution not further comprising an unlabelled preparation of any compound or salt of any of claim 2 at a concentration greater than or equal to said first measured concentration;
washing the at least one control sample to remove unbound selected compound or salt to produce at least one washed control sample;
washing the at least one experimental sample to remove unbound selected compound or salt to produce at least one washed experimental sample;
measuring the amount of detectable label of any remaining bound detectably-labeled selected compound or salt in the at least one washed control sample;
measuring the amount detectable label of any remaining bound detectably-labeled selected compound or salt in the at least one washed experimental sample;
comparing the amount of detectable label measured in each of the at least one washed experimental sample to the amount of detectable label measured in each of the at least one washed control sample;
wherein, a comparison that indicates the detection of a greater amount of detectable label in the at least one washed experimental sample than is detected in any of the at least one washed control samples demonstrates the presence of CRF receptors in that experimental sample.

112. A method of inhibiting the binding of CRF to a CRF1 Receptor, which method comprises:
contacting a solution comprising CRF and a compound or salt of claim 2 with a cell expressing the CRF receptor, wherein the compound or salt is present in the solution at a concentration sufficient to inhibit in vitro CRF binding to IMR32 cells.

113. The method of claim 111 wherein the cell expressing the CRF receptor is a neuronal cell that is contacted in vivo in an animal, and wherein the solution is a body fluid of said animal.

114. The method of claim 111 wherein the animal is a human patient.

115. A pharmaceutical composition comprising a pharmaceutically acceptable carrier and a compound or salt of claim 2.

116. A package comprising a pharmaceutical composition of claim 115 in a container and further comprising indicia comprising at least one of:
instructions for using the composition to treat a patient suffering from an anxiety disorder, or
instructions for using the composition to treat a patient suffering from a stress-related disorder, or
instructions for using the composition to treat a patient suffering from an eating disorder.

117. A package comprising a pharmaceutical composition of claim 116 in a container and further comprising indicia comprising at least one of: instructions for using the composition to treat a patient suffering from depression or instructions for using the composition to treat a patient suffering from a bipolar disorder

118. A compound or salt according to claim 64 wherein, in a standard in vitro CRF receptor binding assay the compound exhibits an IC₅₀ value for CRF receptors of less than or equal to 1 micromolar.

119. A compound or salt according to claim 64 wherein, in a standard in vitro CRF receptor binding assay the compound exhibits an IC₅₀ value for CRF receptors of less than or equal to 100 nanomolar.

120. A compound or salt according to claim 64 wherein, in a standard in vitro CRF receptor binding assay, the compound exhibits an IC₅o value for CRF receptors of less than or equal to 10 nanomolar.

121. A compound or salt according to claim 102 wherein, in a standard in vitro CRF receptor binding assay the compound exhibits an IC₅₀ value for CRF receptors of less than or equal to 1 micromolar.

123. A compound or salt according to claim 102 wherein, in a standard in vitro CRF receptor binding assay the compound exhibits an IC₅₀ value for CRF receptors of less than or equal to 100 nanomolar.

124. A compound or salt according to claim 102 wherein, in a standard in vitro CRF receptor binding assay, the compound exhibits an IC₅₀ value for CRF receptors of less than or equal to 10 nanomolar.
